SEQUENCE LISTING (40 sequences)

SEQ ID NO: 1 - protein, 9 aa, synthetic construct
  REGION 1..9: note = Synthetic
GYSITSGYY

SEQ ID NO: 2 - protein, 7 aa, synthetic construct
  REGION 1..7: note = Synthetic
ITFDGAN

SEQ ID NO: 3 - protein, 12 aa, synthetic construct
  REGION 1..12: note = Synthetic
TRSSYDYDVLDY

SEQ ID NO: 4 - protein, 6 aa, synthetic construct
  REGION 1..6: note = Synthetic
QDISNF

SEQ ID NO: 5 - skipped

SEQ ID NO: 6 - protein, 9 aa, synthetic construct
  REGION 1..9: note = Synthetic
QQGHTLPYT

SEQ ID NO: 7 - protein, 6 aa, synthetic construct
  REGION 1..6: note = Synthetic
SGYYWN

SEQ ID NO: 8 - protein, 16 aa, synthetic construct
  REGION 1..16: note = Synthetic
YITFDGANNYNPSLKN

SEQ ID NO: 9 - protein, 10 aa, synthetic construct
  REGION 1..10: note = Synthetic
SSYDYDVLDY

SEQ ID NO: 10 - protein, 11 aa, synthetic construct
  REGION 1..11: note = Synthetic
RASQDISNFLN

SEQ ID NO: 11 - protein, 7 aa, synthetic construct
  REGION 1..7: note = Synthetic
YTSRLHS

SEQ ID NO: 12 - protein, 8 aa, synthetic construct
  REGION 1..8: note = Synthetic
GYSITSGY

SEQ ID NO: 13 - skipped

SEQ ID NO: 14 - protein, 8 aa, synthetic construct
  REGION 1..8: note = Synthetic
SYDYDVLD

SEQ ID NO: 15 - protein, 7 aa, synthetic construct
  REGION 1..7: note = Synthetic
SQDISNF

SEQ ID NO: 16 - protein, 6 aa, synthetic construct
  REGION 1..6: note = Synthetic
GHTLPY

SEQ ID NO: 17 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 30 bp, synthetic construct
  misc_feature 1..30: note = Synthetic
  source 1..30: mol_type = other DNA
ctccaacatcaaggaagatggcatttctag

SEQ ID NO: 22 - DNA, 30 bp, synthetic construct
  misc_feature 1..30: note = Synthetic
  source 1..30: mol_type = other DNA
ctagaaatgccatcttccttgatgttggag

SEQ ID NO: 23 - DNA, 13993 bp, Homo sapiens
  source 1..13993: mol_type = genomic DNA
tcctggcatcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcaggtcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttcaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttac

SEQ ID NO: 24 - DNA, 233 bp, Homo sapiens
  source 1..233: mol_type = genomic DNA
ctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaag

SEQ ID NO: 25 - DNA, 182 bp, Homo sapiens
  source 1..182: mol_type = genomic DNA
atgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacag

SEQ ID NO: 26 - DNA, 213 bp, Homo sapiens
  source 1..213: mol_type = genomic DNA
gctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcag

SEQ ID NO: 27 - DNA, 173 bp, Homo sapiens
  source 1..173: mol_type = genomic DNA
aatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagg

SEQ ID NO: 28 - DNA, 148 bp, Homo sapiens
  source 1..148: mol_type = genomic DNA
gcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaag

SEQ ID NO: 29 - DNA, 148 bp, Homo sapiens
  source 1..148: mol_type = genomic DNA
gctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaag

SEQ ID NO: 30 - DNA, 109 bp, Homo sapiens
  source 1..109: mol_type = genomic DNA
aggaagttagaagatctgagctctgagtggaaggcggtaaaccgtttacttcaagagctgagggcaaagcagcctgacctagctcctggactgaccactattggagcct

SEQ ID NO: 31 - DNA, 176 bp, Homo sapiens
  source 1..176: mol_type = genomic DNA
gaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagag

SEQ ID NO: 32 - DNA, 118 bp, Homo sapiens
  source 1..118: mol_type = genomic DNA
gcaacaatgcaggatttggaacagaggcgtccccagttggaagaactcattaccgctgcccaaaatttgaaaaacaagaccagcaatcaagaggctagaacaatcattacggatcgaa

SEQ ID NO: 33 - DNA, 212 bp, Homo sapiens
  source 1..212: mol_type = genomic DNA
ttgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaag

SEQ ID NO: 34 - DNA, 190 bp, Homo sapiens
  source 1..190: mol_type = genomic DNA
ggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaa

SEQ ID NO: 35 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 36 - DNA, 25 bp, synthetic construct
  misc_feature 1..25: note = Synthetic
  source 1..25: mol_type = other DNA
ggccaaacctcggcttacctgaaat

SEQ ID NO: 37 - DNA, 176 bp, Homo sapiens; identical to SEQ ID NO: 31
  source 1..176: mol_type = genomic DNA

SEQ ID NO: 38 - DNA, 25 bp, synthetic construct
  misc_feature 1..25: note = Synthetic
  source 1..25: mol_type = other DNA
atttcaggtaagccgaggtttggcc

SEQ ID NO: 39 - DNA, 21 bp, synthetic construct
  misc_feature 1..21: note = Synthetic
  source 1..21: mol_type = other DNA
cacatctttgatggtgtgagg

SEQ ID NO: 40 - DNA, 22 bp, synthetic construct
  misc_feature 1..22: note = Synthetic
  source 1..22: mol_type = other DNA
caacttcagccatccatttctg

(19) United States
(12) Patent Application Publication
(10) Pub. No.: US 2025/0018050 A1
(43) Pub. Date: Jan. 16, 2025

(54) DOSING OF MUSCLE TARGETING COMPLEXES FOR TREATING DYSTROPHINOPATHIES

(71) Applicant: Dyne Therapeutics, Inc., Waltham, MA (US)

(72) Inventors: Cody A. Desjardins, Waltham, MA (US); Kim Tang, Waltham, MA (US); Oxana Beskrovnaya, Waltham, MA (US); John Davis, Waltham, MA (US); Chris Mix, Waltham, MA (US); Timothy Weeden, Waltham, MA (US); Scott Hilderbrand, Waltham, MA (US); Sean Spring, Waltham, MA (US); Peiyi Shen, Waltham, MA (US); Romesh R. Subramanian, Framingham, MA (US); Mohammed T. Qatanani, Waltham, MA (US); John Najim, Waltham, MA (US); Brendan Quinn, Waltham, MA (US)

(73) Assignee: Dyne Therapeutics, Inc., Waltham, MA (US)

(21) Appl. No.: 18/692,415

(22) PCT Filed: Sep. 15, 2022
(86) PCT No.: PCT/US2022/076516

§ 371 Date: Mar. 15, 2024

Related U.S. Application Data
(60) Provisional application No. 63/348,876, filed on Jun. 3, 2022.
(60) Provisional application No. 63/293,619, filed on Dec. 23, 2021.
(60) Provisional application No. 63/250,177, filed on Sep. 29, 2021.
(60) Provisional application No. 63/245,162, filed on Sep. 16, 2021.

(51) Int. Cl. A61K 47/68; A61P 9/00; A61P 21/00; C07K 16/28; C12N 15/113

(52) U.S. Cl. CPC A61K 47/6807; A61K 47/6849; A61P 9/00; A61P 21/00; C07K 16/2881; C12N 15/113; C12N 2310/11; C12N 2310/3233

(57) ABSTRACT

Aspects of the disclosure relate to methods of promoting expression or activity of a dystrophin protein and/or methods of treating DMD in a subject. In some embodiments, the methods comprise administering to the subject a composition comprising complexes (e.g., muscle targeting complexes) comprising a phosphorodiamidate morpholino oligomer (e.g., useful for targeting DMD) covalently linked to an antibody (e.g., anti-TfR1 antibody).

### RELATED APPLICATIONS

This application claims the benefit under 35 U.S.C § 119(e) of the filing date of U.S. Provisional Application No. 63/348,876, entitled “DOSING OF MUSCLE TARGETING COMPLEXES FOR TREATING DYSTROPHINOPATHIES”, filed Jun. 3, 2022; U.S. Provisional Application No. 63/293,619, entitled “DOSING OF MUSCLE TARGETING COMPLEXES FOR TREATING DYSTROPHINOPATHIES”, filed Dec. 23, 2021; U.S. Provisional Application No. 63/250,177, entitled “DOSING OF MUSCLE TARGETING COMPLEXES FOR TREATING DYSTROPHINOPATHIES”, filed Sep. 29, 2021; and U.S. Provisional Application No. 63/245,162, entitled “DOSING OF MUSCLE TARGETING COMPLEXES FOR TREATING DYSTROPHINOPATHIES”, filed Sep. 16, 2021; the contents of each of which are incorporated herein by reference in their entirety.

### FIELD OF THE INVENTION

The present application relates to targeting complexes for delivering an effective amount of oligonucleotide molecular payloads to cells and uses thereof, particularly uses relating to treatment of disease.

### REFERENCE TO ELECTRONIC SEQUENCE LISTING

The contents of the electronic sequence listing (D082470071WO00-SEQ-CBD.xml; Size: 60,636 bytes; and Date of Creation: Aug. 31, 2022) is herein incorporated by reference in its entirety.

### BACKGROUND

Dystrophinopathies are a group of distinct neuromuscular diseases that result from mutations in dystrophin gene. Dystrophinopathies include Duchenne muscular dystrophy, Becker muscular dystrophy, and X-linked dilated cardiomyopathy. Dystrophin (DMD) is a large gene, containing 79 exons and approximately 2.6 million total base pairs. Numerous mutations in DMD, including exonic frameshift, deletion, substitution, and duplicative mutations, are able to diminish the expression of functional dystrophin, leading to dystrophinopathies.

### SUMMARY OF INVENTION

According to some aspects, the present disclosure provides methods of promoting expression or activity of a dystrophin protein (e.g., a truncated dystrophin protein) and/or methods of treating Duchenne Muscular Dystrophy (DMD) in a subject. The truncated dystrophin protein is functional (e.g., retains activities of a wild-type dystrophin protein). In some embodiments, the truncated dystrophin protein retains partial function of a wild-type dystrophin protein. In some embodiments, the methods described herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 0.5 mg to 5 mg (e.g., 0.7 mg, 1.4 mg, or 2.8 mg) of the oligonucleotides of the complexes per kg of the subject. In some embodiments, the methods described herein comprises administering to the subject a composition comprising an effective amount of muscle targeting complexes, each complex comprising an anti-transferrin receptor 1 (TfR1) antibody covalently linked to one or more oligonucleotides, wherein the effective amount provides to the subject 5 mg to 80 mg (e.g., 10 mg, 30 mg, or 60 mg) of the oligonucleotides of the complexes per kg of the subject. In some embodiments, the effective amount provides to the subject 5 mg to 40 mg (e.g., about 5 mg, 10 mg, 20 mg, 30 mg, or 40 mg) of the oligonucleotides of the complexes per kg of the subject. In some embodiments, the effective amount provides to the subject 1 mg to 8 mg (e.g., 1 mg, 1.5 mg, 2 mg, 3 mg, 4 mg, 5 mg, 6 mg, 7 mg, or 8 mg) of the anti-TfR1 antibody (e.g., Fab) of the complexes per kg of the subject. In some embodiments, the effective amount provides to the subject 5 mg to 120 mg (e.g., about 11 mg, 22 mg, 44 mg, 66 mg, or 88 mg) of the anti-TfR1 antibody (e.g., Fab) of the complexes per kg of the subject. In some embodiments, the administration is once every two weeks to once every three months (e.g., once every two weeks, once every four weeks, once every two months, or once every three months). In some embodiments, the subject has a mutated DMD allele associated with Duchenne Muscular Dystrophy (e.g., wherein the mutated DMD allele comprises a mutation amenable to exon skipping).

Some aspects of the present disclosure provide methods of promoting expression or activity of a dystrophin protein in a subject, comprising administering to the subject a composition comprising an effective amount of complexes, each complex comprising an anti-transferrin receptor 1 (TfR1) antibody covalently linked to one or more oligonucleotides, wherein the effective amount provides to the subject 1 mg to 90 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the nucleotide sequence of CTCCAACATCAAGGAAGATGGCATTTCTAG (SEQ ID NO: 21) and is a phosphorodiamidate morpholino oligomer (PMO).

Some aspects of the present disclosure provide methods of treating Duchenne Muscular Dystrophy (DMD) in a subject, comprising administering to the subject a composition comprising an effective amount of complexes, each complex comprising an anti-transferrin receptor 1 (TfR1) antibody covalently linked to one or more oligonucleotides, wherein the effective amount provides to the subject 1 mg to 90 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the nucleotide sequence of CTCCAACATCAAGGAAGATGGCATTTCTAG (SEQ ID NO: 21) and is a phosphorodiamidate morpholino oligomer (PMO).

In some embodiments, each complex comprises a structure of formula (I): [R¹]ₙ₁—R², wherein:
each R¹ comprises a group of the formula (Tb):

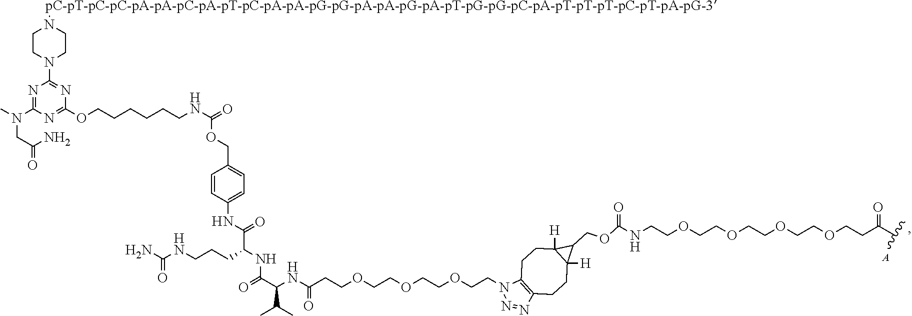

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody. In some embodiments, the average value of n1 of the complexes of the composition is in the range of 1 to 5.

In some embodiments, each complex comprises a structure of formula (I): [R¹]ₙ₁—R², wherein:
each R¹ comprises a group of the formula (Ic):

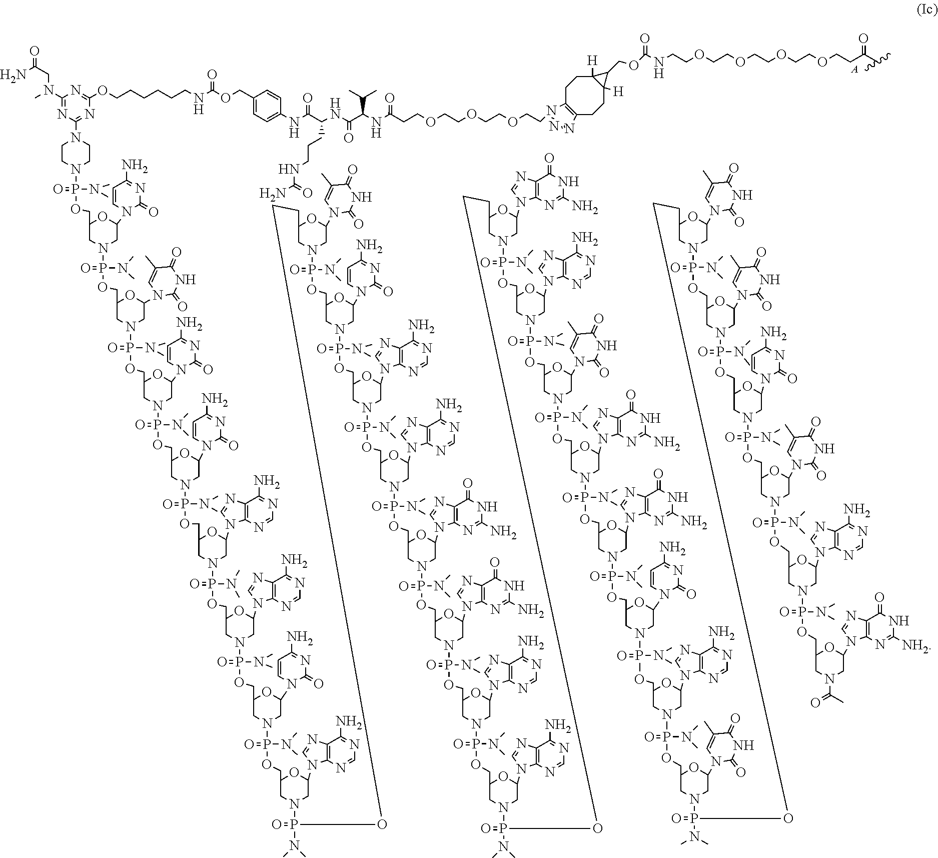

R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody. In some embodiments, the average value of n1 of the complexes of the composition is in the range of 1 to 5.

In some embodiments, the anti-TfR1 antibody is a Fab fragment. In some embodiments, the anti-TfR1 antibody comprises a heavy chain variable region (VH) comprising the amino acid sequence of SEQ ID NO: 17 and a light chain variable region (VL) comprising the amino acid sequence of SEQ ID NO: 18. In some embodiments, the anti-TfR1 antibody comprises a heavy chain comprising the amino acid sequence of SEQ ID NO: 19 and a light chain comprising the amino acid sequence of SEQ ID NO: 20.

In some embodiments, the effective amount of each administration provides to the subject 1-8 mg of the anti-TfR1 antibody of the complexes per kg of the subject. In some embodiments, the effective amount of each administration provides to the subject 1-2 mg of the anti-TfR1 antibody of the complexes per kg of the subject. In some embodiments, the effective amount of each administration provides to the subject 1.5 mg of the anti-TfR1 antibody of the complexes per kg of the subject. In some embodiments, the effective amount of each administration provides to the subject 2-4 mg of the anti-TfR1 antibody of the complexes per kg of the subject. In some embodiments, the effective amount of each administration provides to the subject 3 mg of the anti-TfR1 antibody of the complexes per kg of the subject. In some embodiments, the effective amount of each administration provides to the subject 4-8 mg of the anti-TfR1 antibody of the complexes per kg of the subject. In some embodiments, the effective amount of each administration provides to the subject 6 mg of the anti-TfR1 antibody of the complexes per kg of the subject.

In some embodiments, the effective amount of each administration provides to the subject 3-52 mg of the anti-TfR1 antibody of the complexes per kg of the subject. In some embodiments, the effective amount of each administration provides to the subject 5-40 mg of the anti-TfR1 antibody of the complexes per kg of the subject. In some embodiments, the effective amount of each administration provides to the subject 7-15 mg of the anti-TfR1 antibody of the complexes per kg of the subject. In some embodiments, the effective amount of each administration provides to the subject 11 mg of the anti-TfR1 antibody of the complexes per kg of the subject. In some embodiments, the effective amount of each administration provides to the subject 15-30 mg of the anti-TfR1 antibody of the complexes per kg of the subject. In some embodiments, the effective amount of each administration provides to the subject 22 mg of the anti-TfR1 antibody of the complexes per kg of the subject. In some embodiments, the effective amount of each administration provides to the subject 30-59 mg of the anti-TfR1 antibody of the complexes per kg of the subject. In some embodiments, the effective amount of each administration provides to the subject 44 mg of the anti-TfR1 antibody of the complexes per kg of the subject. In some embodiments, the effective amount of each administration provides to the subject 61-117 mg of the anti-TfR1 antibody of the complexes per kg of the subject. In some embodiments, the effective amount of each administration provides to the subject 88 mg of the anti-TfR1 antibody of the complexes per kg of the subject.

In some embodiments, in the composition is administered once every 2 weeks, once every 4 weeks, once every 8 weeks or once every 12 weeks. In some embodiments, the composition is administered once every 4 weeks. In some embodiments, the composition is administered once every 8 weeks.

In some embodiments, the composition is in an aqueous solution and further comprises histidine and sucrose. In some embodiments, the histidine is present in the aqueous solution at a concentration of 25 mM. In some embodiments, the sucrose is present in the aqueous solution at a concentration of 10 w/v %. In some embodiments, the aqueous solution is at a pH of 6.0.

In some embodiments, the subject has a mutated dystrophin allele comprising a mutation amenable to exon 51 skipping or the mutated dystrophin allele comprises a frameshift mutation in exon 51. In some embodiments, the complex promotes expression or activity of a truncated dystrophin protein in the subject.

In some embodiments, the subject is a human subject. In some embodiments, the human subject is between 2-60 years of age.

### BRIEF DESCRIPTION OF THE DRAWINGS

FIGS. 1A-1B show activities of anti-TfR1 Fab-oligonucleotide conjugate in inducing DMD exon 51 skipping in DMD patient myotubes. The anti-TfR1 Fab-oligonucleotide conjugate contains an anti-TfR1 Fab having the VH/VL sequences shown in Table 2 covalently linked (through lysine conjugation) via a linker comprising a valine-citrulline sequence to a DMD exon 51-skipping oligonucleotide (SEQ ID NO: 21). FIG. 1A shows that a composition comprising anti-TfR1 Fab-oligonucleotide conjugate resulted in enhanced exon skipping compared to the same DMD exon 51-skipping oligonucleotide that is not covalently linked to a Fab in DMD patient myotubes. FIG. 1B shows that anti-TfR1 Fab-oligonucleotide conjugate resulted in dose-dependent exon 51 skipping following treatment with anti-TfR1 Fab conjugate to a final concentration of 2.5 μM (low), 5 μM (medium), and 10 μM (high) oligonucleotide equivalent.

FIGS. 2A-2E show the levels of oligonucleotide in quadriceps (FIG. 2A), diaphragm (FIG. 2B), heart (FIG. 2C), gastrocnemius (FIG. 2D), and tibialis anterior (FIG. 2E) of mdx mice, following administration of a single dose of an anti-TfR1 Fab-oligonucleotide (Fab-oligo) conjugate at a dose equivalent to 10 mg/kg oligonucleotide or 30 mg/kg oligonucleotide. The anti-TfR1 Fab-oligo conjugate contains an anti-mouse TfR1 (R17-217) Fab covalently linked via a linker comprising a valine-citrulline sequence to a DMD exon 23-skipping oligonucleotide.

FIGS. 3A-3E show levels of exon 23 skipping in quadriceps (FIG. 3A), diaphragm (FIG. 3B), heart (FIG. 3C), gastrocnemius (FIG. 3D), and tibialis anterior (FIG. 3E) of mdx mice, following administration of a single dose of the anti-TfR1 Fab-oligo conjugate described in FIGS. 2A-2E at a dose equivalent to 10 mg/kg oligonucleotide or 30 mg/kg oligonucleotide.

FIGS. 4A-4E show levels of dystrophin expression by quantitative Western blot in quadriceps (FIG. 4A), diaphragm (FIG. 4B), heart (FIG. 4C), gastrocnemius (FIG. 4D), and tibialis anterior (FIG. 4E) of mdx mice, following administration of a single dose of the anti-TfR1 Fab-oligo conjugate described in FIGS. 2A-2E at a dose equivalent to 10 mg/kg oligonucleotide or 30 mg/kg oligonucleotide. FIGS. 4A-4C also show examples of Western blot images of dystrophin expression in quadriceps at 8 weeks post dose (FIG. 4A), diaphragm at 4 weeks post dose (FIG. 4B), and heart at 4 weeks post dose (FIG. 4C) of mdx mice, following administration of a single dose of the anti-TfR1 Fab-oligo conjugate described in FIGS. 2A-2E at a dose equivalent to 30 mg/kg oligonucleotide.

FIGS. 5A-5C show immunofluorescence (IF) images visualizing dystrophin localization to sarcolemma in quadriceps at 4 weeks, 8 weeks, and 12 weeks post dose (FIG. 5A), diaphragm at 4 weeks post dose (FIG. 5B), and heart at 4 weeks post dose (FIG. 5C) of mdx mice, following administration of a single dose of the anti-TfR1 Fab-oligo conjugate described in FIGS. 2A-2E at a dose equivalent to 10 mg/kg oligonucleotide or 30 mg/kg oligonucleotide. FIG. 5A also shows quantification of the IF data in quadriceps, and indicates percent dystrophin positive fibers (PDPF).

FIGS. 6A-6B show levels of exon 51 skipping in heart (FIG. 6A) and diaphragm (FIG. 6B) of cynomolgus monkeys, following administration of a single dose of anti-TfR1 Fab-oligo conjugate at a dose equivalent to 60 mg/kg oligonucleotide or two doses (on days 1 and 15) of anti-TfR1 Fab-oligo conjugate at a dose equivalent to 30 mg/kg oligonucleotide. Levels of exon skipping were analyzed two weeks post the single 60 mg/kg dose (day 15) or 2 weeks post the second 30 mg/kg dose (day 29). Anti-TfR1 Fab-oligo conjugate contains an anti-TfR1 Fab having the VH/VL sequences shown in Table 2, in which the Fab is covalently linked (through lysine conjugation) via a linker comprising a valine-citrulline sequence to a DMD exon 51-skipping oligonucleotide (SEQ ID NO: 21).

FIG. 7A-7C show levels of exon 51 skipping in quadriceps (FIG. 7A), diaphragm (FIG. 7B), and heart (FIG. 7C) of cynomolgus monkeys, following administration of (i) two doses (on days 1 and 15) of anti-TfR1 Fab-oligonucleotide conjugate at a dose equivalent to 30 mg/kg oligonucleotide (“2×30” and measured at 4 weeks), (ii) weekly doses for four weeks of anti-TfR1 Fab-oligo conjugate at a dose equivalent to 30 mg/kg oligonucleotide (“4×30” and measured at 4 weeks), or (iii) weekly doses for five weeks of anti-TfR1 Fab-oligonucleotide conjugate at a dose equivalent to 30 mg/kg oligonucleotide (“5×30” and measured at 8 weeks). Anti-TfR1 Fab-oligonucleotide conjugate contains an anti-TfR1 Fab having the VH/VL sequences shown in Table 2, in which the Fab is covalently linked (through lysine conjugation) via a linker comprising a valine-citrulline sequence to a DMD exon 51-skipping oligonucleotide (SEQ ID NO: 21).

FIGS. 8A-8E show that a single dose of anti-TfR1 Fab-oligonucleotide conjugate, but not of unconjugated exon 23-skipping oligonucleotide, induces dose-dependent levels of oligonucleotide in skeletal and cardiac muscles of mdx mice. Five-week-old mdx mice were injected via tail vein with vehicle, 30 mg/kg unconjugated exon 23-skipping oligonucleotide, or anti-TfR1 Fab-oligonucleotide conjugate at a dose equivalent to 10 mg/kg or 30 mg/kg oligonucleotide and sacrificed at the indicated time points. Levels of oligonucleotide in indicated skeletal and cardiac muscle was determined using hELISA. Data represent mean±SD. * p<0.05, ** p<0.01, **** p<0.0001. hELISA, hybridization enzyme-linked immunosorbent assay; PMO, phosphorodiamidate morpholino oligomer; WT, wild type.

FIGS. 9A-9E show that a single dose of anti-TfR1 Fab-oligonucleotide conjugate, resulted in enhanced dose-dependent Dmd exon 23 skipping in skeletal and cardiac muscles of mdx mice, compared to a single dose of the unconjugated exon 23-skipping oligonucleotide. Five-week-old mdx mice were injected via tail vein with vehicle, 30 mg/kg unconjugated exon 23-skipping oligonucleotide, or anti-TfR1 Fab-oligonucleotide conjugate at a dose equivalent to 10 mg/kg or 30 mg/kg oligonucleotide and sacrificed at the indicated time points. Exon 23 skipping was measured by RT-PCR and capillary electrophoresis. Percent skipping was calculated as described in Materials and Methods. Data represent mean±SD. * p<0.05, ** p<0.01, **** p<0.0001. PMO, phosphorodiamidate morpholino oligomer; RT-PCR, reverse transcription polymerase chain reaction; WT, wild type.

FIGS. 10A-10F show that a single dose of anti-TfR1 Fab-oligonucleotide conjugate resulted in enhanced dose-dependent dystrophin protein expression in skeletal and cardiac muscles of mdx mice, compared to a single dose of the unconjugated exon 23-skipping oligonucleotide. Five-week-old mdx mice were injected via tail vein with vehicle, 30 mg/kg unconjugated exon 23-skipping oligonucleotide, or anti-TfR1 Fab-oligonucleotide conjugate at a dose equivalent to 10 mg/kg or 30 mg/kg oligonucleotide and sacrificed at the indicated time points. FIG. 10A shows representative western blot images of dystrophin expression 28 days after a single dose of anti-TfR1 Fab-oligonucleotide conjugate at a dose equivalent to 30 mg/kg oligonucleotide or a matched 30 mg/kg dose of unconjugated exon 23-skipping oligonucleotide. Dystrophin levels in these samples exceed the upper limit of the standard curve and these western blots were not used for quantification. Additional western blotting was performed on samples diluted to within the range of the standard curve and were used for quantification. All bands were quantified based on a standard curve run on the same gel and created using the same muscle tissue matrix FIGS. 10B-10F show quantification of dystrophin protein levels by fluorimetry analysis of western blot images. Data represent mean±SD. * p<0.05, ** p<0.01, **** p<0.0001. PMO, phosphorodiamidate morpholino oligomer; WT, wild type.

FIGS. 11A-11D show that a single dose of anti-TfR1 Fab-oligonucleotide conjugate is sufficient to restore dystrophin localization to the sarcolemma in skeletal and cardiac muscles of mdx mice. Five-week-old mdx mice were injected via tail vein with vehicle or anti-TfR1 Fab-oligonucleotide conjugate at a dose equivalent to 10 mg/kg or 30 mg/kg oligonucleotide and sacrificed at the indicated time points. FIG. 11A shows representative immunofluorescence images with dystrophin (green) and laminin (red) staining of quadriceps cross-sections isolated 4-, 8-, and 12-weeks post-dose from vehicle-treated wild-type or mdx mice, or from mdx mice treated with anti-TfR1 Fab-oligonucleotide conjugate at a dose equivalent to 30 mg/kg oligonucleotide. Individual fields quantified for 30 mg/kg oligonucleotide group at 8 weeks are shown in FIG. 15. FIG. 11B shows quantification of dystrophin positive fibers in quadriceps of mdx mice. FIG. 11C and FIG. 11D show representative immunofluorescence images with dystrophin (green) and laminin (red) staining of diaphragm (FIG. 11C) and heart (FIG. 11D) cross-sections isolated 4- or 8-weeks post-dose from vehicle-treated mdx mice, or from mdx mice treated with anti-TfR1 Fab-oligonucleotide conjugate at a dose equivalent to 30 mg/kg oligonucleotide. Data represent mean±SD. **** p<0.0001. PMO, phosphorodiamidate morpholino oligomer; WT, wild type.

FIG. 12 shows that dystrophin restored by anti-TfR1 Fab-oligonucleotide conjugate is localized to the muscle membranes of mdx mice. Five-week-old mdx mice were injected via tail vein with vehicle or anti-TfR1 Fab-oligonucleotide conjugate at a dose equivalent to 30 mg/kg oligonucleotide and sacrificed 4 weeks post-dose. Diaphragm was isolated and longitudinal cross-sections were stained with dystrophin (green) and laminin (red) to image distribution of membrane-localized dystrophin along the entire length of the myofibers. PMO, phosphorodiamidate morpholino oligomer.

FIGS. 13A-13C show that treatment with anti-TfR1 Fab-oligonucleotide conjugate, but not with unconjugated exon 23-skipping oligonucleotide, leads to improved functional outcomes in mdx mice. Functional assessments performed 2 weeks following administration of 5-week-old wild-type or mdx mice with vehicle or mdx mice injected with 30 mg/kg unconjugated exon 23-skipping oligonucleotide or anti-TfR1 Fab-oligonucleotide conjugate at a dose equivalent to 30 mg/kg oligonucleotide. FIG. 13A shows serum creatine kinase (CK) activity. FIG. 13B shows total distance traveled on a running wheel for an uninterrupted 24-hour period. FIG. 13C shows percent change in total distance traveled in an open field before and after hind limb fatigue challenge. Data are expressed as mean±SD. *p<0.05, **p<0.01, ***p<0.001, ****p<0.0001. n.s., not significant; PMO, phosphorodiamidate morpholino oligomer; WT, wild type.

FIGS. 14A-14B show representative fluorescent western blot image and standard curve quantification. FIG. 14A shows a representative fluorescent western blot image in which each gel was run with a standard curve of wild-type mouse protein diluted into mdx mouse protein. Raw fluorescence was measured in the dystrophin and alpha-actinin channels for each standard and unknown sample. FIG. 14B shows standard curve quantification. The dystrophin/alpha-actinin ratio was generated and plotted against the known percent wild-type protein in each standard to generate an equation. Unknown samples were then interpolated from their dystrophin/alpha-actinin ratio based on the equation of the standard curve. If the raw dystrophin signal of the unknown sample was above the standard curve, the sample was diluted until it fell within the standard curve. WT, wild type.

FIG. 15 shows immunofluorescent micrographs used to quantify percent positive myofibers in quadriceps of mdx mice (see also FIGS. 11A-11B). Five-week-old mdx mice were injected via tail vein with anti-TfR1 Fab-oligonucleotide conjugate at a dose equivalent to 30 mg/kg oligonucleotide. Immunofluorescence images with dystrophin (green) and laminin (red) staining of quadriceps isolated 8 weeks post-dose.

FIGS. 16A-16B show treatment with anti-TfR1 Fab-oligonucleotide conjugate reduces serum creatine kinase activity in mdx mice. Five-week-old mdx mice injected via tail vein with anti-TfR1 Fab-oligonucleotide conjugate at a dose equivalent to 10 mg/kg or 30 mg/kg oligonucleotide were sacrificed and serum creatine kinase (CK) was assessed 28 (FIG. 16A) or 56 (FIG. 16B) days post-dose. Data are normalized to the mean serum CK of vehicle-treated mdx mice at the matched time points. Data represent mean±SD. * p<0.05; ** p<0.01. CK, creatine kinase; PMO, phosphorodiamidate morpholino oligomer.

FIGS. 17A-17B show anti-TfR1 Fab-oligonucleotide conjugate-mediated dystrophin restoration leads to improved functional outcomes in mdx mice. Functional assessments were performed 4 weeks following administration of 5-week-old wild-type or mdx mice with vehicle or mdx mice with 30 mg/kg unconjugated exon 23-skipping oligonucleotide or anti-TfR1 Fab-oligonucleotide conjugate at a dose equivalent to 30 mg/kg oligonucleotide. FIG. 17A shows total distance traveled on a running wheel for an uninterrupted 24-h period. FIG. 17B shows percent change in total distance traveled in an open field before and after hind limb fatigue challenge. Data are expressed as mean±SD. *p≤0.05, **p≤0.01. n.s., not significant; PMO, phosphorodiamidate morpholino oligomer; WT, wild type.

### DETAILED DESCRIPTION OF INVENTION

According to some aspects, the present disclosure provides methods of promoting expression or activity of a dystrophin protein (e.g., a truncated dystrophin protein) and/or methods of treating Duchenne Muscular Dystrophy (DMD) in a subject. The truncated dystrophin protein is functional (e.g., retains activities of a wild-type dystrophin protein). In some embodiments, the truncated dystrophin protein retains partial function of a wild-type dystrophin protein. In some embodiments, the methods described herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 0.5 mg to 5 mg (e.g., about 0.7 mg, 1.4 mg, or 2.8 mg) of the oligonucleotides of the complexes per kg of the subject. In some embodiments, the methods described herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 5 mg to 80 mg (e.g., 10 mg, 30 mg, or 60 mg) of the oligonucleotides of the complexes per kg of the subject. In some embodiments, the effective amount provides to the subject 5 mg to 40 mg (e.g., about 5 mg, 10 mg, 20 mg, 30 mg, or 40 mg) of the oligonucleotides of the complexes per kg of the subject. In some embodiments, the effective amount provides to the subject 1 mg to 8 mg (e.g., about 1.5 mg, 3.0 mg, or 6.0 mg) of the anti-TfR1 antibody (e.g., Fab) of the complexes per kg of the subject. In some embodiments, the effective amount provides to the subject 5 mg to 120 mg (e.g., about 11 mg, 22 mg, 44 mg, 66 mg, or 88 mg) of the anti-TfR1 antibody (e.g., Fab) of the complexes per kg of the subject. In some embodiments, the administration is once every two weeks to once every three months (e.g., once every two weeks, once every four weeks, once every two months, or once every three months). In some embodiments, the subject has a mutated DMD allele associated with Duchenne Muscular Dystrophy (e.g., wherein the mutated DMD allele comprises a mutation amenable to exon skipping).

Further aspects of the disclosure, including a description of defined terms, are provided below.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

DMD: As used herein, the term “DMD” refers to a gene that encodes dystrophin protein, a key component of the dystrophin-glycoprotein complex, which bridges the inner cytoskeleton and the extracellular matrix in muscle cells, particularly muscle fibers. Deletions, duplications, and point mutations in DMD may cause dystrophinopathies, such as Duchenne muscular dystrophy, Becker muscular dystrophy, or cardiomyopathy. Alternative promoter usage and alternative splicing result in numerous distinct transcript variants and protein isoforms for this gene. In some embodiments, a dystrophin gene may be a human (Gene ID: 1756), non-human primate (e.g., Gene ID: 465559), or rodent gene (e.g., Gene ID: 13405; Gene ID: 24907). In addition, multiple human transcript variants (e.g., as annotated under GenBank RefSeq Accession Numbers: NM_000109.3, NM_004006.2 (SEQ ID NO: 24), NM_004009.3, NM_004010.3 and NM_004011.3) have been characterized that encode different protein isoforms.

DMD allele: As used herein, the term “DMD allele” refers to any one of alternative forms (e.g., wild-type or mutant forms) of a DMD gene. In some embodiments, a DMD allele may encode for dystrophin that retains its normal and typical functions. In some embodiments, a DMD allele may comprise one or more mutations that results in muscular dystrophy. Common mutations that lead to Duchenne muscular dystrophy involve frameshift, deletion, substitution, and duplicative mutations of one or more of 79 exons present in a dystrophin allele, e.g., exon 8, exon 23, exon 41, exon 44, exon 50, exon 51, exon 52, exon 53, or exon 55. Further examples of DMD mutations are disclosed, for example, in Flanigan K M, et al., Mutational spectrum of DMD mutations in dystrophinopathy patients: application of modern diagnostic techniques to a large cohort. Hum Mutat. 2009 December; 30 (12):1657-66, the contents of which are incorporated herein by reference in its entirety.

Dystrophinopathy: As used herein, the term “dystrophinopathy” refers to a muscle disease that results from one or more mutated DMD alleles. Dystrophinopathies include a spectrum of conditions (ranging from mild to severe) that includes Duchenne muscular dystrophy, Becker muscular dystrophy, and DMD-associated dilated cardiomyopathy (DCM). In some embodiments, at one end of the spectrum, dystrophinopathy is phenotypically associated with an asymptomatic increase in serum concentration of creatine phosphokinase (CK) and/or (e.g., and) muscle cramps with myoglobinuria. In some embodiments, at the other end of the spectrum, dystrophinopathy is phenotypically associated with progressive muscle diseases that are generally classified as Duchenne or Becker muscular dystrophy when skeletal muscle is primarily affected and as DMD-associated dilated cardiomyopathy (DCM) when the heart is primarily affected. Symptoms of Duchenne muscular dystrophy include muscle loss or degeneration, diminished muscle function, pseudohypertrophy of the tongue and calf muscles, higher risk of neurological abnormalities, and a shortened lifespan. Duchenne muscular dystrophy is associated with Online Mendelian Inheritance in Man (OMIM) Entry #310200. Becker muscular dystrophy is associated with OMIM Entry #300376. Dilated cardiomyopathy is associated with OMIM Entry X #302045.

Exonic splicing enhancer (ESE): As used herein, the term “exonic splicing enhancer” or “ESE” refers to a nucleic acid sequence motif within an exon of a gene, pre-mRNA, or mRNA that directs or enhances splicing of pre-mRNA into mRNA, e.g., as described in Blencowe et al., Trends Biochem Sci 25, 106-10. (2000), incorporated herein by reference. ESEs are splicing features. ESEs may direct or enhance splicing, for example, to remove one or more introns and/or one or more exons from a gene transcript. ESE motifs are typically 6-8 nucleobases in length. SR proteins (e.g., proteins encoded by the gene SRSF1, SRSF2, SRSF3, SRSF4, SRSF5, SRSF6, SRSF7, SRSF8, SRSF9, SRSF10, SRSF11, SRSF12, TRA2A or TRA2B) bind to ESEs through their RNA recognition motif region to facilitate splicing. ESE motifs can be identified through a number of methods, including those described in Cartegni et al., Nucleic Acids Research, 2003, Vol. 31, No. 13, 3568-3571, incorporated herein by reference.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Morpholinos: As used herein, the term “morpholino”, also referred to as a “phosphorodiamidate morpholino oligomer”, refers to a molecular structure that contains nucleobases attached to a backbone of methylenemorpholine rings linked through a phosphorodiamidate group. In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a mutated DMD gene sequence, e.g., a mutation in an exon of a DMD gene sequence. In some embodiments, a subject has a dystrophinopathy, e.g., Duchenne muscular dystrophy.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

Ranges: All ranges provided in the present disclosure are inclusive of the end points. Complexes

Provided herein are methods of promoting expression or activity of a dystrophin protein and/or treating Duchenne Muscular Dystrophy (DMD) in a subject comprising administering to the subject an effective amount of muscle targeting complexes, wherein the complexes comprise a targeting agent, e.g., an antibody, covalently linked to an oligonucleotide. In some embodiments, a complex comprises a muscle-targeting antibody covalently linked to one or more oligonucleotides. In some embodiments, the oligonucleotide is a PMO. In some embodiments, the oligonucleotide is an oligonucleotide that targets a mutated DMD allele to promote exon skipping.

Complexes used in the methods described herein generally comprise a linker that covalently links an antibody (e.g., any one of the anti-TfR1 antibodies) described herein to an oligonucleotide (e.g., a PMO). A linker comprises at least one covalent bond.

In some embodiments, complexes used in the methods described herein comprise a structure of formula (I): [R¹]ₙ₁—R², in which each R¹ independently comprises a compound comprising an oligonucleotide (e.g., a PMO) and R² comprises an antibody (e.g., anti-TfR1 antibody), and wherein in each complex n1 is independently an integer (e.g., of one or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² is an antibody (e.g., anti-TfR1 antibody). In some embodiments, R² is an anti-TfR1 Fab.

In some embodiments, in each complex n1 is independently an integer of one or greater. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv. In some embodiments, the antibody is a Fab fragment.

In some embodiments, the value of n1 of each or any complex (e.g., any complex in any of the compositions or methods disclosed herein) is an integer from one up to the number of amino acid residues in the antibody to which conjugation is desired or targeted (e.g., the number of lysine residues). In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex the value of n1 is independently in the range of 1-27, 1-26, 1-10, 1-5, or 1-3.

In some embodiments, complexes used in the methods described herein are presented as compositions (e.g., in aqueous solutions) for administration to a subject. In some embodiments, the composition comprises a plurality of complexes and further comprises histidine and/or sucrose. In some embodiments, the plurality of different complexes comprise a common targeting agent (e.g. an antibody) and a common oligonucleotide (PMO). In such embodiments, different complex types are characterized by having different numbers of oligonucleotides covalently linked to an antibody. For example, in some embodiments, a composition for administration to a subject comprises a plurality of complex types in which each complex type comprises a structure of formula (I): [R¹]ₙ₁—R², in which each R¹ independently comprises a compound comprising an oligonucleotide (e.g., a PMO) and R² comprises an antibody (e.g., anti-TfR1 antibody), and in which in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, a composition for administration to a subject in the methods described herein comprises unconjugated antibody (e.g., in trace amounts) and antibody conjugated to one or more oligonucleotides. In some embodiments, unconjugated antibody may be referred to as a compound of a structure of formula (I): [R¹]ₙ₁—R², for which n1 is zero. Accordingly, in some embodiments, a composition for administration to a subject in the methods described herein comprises compounds (e.g., complexes) of the structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds of the structure formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%.

In some embodiments, each instance of R¹ in a complex is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid.

In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, R¹ is not covalently linked to an amino acid residue residing in a CDR region of the antibody.

In some embodiments, complexes used in the methods described herein comprise a structure of formula (I): [R¹]ₙ₁—R², in which each R¹ independently comprises a group of the formula (Ia):

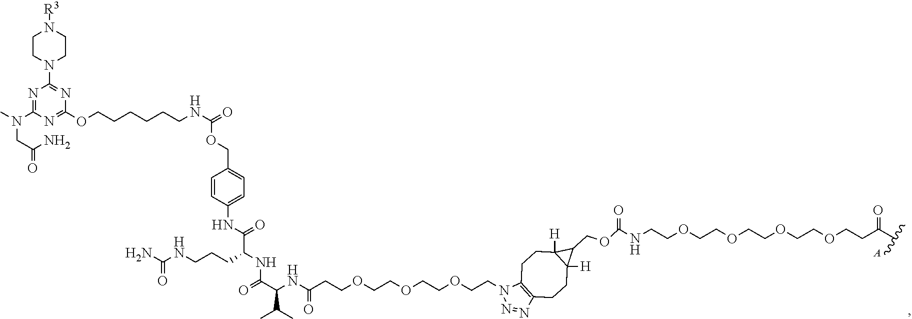

in which R³ is an oligonucleotide, e.g., a phosphorodiamidate morpholino oligomer (PMO); wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, R³ is an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, R² comprises a Fab and each R¹ is covalently linked (e.g., indirectly or directly linked, e.g., directly linked) at attachment point A to a different amino acid residue of the Fab, optionally wherein each different amino acid residue is a lysine. In some embodiments, in each complex n1 is independently an integer (e.g., an integer in the range of 1-27, 1-26, 1-10, 1-5, or 1-3).

In some embodiments, complexes used in the methods described herein comprise a structure of formula (I): [R¹]ₙ₁—R², in which each R¹ comprises a group of the formula (Ib):

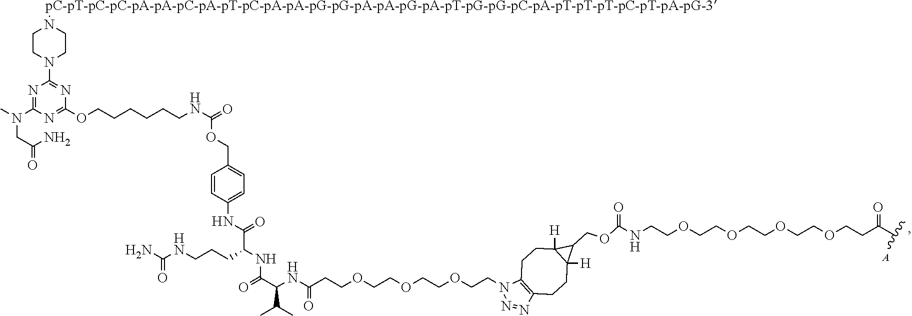

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21), wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked (e.g., indirectly or directly linked, e.g., directly linked) at attachment point A to a different amino acid residue of the Fab, optionally wherein each different amino acid residue is a lysine.

In some embodiments, complexes used in the methods described herein comprise a structure of formula (I): [R¹]ₙ₁—R², in which each R¹ comprises a group of the formula (Ic):

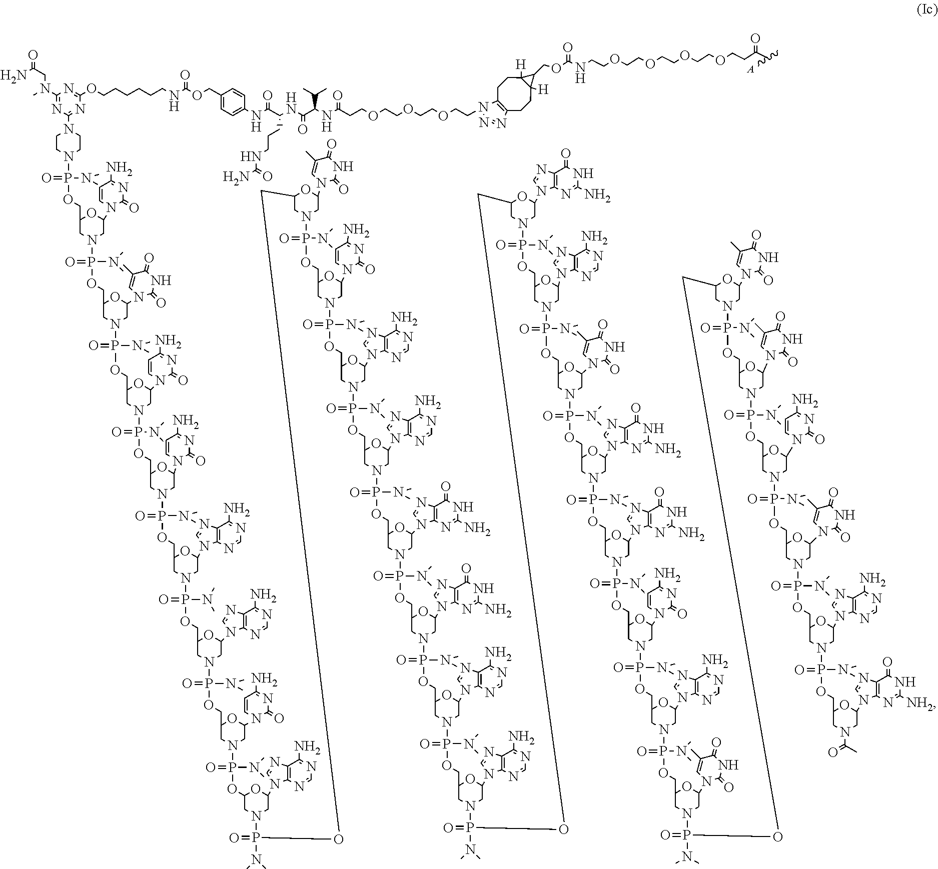

wherein in each complex n1 is independently an integer (e.g., of one or greater) representing the number of instances of R¹ in each complex, wherein each R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises a Fab and each R¹ is covalently linked (e.g., indirectly or directly linked, e.g., directly linked) at attachment point A to a different amino acid residue of the Fab, optionally wherein each different amino acid residue is a lysine.

In some embodiments, complexes used in the methods described herein comprise a structure of formula (Id):

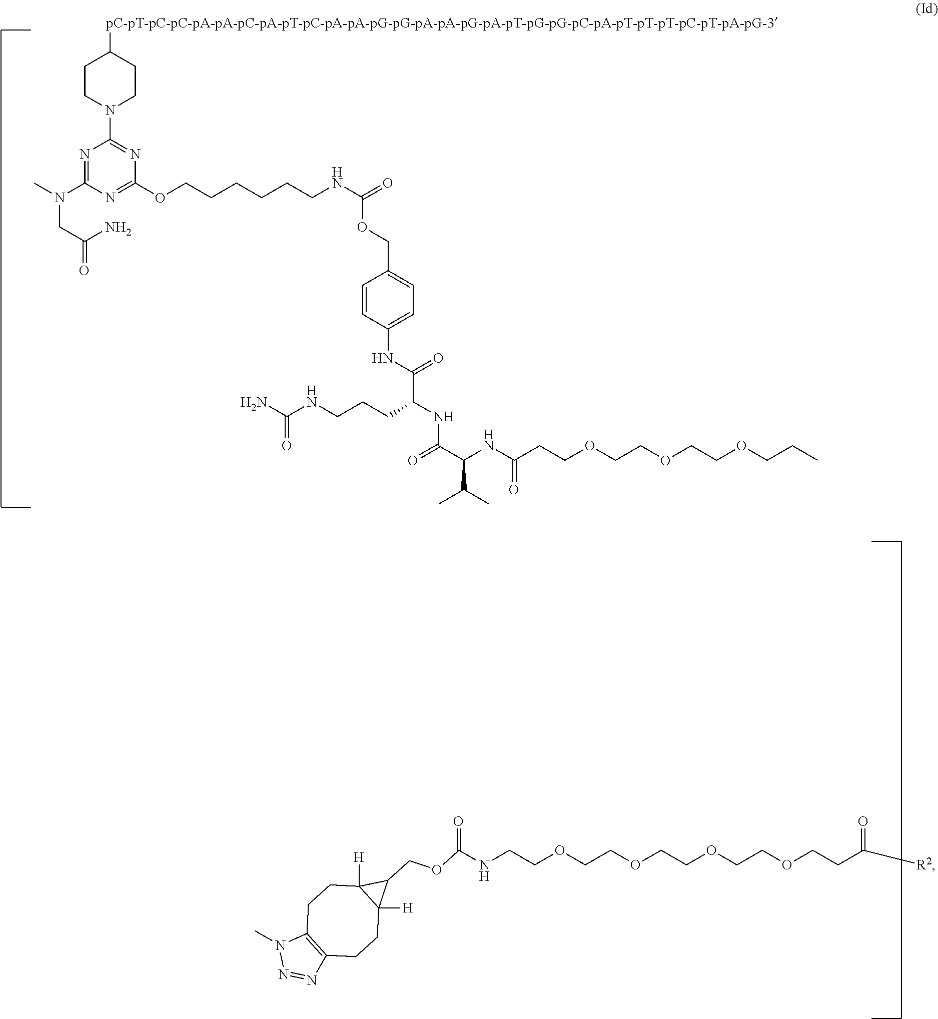

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21); wherein R² comprises an antibody (e.g., a Fab) comprising a CDR-H1, a CDR-H2, a CDR-H3, a CDR-L1, a CDR-L2, and a CDR-L3 selected from Table 2, optionally wherein the antibody (e.g., a Fab) comprises a VH comprising the amino acid sequence of SEQ ID NO: 17 and a VL comprising the amino acid sequence of SEQ ID NO: 18, further optionally wherein the antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a different amino acid residue of the antibody (e.g. a Fab), optionally wherein each different amino acid residue is a lysine. In some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, R² comprises an antibody (e.g., a Fab) that is covalently linked (e.g., indirectly or directly linked, e.g., directly linked) via different amino acid residue of the antibody (e.g., Fab), optionally wherein each different amino acid residue is a lysine.

In some embodiments, complexes described herein comprise a structure of formula (A):

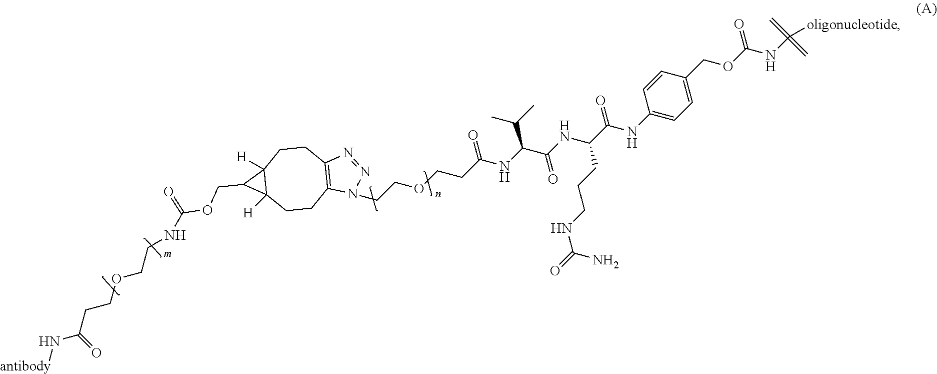

wherein n is 0-15 (e.g., 3) and m is 0-15 (e.g., 4). In some embodiments, the antibody is an an TfR1 antibody (e.g., the anti-TfR1 antibody provided in Table 2). In some embodiments, the oligonucleotide is a PMO and comprises the nucleotide sequence of SEQ ID NO: 21. In some embodiments, the amide shown adjacent to the anti-TfR1 antibody in the structure results from a reaction with an amine of the anti-TfR1 antibody, such as a lysine epsilon amine. In some embodiments, a complex described herein comprises an anti-TfR1 Fab covalently linked via a lysine of the Fab to the 5′ end of a PMO. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

### Antibodies

In some embodiments, complexes used in the methods described herein comprise an antibody that binds human transferrin receptor 1 (TfR1). An example human transferrin receptor 1 amino acid sequence, corresponding to NCBI sequence NP_003225.2 (transferrin receptor protein 1 isoform 1, Homo sapiens) is as follows:

(SEQ ID NO: 35)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLQWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

Table 2 provides examples of sequences of an anti-TfR1 antibody useful in the complexes provided herein.

TABLE 2
Examples of anti-TfR1 antibody sequences

| antibody | IMGT | Kabat | Chothia |
|---|---|---|---|
| CDR-H1 | GYSITSGYY | SGYYWN (SEQ ID | GYSITSGY (SEQ ID NO: 12) |
| | (SEQ ID NO: 1) | NO: 7) | |
| CDR-H2 | ITFDGAN | YITFDGANNYNPSL | FDG (SEQ ID NO: 13) |
| | (SEQ ID NO: 2) | KN (SEQ ID NO: 8) | |
| CDR-H3 | TRSSYDYDVL | SSYDYDVLDY | SYDYDVLD (SEQ ID NO: 14) |
| | DY (SEQ ID | (SEQ ID NO: 9) | |
| | NO: 3) | | |
| CDR-L1 | QDISNF (SEQ | RASQDISNFLN | SQDISNF (SEQ ID NO: 15) |
| | ID NO: 4) | (SEQ ID NO: 10) | |
| CDR-L2 | YTS (SEQ ID | YTSRLHS (SEQ ID | YTS (SEQ ID NO: 5) |
| | NO: 5) | NO: 11) | |
| CDR-L3 | QQGHTLPYT | QQGHTLPYT (SEQ | GHTLPY (SEQ ID NO: 16) |
| | (SEQ ID NO: 6) | ID NO: 6) | |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure comprises a light chain comprising the amino acid sequence of SEQ ID NO: 20. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Oligonucleotides

In some embodiments, an oligonucleotide of the complexes used in the methods described herein is a single stranded oligonucleotide. In some embodiments, the oligonucleotide is useful for targeting DMD (e.g., for exon skipping). In some embodiments, an oligonucleotide that is useful for targeting DMD (e.g., for exon skipping) targets a DMD allele (e.g., a mutated DMD allele). In some embodiments, an oligonucleotide useful for targeting DMD (e.g., for exon skipping) targets a region of a DMD RNA (e.g., the Dp427m transcript of SEQ ID NO: 24). In some embodiments, an oligonucleotide useful for targeting DMD (e.g., for exon skipping) comprises a region of complementarity to a DMD RNA (e.g., the Dp427m transcript of SEQ ID NO: 23). In some embodiments, an oligonucleotide useful for targeting DMD (e.g., for exon skipping) comprises a region of complementarity to an exon (e.g., exons 8, 23, 43, 44, 45, 46, 50, 51, 52, 53, or 55) or an intron of a DMD RNA. In some embodiments, an oligonucleotide useful for targeting DMD (e.g., for exon skipping) targets a splicing donor site, a splicing acceptor site, a branch point, or an exonic splicing enhancer (ESE) of a DMD RNA (e.g., a DMD pre-mRNA encoded by Homo sapiens dystrophin (DMD) gene (e.g., NCBI Accession No. NG_012232.1). In some embodiments, an oligonucleotide useful for targeting DMD (e.g., for exon skipping) targets an exonic splicing enhancer (ESE) sequence in DMD (e.g., an ESE sequence of exon 23, 44, 45, 46, 50, 51, 52, 53, or 55).

Examples of DMD RNA sequences and exon sequences that may be targeted by an oligonucleotide of a complex are provided below.

Homo sapiens dystrophin (DMD), transcript variant Dp427m, mRNA (NCBI
Reference Sequence: NM_004006.2).
(SEQ ID NO: 23)
Homo sapiens dystrophin (DMD), transcript variant Dp427m, exon 51 (nucleotide
positions 7554-7786 of NCBI Reference Sequence: NM_004006.2)
(SEQ ID NO: 24)
CTCCTACTCAGACTGTTACTCTGGTGACACAACCTGTGGTTACTAAGGAAACTGCCATC
TCCAAACTAGAAATGCCATCTTCCTTGATGTTGGAGGTACCTGCTCTGGCAGATTTCAA
CCGGGCTTGGACAGAACTTACCGACTGGCTTTCTCTGCTTGATCAAGTTATAAAATCAC
AGAGGGTGATGGTGGGTGACCTTGAGGATATCAACGAGATGATCATCAAGCAGAAG
Homo sapiens dystrophin (DMD), transcript variant Dp427m, exon 8 (nucleotide
positions 894-1075 of NCBI Reference Sequence: NM_004006.2)
(SEQ ID NO: 25)
ATGTTGATACCACCTATCCAGATAAGAAGTCCATCTTAATGTACATCACATCACTCTTC
CAAGTTTTGCCTCAACAAGTGAGCATTGAAGCCATCCAGGAAGTGGAAATGTTGCCAA
GGCCACCTAAAGTGACTAAAGAAGAACATTTTCAGTTACATCATCAAATGCACTATTCT
CAACAG
Homo sapiens dystrophin (DMD), transcript variant Dp427m, exon 23 (nucleotide
positions 3194-3406 of NCBI Reference Sequence: NM_004006.2)
(SEQ ID NO: 26)
GCTTTACAAAGTTCTCTGCAAGAGCAACAAAGTGGCCTATACTATCTCAGCACCACTGT
GAAAGAGATGTCGAAGAAAGCGCCCTCTGAAATTAGCCGGAAATATCAATCAGAATTT
GAAGAAATTGAGGGACGCTGGAAGAAGCTCTCCTCCCAGCTGGTTGAGCATTGTCAAA
AGCTAGAGGAGCAAATGAATAAACTCCGAAAAATTCAG
Homo sapiens dystrophin (DMD), transcript variant Dp427m, exon 43 (nucleotide
positions 6362-6534 of NCBI Reference Sequence: NM_004006.2)
(SEQ ID NO: 27)
AATATAAAAGATAGTCTACAACAAAGCTCAGGTCGGATTGACATTATTCATAGCAAGA
AGACAGCAGCATTGCAAAGTGCAACGCCTGTGGAAAGGGTGAAGCTACAGGAAGCTC
TCTCCCAGCTTGATTTCCAATGGGAAAAAGTTAACAAAATGTACAAGGACCGACAAGG
Homo sapiens dystrophin (DMD), transcript variant Dp427m, exon 44 (nucleotide
positions 6535-6682 of NCBI Reference Sequence: NM_004006.2)
(SEQ ID NO: 28)
GCGATTTGACAGATCTGTTGAGAAATGGCGGCGTTTTCATTATGATATAAAGATATTTA
ATCAGTGGCTAACAGAAGCTGAACAGTTTCTCAGAAAGACACAAATTCCTGAGAATTG
GGAACATGCTAAATACAAATGGTATCTTAAG
Homo sapiens dystrophin (DMD), transcript variant Dp427m, exon 45 (nucleotide
positions 6683-6858 of NCBI Reference Sequence: NM_004006.2)
(SEQ ID NO: 37)
GAACTCCAGGATGGCATTGGGCAGCGGCAAACTGTTGTCAGAACATTGAATGCAACTG
GGGAAGAAATAATTCAGCAATCCTCAAAAACAGATGCCAGTATTCTACAGGAAAAATT
GGGAAGCCTGAATCTGCGGTGGCAGGAGGTCTGCAAACAGCTGTCAGACAGAAAAAA
GAG
Homo sapiens dystrophin (DMD), transcript variant Dp427m, exon 46 (nucleotide
positions 6859-7006 of NCBI Reference Sequence: NM_004006.2)
(SEQ ID NO: 29)
GCTAGAAGAACAAAAGAATATCTTGTCAGAATTTCAAAGAGATTTAAATGAATTTGTT
TTATGGTTGGAGGAAGCAGATAACATTGCTAGTATCCCACTTGAACCTGGAAAAGAGC
AGCAACTAAAAGAAAAGCTTGAGCAAGTCAAG
Homo sapiens dystrophin (DMD), transcript variant Dp427m, exon 50 (nucleotide
positions 7445-7553 of NCBI Reference Sequence: NM_004006.2)
(SEQ ID NO: 30)
AGGAAGTTAGAAGATCTGAGCTCTGAGTGGAAGGCGGTAAACCGTTTACTTCAAGAGC
TGAGGGCAAAGCAGCCTGACCTAGCTCCTGGACTGACCACTATTGGAGCCT
Homo sapiens dystrophin (DMD), transcript variant Dp427m, exon 51 (nucleotide
positions 7554-7786 of NCBI Reference Sequence: NM_004006.2)
(SEQ ID NO: 31)
CTCCTACTCAGACTGTTACTCTGGTGACACAACCTGTGGTTACTAAGGAAACTGCCATC
TCCAAACTAGAAATGCCATCTTCCTTGATGTTGGAGGTACCTGCTCTGGCAGATTTCAA
CCGGGCTTGGACAGAACTTACCGACTGGCTTTCTCTGCTTGATCAAGTTATAAAATCAC
AGAGGGTGATGGTGGGTGACCTTGAGGATATCAACGAGATGATCATCAAGCAGAAG
Homo sapiens dystrophin (DMD), transcript variant Dp427m, exon 52 (nucleotide
positions 7787-7904 of NCBI Reference Sequence: NM_004006.2)
(SEQ ID NO: 32)
GCAACAATGCAGGATTTGGAACAGAGGCGTCCCCAGTTGGAAGAACTCATTACCGCTG
CCCAAAATTTGAAAAACAAGACCAGCAATCAAGAGGCTAGAACAATCATTACGGATCG
AA
Homo sapiens dystrophin (DMD), transcript variant Dp427m, exon 53 (nucleotide
positions 7905-8116 of NCBI Reference Sequence: NM_004006.2)
(SEQ ID NO: 33)
TTGAAAGAATTCAGAATCAGTGGGATGAAGTACAAGAACACCTTCAGAACCGGAGGC
AACAGTTGAATGAAATGTTAAAGGATTCAACACAATGGCTGGAAGCTAAGGAAGAAG
CTGAGCAGGTCTTAGGACAGGCCAGAGCCAAGCTTGAGTCATGGAAGGAGGGTCCCTA
TACAGTAGATGCAATCCAAAAGAAAATCACAGAAACCAAG
Homo sapiens dystrophin (DMD), transcript variant Dp427m, exon 55 (nucleotide
positions 8272-8461 of NCBI Reference Sequence: NM_004006.2)
(SEQ ID NO: 34)
GGTGAGTGAGCGAGAGGCTGCTTTGGAAGAAACTCATAGATTACTGCAACAGTTCCCC
CTGGACCTGGAAAAGTTTCTTGCCTGGCTTACAGAAGCTGAAACAACTGCCAATGTCCT
ACAGGATGCTACCCGTAAGGAAAGGCTCCTAGAAGACTCCAAGGGAGTAAAAGAGCT
GATGAAACAATGGCAA

In some embodiments, an oligonucleotide useful for targeting DMD (e.g., for exon skipping) is 15-40 (e.g., 15-40, 15-35, 15-30, 15-25, 15-20, 20-40, 20-35, 20-30, 20-25, 25-40, 25-35, 25-30, 25-28, 28-30, 30-40, 30-32, 32-35, 30-35, or 35-40) nucleotides in length. In some embodiments, an oligonucleotide useful for targeting DMD (e.g., for exon skipping) is 15, 16, 17, 18, 19, 20, 21, 22, 23, 24, 25, 26, 27, 28, 29, or 30 nucleotides in length, optionally 20-35, or 30 nucleotides in length.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD RNA. In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to an exon of a DMD RNA.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a DMD sequence as set forth in any one of SEQ ID NOs: 23-34.

In some embodiments, an oligonucleotide useful for targeting DMD (e.g., for exon skipping) comprises a region of complementarity of at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides to a target sequence as set forth in SEQ ID NO: 22 (CTAGAAATGCCATCTTCCTTGATGTTGGAG). In some embodiments, an oligonucleotide useful for targeting DMD (e.g., for exon skipping) comprises at least 8 (e.g., at least 8, 9, 10, 11, 12, 13, 14, 15, 16, 17, 18, 19, 20, 21, 22, 23, 24, 25, 26, 27, 28, 29, or 30) consecutive nucleotides of a sequence as set forth in SEQ ID NO: 21 (CTCCAACATCAAGGAAGATGGCATTTCTAG).

In some embodiments, an oligonucleotide useful for targeting DMD (e.g., for exon skipping) comprises the nucleotide sequence of SEQ ID NO: 21. In some embodiments, any one of the oligonucleotides provided herein is a PMO.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotide as set forth in SEQ ID NO: 21) may independently and optionally be uracil bases (U's), and/or any one or more of the U's in the oligonucleotides provided herein may independently and optionally be T's.

### Compositions

In some embodiments, compositions comprising complexes (i.e., a plurality of complexes) are formulated in a manner suitable for the methods described herein. In some embodiments, compositions comprising muscle-targeting complexes are delivered to a subject using a formulation that minimizes degradation, facilitates delivery and/or (e.g., and) uptake, or provides another beneficial property to the complexes in the formulation. Accordingly, in some embodiments, compositions comprising complexes (e.g., a plurality of complexes comprising a PMO covalently linked with a Fab) are formulated with histidine and/or sucrose. In some embodiments, compositions comprising muscle-targeting complexes (e.g., complexes comprising a PMO covalently linked with a Fab) are formulated with histidine and/or sucrose in aqueous solutions. In some embodiments, compositions comprising a plurality of the complexes, histidine, and sucrose can be lyophilized (e.g., for storage). In some embodiments, the lyophilized composition may be reconstituted (e.g., with water) for administration to a subject. The compositions (e.g., in aqueous solutions or in lyophilized compositions) can be suitably prepared such that when administered to a subject, either into the immediate environment of a target cell or systemically, a sufficient amount of the complexes enter target muscle cells.

In some embodiments, compositions (e.g., in aqueous solutions) for administration to a subject in the methods described herein comprise complexes (i.e., a plurality of complexes), each of which complex comprises a phosphorodiamidate morpholino oligomer (PMO) covalently linked to an antibody. In some embodiments, compositions (e.g., in aqueous solutions) for administration to a subject in the methods described herein comprise complexes, in which each complex comprises a phosphorodiamidate morpholino oligomer (PMO) covalently linked to an anti-TfR1 antibody, optionally wherein the antibody of such complexes comprises a CDR-H1, CDR-H2, CDR-H3, CDR-L1, CDR-L2, and CDR-L3 as set forth in Table 2, and further, in some embodiments, wherein the composition further comprises histidine (e.g., L-histidine) and sucrose. In some embodiments, the antibody is an anti-TfR1 Fab.

In some embodiments, compositions (e.g., in aqueous solutions) for administration to a subject in the methods described herein comprise complexes (i.e., a plurality of complexes) wherein each complex is a structure of the formula (I): [R¹]ₙ₁—R², in which each R¹ independently comprises a compound comprising an oligonucleotide (e.g., a PMO) and is covalently linked to R², wherein R² comprises an antibody (e.g., anti-TfR1 antibody), and in which in each complex n1 is independently an integer of one or greater representing the number of instances of R¹ in each complex.

In some embodiments, the value of n1 of each complex in the composition is independently and optionally an integer from one up to the number of amino acid residues to which conjugation is desired or targeted (e.g., the number of lysine residues) in the antibody (R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 3, 1 to 5, 1 to 10, 1 to 26, or 1 to 27.

In some embodiments, a composition for administration to a subject in the methods described herein comprises unconjugated antibody (e.g., in trace amounts) and antibody conjugated to one or more oligonucleotides. In some embodiments, unconjugated antibody may be referred to as a compound of the structure of formula (I): [R¹]ₙ₁—R², for which n1 is zero. Accordingly, in some embodiments, a composition for administration to a subject in the methods described herein comprises compounds (e.g., complexes) of the structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds of the structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%.

In some embodiments, each instance of R¹ in a complex herein (e.g., a complex of a composition provided herein) is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, formulations are provided in which complexes for which R¹ is covalently linked to an amino acid residue residing in a CDR region of the antibody are present in only trace amounts, or in undetectable amount, or not at all. In some embodiments, formulations are provided in which complexes for which R¹ is covalently linked to an amino acid residue residing in a CDR region of the antibody are not detectable in the formulation using standard detection techniques.

In some embodiments, compositions (e.g., in aqueous solutions) for administration to a subject in the methods described herein comprise complexes that comprise a structure of the formula (I): [R¹]ₙ₁—R², wherein each R¹ in a complex of a composition provided herein independently comprises a group of the formula (Ia):

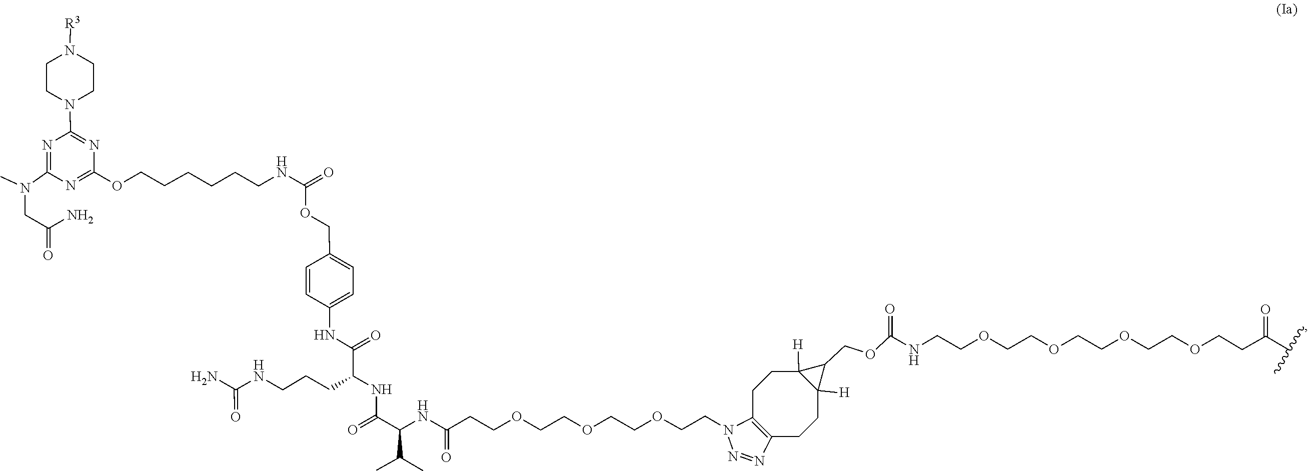

in which R³ is an oligonucleotide, e.g., a phosphorodiamidate morpholino oligomer (PMO); wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, R³ is an oligonucleotide, e.g., a phosphorodiamidate morpholino oligomer (PMO) comprising the base sequence of CTCCAACATCAAGGAAGATGGCATTTCTAG (SEQ ID NO: 21). In some embodiments, R² comprises an antibody (e.g., a Fab) and each R¹ is covalently linked (e.g., indirectly or directly linked, e.g., directly linked) at attachment point A to a different amino acid residue of the antibody (e.g., Fab), optionally wherein each different amino acid residue is a lysine. In some embodiments, in each complex n1 is independently an integer (e.g., an integer in the range of 1-27, 1-26, 1-10, 1-5, or 1-3). In some embodiments, compositions (e.g., in aqueous solutions) for administration to a subject in the methods described herein comprise complexes that comprise a structure of formula (I): [R¹]ₙ₁—R², wherein n1 is 0.

In some embodiments, compositions (e.g., in aqueous solutions) for administration to a subject in the methods described herein comprise complexes that comprise a structure of formula (I): [R¹]ₙ₁—R², in which each instance of R¹ in a complex of a composition provided herein comprises a group of the formula (Ib):

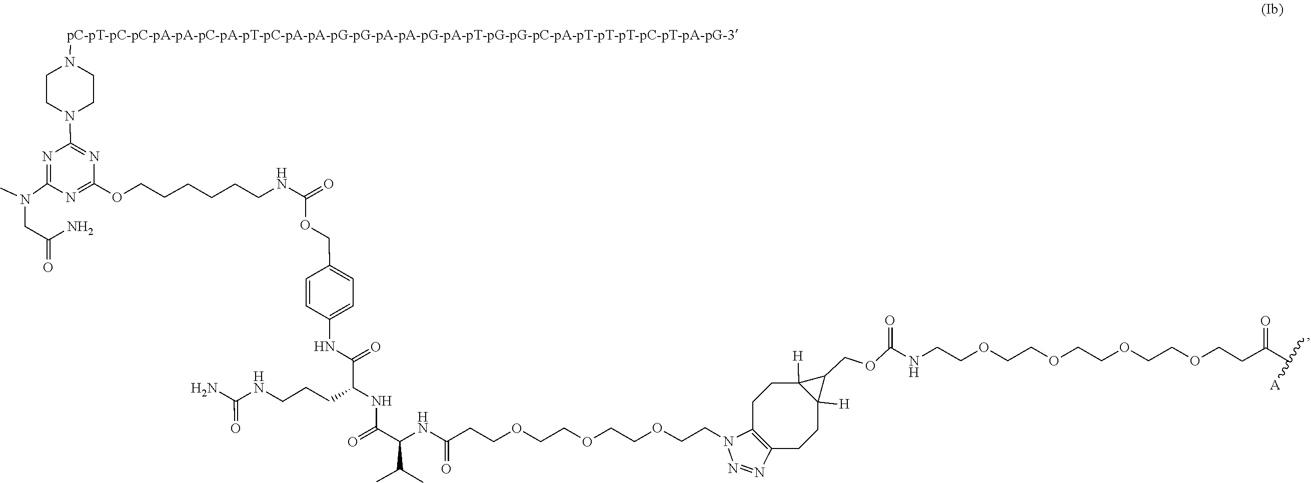

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21), wherein in each complex n1 is independently an integer (e.g., of one or greater) representing the number of instances of R¹ in each complex, and each R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises an antibody (e.g., a Fab) and each R¹ is covalently linked (e.g., indirectly or directly linked, e.g., directly linked) at attachment point A to a different amino acid residue of the antibody (e.g., Fab), optionally wherein each different amino acid residue is a lysine. In some embodiments, compositions (e.g., in aqueous solutions) for administration to a subject in the methods described herein further comprise complexes that comprise a structure of formula (I): [R¹]ₙ₁—R², wherein n1 is 0.

In some embodiments, compositions (e.g., in aqueous solutions) for administration to a subject in the methods described herein comprise complexes that comprise a structure of formula (I): [R₁]ₙ₁—R², in which each instance R¹ in a complex of a composition provided herein comprises a group of the formula (Ic):

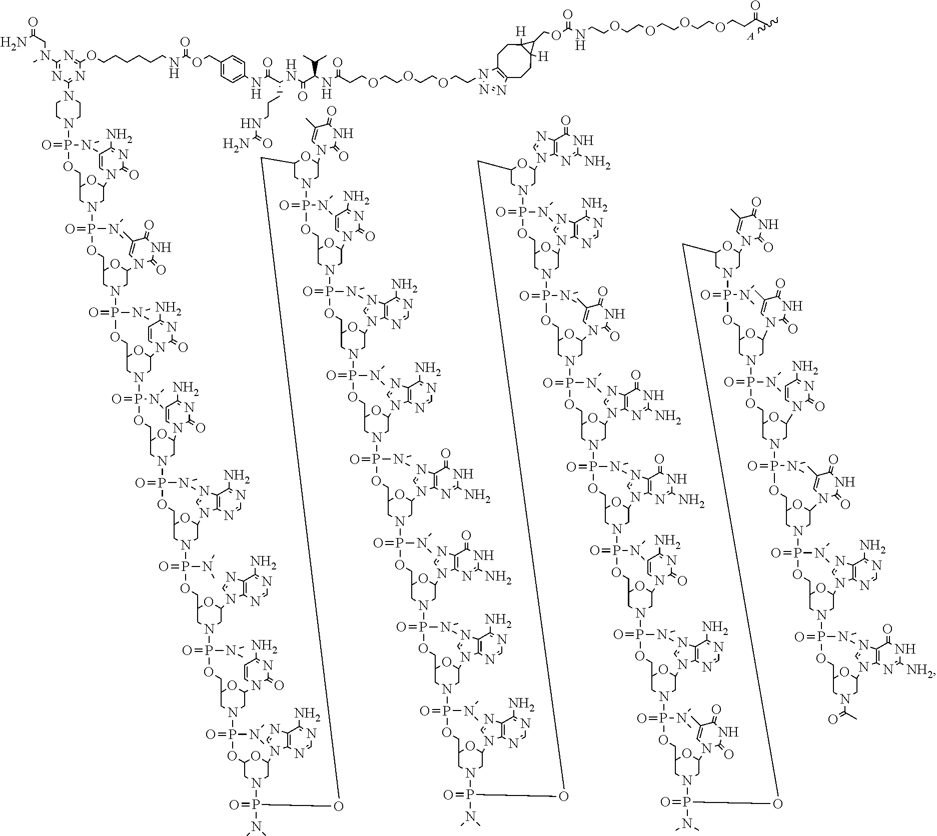

wherein in each complex n is independently an integer (e.g., of one or greater) representing the number of instances of R¹ in each complex, wherein each R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)2 fragment, an scFv, or an Fv. In some embodiments, R² comprises an antibody that is a Fab fragment. In some embodiments, in each complex n1 is independently an integer (e.g., an integer in the range of 1-27, 1-26, 1-10, 1-5, or 1-3). In some embodiments, R² comprises an antibody (e.g., a Fab) and each R¹ is covalently linked (e.g., indirectly or directly linked, e.g., directly linked) at attachment point A to a different amino acid residue of the antibody (e.g., Fab), optionally wherein each different amino acid residue is a lysine. In some embodiments, compositions (e.g., in aqueous solutions) for administration to a subject in the methods described herein further comprise complexes that comprise a structure of formula (I): [R¹]ₙ₁—R², wherein n1 is 0.

In some embodiments, compositions (e.g., in aqueous solutions) for administration to a subject in the methods described herein comprise complexes that comprise a structure of formula (Id):

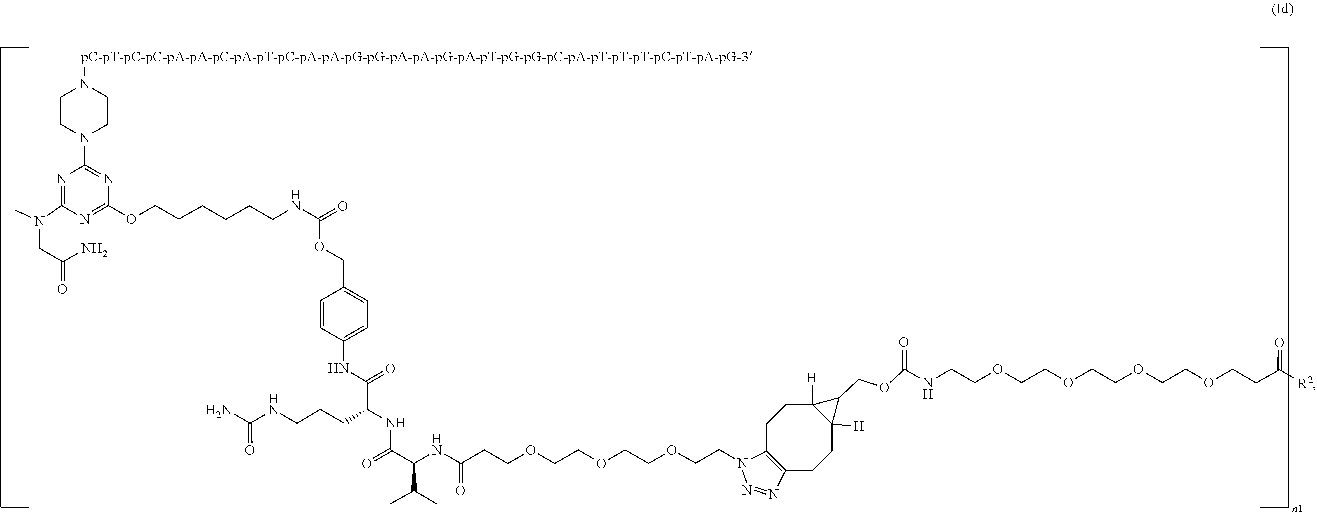

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21); wherein R² comprises an antibody (e.g., a Fab) comprising a CDR-H1, a CDR-H2, a CDR-H3, a CDR-L1, a CDR-L2, and a CDR-L3 selected from Table 2, optionally wherein the antibody (e.g., a Fab) comprises a VH comprising the amino acid sequence of SEQ ID NO: 17 and a VL comprising the amino acid sequence of SEQ ID NO: 18, further optionally wherein the antibody (e.g., a Fab) comprises a heavy chain comprising the amino acid sequence of SEQ ID NO: 19 and a light chain comprising the amino acid sequence of SEQ ID NO: 20; and wherein in each complex n1 is independently an integer (e.g., of one or greater) representing the number of instances of the group enclosed by square brackets, wherein each instance of the group enclosed by square brackets is covalently linked to a different amino acid residue of the antibody (e.g., a Fab), optionally wherein each different amino acid residue is a lysine. In some embodiments, R² comprises an antibody (e.g., a Fab) comprising a sequence as set forth in Table 2. For example, in some embodiments, R² comprises an antibody (e.g., a Fab) comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, R² comprises an antibody (e.g., a Fab) comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody (e.g., a Fab) comprising a VH comprising the amino acid sequence of SEQ ID NO: 17 and/or comprising a VL comprising the amino acid sequence of SEQ ID NO: 18. In some embodiments, R² comprises an antibody (e.g., a Fab) comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody (e.g., a Fab) comprising a heavy chain comprising the amino acid sequence of SEQ ID NO: 19 and/or comprising a light chain comprising the amino acid sequence of SEQ ID NO: 20. In some embodiments, in each complex n1 is independently an integer (e.g., an integer in the range of 1-27, 1-26, 1-10, 1-5, or 1-3). In some embodiments, in each complex n1 is independently an integer of one or greater. In some embodiments, R² comprises an antibody (e.g., a Fab) that is covalently linked (e.g., indirectly or directly linked, e.g., directly linked) via different amino acid residue of the antibody (e.g., Fab), optionally wherein each different amino acid residue is a lysine. In some embodiments, compositions (e.g., in aqueous solutions) for administration to a subject in the methods described herein further comprise complexes in which n1 is 0.

In some embodiments, compositions (e.g., aqueous solutions) for administration to a subject in the methods described herein comprise a structure of formula (A):

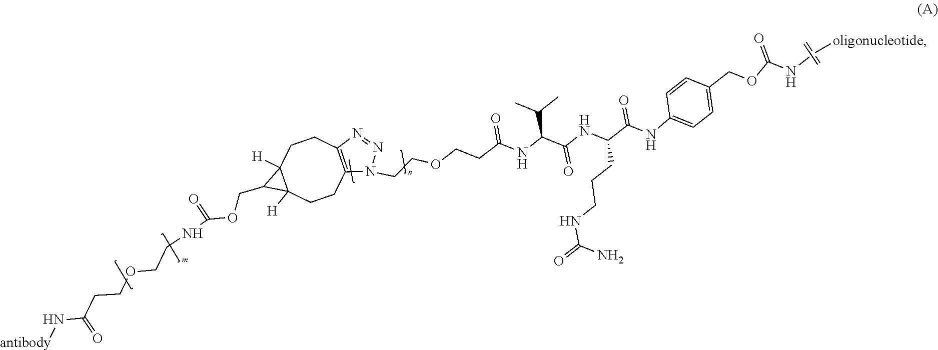

wherein n is 0-15 (e.g., 3) and m is 0-15 (e.g., 4). In some embodiments, the antibody is an an TfR1 antibody (e.g., the anti-TfR1 antibody provided in Table 2). In some embodiments, the oligonucleotide is a PMO and comprises the base sequence of SEQ ID NO: 21. In some embodiments, the amide shown adjacent to the antibody in the structure results from a reaction with an amine of the antibody, such as a lysine epsilon amine. In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)2 fragment, an scFv, or an Fv.

In some embodiments, compositions (e.g., in aqueous solutions) for administration to a subject in the methods described herein comprise complexes, wherein a concentration of the complexes in the formulation therein is between 1-50 mg/mL of the complex, optionally 10-50 mg/ml or 20-35 mg/mL (e.g., 1-10 mg/mL, 10⁻¹⁵ mg/mL, 15-20 mg/mL, 20-22 mg/mL, 22-24 mg/ml, 24-26 mg/ml, 24-25 mg/ml, 25-26 mg/ml, 22-25 mg/mL, 25-27 mg/mL, 27-29 mg/mL, 29-30 mg/mL, 25-30 mg/mL, 29-31 mg/ml, 30-31 mg/ml, 31-32 mg/ml, 30-32 mg/mL, 32-33 mg/ml, 32-35 mg/mL, 30-35 mg/mL, 35-40 mg/mL, 40-45 mg/mL, 45-50 mg/mL), optionally approximately 25 mg/mL (e.g., 25 mg/mL) or approximately 30 mg/mL (e.g., 30 mg/mL).

In some embodiments, any one or a plurality of the complexes for use in the methods described herein is formulated with the histidine (e.g., L-histidine) and the sucrose in an aqueous solution or in a lyophilized form (e.g., lyophilized powder).

In some embodiments, any one or a plurality of the complexes for use in the methods described herein is formulated with the histidine (e.g., L-histidine) and the sucrose in an aqueous solution. In some embodiments, the histidine (e.g., L-histidine) is present in the aqueous solution at a concentration in the range of 10-50 mM, 10-20 mM, 20 mM to 30 mM, or 20 mM to 40 mM, e.g., 20-22 mM, 22-24 mM, 24-25 mM, 25-26 mM, 24-26 mM, 26-27 mM, 24-27 mM, 27-28 mM, 28-29 mM, 29-30 mM, 27-30 mM, approximately 22-27 mM, approximately 23-26 mM, approximately 24-26 mM, approximately 26-28 mM, approximately 28-30 mM, approximately 30-32 mM, approximately 32-35 mM, approximately 35-40 mM, 40-45 mM, 45-50 mM, approximately 25 mM, or optionally, 25 mM. In some embodiments, the sucrose is present in the aqueous solution at a concentration in the range of 5% to 15% weight per volume (w/v %), for example, 8-15% w/v %, 9-15% w/v %, 9-11% w/v %, 9.5-11% w/v %, or for example, in the range of 5-6 w/v %, 6-7 w/v %, 7-8 w/v %, 8-9 w/v %, 9-10 w/v %, 10-11 w/v %, 11-12% w/v %, 10-12 w/v %, 12-13% w/v %, 13-14% w/v %, 12-14 w/v %, 14-15 w/v %, or 8-12 w/v %. In some embodiments, the sucrose is present in the aqueous solution at a concentration in the range of 8-12 w/v % (e.g., 10 w/v %). In some embodiments, the aqueous solution has a pH in the range of 5.0 to 7.0, for example, 5.0-5.2, 5.2-5.4, 5.4-5.6, 5.6-5.8, 5.8-6.0, 5.9-6.0, 5.9-6.1, 6.0-6.1; for example, 5.5 to 6.5, or for example, in the pH range of 5.5-5.8, 5.8-6.0, 5.9-6.1, 6.0-6.1, 6.0-6.2, 6.2-6.4, 6.4-6.5, 6.5-6.7, 6.7-6.8, 6.8-6.9, 6.9-7.0, 7.0-7.1, or 5.8-6.2. In some embodiments, the aqueous solution has a pH in the range of 5.8-6.2 (e.g., 5.8-6.0, 5.8-6.1, 5.9-6.1). In some embodiments, the aqueous solution has a pH in the range of 5.9-6.2. In some embodiments, the aqueous solution has a pH in the range of 6.0-6.1 (e.g., about 6.0, or 6.0).

In some embodiments, any one of the compositions (e.g., aqueous solution) for use in the methods described herein comprises one or a plurality of complexes, histidine, and sucrose, wherein the histidine (e.g., L-histidine) is present in the compositions (e.g., aqueous solution) at a concentration of 25 mM, wherein the sucrose is present in the compositions (e.g., aqueous solution) at a concentration of 10 w/v %, and wherein the compositions (e.g., aqueous solution) is at a pH of about 6.0 (e.g., 6.0, 5.9-6.1).

In some embodiments, any one of the compositions (e.g., aqueous solution) for use in the methods described herein comprises one or a plurality of complexes, histidine, and sucrose, wherein the histidine (e.g., L-histidine) is present in the aqueous solution at a concentration of 25 mM, wherein the sucrose is present in the aqueous solution at a concentration of 10 w/v %, and wherein the pH of about 6.0 (e.g., 6.0, 5.9-6.1), and the concentration of complexes in the formulation is 10-50 mg/ml or 20-35 mg/mL (e.g., 1-10 mg/mL, 10⁻¹⁵ mg/mL, 15-20 mg/mL, 20-22 mg/mL, 22-24 mg/ml, 24-26 mg/ml, 22-25 mg/mL, 25-27 mg/mL, 27-29 mg/mL, 29-31 mg/ml, 29-30 mg/mL, 30-31 mg/ml, 31-32 mg/ml, 25-30 mg/mL, 30-32 mg/mL, 32-35 mg/mL, 30-35 mg/mL, 35-40 mg/mL, 40-45 mg/mL, 45-50 mg/mL), optionally 25 mg/mL or 30 mg/mL.

As described herein, in some embodiments, compositions for use in the methods described herein are formulated in aqueous solutions that comprise sucrose. In some embodiments, the sucrose serves at least in part as a lyoprotectant. In some embodiments, the sucrose is from a plant, e.g., grass, fruit, or vegetable (e.g., root vegetable) source (e.g., beet (e.g., sugar beet, for example, Saccharum spp.)), sugarcane (e.g., Beta vulgaris), dates, sugar maple, sweet sorghum, apples, oranges, carrots, molasses, maple syrup, corn sweeteners) or animal product (e.g., honey). In some embodiments, the sucrose is from beet or sugarcane (e.g., beet sucrose, sugarcane sucrose). In some embodiments, a lyoprotectant other than sucrose may be used, e.g., trehalose, mannitol, lactose, polyethylene glycol, or polyvinyl pyrrolidone. However, in some embodiments, a collapse temperature modifier (e.g., dextran, ficoll, or gelatin) may be provided in a composition.

In some embodiments, provided is a product (e.g., lyophilized composition described herein), produced by a process comprising lyophilizing an aqueous solution of a composition (e.g., in aqueous form) described herein.

In some embodiments, a composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment/Dosing

Complexes comprising an anti-TfR1 antibody (e.g., Fab) covalently linked to a molecular payload (e.g., oligonucleotide, e.g., phosphorodiamidate morpholino oligomer (PMO)) as described herein are effective in treating a subject having a dystrophinopathy, e.g., Duchenne Muscular Dystrophy. In some embodiments, complexes comprise a molecular payload that is an oligonucleotide, e.g., an oligonucleotide that facilitates exon skipping of an mRNA expressed from a mutated DMD allele.

In some embodiments, a subject may be a human subject, a non-human primate subject (e.g., cynomolgus monkey), a rodent subject, or any suitable mammalian subject. In some embodiments, the subject is human. In some embodiments, the subject is a human subject that is between 2-60 (e.g., 2-60, 2-50, 2-40, 2-30, 2-20, 2-10) years of age. In some embodiments, the subject is a human subject that is between 5-30 (e.g., 5, 6, 7, 8, 9, 10, 11, 12, 13, 14, 15, 16, 17, 18, 19, 20, 21, 22, 23, 24, 25, 26, 27, 28, 29, or 30) years old. In some embodiments, the subject is a human subject that is between 5-12 (e.g., 5, 6, 7, 8, 9, 10, 11, or 12) years of age. In some embodiments, the subject is a human subject that is between 4-16 (e.g., 4-16, 5-16, 6-16, 7-16, 8-16, 9-16, 10-16, 11-16, 12-16, 13-16, 14-16, 15-16, 4-15, 5-15, 6-15, 7-15, 8-15, 9-15, 10-15, 11-15, 12-15, 13-15, 14-15, 4-14, 5-14, 6-14, 7-14, 8-14, 9-14, 10-14, 11-14, 12-14, 13-14, 4-13, 5-13, 6-13, 7-13, 8-13, 9-13, 10-13, 11-13, 12-13, 4-12, 5-12, 6-12, 7-12, 8-12, 9-12, 10-12, 11-12, 4-11, 5-11, 6-11, 7-11, 8-11, 9-16, 10-11, 4-10, 5-10, 6-10, 7-10, 8-10, 9-10, 4-9, 5-9, 6-9, 7-9, 8-9, 4-9, 5-9, 6-9, 7-9, 8-9, 4-8, 5-8, 6-8, 7-8, 4-7, 5-7, 6-7, 4-6, 5-6, or 4-5) years of age. In some embodiments, the subject is a human subject that is about 4, 5, 6, 7, 8, 9, 10, 11, 12, 13, 14, 15, or 16 years of age.

In some embodiments, a subject may have Duchenne muscular dystrophy or other dystrophinopathy. In some embodiments, a subject has a mutated DMD allele, which may optionally comprise at least one mutation in a DMD exon that causes a frameshift mutation and leads to improper RNA splicing/processing. In some embodiments, a subject is suffering from symptoms of a severe dystrophinopathy, e.g., muscle atrophy or muscle loss. In some embodiments, a subject has an asymptomatic increase in serum concentration of creatine phosphokinase (CK) and/or (e.g., and) muscle cramps with myoglobinuria. In some embodiments, a subject has a progressive muscle disease, such as Duchenne or Becker muscular dystrophy or DMD-associated dilated cardiomyopathy (DCM). In some embodiments, a subject is not suffering from symptoms of a dystrophinopathy. In some embodiments, a subject is ambulant. In some embodiments, a subject is non-ambulant. In some embodiments, a subject is ambulatory. In some embodiments, a subject is non-ambulatory. In some embodiments, a subject is non-ambulatory and have been non-ambulatory for less than 2 years prior to being treated with a method described herein.

In some embodiments, a subject has a mutation in a DMD gene that is amenable to exon 51 skipping. In some embodiments, a complex as described herein is effective in treating a subject having a mutation in a DMD gene that is amenable to exon 51 skipping. In some embodiments, a complex comprises an oligonucleotide, e.g., an oligonucleotide that facilitates skipping of exon 51 of a pre-mRNA, such as in a pre-mRNA encoded from a mutated DMD gene (e.g., a mutated DMD gene that is amenable to exon 51 skipping).

In some embodiments, a subject has a Brooke Upper Extremity Scale score of 1 or 2. The Brooke Upper Extremity Scale uses a scale from 1 to 6, with 1 indicating an individual's full capability of lifting the arms in a full circle until they touch and 6 indicating that an individual cannot raise hands to mouth and has no useful function of the hands (see, e.g., Brooke et al. (1981). Muscle Nerve 4(3): 186-197, incorporated herein by reference).

In some embodiments, a subject is not receiving or has not received treatment with glucocorticoids (e.g., prednisone, prednisolone, deflazacort). In some embodiments, a subject is also receiving or has received treatment with glucocorticoids. In some embodiments, a subject is receiving or has received a stable dosage of glucocorticoids (e.g., prednisone, prednisolone, deflazacort). In some embodiments, a subject has been receiving stable dosages of glucocorticoids (e.g., prednisone, prednisolone, deflazacort) for at least 12 weeks (e.g., at least 12 weeks, at least 16 weeks, at least 20 weeks, at least 24 weeks, at least 7 months, at least 8 months, at least 9 months, at least 10 months, at least 11 months, at least 12 month or longer) prior to being treated with a method described herein.

An aspect of the disclosure includes methods involving administering to a subject a composition (e.g., aqueous solution) comprising an effective amount of complex(es) as described herein. In some embodiments, an effective amount of a composition (e.g., aqueous solution) that comprises complex(es) comprising an antibody (e.g., Fab) described herein covalently linked to an oligonucleotide (e.g., PMO) described herein can be administered to a subject in need of treatment. In some embodiments, a composition (e.g., aqueous solution) is administered systemically. In some embodiments, a pharmaceutical composition comprising complex(es) as described herein may be administered by a suitable route, which may include intravenous administration, e.g., as a bolus or by continuous infusion over a period of time. In some embodiments, administration may be performed by intravenous, intramuscular, intraperitoneal, intracerebrospinal, subcutaneous, intra-articular, intrasynovial, or intrathecal routes. In some embodiments, a composition (e.g., aqueous solution) comprising complex(es) as described herein is administered by infusion (e.g., intravenous infusion).

In some embodiments, a composition comprising a plurality of complexes described herein may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

In some embodiments, provided are methods of and/or uses for treating a subject having a mutated DMD allele associated with Duchenne Muscular Dystrophy (DMD), comprising administering to the subject a composition comprising a complex or plurality of complexes described herein with an effective amount of the complex(es). In some embodiments, provided are methods of and/or uses for promoting the expression or activity of a dystrophin protein in a subject, the methods comprising contacting the cell with the composition comprising a plurality of complexes described herein with an effective amount of the complex(es). In some embodiments, the dystrophin protein is a truncated dystrophin protein. The truncated dystrophin protein is functional (e.g., retains activities of a wild-type dystrophin protein). In some embodiments, the truncated dystrophin protein retains partial function of a wild-type dystrophin protein. In some embodiments, the method comprises administering a lyophilized form (e.g., lyophilized powder) of a composition comprising a plurality of complexes described herein, comprising reconstituting a lyophilized form of the composition in an aqueous solution, and administering the aqueous solution to a subject in need thereof. For example, in some embodiments, a lyophilized form of the composition comprising a complex or plurality of complexes is shipped and/or stored in the lyophilized form, reconstituted in an aqueous solution at a location (e.g., healthcare provider location) for administration, and administered in the reconstituted form (e.g., as an aqueous solution) by injection or intravenously, e.g., by infusion. In some embodiments, the subject has a mutated DMD allele comprises a mutation amenable to exon 51 skipping. In some embodiments, the mutated DMD allele comprises a frameshift mutation in exon 51.

In some embodiments, a composition is administered via site-specific or local delivery techniques. Examples of these techniques include implantable depot sources of the complex, local delivery catheters, site specific carriers, direct injection, or direct application.

In some embodiments, in any one of the methods described herein, the effective amount provides to the subject 1 mg to 110 mg (e.g., 1 mg to 110 mg, 1 mg to 100 mg, 1 mg to 90 mg, 1 mg to 80 mg, 1 mg to 70 mg, 1 mg to 60 mg, 1 mg to 50 mg, 1 mg to 40 mg, 1 mg to 30 mg, 1 mg to 20 mg, 1 mg to 10 mg, 5 mg to 110 mg, 5 mg to 100 mg, 5 mg to 90 mg, 5 mg to 80 mg, 5 mg to 70 mg, 5 mg to 60 mg, 5 mg to 50 mg, 5 mg to 40 mg, 5 mg to 30 mg, 5 mg to 20 mg, 5 mg to 10 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 100 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 100 mg, 30 mg to 90 mg, 30 mg to 80 mg, 30 mg to 70 mg, 30 mg to 60 mg, 30 mg to 50 mg, 30 mg to 40 mg, 40 mg to 100 mg, 40 mg to 90 mg, 40 mg to 80 mg, 40 mg to 70 mg, 40 mg to 60 mg, 40 mg to 50 mg, 50 mg to 100 mg, 50 mg to 90 mg, 50 mg to 80 mg, 50 mg to 70 mg, 50 mg to 60 mg, 60 mg to 100 mg, 60 mg to 90 mg, 60 mg to 80 mg, 60 mg to 70 mg, 70 mg to 100 mg, 70 mg to 90 mg, 70 mg to 80 mg, 80 mg to 100 mg, 80 mg to 90 mg, or 90 mg to 100 mg) of the anti-TfR1 antibody (e.g., Fab) of the complexes per kg of the subject.

In some embodiments, in any one of the methods described herein, the effective amount provides to the subject 1 mg to 25 mg (e.g., 1 mg to 25 mg, 1 mg to 20 mg, 1 mg to 15 mg, 1 mg to 11 mg, 1 mg to 10 mg, 1 mg to 8 mg, 1 mg to 5 mg, 1.5 mg to 25 mg, 1.5 mg to 20 mg, 1.5 mg to 15 mg, 1.5 mg to 10 mg, 1.5 mg to 6 mg, 1.5 mg to 5 mg, 2 mg to 25 mg, 2 mg to 20 mg, 2 mg to 15 mg, 2 mg to 11 mg, 2 mg to 10 mg, 2 mg to 5 mg, 5 mg to 25 mg, 5 mg to 20 mg, 5 mg to 15 mg, 5 mg to 11 mg, 5 mg to 10 mg, 10 mg to 25 mg, 10 mg to 20 mg, 10 mg to 15 mg, 15 mg to 25 mg, 15 mg to 20 mg, or 20 mg to 25 mg) of the anti-TfR1 antibody (e.g., Fab) of the complexes per kg of the subject. In some embodiments, in any one of the methods described herein, the effective amount provides to the subject about 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg of the anti-TfR1 antibody (e.g., Fab) of the complexes per kg of the subject.

In some embodiments, in any one of the methods described herein, the effective amount provides to the subject about 1.09 mg, 1.31 mg, 1.53 mg, 1.75 mg, 1.97 mg, 2.19 mg, 2.40 mg, 2.62 mg, 2.84 mg, 3.06 mg, 3.28 mg, 3.50 mg, 3.72 mg, 3.93 mg, 4.15 mg, 4.37 mg, 4.59 mg, 4.81 mg, 5.03 mg, 5.25 mg, 5.46 mg, 5.68 mg, 5.90 mg, 6.12 mg, 6.34 mg, 6.56 mg, 6.77 mg, 6.99 mg, 7.21 mg, 7.43 mg, 7.65 mg, 7.87 mg, 8.09 mg, 8.30 mg, 8.52 mg, 8.74 mg, 8.96 mg, 9.18 mg, 9.40 mg, 9.62 mg, 9.83 mg, 10.05 mg, 10.27 mg, 10.49 mg, 10.71 mg, 10.93 mg, 13.11 mg, 15.30 mg, 17.48 mg, 19.67 mg, 21.85 mg, or 24.04 mg of the anti-TfR1 antibody (e.g., Fab) of the complexes per kg of the subject.

In some embodiments, one of the methods described herein, the effective amount provides to the subject about 1.19 mg, 1.24 mg, 1.29 mg, 1.34 mg, 1.40 mg, 1.46 mg, 1.53 mg, 1.61 mg, 1.69 mg, 1.78 mg, 1.89 mg, 2.01 mg, 2.38 mg, 2.47 mg, 2.57 mg, 2.68 mg, 2.79 mg, 2.92 mg, 3.06 mg, 3.21 mg, 3.38 mg, 3.57 mg, 3.78 mg, 4.02 mg, 4.76 mg, 4.94 mg, 5.14 mg, 5.35 mg, 5.59 mg, 5.84 mg, 6.12 mg, 6.43 mg, 6.76 mg, 7.14 mg, 7.56 mg, 8.03 mg of the anti-TfR1 antibody (e.g., Fab) of the complexes per kg of the subject.

In some embodiments, in any one of the methods described herein, the effective amount provides to the subject about 1.5 mg, 3 mg, or 6 mg of the anti-TfR1 antibody (e.g., Fab) of the complexes per kg of the subject. In some embodiments, in any one of the methods described herein, the effective amount provides to the subject about 1.53 mg, 3.06 mg, or 6.12 mg of the anti-TfR1 antibody (e.g., Fab) of the complexes per kg of the subject.

In some embodiments, in any one of the methods described herein, the effective amount provides to the subject 5 mg to 120 mg (e.g., 5 mg to 120 mg, 5 mg to 110 mg, 5 mg to 100 mg, 5 mg to 90 mg, 5 mg to 80 mg, 5 mg to 70 mg, 5 mg to 60 mg, 5 mg to 50 mg, 5 mg to 40 mg, 5 mg to 30 mg, 5 mg to 20 mg, 5 mg to 10 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 30 mg, 10 mg to 20 mg, 20 mg to 100 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 100 mg, 30 mg to 90 mg, 30 mg to 80 mg, 30 mg to 70 mg, 30 mg to 60 mg, 30 mg to 50 mg, 30 mg to 40 mg, 40 mg to 100 mg, 40 mg to 90 mg, 40 mg to 80 mg, 40 mg to 70 mg, 40 mg to 60 mg, 40 mg to 50 mg, 50 mg to 100 mg, 50 mg to 90 mg, 50 mg to 80 mg, 50 mg to 70 mg, 50 mg to 60 mg, 60 mg to 100 mg, 60 mg to 90 mg, 60 mg to 80 mg, 60 mg to 70 mg, 70 mg to 100 mg, 70 mg to 90 mg, 70 mg to 80 mg, 80 mg to 100 mg, 80 mg to 90 mg, or 90 mg to 100 mg) of the anti-TfR1 antibody (e.g., Fab) of the complexes per kg of the subject. In some embodiments, in any one of the methods described herein, the effective amount provides to the subject about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, or 120 mg of the anti-TfR1 antibody (e.g., Fab) of the complexes per kg of the subject.

In some embodiments, in any one of the methods described herein, the effective amount provides to the subject about 8.50 mg, 8.83 mg, 9.18 mg, 9.56 mg, 9.98 mg, 10.43 mg, 10.93 mg, 11.47 mg, 12.08 mg, 12.75 mg, 13.50 mg, 14.34 mg, 17.00 mg, 17.65 mg, 18.36 mg, 19.12 mg, 19.95 mg, 20.86 mg, 21.85 mg, 22.95 mg, 24.15 mg, 25.50 mg, 27.00 mg, 28.68 mg, 34.00 mg, 35.30 mg, 36.71 mg, 38.24 mg, 39.91 mg, 41.72 mg, 43.71 mg, 45.89 mg, 48.31 mg, 50.99 mg, 52.95 mg, 53.99 mg, 55.07 mg, 57.37 mg, 57.37 mg, 59.86 mg, 62.58 mg, 65.56 mg, 67.99 mg, 68.84 mg, 70.61 mg, 72.46 mg, 73.43 mg, 76.49 mg, 79.82 mg, 80.99 mg, 83.44 mg, 86.05 mg, 87.42 mg, 91.79 mg, 96.62 mg, 101.99 mg, 107.99 mg, 114.73 mg of the anti-TfR1 antibody (e.g., Fab) of the complexes per kg of the subject. In some embodiments, in any one of the methods described herein, the effective amount provides to the subject about 11 mg, 22 mg, 44 mg, 66 mg, or 88 mg of the anti-TfR1 antibody (e.g., Fab) of the complexes per kg of the subject. In some embodiments, in any one of the methods described herein, the effective amount provides to the subject about 10.93 mg, 21.85 mg, 43.71 mg, 65.56 mg, or 87.42 mg of the anti-TfR1 antibody (e.g., Fab) of the complexes per kg of the subject. In some embodiments, in any one of the methods described herein, the effective amount provides to the subject about 10.93 mg, 21.86 mg, 43.72 mg, 65.56 mg, or 87.43 mg of the anti-TfR1 antibody (e.g., Fab) of the complexes per kg of the subject.

In some embodiments, in any one of the methods described herein, the effective amount provides to the subject 0.5 mg to 10 mg of the oligonucleotides of the complexes per kg of the subject. For example, in some embodiments, in any one of the methods described herein, the effective amount provides to the subject 0.5 mg to 10 mg, 0.5 mg to 7.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 10 mg, 1 mg to 7.5 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 10 mg, 2 mg to 7.5 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, 4 mg to 10 mg, 4 mg to 7.5 mg, or 4 mg to 5 mg of the oligonucleotides of the complexes per kg of the subject.

In some embodiments, in any one of the methods described herein, the effective amount provides to the subject about 0.5 mg, 1 mg, 2 mg, 3 mg, 4 mg, 5 mg, 6 mg, 7 mg, 8 mg, 9 mg, or 10 mg of the oligonucleotides of the complexes per kg of the subject. In some embodiments, in any one of the methods described herein, the effective amount provides to the subject about 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 3 mg, 4 mg, 4.1 mg, 4.2 mg, 4.3 mg, 4.4 mg, 4.5 mg, 4.6 mg, 4.7 mg, 4.8 mg, 4.9 mg, 5 mg, 6 mg, 7 mg, 8 mg, 9 mg, 10 mg of the complexes per kg of the subject. In some embodiments, in any one of the methods described herein, the effective amount provides to the subject about 0.7 mg, 1.4 mg, or 2.8 mg, of the oligonucleotides of the complexes per kg of the subject.

In some embodiments, in any one of the methods described herein, the effective amount provides to the subject about 0.52 mg, 0.56 mg, 0.59 mg, 0.62 mg, 0.65 mg, 0.69 mg, 0.72 mg, 0.75 mg, 0.78 mg, 0.82 mg, 0.85 mg, 0.88 mg, 1.05 mg, 1.11 mg, 1.18 mg, 1.24 mg, 1.31 mg, 1.37 mg, 1.44 mg, 1.50 mg, 1.57 mg, 1.63 mg, 1.70 mg, 1.76 mg, 2.09 mg, 2.22 mg, 2.35 mg, 2.48 mg, 2.61 mg, 2.75 mg, 2.88 mg, 3.01 mg, 3.14 mg, 3.27 mg, 3.40 mg, 3.53 mg of the oligonucleotides of the complexes per kg of the subject. In some embodiments, in any one of the methods described herein, the effective amount provides to the subject about 0.69 mg, 1.37 mg, or 2.75 mg of the oligonucleotides of the complexes per kg of the subject.

In some embodiments, in any one of the methods described herein, the effective amount provides to the subject 3 mg to 100 mg of the oligonucleotides of the complexes per kg of the subject. For example, in some embodiments, in any one of the methods described herein, the effective amount provides to the subject 3 mg to 100 mg, 3 mg to 55 mg, 3 mg to 50 mg, 3 mg to 10 mg, 5 mg to 80 mg, 5 mg to 50 mg, 5 mg to 40 mg, 10 mg to 70 mg, 10 mg to 50 mg, 10 mg to 30 mg, 20 mg to 60 mg, 20 mg to 40 mg, or 30 mg to 50 mg of the oligonucleotides of the complexes per kg of the subject. In some embodiments, in any one of the methods described herein, the effective amount provides to the subject 3 mg to 52 mg (e.g., about 5 mg, 10 mg, 20 mg, 30 mg, or 40 mg) of the oligonucleotides of the complexes per kg of the subject.

In some embodiments, in any one of the methods described herein, the effective amount provides to the subject about 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg of the oligonucleotides of the complexes per kg of the subject. In some embodiments, in any one of the methods described herein, the effective amount provides to the subject about 3.83 mg, 4.07 mg, 4.31 mg, 4.55 mg, 4.79 mg, 5.03 mg, 5.27 mg, 5.51 mg, 5.75 mg, 5.99 mg, 6.23 mg, 6.47 mg, 7.67 mg, 8.15 mg, 8.63 mg, 9.11 mg, 9.59 mg, 10.07 mg, 10.55 mg, 11.03 mg, 11.50 mg, 11.98 mg, 12.46 mg, 12.94 mg, 15.34 mg, 16.30 mg, 17.26 mg, 18.22 mg, 19.17 mg, 20.13 mg, 21.09 mg, 22.05 mg, 23.01 mg, 23.97 mg, 24.45 mg, 24.93 mg, 25.89 mg, 27.32 mg, 28.76 mg, 30.20 mg, 30.68 mg, 31.64 mg, 32.60 mg, 33.08 mg, 34.51 mg, 35.95 mg, 36.43 mg, 37.39 mg, 38.35 mg, 38.83 mg, 40.27 mg, 42.18 mg, 44.10 mg, 46.02 mg, 47.94 mg, 49.85 mg, or 51.77 mg of the oligonucleotides of the complexes per kg of the subject. In some embodiments, in any one of the methods described herein, the effective amount provides to the subject about 5.03 mg, 10.06 mg, 20.13 mg, 30.2 mg, or 40.27 mg of the oligonucleotides of the complexes per kg of the subject.

In some embodiments, in any one of the methods described herein, the effective amount provides to the subject 5 mg to 100 mg of the oligonucleotides of the complexes per kg of the subject. For example, in some embodiments, in any one of the methods described herein, the effective amount provides to the subject 5 mg to 100 mg, 5 mg to 90 mg, 5 mg to 80 mg, 5 mg to 70 mg, 5 mg to 60 mg, 5 mg to 50 mg, 5 mg to 40 mg, 5 mg to 30 mg, 5 mg to 20 mg, 5 mg to 10 mg, 8 mg to 32 mg, 8 mg to 31 mg, 8 mg to 30 mg, 8 mg to 29 mg, 8 mg to 28 mg, 9 mg to 32 mg, 9 mg to 31 mg, 9 mg to 30 mg, 9 mg to 29 mg, 9 mg to 28 mg, 10 mg to 100 mg, 10 mg to 90 mg, 10 mg to 80 mg, 10 mg to 70 mg, 10 mg to 60 mg, 10 mg to 50 mg, 10 mg to 40 mg, 10 mg to 32 mg, 10 mg to 31 mg, 10 mg to 30 mg, 10 mg to 29 mg, 10 mg to 28 mg, 10 mg to 20 mg, 11 mg to 32 mg, 11 mg to 31 mg, 11 mg to 30 mg, 11 mg to 29 mg, 11 mg to 28 mg, 12 mg to 32 mg, 12 mg to 31 mg, 12 mg to 30 mg, 12 mg to 29 mg, 12 mg to 28 mg, 20 mg to 100 mg, 20 mg to 90 mg, 20 mg to 80 mg, 20 mg to 70 mg, 20 mg to 60 mg, 20 mg to 50 mg, 20 mg to 40 mg, 20 mg to 30 mg, 30 mg to 100 mg, 30 mg to 90 mg, 30 mg to 80 g, 30 mg to 70 mg, 30 mg to 60 mg, 30 mg to 50 mg, 30 mg to 40 mg, 40 mg to 100 mg, 40 mg to 90 mg, 40 mg to 80 mg, 40 mg to 70 mg, 40 mg to 60 mg, 40 mg to 50 mg, 50 mg to 100 mg, 50 mg to 90 mg, 50 mg to 80 mg, 50 mg to 70 mg, 50 mg to 60 mg, 60 mg to 100 mg, 60 mg to 90 mg, 60 mg to 80 mg, 60 mg to 70 mg, 70 mg to 100 mg, 70 mg to 90 mg, 70 mg to 80 mg, 80 mg to 100 mg, 80 mg to 90 mg, or 90 mg to 100 mg of the oligonucleotides of the complexes per kg of the subject. In some embodiments, in any one of the methods described herein, the effective amount provides to the subject about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg of the oligonucleotides of the complexes per kg of the subject. The amount of complexes administered to the subject such that the subject is provided with the effective amount of oligonucleotides as described herein is more per kg of the subject's weight since the complex also includes an antibody covalently linked to the oligonucleotide.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, and administering approximately 100 mg of complex per kg of the subject provides 30 mg of oligonucleotide per kg of the subject. In some embodiments, a method provided herein comprises administering to the subject a composition comprising complexes comprising any one of the structures of formula (I): [R¹]ₙ₁—R² as described herein, wherein the average value of n1 of the complexes of the composition is 2, and administering approximately 30 mg of complex per kg of the subject provides 10 mg of oligonucleotide per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, and administering approximately 16 mg of complex per kg of the subject provides 5 mg of oligonucleotide per kg of the subject. In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, and administering approximately 32 mg of complex per kg of the subject provides 10 mg of oligonucleotide per kg of the subject. In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, and administering approximately 64 mg of complex per kg of the subject provides 20 mg of oligonucleotide per kg of the subject. In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, and administering approximately 96 mg of complex per kg of the subject provides 30 mg of oligonucleotide per kg of the subject. In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, and administering approximately 128 mg of complex per kg of the subject provides 40 mg of oligonucleotide per kg of the subject.

In some aspects, provided herein are methods of promoting expression or activity of a dystrophin protein in a subject. In some aspects, provided herein are methods of treating Duchenne Muscular Dystrophy (DMD) in a subject. In some embodiments, methods provided herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 0.5 mg to 100 mg (e.g., 0.5 mg to 100 mg, 0.5 mg to 90 mg, 0.5 mg to 80 mg, 0.5 mg to 70 mg, 0.5 mg to 60 mg, 0.5 mg to 50 mg, 0.5 mg to 40 mg, 0.5 mg to 30 mg, 0.5 mg to 20 mg, 0.5 mg to 10 mg, 0.5 mg to 7.5 mg, 0.5 to 5 mg, 5 mg to 100 mg, 5 mg to 80 mg, 5 mg to 50 mg, 5 mg to 40 mg, 5 mg to 30 mg, 5 mg to 20 mg, 10 mg to 60 mg, 10 mg to 50 mg, 10 mg to 40 mg, 10 mg to 30 mg, or 30 mg to 60 mg) of the oligonucleotides of the complexes per kg of the subject, wherein the antibody (e.g., Fab) comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the base sequence of CTCCAACATCAAGGAAGATGGCATTTCTAG (SEQ ID NO: 21) and is a phosphorodiamidate morpholino oligomer (PMO), optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20.

In some embodiments, methods provided herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 0.7 mg, 1.4 mg, 2.8 mg, 5 mg, 10 mg, 20 mg, or 40 mg of the oligonucleotides of the complexes per kg of the subject. In some embodiments, methods provided herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 1 mg to 90 mg (e.g., 1 mg to 90 mg, 5 mg to 80 mg, 5 mg to 50 mg, 5 mg to 40 mg, 5 mg to 30 mg, 5 mg to 20 mg, 10 mg to 60 mg, 10 mg to 50 mg, 10 mg to 40 mg, 10 mg to 30 mg, or 30 mg to 60 mg) of the anti-TfR1 antibody (e.g., Fab) of the complexes per kg of the subject, wherein the antibody (e.g., Fab) comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the base sequence of CTCCAACATCAAGGAAGATGGCATTTCTAG (SEQ ID NO: 21) and is a phosphorodiamidate morpholino oligomer (PMO), optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20. In some embodiments, the effective amount of muscle targeting complexes is 1.5 mg, 3 mg, 6 mg, 11 mg, 22 mg, 44 mg, or 88 mg of the anti-TfR1 antibody (e.g., Fab) of the complexes per kg of the subject.

In some aspects, provided herein are methods of promoting expression or activity of a dystrophin protein in a subject. In some aspects, provided herein are methods of treating Duchenne Muscular Dystrophy (DMD) in a subject. In some embodiments, methods provided herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 5 mg to 100 mg (e.g., 5 mg to 100 mg, 5 mg to 80 mg, 5 mg to 50 mg, 5 mg to 40 mg, 5 mg to 30 mg, 5 mg to 20 mg, 10 mg to 60 mg, 10 mg to 50 mg, 10 mg to 40 mg, 10 mg to 30 mg, or 30 mg to 60 mg) of the oligonucleotides of the complexes per kg of the subject, wherein the antibody (e.g., Fab) comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the base sequence of CTCCAACATCAAGGAAGATGGCATTTCTAG (SEQ ID NO: 21) and is a phosphorodiamidate morpholino oligomer (PMO), optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20.

In some embodiments, methods provided herein comprise administering to the subject a composition comprising an effective amount of muscle targeting complexes, each complex comprising an anti-transferrin receptor 1 (TfR1) antibody covalently linked to one or more oligonucleotides, wherein the effective amount provides to the subject 1 mg to 90 mg (e.g., 1 mg to 90 mg, 5 mg to 80 mg, 5 mg to 50 mg, 5 mg to 40 mg, 5 mg to 30 mg, 5 mg to 20 mg, 10 mg to 60 mg, 10 mg to 50 mg, 10 mg to 40 mg, 10 mg to 30 mg, or 30 mg to 60 mg) of the anti-TfR1 antibody (e.g., Fab) of the complexes per kg of the subject, wherein the antibody (e.g., Fab) comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the base sequence of CTCCAACATCAAGGAAGATGGCATTTCTAG (SEQ ID NO: 21) and is a phosphorodiamidate morpholino oligomer (PMO), optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20. In some embodiments, the effective amount of muscle targeting complexes is 1.5 mg, 3 mg, 6 mg, 11 mg, 22 mg, 44 mg, or 88 mg of the anti-TfR1 antibody (e.g., Fab) of the complexes per kg of the subject.

In some embodiments, methods provided herein comprise administering to the subject a composition comprising an effective amount of muscle targeting complexes, wherein the effective amount provides to the subject 0.5 mg to 10 mg (e.g., 0.5 mg to 10 mg, 0.5 mg to 7.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 10 mg, 1 mg to 7.5 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 10 mg, 2 mg to 7.5 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, 4 mg to 10 mg, 4 mg to 7.5 mg, or 4 mg to 5 mg) of the oligonucleotides of the complexes per kg of the subject, wherein each complex comprises a structure of formula (I): [R¹]ₙ₁—R², wherein:
each R¹ comprises a group of the formula (Ib):

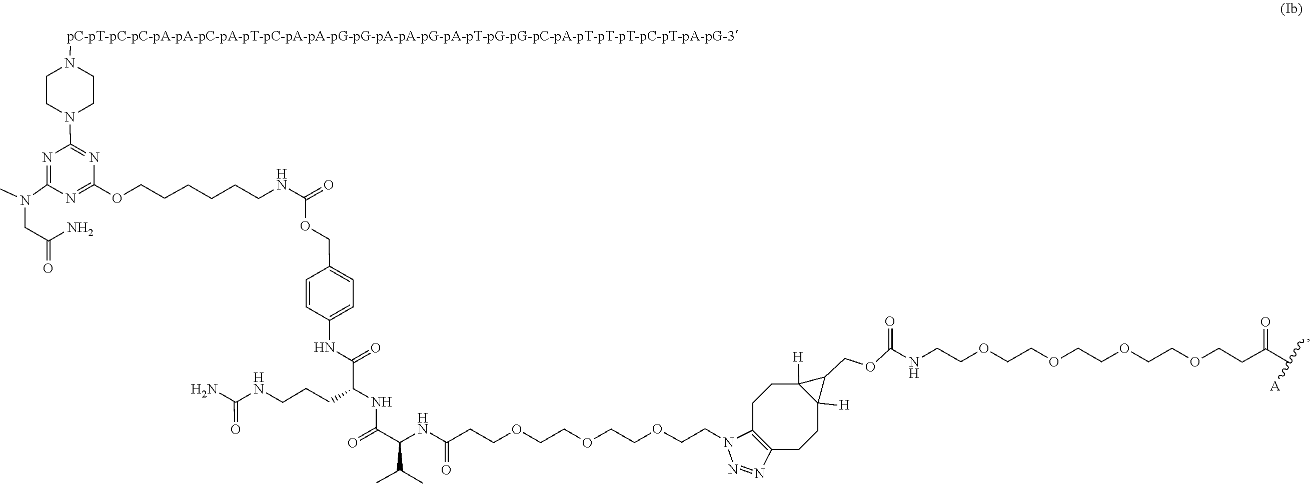

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked (e.g., indirectly or directly linked, e.g., directly linked) at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, the effective amount provides to the subject 0.7 mg, 1.4 mg, or 2.8 mg of the oligonucleotides of the complexes per kg of the subject. In some embodiments, the effective amount provides to the subject 0.69 mg, 1.37 mg, or 2.75 mg of the oligonucleotides of the complexes per kg of the subject. In some embodiments, compositions (e.g., in aqueous solutions) for administration to a subject in the methods described herein further comprise complexes that comprise a structure of formula (I): [R¹]ₙ₁—R², wherein n1 is 0 and wherein each R¹ and R² are defined herein.

In some embodiments, methods provided herein comprise administering to the subject a composition comprising an effective amount of muscle targeting complexes, wherein the effective amount provides to the subject 0.5 mg to 10 mg (e.g., 0.5 mg to 10 mg, 0.5 mg to 7.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 10 mg, 1 mg to 7.5 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 10 mg, 2 mg to 7.5 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, 4 mg to 10 mg, 4 mg to 7.5 mg, or 4 mg to 5 mg) of the oligonucleotides of the complexes per kg of the subject, wherein each complex comprises a structure of formula (I): [R¹]ₙ₁—R², wherein:
each R¹ comprises a group of the formula (Ic):

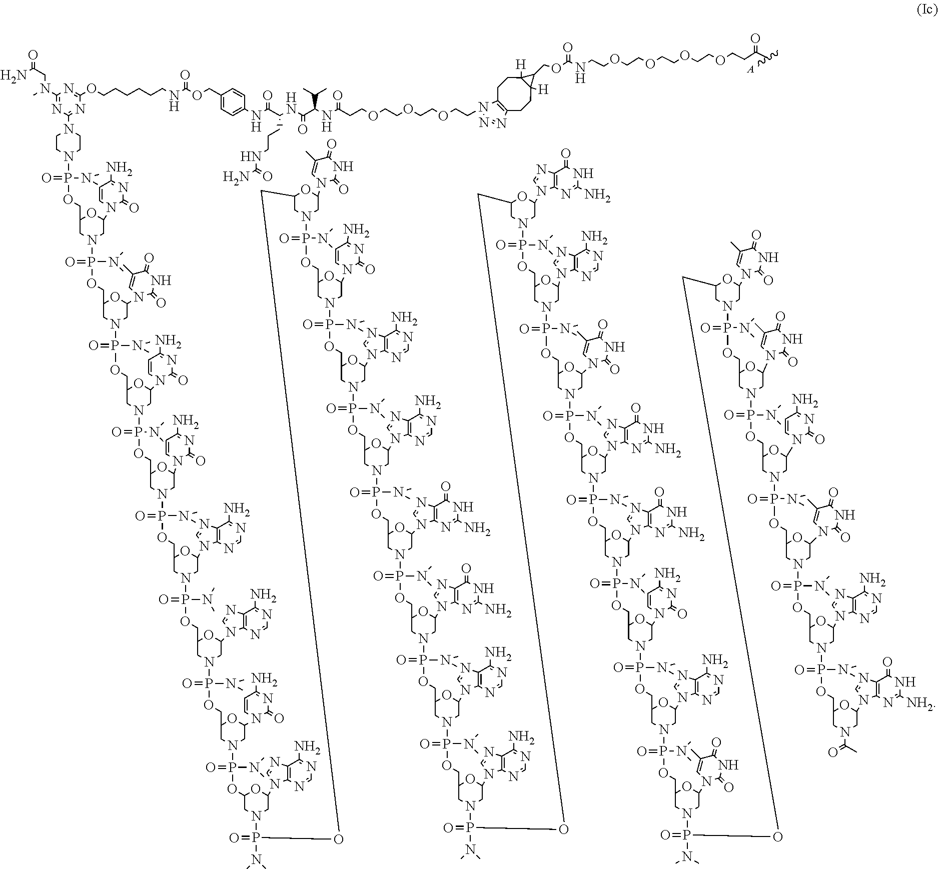

R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ TD NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked (e.g., indirectly or directly linked, e.g., directly linked) at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, the effective amount provides to the subject 0.7 mg, 1.4 mg, or 2.8 mg of the oligonucleotides of the complexes per kg of the subject. In some embodiments, the effective amount provides to the subject 0.69 mg, 1.37 mg, or 2.75 mg of the oligonucleotides of the complexes per kg of the subject. In some embodiments, compositions (e.g., in aqueous solutions) for administration to a subject in the methods described herein further comprise complexes that comprise a structure of formula (I): [R¹]ₙ₁—R², wherein n1 is 0 and wherein each R¹ and R² are defined herein.

In some embodiments, methods provided herein comprise administering to the subject a composition comprising an effective amount of muscle targeting complexes, wherein the effective amount provides to the subject 0.5 mg to 10 mg (e.g., 0.5 mg to 10 mg, 0.5 mg to 7.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 10 mg, 1 mg to 7.5 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 10 mg, 2 mg to 7.5 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, 4 mg to 10 mg, 4 mg to 7.5 mg, or 4 mg to 5 mg) of the oligonucleotides of the complexes per kg of the subject, wherein each complex comprises a structure of formula (Id):

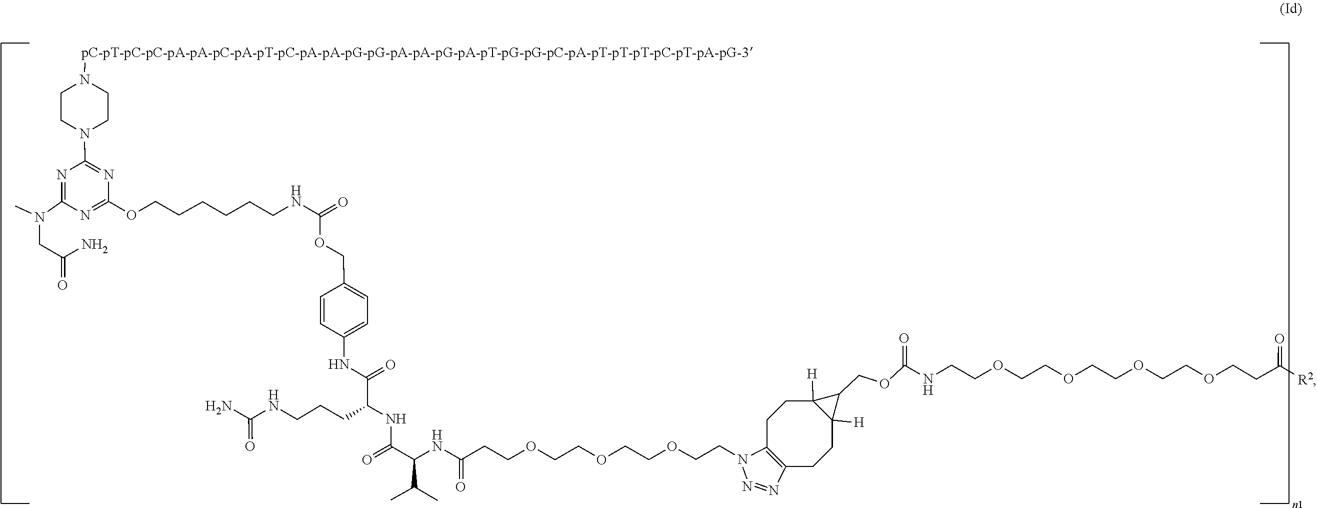

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21);
R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20; and wherein in each complex n1 is independently an integer of one or greater, optionally wherein the average value of n1 of the complexes of the composition is in the range of 1 to 5, optionally wherein the anti-TfR1 antibody (e.g., Fab) covalently linked (e.g., indirectly or directly linked, e.g., directly linked) via different amino acid residue of the antibody (e.g., Fab), optionally wherein each different amino acid residue is a lysine. In some embodiments, the effective amount provides to the subject 0.7, 1.4, or 2.8 mg of the oligonucleotides of the complexes per kg of the subject. In some embodiments, the effective amount provides to the subject 0.69 mg, 1.37 mg, or 2.75 mg of the oligonucleotides of the complexes per kg of the subject. In some embodiments, compositions (e.g., in aqueous solutions) for administration to a subject in the methods described herein further comprise complexes in which n1 is 0.

In some embodiments, methods provided herein comprise administering to the subject a composition comprising an effective amount of muscle targeting complexes, wherein the effective amount provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject, wherein each complex comprises a structure of formula (I): [R¹]ₙ₁—R², wherein:
each R¹ comprises a group of the formula (Ib):

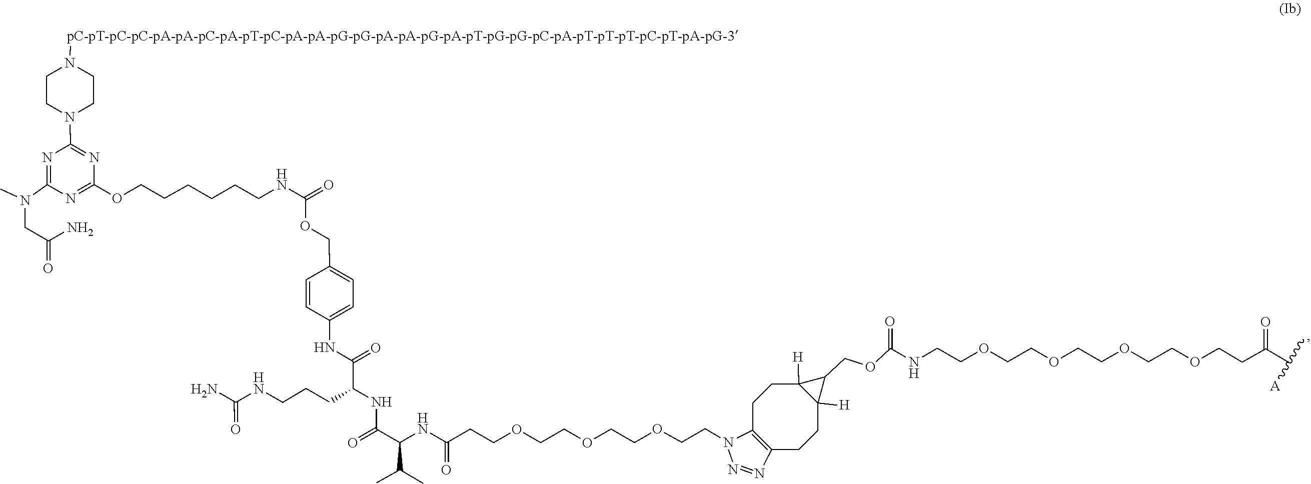

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked (e.g., indirectly or directly linked, e.g., directly linked) at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, compositions (e.g., in aqueous solutions) for administration to a subject in the methods described herein further comprise complexes that comprise a structure of formula (I): [R¹]ₙ₁—R², wherein n1 is 0 and wherein each R¹ and R² are defined herein.

In some embodiments, methods provided herein comprise administering to the subject a composition comprising an effective amount of muscle targeting complexes, wherein the effective amount provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject, wherein each complex comprises a structure of formula (I): [R¹]ₙ₁—R², wherein:
each R¹ comprises a group of the formula (Ic):

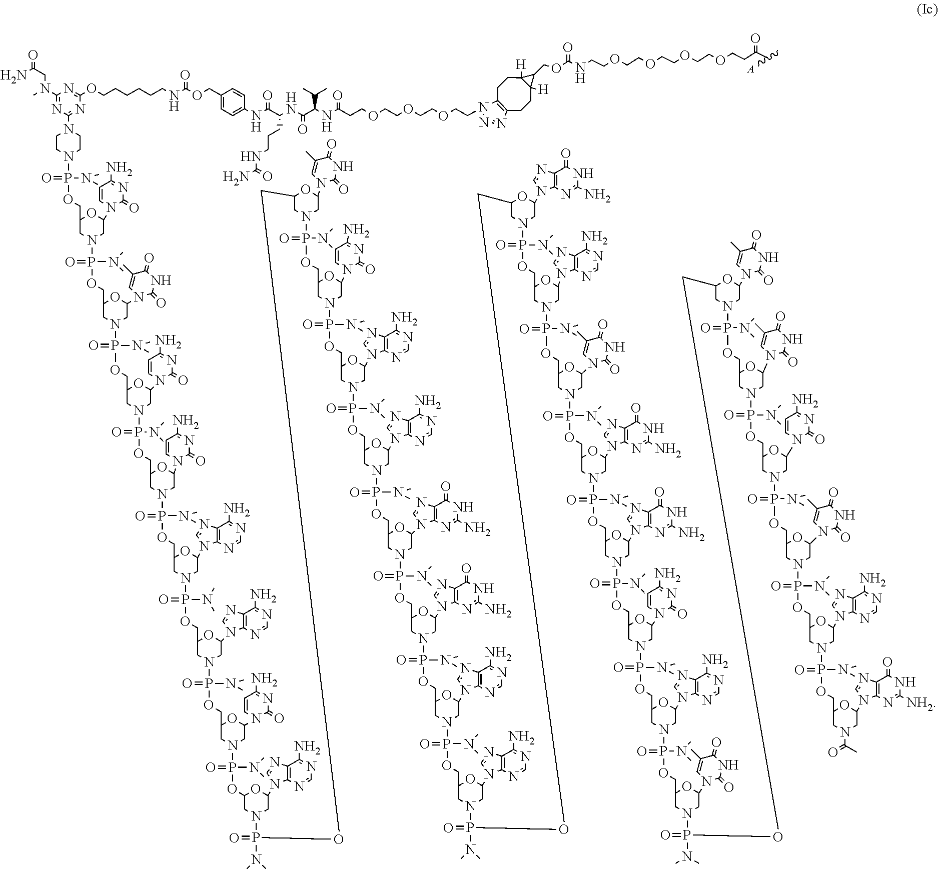

R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked (e.g., indirectly or directly linked, e.g., directly linked) at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, compositions (e.g., in aqueous solutions) for administration to a subject in the methods described herein further comprise complexes that comprise a structure of formula (I): [R¹]ₙ₁—R², wherein n1 is 0 and wherein each R¹ and R² are defined herein.

In some embodiments, methods provided herein comprise administering to the subject a composition comprising an effective amount of muscle targeting complexes, wherein the effective amount provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject, wherein each complex comprises a structure of formula (Id):

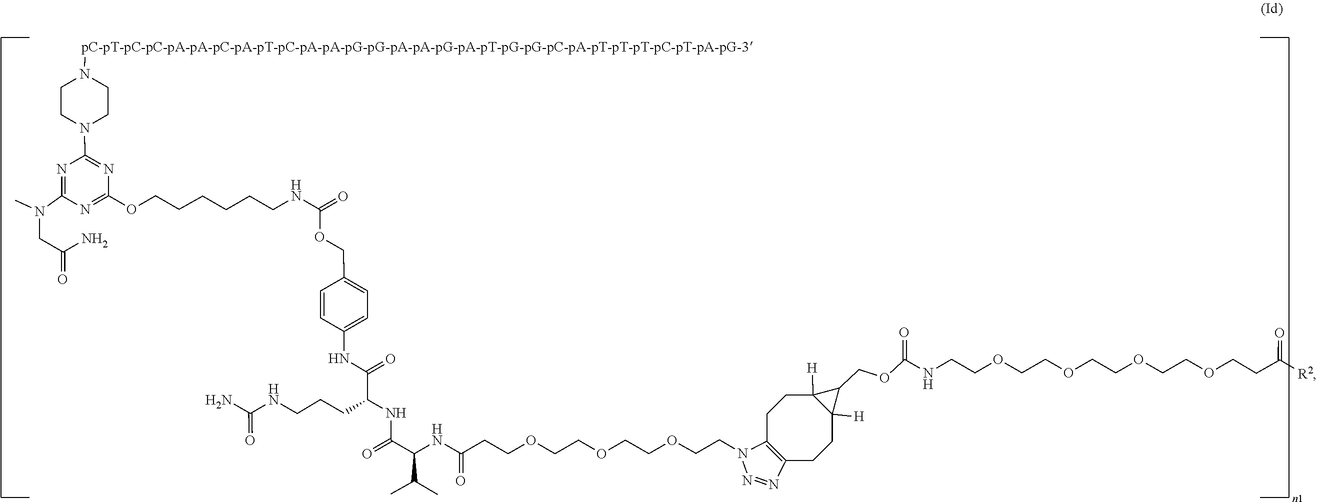

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21);
   R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20; and
   wherein in each complex n1 is independently an integer of one or greater, optionally wherein the average value of n1 of the complexes of the composition is in the range of 1 to 5, optionally wherein the anti-TfR1 antibody (e.g., Fab) covalently linked (e.g., indirectly or directly linked, e.g., directly linked) via different amino acid residue of the antibody (e.g., Fab), optionally wherein each different amino acid residue is a lysine. In some embodiments, compositions (e.g., in aqueous solutions) for administration to a subject in the methods described herein further comprise complexes in which n1 is 0.

In some embodiments, methods provided herein comprise administering to the subject a composition comprising an effective amount of muscle targeting complexes, wherein the effective amount provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject, wherein each complex comprises a structure of formula (I): [R¹]ₙ₁—R², wherein:
each R¹ comprises a group of the formula (Tb):

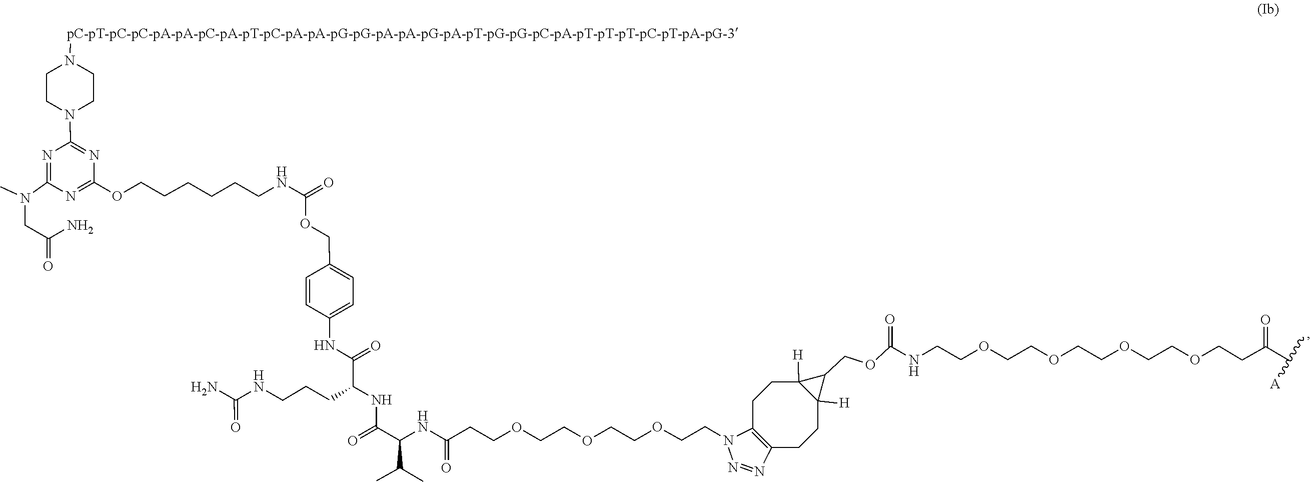

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody comprises a VH comprising the amino acid sequence of SEQ ID NO: 17 and a VL comprising the amino acid sequence of SEQ ID NO: 18, further optionally wherein the antibody comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked (e.g., indirectly or directly linked, e.g., directly linked) at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, the effective amount provides to the subject 5 mg, 10 mg, 20 mg, 30 mg, or 40 mg of the oligonucleotides of the complexes per kg of the subject. In some embodiments, the effective amount provides to the subject 5.03 mg, 10.06 mg, 20.13 mg, 30.2 mg, or 40.27 mg of the oligonucleotides of the complexes per kg of the subject. In some embodiments, compositions (e.g., in aqueous solutions) for administration to a subject in the methods described herein further comprise complexes that comprise a structure of formula (I): [R¹]ₙ₁—R², wherein n1 is 0 and wherein each R¹ and R² are defined herein.

In some embodiments, methods provided herein comprise administering to the subject a composition comprising an effective amount of muscle targeting complexes, wherein the effective amount provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject, wherein each complex comprises a structure of formula (I): [R¹]ₙ₁—R², wherein:
each R¹ comprises a group of the formula (Ic):

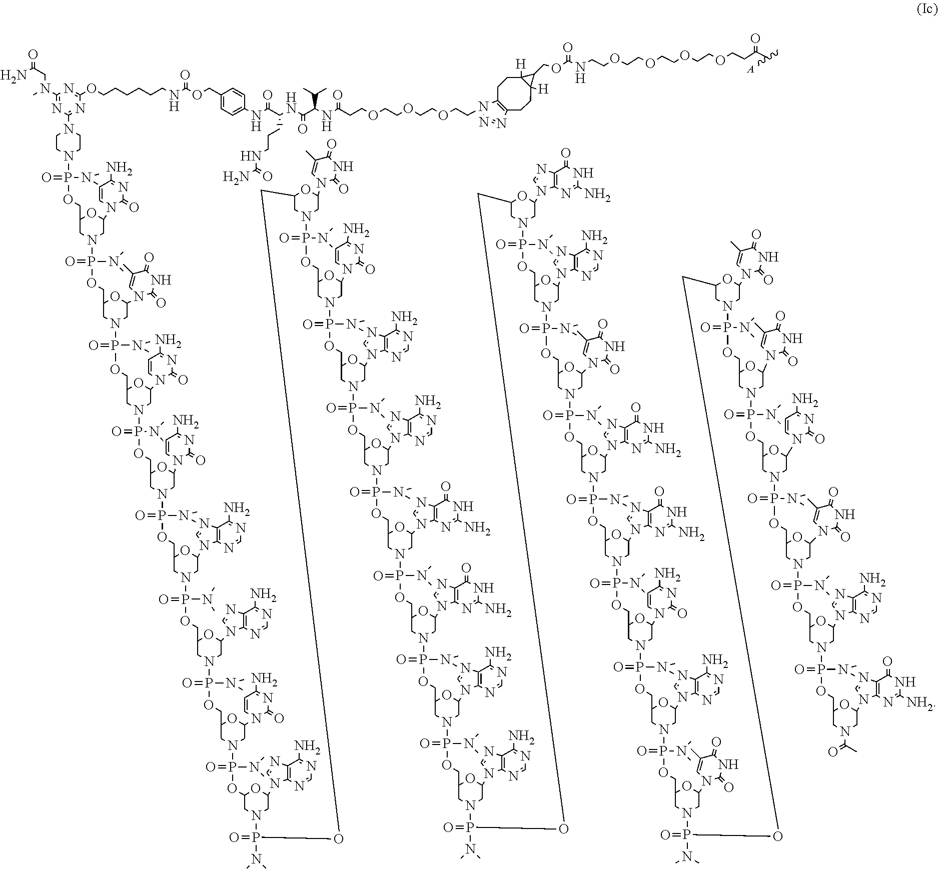

R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20;
wherein each R¹ is covalently linked to R² at attachment point A, optionally wherein each R¹ is covalently linked (e.g., indirectly or directly linked, e.g., directly linked) at attachment point A to a different amino acid residue of the antibody (e.g. Fab) of R², optionally wherein each different amino acid residue is a lysine; and
wherein in each complex n1 is independently an integer of one or greater representing the number of instances of R¹, optionally wherein the average value of n1 of the complexes of the composition is in the range of 1 to 5. In some embodiments, the effective amount provides to the subject 5 mg, 10 mg, 20 mg, 30 mg, or 40 mg of the oligonucleotides of the complexes per kg of the subject. In some embodiments, the effective amount provides to the subject 5.03 mg, 10.06 mg, 20.13 mg, 30.2 mg, or 40.27 mg of the oligonucleotides of the complexes per kg of the subject. In some embodiments, compositions (e.g., in aqueous solutions) for administration to a subject in the methods described herein further comprise complexes that comprise a structure of formula (I): [R¹]ₙ₁—R², wherein n1 is 0 and wherein each R¹ and R² are defined herein.

In some embodiments, methods provided herein comprise administering to the subject a composition comprising an effective amount of muscle targeting complexes, wherein the effective amount provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject, wherein each complex comprises a structure of formula (Id):

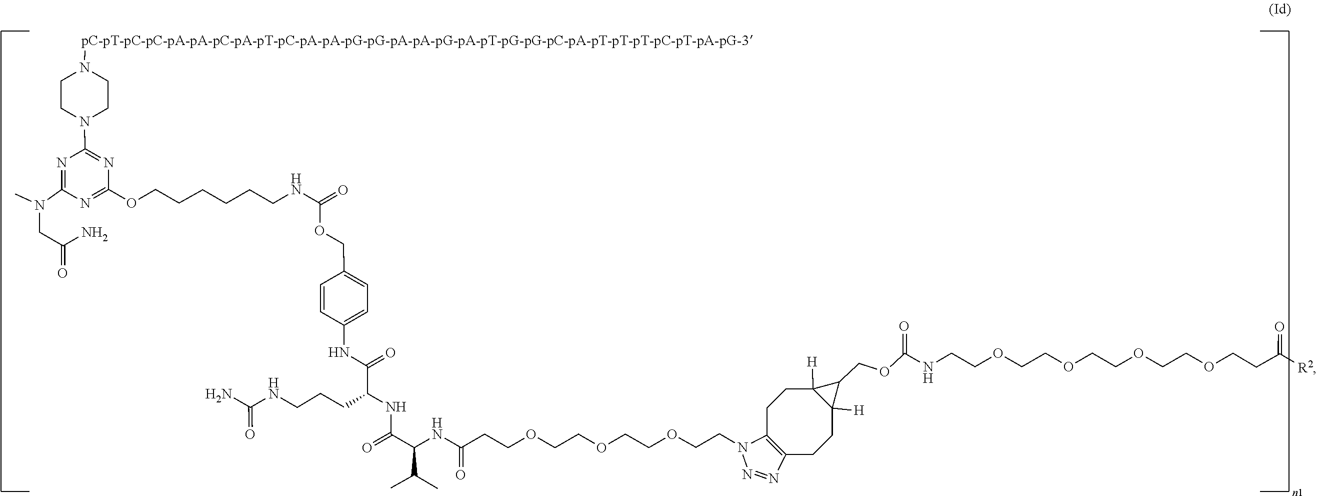

in which -pN indicates a base position of a phosphorodiamidate morpholino oligomer (PMO); wherein -p reflects a phosphorodiamidate linkage, and wherein N corresponds to a nucleobase of adenine (A), cytosine (C), guanine (G), or thymine (T), such that the PMO comprises a base sequence of CTCCAACATCAAGGAAGATGGCATTTCTAG (SEQ ID NO: 21);
   R² comprises an anti-TfR1 antibody (e.g., Fab) comprising: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, optionally wherein the antibody (e.g., Fab) comprises a VH comprising the amino acid sequence of SEQ ID NO: 17 and a VL comprising the amino acid sequence of SEQ ID NO: 18, further optionally wherein the antibody (e.g., Fab) comprises a heavy chain comprising the amino acid sequence of SEQ ID NO: 19 and a light chain comprising the amino acid sequence of SEQ ID NO: 20; and
wherein in each complex n1 is independently an integer of one or greater, optionally wherein the average value of n1 of the complexes of the composition is in the range of 1 to 5, optionally wherein the anti-TfR1 antibody (e.g., Fab) covalently linked (e.g., indirectly or directly linked, e.g., directly linked) via different amino acid residue of the antibody (e.g., Fab), optionally wherein each different amino acid residue is a lysine. In some embodiments, the effective amount provides to the subject 5 mg, 10 mg, 20 mg, 30 mg, or 40 mg of the oligonucleotides of the complexes per kg of the subject. In some embodiments, the effective amount provides to the subject 5.03 mg, 10.06 mg, 20.13 mg, 30.2 mg, or 40.27 mg of the oligonucleotides of the complexes per kg of the subject. In some embodiments, compositions (e.g., in aqueous solutions) for administration to a subject in the methods described herein further comprise complexes in which n1 is 0.

In some embodiments, in any one of the methods described herein, the composition is in an aqueous solution and further comprises histidine and sucrose, wherein the histidine is present in the aqueous solution at a concentration of 25 mM, the sucrose is present in the aqueous solution at a concentration of 10 w/v %, and the composition is at a pH of 6.0. In some embodiments, the complexes are present in the composition at a concentration in the range of 10 mg/mL to 50 mg/mL (e.g., 10 mg/mL, 15 mg/mL, 20 mg/mL, 25 mg/mL, 30 mg/mL, 35 mg/mL, 40 mg/mL, 45 mg/mL, 50 mg/mL).

In some embodiments, the subject is administered a single dose of any one of the compositions comprising an effective amount (e.g., providing to the subject 0.5 mg to 10 mg (e.g., 0.5 mg to 10 mg, 0.5 mg to 7.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 10 mg, 1 mg to 7.5 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 10 mg, 2 mg to 7.5 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, 4 mg to 10 mg, 4 mg to 7.5 mg, or 4 mg to 5 mg) of the oligonucleotides of the complexes per kg of the subject) of the complexes described herein. In some embodiments, the subject is administered a single dose of any one of the compositions comprising an effective amount (e.g., providing to the subject 0.5 mg to 5 mg (e.g., 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 3 mg, 4 mg, 4.1 mg, 4.2 mg, 4.3 mg, 4.4 mg, 4.5 mg, 4.6 mg, 4.7 mg, 4.8 mg, 4.9 mg, 5 mg) of the oligonucleotides of the complexes per kg of the subject) of the complexes described herein. In some embodiments, the subject is administered a single dose of any one of the compositions comprising an effective amount (e.g., providing to the subject 0.7 mg, 1.4, mg, or 2.8 mg of the oligonucleotides of the complexes per kg of the subject) of the complexes described herein. In some embodiments, the subject is administered a single dose of any one of the compositions comprising an effective amount (e.g., providing to the subject 0.69 mg, 1.37 mg, or 2.75 mg of the oligonucleotides of the complexes per kg of the subject) of the complexes described herein. In some embodiments, the subject is administered a single dose of any one of the compositions comprising an effective amount (e.g., providing to the subject 5 mg to 100 mg (e.g., 5 mg, 10 mg, 20 mg, 30 mg, 40 mg, 50 mg, 60 mg, 70 mg, 80 mg, 90 mg, or 100 mg) of the oligonucleotides of the complexes per kg of the subject) of the complexes described herein. In some embodiments, the subject is administered a single dose of any one of the compositions comprising an effective amount (e.g., providing to the subject about 3 mg to 52 mg (e.g., 3.83 mg, 4.07 mg, 4.31 mg, 4.55 mg, 4.79 mg, 5.03 mg, 5.27 mg, 5.51 mg, 5.75 mg, 5.99 mg, 6.23 mg, 6.47 mg, 7.67 mg, 8.15 mg, 8.63 mg, 9.11 mg, 9.59 mg, 10.07 mg, 10.55 mg, 11.03 mg, 11.50 mg, 11.98 mg, 12.46 mg, 12.94 mg, 15.34 mg, 16.30 mg, 17.26 mg, 18.22 mg, 19.17 mg, 20.13 mg, 21.09 mg, 22.05 mg, 23.01 mg, 23.97 mg, 24.45 mg, 24.93 mg, 25.89 mg, 27.32 mg, 28.76 mg, 30.20 mg, 30.68 mg, 31.64 mg, 32.60 mg, 33.08 mg, 34.51 mg, 35.95 mg, 36.43 mg, 37.39 mg, 38.35 mg, 38.83 mg, 40.27 mg, 42.18 mg, 44.10 mg, 46.02 mg, 47.94 mg, 49.85 mg, or 51.77 mg) of the oligonucleotides of the complexes per kg of the subject) of the complexes described herein. In some embodiments, the subject is administered a single dose of any one of the compositions comprising an effective amount (e.g., providing to the subject 5.03 mg, 10.06 mg, 20.13 mg, 30.2 mg, or 40.27 mg of the oligonucleotides of the complexes per kg of the subject) of the complexes described herein.

In some embodiments, the subject is administered multiple doses of any one of the compositions comprising the complexes described herein. In some embodiments, a method described herein comprises administering to the subject a composition comprising an effective amount (e.g., providing to the subject 0.5 mg to 10 mg (e.g., 0.5 mg to 10 mg, 0.5 mg to 7.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 10 mg, 1 mg to 7.5 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 10 mg, 2 mg to 7.5 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, 4 mg to 10 mg, 4 mg to 7.5 mg, or 4 mg to 5 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, the subject is administered multiple doses of any one of the compositions comprising the complexes described herein.

In some embodiments, a method described herein comprises administering to the subject a composition comprising an effective amount (e.g., providing to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg)) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 0.7 mg, 1.4 mg, or 2.8 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 0.69 mg, 1.37 mg, or 2.75 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime.

In some embodiments, a method described herein comprises administering to the subject a composition comprising an effective amount of complexes (e.g., muscle targeting complexes) described herein once every 2 weeks, wherein the effective amount for each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount of complexes (e.g., muscle targeting complexes) described herein once every 4 weeks, wherein the effective amount for each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 10 mg, 3 mg to 7.5 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount of complexes (e.g., muscle targeting complexes) described herein once every 8 weeks, wherein the effective amount for each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount of complexes (e.g., muscle targeting complexes) described herein once every 12 weeks, wherein the effective amount for each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%).

In some embodiments, the subject is administered multiple doses of any one of the compositions comprising the complexes described herein. In some embodiments, a method described herein comprises administering to the subject a composition comprising an effective amount (e.g., providing to the subject 5 mg to 100 mg (e.g., 5 mg to 100 mg, 5 mg to 80 mg, 10 mg to 60 mg, 10 mg to 30 mg, or 30 mg to 60 mg) of the oligonucleotides of the complexes per kg of the subject) of complexes (e.g., muscle targeting complexes) described herein once a week to once every four months. For example, in some embodiments, a method described herein comprises administering to the subject a composition comprising an effective amount (e.g., providing to the subject 5 mg to 100 mg (e.g., 5 mg to 100 mg, 5 mg to 80 mg, 10 mg to 60 mg, 10 mg to 30 mg, or 30 mg to 60 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, the subject is administered multiple doses of any one of the compositions comprising the complexes described herein. In some embodiments, a method described herein comprises administering to the subject a composition comprising an effective amount (e.g., providing to the subject 3 mg to 52 mg (e.g., 3 mg to 52 mg, 5 mg to 50 mg, 10 mg to 40 mg, 20 mg to 30 mg) of the oligonucleotides of the complexes per kg of the subject) of complexes (e.g., muscle targeting complexes) described herein once a week to once every four months. For example, in some embodiments, a method described herein comprises administering to the subject a composition comprising an effective amount (e.g., providing to the subject 3 mg to 52 mg (e.g., 3 mg to 52 mg, 5 mg to 50 mg, 10 mg to 40 mg, 20 mg to 30 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime.

In some embodiments, a method described herein comprises administering to the subject a composition comprising an effective amount (e.g., providing to the subject about 3.83 mg, 4.07 mg, 4.31 mg, 4.55 mg, 4.79 mg, 5.03 mg, 5.27 mg, 5.51 mg, 5.75 mg, 5.99 mg, 6.23 mg, 6.47 mg, 7.67 mg, 8.15 mg, 8.63 mg, 9.11 mg, 9.59 mg, 10.07 mg, 10.55 mg, 11.03 mg, 11.50 mg, 11.98 mg, 12.46 mg, 12.94 mg, 15.34 mg, 16.30 mg, 17.26 mg, 18.22 mg, 19.17 mg, 20.13 mg, 21.09 mg, 22.05 mg, 23.01 mg, 23.97 mg, 24.45 mg, 24.93 mg, 25.89 mg, 27.32 mg, 28.76 mg, 30.20 mg, 30.68 mg, 31.64 mg, 32.60 mg, 33.08 mg, 34.51 mg, 35.95 mg, 36.43 mg, 37.39 mg, 38.35 mg, 38.83 mg, 40.27 mg, 42.18 mg, 44.10 mg, 46.02 mg, 47.94 mg, 49.85 mg, or 51.77 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 5.03 mg, 10.06 mg, 20.13 mg, 30.2 mg or 40.27 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 5 mg, 10 mg, 20 mg, or 40 mg) of the oligonucleotides of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime.

In some embodiments, a method described herein comprises administering to the subject a composition comprising an effective amount of complexes (e.g., muscle targeting complexes) described herein once every 2 weeks, wherein the effective amount for each administration provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject. In some embodiments, a method described herein comprises administering to the subject a composition comprising an effective amount of complexes (e.g., muscle targeting complexes) described herein once every 2 weeks, wherein the effective amount for each administration provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject. In some embodiments, a method described herein comprises administering to the subject a composition comprising an effective amount (e.g., providing to the subject about 0.7 mg, 1.4 mg, 2.8 mg, 5 mg, 10 mg, 20 mg, or 40 mg) of the oligonucleotides of the complexes per kg of the subject) of complexes (e.g., muscle targeting complexes) described herein once every 2 weeks.

In some embodiments, a method described herein comprises administering to the subject a composition comprising an effective amount of complexes (e.g., muscle targeting complexes) described herein once every 4 weeks, wherein the effective amount for each administration provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject. In some embodiments, a method described herein comprises administering to the subject a composition comprising an effective amount (e.g., providing to the subject about 0.7 mg, 1.4 mg, 2.8 mg, 5 mg, 10 mg, 20 mg, or 40 mg) of the oligonucleotides of the complexes per kg of the subject) of complexes (e.g., muscle targeting complexes) described herein once every 4 weeks.

In some embodiments, a method described herein comprises administering to the subject a composition comprising an effective amount of complexes (e.g., muscle targeting complexes) described herein once every 8 weeks, wherein the effective amount for each administration provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject. In some embodiments, a method described herein comprises administering to the subject a composition comprising an effective amount (e.g., providing to the subject about 0.7 mg, 1.4 mg, 2.8 mg, 5 mg, 10 mg, 20 mg, or 40 mg) of the oligonucleotides of the complexes per kg of the subject) of complexes (e.g., muscle targeting complexes) described herein once every 8 weeks.

In some embodiments, a method described herein comprises administering to the subject a composition comprising an effective amount of complexes (e.g., muscle targeting complexes) described herein once every 12 weeks, wherein the effective amount for each administration provides to the subject 8 mg to 36 mg (e.g., 8 mg to 36 mg, 8 mg to 30 mg, 8 mg to 25 mg, 8 mg to 20 mg, 8 mg to 15 mg, 8 mg to 12 mg, 8 mg to 10 mg, 10 mg to 36 mg, 10 mg to 30 mg, 10 mg to 25 mg, 10 mg to 20 mg, 10 mg to 15 mg, 15 mg to 36 mg, 15 mg to 30 mg, 15 mg to 25 mg, 15 mg to 20 mg, 20 mg to 36 mg, 20 mg to 30 mg, 20 mg to 25 mg, 25 mg to 36 mg, 25 mg to 20 mg, 25 mg to 30 mg, or 24 mg to 36 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject 3 mg to 52 mg (e.g., 3 mg to 52 mg, 3 mg to 50 mg, 3 mg to 40 mg, 3 mg to 30 mg, 3 mg to 20 mg, 3 mg to 10 mg, 3 mg to 5 mg, 5 mg to 52 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 52 mg, 10 mg to 50 mg, 10 mg to 40 mg, 10 mg to 30 mg, 10 mg to 20 mg, 20 mg to 52 mg, 20 mg to 50 mg, 20 mg to 40 mg, 20 mg to 30 mg, 30 mg to 50 mg, 30 mg to 40 mg, 40 mg to 52 mg, or 40 mg to 50 mg) of the oligonucleotides of the complexes per kg of the subject. In some embodiments, a method described herein comprises administering to the subject a composition comprising an effective amount (e.g., providing to the subject about 0.7 mg, 1.4 mg, 2.8 mg, 5 mg, 10 mg, 20 mg, or 40 mg) of the oligonucleotides of the complexes per kg of the subject) of complexes (e.g., muscle targeting complexes) described herein once every 12 weeks.

In some embodiments, a method described herein comprises administering to the subject a composition comprising an effective amount of complexes (e.g., muscle targeting complexes) described herein, wherein the effective amount for each administration provides to the subject 0.3 mg to 7 mg, (e.g., 0.35 mg, 0.49 mg, 0.5 mg, 0.65 mg, 0.7 mg, 0.91 mg, 0.98 mg, 1.4 mg, 1.82 mg, 1.96 mg, 2.10 mg, 2.8 mg, 3.0 mg, 3.50 mg, 3.64 mg, 3.90 mg, 5.0 mg, or 6.50 mg) of the oligonucleotides of the complexes per kg of the subject. In some embodiments, a method described herein comprises administering to the subject a composition comprising an effective amount of complexes (e.g., muscle targeting complexes) described herein once, wherein the effective amount for each administration provides to the subject 0.7 mg to 2.8 mg (e.g., 0.7 mg, 1.4 mg, or 2.8 mg) of the oligonucleotides of the complexes per kg of the subject. In some embodiments, a method described herein comprises administering to the subject a composition comprising an effective amount of complexes (e.g., muscle targeting complexes) described herein once, wherein the effective amount for each administration provides to the subject about 0.7 mg, 1.4 mg, or 2.8 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes (e.g., muscle targeting complexes) described herein, wherein the effective amount for each administration provides to the subject 0.49 mg to 0.91 mg (e.g., 0.49 mg, 0.5 mg, 0.65 mg, 0.7 mg, 0.91 mg), of the oligonucleotides of the complexes per kg of the subject. In some embodiments, a method described herein comprises administering to the subject a composition comprising an effective amount of complexes (e.g., muscle targeting complexes) described herein, wherein the effective amount for each administration provides to the subject 0.7 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes (e.g., muscle targeting complexes) described herein, wherein the effective amount for each administration provides to the subject 0.98 mg to 1.82 mg (e.g., 0.98 mg, 1.4 mg, 1.82 mg), of the oligonucleotides of the complexes per kg of the subject. In some embodiments, a method described herein comprises administering to the subject a composition comprising an effective amount of complexes (e.g., muscle targeting complexes) described herein, wherein the effective amount for each administration provides to the subject 1.4 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes (e.g., muscle targeting complexes) described herein, wherein the effective amount for each administration provides to the subject 1.96 mg to 3.64 mg (e.g., 1.96 mg, 2.10 mg, 2.8 mg, 3.0 mg, 3.50 mg, 3.64 mg), of the oligonucleotides of the complexes per kg of the subject. In some embodiments, a method described herein comprises administering to the subject a composition comprising an effective amount of complexes (e.g., muscle targeting complexes) described herein, wherein the effective amount for each administration provides to the subject 2.8 mg of the oligonucleotides of the complexes per kg of the subject. In some embodiments, with respect to any of the preceding amounts of oligonucleotides, the values can vary by up to 30% (e.g., ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes (e.g., muscle targeting complexes) described herein once every 4 weeks, wherein the effective amount for each administration provides to the subject 0.5 mg to 3 mg (e.g., 0.5 mg to 3 mg, 0.5 mg to 2 mg, 0.5 mg to 1 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 0.7 mg, 1.4 mg, 2.8 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 0.69 mg, 1.37 mg, 2.75 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 3 mg to 7 mg (e.g., 3 mg to 7 mg, 3 mg to 6 mg, 3 mg to 5 mg, 3 mg to 4 mg, 4 mg to 7 mg, 4 mg to 6 mg, 4 mg to 5 mg, 5 mg to 7 mg, 5 mg to 6 mg, or 6 mg to 7 mg,) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 3.83 mg, 4.07 mg, 4.31 mg, 4.55 mg, 4.79 mg, 5.03 mg, 5.27 mg, 5.51 mg, 5.75 mg, 5.99 mg, 6.23 mg, or 6.47 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 5.03 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 8 mg to 12 mg (e.g., 8 mg to 12 mg, 8 mg to 11 mg, 8 mg to 10 mg, 8 mg to 9 mg, 9 mg to 12 mg, 9 mg to 11 mg, 9 mg to 10 mg, 10 mg to 12 mg, 10 mg to 11 mg, or 11 mg to 12 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 8 mg, 9 mg, 10 mg, 11 mg, or 12 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 10 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 7 mg to 13 mg (e.g., 7 mg to 13 mg, 7 mg to 12 mg, 7 mg to 11 mg, 7 mg to 10 mg, 7 mg to 9 mg, 7 mg to 8 mg, 8 mg to 13 mg, 8 mg to 12 mg, 8 mg to 11 mg, 8 mg to 10 mg, 8 mg to 9 mg, 9 mg to 13 mg, 9 mg to 12 mg, 9 mg to 11 mg, 9 mg to 10 mg, 10 mg to 13 mg, 10 mg to 12 mg, 10 mg to 11 mg, 11 mg to 13 mg, 11 mg to 12 mg, or 12 mg to 13 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 7.67 mg, 8.15 mg, 8.63 mg, 9.11 mg, 9.59 mg, 10.07 mg, 10.55 mg, 11.03 mg, 11.50 mg, 11.98 mg, 12.46 mg, or 12.94 mg, of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 10.07 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 16 mg to 26 mg (e.g., 16 mg to 26 mg, 16 mg to 25 mg, 16 mg to 20 mg, 16 mg to 18 mg, 18 mg to 26 mg, 18 mg to 25 mg, 18 mg to 20 mg, 20 mg to 26 mg, 20 mg to 25 mg, 20 mg to 22 mg, 22 mg to 26 mg, 22 mg to 25 mg, 22 mg to 23 mg, 24 mg to 26 mg, or 24 mg to 25 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 15.34 mg, 16.30 mg, 17.26 mg, 18.22 mg, 19.17 mg, 20.13 mg, 21.09 mg, 22.05 mg, 23.01 mg, 23.97 mg, 24.93 mg, or 25.89 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 20.13 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 24 mg to 36 mg (e.g., 24 mg to 36 mg, 24 mg to 33 mg, 24 mg to 30 mg, 24 mg to 27 mg, 27 mg to 36 mg, 27 mg to 33 mg, 27 mg to 30 mg, 30 mg to 36 mg, 30 mg to 33 mg, or 33 mg to 36 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 24 mg, 25 mg, 26 mg, 27 mg, 28 mg, 29 mg, 30 mg, 31 mg, 32 mg, 33 mg, 34 mg, 35 mg, or 40 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 30 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 23 mg to 39 mg (e.g., 23 mg to 39 mg, 23 mg to 35 mg, 23 mg to 30 mg, 23 mg to 25 mg, 25 mg to 39 mg, 25 mg to 35 mg, 25 mg to 30 mg, 30 mg to 39 mg, 30 mg to 35 mg, or 35 mg to 39 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 23.01 mg, 24.45 mg, 25.89 mg, 27.32 mg, 28.76 mg, 30.20 mg, 31.64 mg, 33.08 mg, 34.51 mg, 35.95 mg, 37.39 mg, 38.83 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 30.2 mg of the oligonucleotides of the complexes per kg of the subject.

In some embodiments, a method described herein comprises administering to the subject a composition comprising an effective amount of complexes (e.g., muscle targeting complexes) described herein once every 4 weeks, wherein the effective amount for each administration provides to the subject 31 mg to 52 mg (e.g., 31 mg to 52 mg, 31 mg to 50 mg, 31 mg to 45 mg, 31 mg to 40 mg, 31 mg to 35 mg, 33 mg to 52 mg, 33 mg to 50 mg, 33 mg to 45 mg, 33 mg to 40 mg, 33 mg to 35 mg, 35 mg to 52 mg, 35 mg to 50 mg, 35 mg to 45 mg, 35 mg to 40 mg, 40 mg to 52 mg, 40 mg to 50 mg, 40 mg to 45 mg, 45 mg to 52 mg, 45 mg to 50 mg, or 50 mg to 52 mg) of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 30.68 mg, 32.60 mg, 34.51 mg, 36.43 mg, 38.35 mg, 40.27 mg, 42.18 mg, 44.10 mg, 46.02 mg, 47.94 mg, 49.85 mg, or 51.77 mg of the oligonucleotides of the complexes per kg of the subject. In some embodiments, a method described herein comprises administering to the subject a composition comprising an effective amount of complexes (e.g., muscle targeting complexes) described herein once every 4 weeks, wherein the effective amount for each administration provides to the subject about 40.27 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject 0.5 mg to 3 mg (e.g., 0.5 mg to 3 mg, 0.5 mg to 2 mg, 0.5 mg to 1 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 0.7 mg, 1.4 mg, 2.8 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 0.69 mg, 1.37 mg, 2.75 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 3 mg to 8 mg (e.g., 3 mg to 8 mg, 3 mg to 7 mg, 3 mg to 6 mg, 3 mg to 5 mg, 3 mg to 4 mg, 4 mg to 8 mg, 4 mg to 7 mg, 4 mg to 6 mg, 4 mg to 5 mg, 5 mg to 8 mg, 5 mg to 7 mg, 5 mg to 6 mg, 6 mg to 8 mg, 6 mg to 7 mg, or 7 mg to 8 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 3.83 mg, 4.07 mg, 4.31 mg, 4.55 mg, 4.79 mg, 5.03 mg, 5.27 mg, 5.51 mg, 5.75 mg, 5.99 mg, 6.23 mg, or 6.47 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 5.03 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 8 mg to 12 mg (e.g., 8 mg to 12 mg, 8 mg to 11 mg, 8 mg to 10 mg, 8 mg to 9 mg, 9 mg to 12 mg, 9 mg to 11 mg, 9 mg to 10 mg, 10 mg to 12 mg, 10 mg to 11 mg, or 11 mg to 12 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 8 mg, 9 mg, 10 mg, 11 mg, or 12 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 10 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 7 mg to 13 mg (e.g., 7 mg to 13 mg, 7 mg to 12 mg, 7 mg to 11 mg, 7 mg to 10 mg, 7 mg to 9 mg, 7 mg to 8 mg, 8 mg to 13 mg, 8 mg to 12 mg, 8 mg to 11 mg, 8 mg to 10 mg, 8 mg to 9 mg, 9 mg to 13 mg, 9 mg to 12 mg, 9 mg to 11 mg, 9 mg to 10 mg, 10 mg to 13 mg, 10 mg to 12 mg, 10 mg to 11 mg, 11 mg to 13 mg, 11 mg to 12 mg, or 12 mg to 13 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 7.67 mg, 8.15 mg, 8.63 mg, 9.11 mg, 9.59 mg, 10.07 mg, 10.55 mg, 11.03 mg, 11.50 mg, 11.98 mg, 12.46 mg, or 12.94 mg, of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 10.07 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 16 mg to 26 mg (e.g., 16 mg to 26 mg, 16 mg to 25 mg, 16 mg to 20 mg, 16 mg to 18 mg, 18 mg to 26 mg, 18 mg to 25 mg, 18 mg to 20 mg, 20 mg to 26 mg, 20 mg to 25 mg, 20 mg to 22 mg, 22 mg to 26 mg, 22 mg to 25 mg, 22 mg to 23 mg, 24 mg to 26 mg, or 24 mg to 25 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 15.34 mg, 16.30 mg, 17.26 mg, 18.22 mg, 19.17 mg, 20.13 mg, 21.09 mg, 22.05 mg, 23.01 mg, 23.97 mg, 24.93 mg, or 25.89 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 20.13 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 24 mg to 36 mg (e.g., 24 mg to 36 mg, 24 mg to 33 mg, 24 mg to 30 mg, 24 mg to 27 mg, 27 mg to 36 mg, 27 mg to 33 mg, 27 mg to 30 mg, 30 mg to 36 mg, 30 mg to 33 mg, or 33 mg to 36 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 24 mg, 25 mg, 26 mg, 27 mg, 28 mg, 29 mg, 30 mg, 31 mg, 32 mg, 33 mg, 34 mg, 35 mg, or 36 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 30 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 23 mg to 39 mg (e.g., 23 mg to 39 mg, 23 mg to 35 mg, 23 mg to 30 mg, 23 mg to 25 mg, 25 mg to 39 mg, 25 mg to 35 mg, 25 mg to 30 mg, 30 mg to 39 mg, 30 mg to 35 mg, or 35 mg to 39 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 23.01 mg, 24.45 mg, 25.89 mg, 27.32 mg, 28.76 mg, 30.20 mg, 31.64 mg, 33.08 mg, 34.51 mg, 35.95 mg, 37.39 mg, 38.83 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 30.2 mg of the oligonucleotides of the complexes per kg of the subject.

In some embodiments, a method described herein comprises administering to the subject a composition comprising an effective amount of complexes (e.g., muscle targeting complexes) described herein once every 8 weeks, wherein the effective amount for each administration provides to the subject 31 mg to 52 mg (e.g., 31 mg to 52 mg, 31 mg to 50 mg, 31 mg to 45 mg, 31 mg to 40 mg, 31 mg to 35 mg, 33 mg to 52 mg, 33 mg to 50 mg, 33 mg to 45 mg, 33 mg to 40 mg, 33 mg to 35 mg, 35 mg to 52 mg, 35 mg to 50 mg, 35 mg to 45 mg, 35 mg to 40 mg, 40 mg to 52 mg, 40 mg to 50 mg, 40 mg to 45 mg, 45 mg to 52 mg, 45 mg to 50 mg, or 50 mg to 52 mg) of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 30.68 mg, 32.60 mg, 34.51 mg, 36.43 mg, 38.35 mg, 40.27 mg, 42.18 mg, 44.10 mg, 46.02 mg, 47.94 mg, 49.85 mg, or 51.77 mg of the oligonucleotides of the complexes per kg of the subject. In some embodiments, a method described herein comprises administering to the subject a composition comprising an effective amount of complexes (e.g., muscle targeting complexes) described herein once every 8 weeks, wherein the effective amount for each administration provides to the subject about 40.27 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject 0.5 mg to 3 mg (e.g., 0.5 mg to 3 mg, 0.5 mg to 2 mg, 0.5 mg to 1 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 0.7 mg, 1.4 mg, 2.8 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 0.69 mg, 1.37 mg, 2.75 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 3 mg to 7 mg (e.g., 3 mg to 7 mg, 3 mg to 6 mg, 3 mg to 5 mg, 3 mg to 4 mg, 4 mg to 7 mg, 4 mg to 6 mg, 4 mg to 5 mg, 5 mg to 7 mg, 5 mg to 6 mg, or 6 mg to 7 mg,) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 3.83 mg, 4.07 mg, 4.31 mg, 4.55 mg, 4.79 mg, 5 mg, 5.03 mg, 5.27 mg, 5.51 mg, 5.75 mg, 5.99 mg, 6.23 mg, or 6.47 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 5.03 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 8 mg to 12 mg (e.g., 8 mg to 12 mg, 8 mg to 11 mg, 8 mg to 10 mg, 8 mg to 9 mg, 9 mg to 12 mg, 9 mg to 11 mg, 9 mg to 10 mg, 10 mg to 12 mg, 10 mg to 11 mg, or 11 mg to 12 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 8 mg, 9 mg, 10 mg, 11 mg, or 12 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 10 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 7 mg to 13 mg (e.g., 7 mg to 13 mg, 7 mg to 12 mg, 7 mg to 11 mg, 7 mg to 10 mg, 7 mg to 9 mg, 7 mg to 8 mg, 8 mg to 13 mg, 8 mg to 12 mg, 8 mg to 11 mg, 8 mg to 10 mg, 8 mg to 9 mg, 9 mg to 13 mg, 9 mg to 12 mg, 9 mg to 11 mg, 9 mg to 10 mg, 10 mg to 13 mg, 10 mg to 12 mg, 10 mg to 11 mg, 11 mg to 13 mg, 11 mg to 12 mg, or 12 mg to 13 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 7.67 mg, 8.15 mg, 8.63 mg, 9.11 mg, 9.59 mg, 10 mg, 10.07 mg, 10.55 mg, 11.03 mg, 11.50 mg, 11.98 mg, 12.46 mg, or 12.94 mg, of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 10.07 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 16 mg to 26 mg (e.g., 16 mg to 26 mg, 16 mg to 25 mg, 16 mg to 20 mg, 16 mg to 18 mg, 18 mg to 26 mg, 18 mg to 25 mg, 18 mg to 20 mg, 20 mg to 26 mg, 20 mg to 25 mg, 20 mg to 22 mg, 22 mg to 26 mg, 22 mg to 25 mg, 22 mg to 23 mg, 24 mg to 26 mg, or 24 mg to 25 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 15.34 mg, 16.30 mg, 17.26 mg, 18.22 mg, 19.17 mg, 20 mg, 20.13 mg, 21.09 mg, 22.05 mg, 23.01 mg, 23.97 mg, 24.93 mg, or 25.89 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 20.13 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 24 mg to 36 mg (e.g., 24 mg to 36 mg, 24 mg to 33 mg, 24 mg to 30 mg, 24 mg to 27 mg, 27 mg to 36 mg, 27 mg to 33 mg, 27 mg to 30 mg, 30 mg to 36 mg, 30 mg to 33 mg, or 33 mg to 36 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 24 mg, 25 mg, 26 mg, 27 mg, 28 mg, 29 mg, 30 mg, 31 mg, 32 mg, 33 mg, 34 mg, 35 mg, or 36 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 30 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 23 mg to 39 mg (e.g., 23 mg to 39 mg, 23 mg to 35 mg, 23 mg to 30 mg, 23 mg to 25 mg, 25 mg to 39 mg, 25 mg to 35 mg, 25 mg to 30 mg, 30 mg to 39 mg, 30 mg to 35 mg, or 35 mg to 39 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 23.01 mg, 24.45 mg, 25.89 mg, 27.32 mg, 28.76 mg, 30.20 mg, 31.64 mg, 33.08 mg, 34.51 mg, 35.95 mg, 37.39 mg, 38.83 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 30.2 mg of the oligonucleotides of the complexes per kg of the subject.

In some embodiments, a method described herein comprises administering to the subject a composition comprising an effective amount of complexes (e.g., muscle targeting complexes) described herein once every 12 weeks, wherein the effective amount for each administration provides to the subject 31 mg to 52 mg (e.g., 31 mg to 52 mg, 31 mg to 50 mg, 31 mg to 45 mg, 31 mg to 40 mg, 31 mg to 35 mg, 33 mg to 52 mg, 33 mg to 50 mg, 33 mg to 45 mg, 33 mg to 40 mg, 33 mg to 35 mg, 35 mg to 52 mg, 35 mg to 50 mg, 35 mg to 45 mg, 35 mg to 40 mg, 40 mg to 52 mg, 40 mg to 50 mg, 40 mg to 45 mg, 45 mg to 52 mg, 45 mg to 50 mg, or 50 mg to 52 mg) of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 30.68 mg, 32.60 mg, 34.51 mg, 36.43 mg, 38.35 mg, 40 mg, 40.27 mg, 42.18 mg, 44.10 mg, 46.02 mg, 47.94 mg, 49.85 mg, or 51.77 mg of the oligonucleotides of the complexes per kg of the subject. In some embodiments, a method described herein comprises administering to the subject a composition comprising an effective amount of complexes (e.g., muscle targeting complexes) described herein once every 12 weeks, wherein the effective amount for each administration provides to the subject about 40.27 mg of the oligonucleotides of the complexes per kg of the subject. In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the two administrations within the first 4 weeks provides to the subject a total amount of the oligonucleotides of the complexes that is equal to the amount of oligonucleotides of the complexes provided by each subsequent administration.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein each of the two administrations within the first 4 weeks provides to the subject an amount of the oligonucleotides of the complexes that is equal to the amount of oligonucleotides of the complexes provided by each subsequent administration. In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for the first two administrations within the first 4 weeks provides to the subject a total of 3-52 mg (e.g., 5.03 mg, 10.06 mg, 20.13 mg, 30.2 mg, or 40.27 mg) of the oligonucleotides of the complexes per kg of the subject, and wherein each subsequent administration provides to the subject 3-52 mg (e.g., 5.03 mg, 10.06 mg, 20.13 mg, 30.2 mg or 40.27 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for each of the first two administrations within the first 4 weeks provides to the subject 3-52 mg (e.g., 5.03 mg, 10.06 mg, 20.13 mg, 30.2 mg, or 40.27 mg) of the oligonucleotides of the complexes per kg of the subject, and wherein each subsequent administration provides to the subject 3-52 mg (e.g., 5.03 mg, 10.06 mg, 20.13 mg, 30.2 mg, or 40.27 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for the first two administrations within the first 4 weeks provides to the subject a total of 5 to 40 mg (e.g., 5 mg, 10 mg, 20 mg, 30 mg, or 40 mg) of the oligonucleotides of the complexes per kg of the subject, and wherein each subsequent administration provides to the subject 5 to 40 mg (e.g., 5 mg, 10 mg, 20 mg, 30 mg, or 40 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for each of the first two administrations within the first 4 weeks provides to the subject 5 to 40 mg (e.g., 5 mg, 10 mg, 20 mg, 30 mg, or 40 mg) of the oligonucleotides of the complexes per kg of the subject, and wherein each subsequent administration provides to the subject 5 to 40 mg (e.g., 5 mg, 10 mg, 20 mg, 30 mg, or 40 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for the first two administrations within the first 4 weeks provides to the subject a total of 8 to 36 mg (e.g., 8 mg, 10 mg, 12 mg, 15 mg, 20 mg, 24 mg, 25 mg, 30 mg, or 36 mg) of the oligonucleotides of the complexes per kg of the subject, and wherein each subsequent administration provides to the subject 8 to 36 mg (e.g., 8 mg, 10 mg, 12 mg, 15 mg, 20 mg, 24 mg, 25 mg, 30 mg, or 36 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for each of the first two administrations within the first 4 weeks provides to the subject 8 to 36 mg (e.g., 8 mg, 10 mg, 12 mg, 15 mg, 20 mg, 24 mg, 25 mg, 30 mg, or 36 mg) of the oligonucleotides of the complexes per kg of the subject, and wherein each subsequent administration provides to the subject 8 to 36 mg (e.g., 8 mg, 10 mg, 12 mg, 15 mg, 20 mg, 24 mg, 25 mg, 30 mg, or 36 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for the first two administrations within the first 4 weeks (week 0 and week 2) provides to the subject a total of 24 mg to 36 mg (e.g., 4 mg for the first dose and 20 mg for the second dose, 8 mg for the first dose and 16 mg for the second dose, 12 mg for the first dose and 12 mg for the second dose, 24 mg for the first dose and 8 mg for the second dose, or 20 mg for the first dose and 4 mg for the second dose, 5 mg for the first dose and 25 mg for the second dose, 10 mg for the first dose and 20 mg for the second dose, 15 mg for the first dose and 15 mg for the second dose, 20 mg for the first dose and 10 mg for the second dose, 25 mg for the first dose and 5 mg for the second dose, 6 mg for the first dose and 30 mg for the second dose, 12 mg for the first dose and 24 mg for the second dose, 18 mg for the first dose and 18 mg for the second dose, 24 mg for the first dose and 12 mg for the second dose, or 30 mg for the first dose and 6 mg for the second dose) of the oligonucleotides of the complexes per kg of the subject, and wherein each subsequent administration provides to the subject 24 mg to 36 mg (e.g., 24 mg, 30 mg, or 36 mg) of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for the first two administrations within the first 4 weeks (week 0 and week 2) provides to the subject a total of 30 mg (e.g., 5 mg for the first dose and 25 mg for the second dose, 10 mg for the first dose and 20 mg for the second dose, 15 mg for the first dose and 15 mg for the second dose, 20 mg for the first dose and 10 mg for the second dose, or 25 mg for the first dose and 5 mg for the second dose) of the oligonucleotides of the complexes per kg of the subject, and wherein each subsequent administration provides to the subject 30 mg of the oligonucleotides of the complexes per kg of the subject.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for each of the administrations within the first 4 weeks provides to the subject 24 mg to 36 mg (e.g., 24 mg, 30 mg, or 36 mg) of the oligonucleotides of the complexes per kg of the subject, and wherein each subsequent administration provides to the subject 24 mg to 36 mg (e.g., 24 mg, 30 mg, or 36 mg) of the oligonucleotides of the complexes per kg of the subject.

It should be understood that once every 4 weeks is substantially similar to once a month, once every 8 weeks is substantially similar to once every two months, once every 12 weeks is substantially similar to once every three months, and once every 16 weeks is substantially similar to once every four months. As such, in some embodiments, once every 4 weeks can mean once a month; once every 8 weeks can mean once every two months; once every 12 weeks can mean once every three months; and once every 16 weeks can mean once every four months. Similarly, in some embodiments, once every 4 weeks can mean 12 times per year; once every 8 weeks can mean 6 times per year; and once every 12 weeks can mean 4 times per year.

In some embodiments, a method described herein comprises administering to the subject a composition comprising an effective amount of complexes (e.g., muscle targeting complexes) described herein once every two weeks in the first 4 weeks (dose in week 0, week 2, week 4), and subsequently once every 4 weeks thereafter, wherein the effective amount for each of the administrations within the first 4 weeks provides to the subject 30 mg of the oligonucleotides of the complexes per kg of the subject, and wherein each subsequent administration provides to the subject 30 mg of the oligonucleotides of the complexes per kg of the subject.

In some embodiments, a method described herein comprises administering via infusion an effective amount of a formulation of a composition comprising complexes (e.g., in an aqueous solution), wherein each complex comprises a structure of formula (I): [R¹]ₙ₁—R², wherein:
each R¹ comprises a group of the formula (Ic):

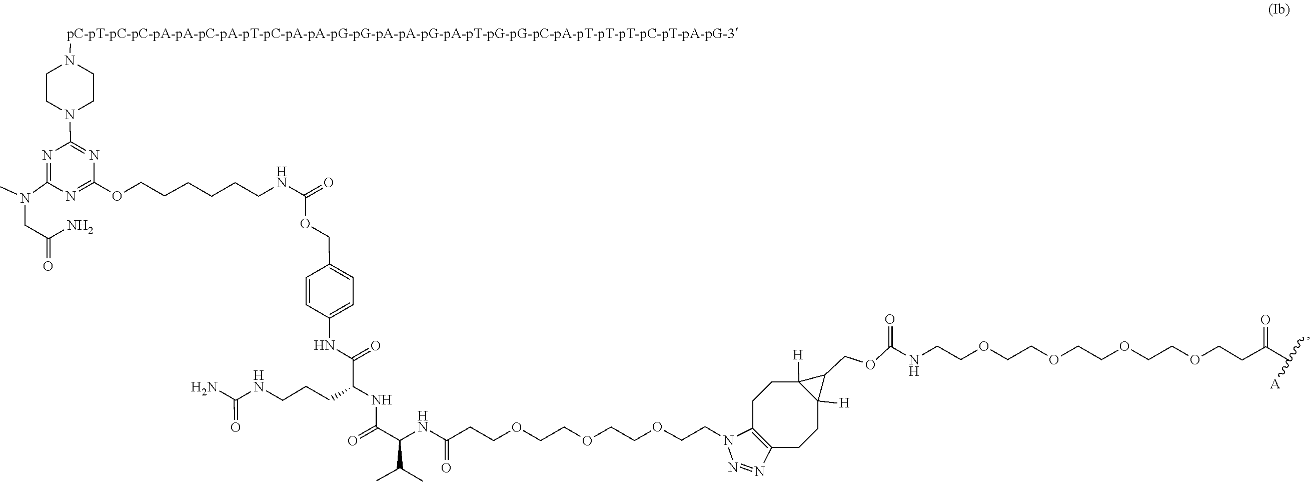

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; and wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 8 mg to 36 mg (e.g., 8 mg, 10 mg, 12 mg, 20 mg, 24 mg, 30 mg, or 36 mg) of the oligonucleotides of the complexes per kg of the subject, optionally wherein the subject is administered the composition once every four weeks, eight weeks, or twelve weeks. In some embodiments, formulation of a composition comprising complexes (e.g., in aqueous solutions) for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a formulation of a composition comprising complexes (e.g., in an aqueous solution), wherein each complex comprises a structure of formula (I): [R¹]ₙ₁—R², wherein:
each R¹ comprises a group of the formula (Ic):

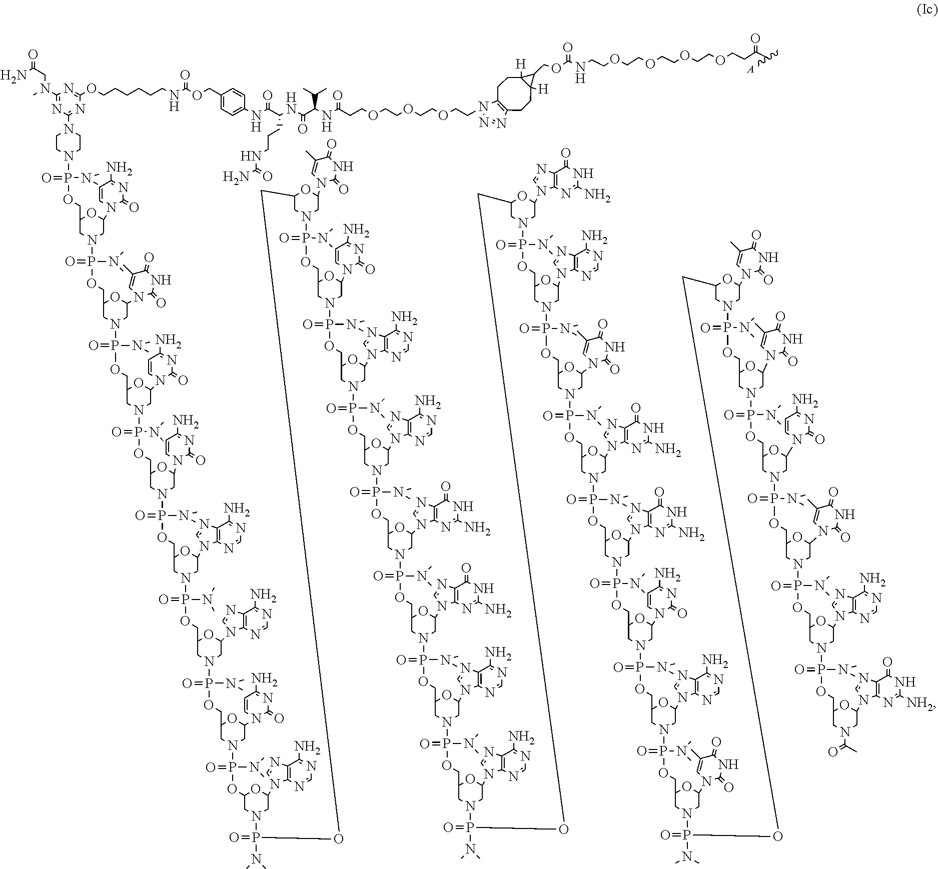

R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 8 mg to 36 mg (e.g., 8 mg, 10 mg, 12 mg, 20 mg, 24 mg, 30 mg, or 36 mg) of the oligonucleotides of the complexes per kg of the subject, optionally wherein the subject is administered the composition once every four weeks, eight weeks, or twelve weeks. In some embodiments, formulation of a composition comprising complexes (e.g., in aqueous solutions) for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a formulation of a composition comprising complexes (e.g., in an aqueous solution), wherein each complex comprises a structure of formula (Id):

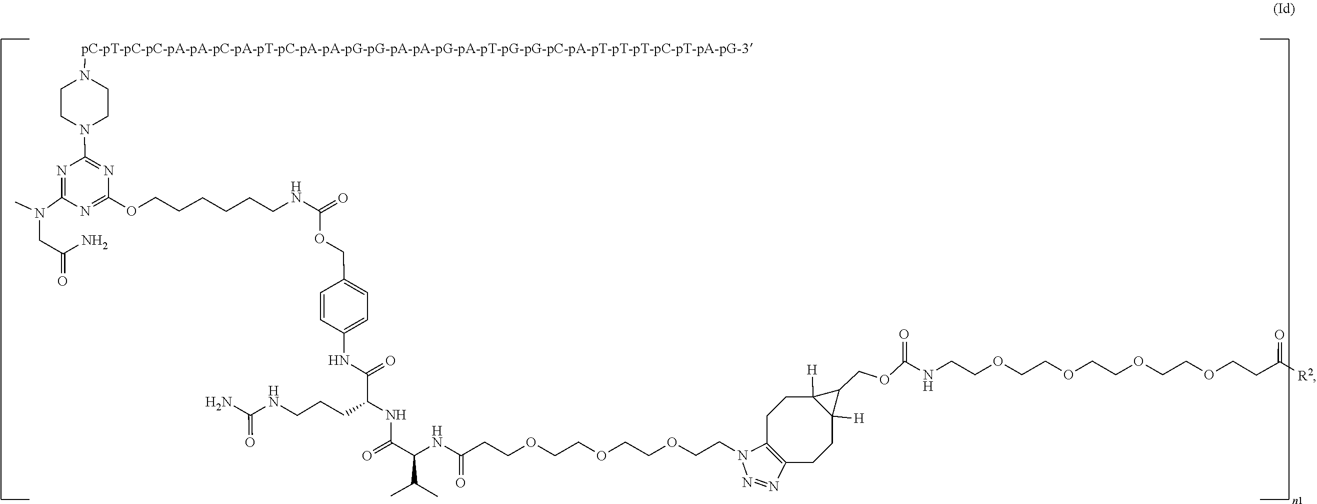

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; optionally wherein the anti-TfR1 antibody (e.g., Fab) is covalently linked (e.g., indirectly or directly linked, e.g., directly linked) via different amino acid residues of the antibody, optionally wherein each different amino acid residue is a lysine; and
wherein in each complex n1 is independently an integer of one or greater, optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 8 mg to 36 mg (e.g., 8 mg, 10 mg, 12 mg, 20 mg, 24 mg, 30 mg, or 36 mg) of the oligonucleotides of the complexes per kg of the subject, optionally wherein the subject is administered the composition once every four weeks, eight weeks, or twelve weeks. In some embodiments, formulation of a composition comprising complexes (e.g., in aqueous solutions) for administration to a subject in the methods described herein further comprise complexes that comprise a structure of formula (I): [R¹]ₙ₁—R², wherein n1 is 0.

In some embodiments, in any one of the methods described herein, variations in the amount of oligonucleotides provided to the subject by administering the composition comprising an effective amount of the complexes described herein are contemplated. Accordingly, in some embodiments, the effective amount provides to the subject approximately 5 mg (e.g., ±up to 20%, ±up to 15%, ±up to 10%, ±up to 5%, ±up to 3%, or ±up to 1%) to approximately 100 mg (e.g., ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 5 mg (e.g., ±up to 20%, ±up to 15%, ±up to 10%, ±up to 5%, ±up to 3%, or ±up to 1%) to approximately 50 mg (e.g., ±up to 20%, ±up to 15%, ±up to 10%, ±up to 5%, ±up to 3%, or ±up to 1%) of the oligonucleotides of the complexes per kg of the subject.

In some embodiments, the effective amount provides to the subject approximately 10 mg (e.g., ±up to 20%, ±up to 15%, ±up to 10%, ±up to 5%, ±up to 3%, or ±up to 1%) to approximately 30 mg (e.g., ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, or 36 mg of the oligonucleotides of the complexes per kg of the subject. With respect to any of the proceeding amounts of the oligonucleotides, the value can vary up to 20% (e.g., ±up to 20%, ±up to 15%, ±up to 10%, ±up to 5%, ±up to 3%, or ±up to 1%).

In some embodiments, the effective amount provides to the subject approximately 10 mg (e.g., ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 30 mg (e.g., ±up to 20%, ±up to 15%, ±up to 10%, ±up to 5%, ±up to 3%, or ±up to 1%) of the oligonucleotides of the complexes per kg of the subject.

In some embodiments, a method described herein comprises administering via infusion an effective amount of a formulation of a composition comprising complexes (e.g., in an aqueous solution), wherein each complex comprises a structure of formula (I): [R¹]ₙ₁—R², wherein:
each R¹ comprises a group of the formula (Ib):

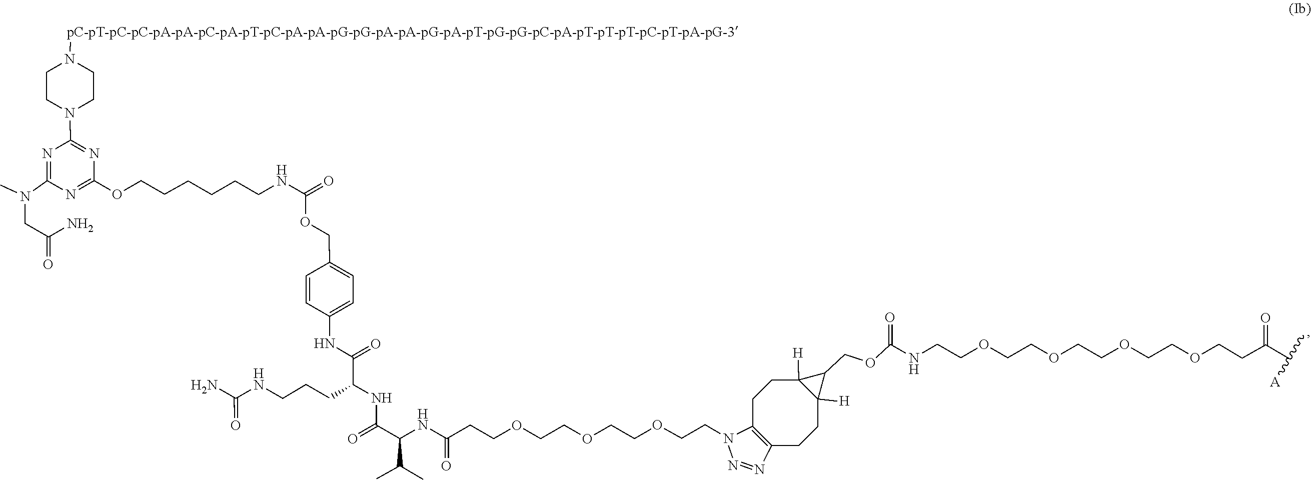

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; and wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 3 mg to 52 mg of the oligonucleotides of the complexes per kg of the subject, optionally wherein the effective amount of each administration provides to the subject 5 mg to 40 mg of the oligonucleotides of the complexes per kg of the subject, optionally wherein the subject is administered the composition once every four weeks, eight weeks, or twelve weeks. In some embodiments, the effective amount of each administration provides to the subject 5 mg, 10 mg, 20 mg, 30 mg, or 40 mg of the oligonucleotides of the complexes per kg of the subject. In some embodiments, the effective amount of each administration provides to the subject 5.03 mg, 10.06 mg, 20.13 mg, 30.2 mg, or 40.27 mg of the oligonucleotides of the complexes per kg of the subject. In some embodiments, formulation of a composition comprising complexes (e.g., in aqueous solutions) for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a formulation of a composition comprising complexes (e.g., in an aqueous solution), wherein each complex comprises a structure of formula (I): [R¹]ₙ₁—R², wherein:
each R¹ comprises a group of the formula (Ic):

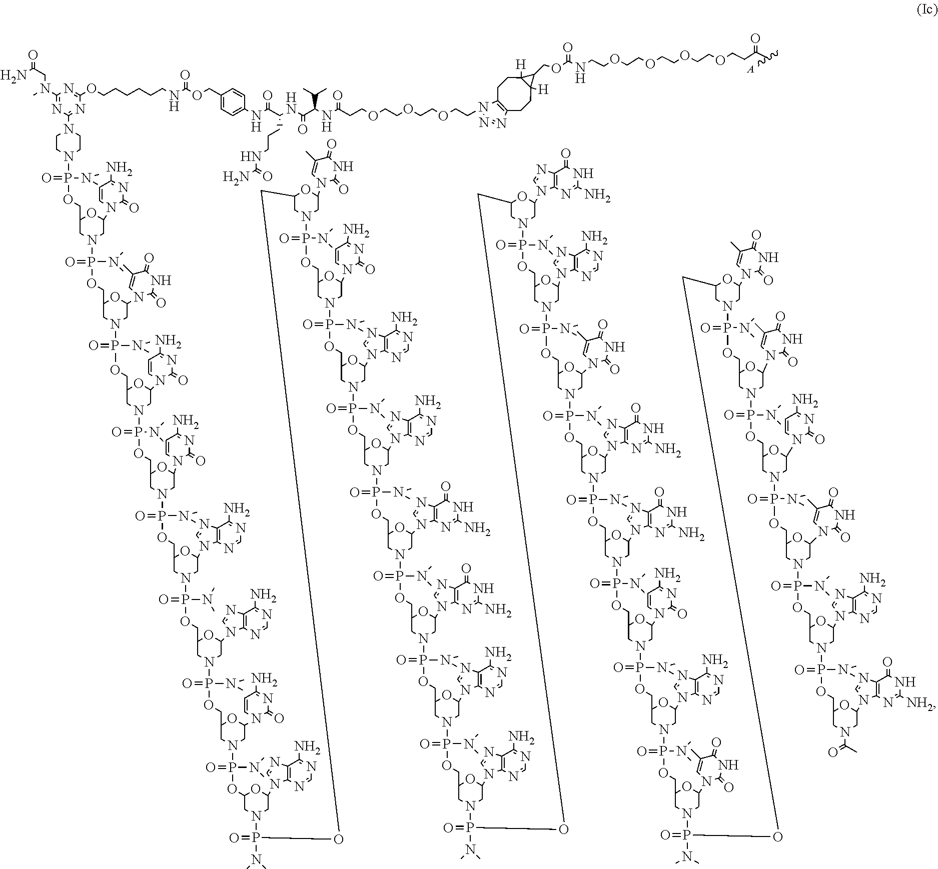

R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 3 mg to 52 mg of the oligonucleotides of the complexes per kg of the subject, optionally wherein the effective amount of each administration provides to the subject 5 mg to 40 mg of the oligonucleotides of the complexes per kg of the subject, optionally wherein the subject is administered the composition once every four weeks, eight weeks, or twelve weeks. In some embodiments, the effective amount of each administration provides to the subject 5 mg, 10 mg, 20 mg, 30 mg, or 40 mg of the oligonucleotides of the complexes per kg of the subject. In some embodiments, the effective amount of each administration provides to the subject 5.03 mg, 10.06 mg, 20.13 mg, 30.2 mg, or 40.27 mg of the oligonucleotides of the complexes per kg of the subject. In some embodiments, formulation of a composition comprising complexes (e.g., in aqueous solutions) for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a formulation of a composition comprising complexes (e.g., in an aqueous solution), wherein each complex comprises a structure of formula (Id)

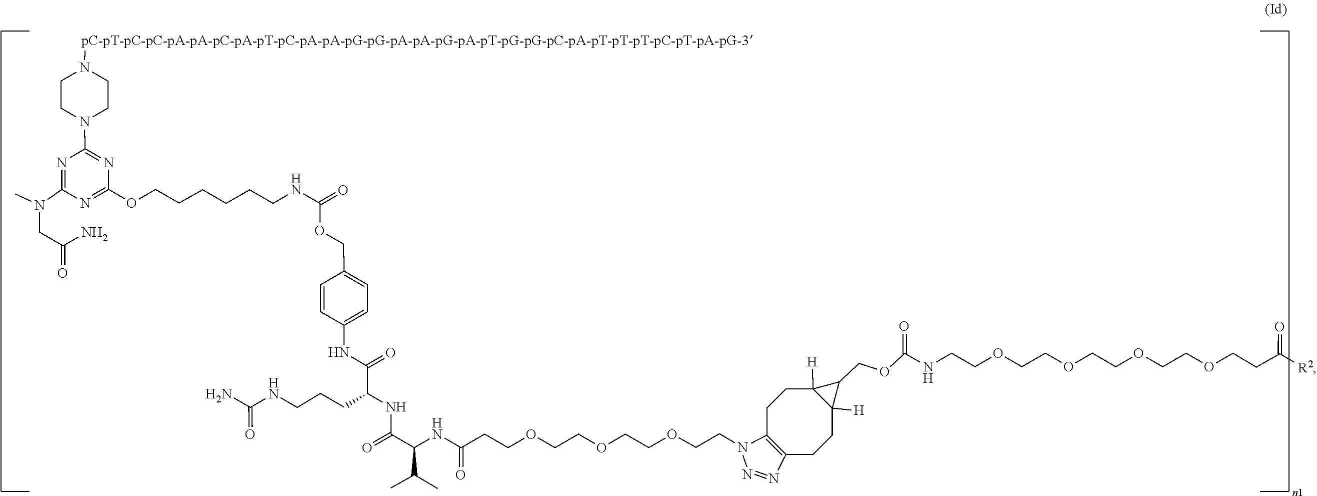

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21); R² comprises an anti-TfR1 antibody (e.g., Fab) comprising a heavy chain comprising the amino acid sequence of SEQ ID NO: 19 and a light chain comprising the amino acid sequence of SEQ ID NO: 20; optionally wherein the anti-TfR1 antibody (e.g., Fab) is covalently linked (e.g., indirectly or directly linked, e.g., directly linked) via different amino acid residues of the antibody, optionally wherein each different amino acid residue is a lysine; and wherein in each complex n1 is independently an integer of one or greater, optionally wherein the average value of n1 of the complexes of the composition is in the range of 1 to 5; wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 3 mg to 52 mg of the oligonucleotides of the complexes per kg of the subject, optionally wherein the effective amount of each administration provides to the subject 5 mg to 40 mg of the oligonucleotides of the complexes per kg of the subject, optionally wherein the subject is administered the composition once every four weeks, eight weeks, or twelve weeks. In some embodiments, the effective amount of each administration provides to the subject 5 mg, 10 mg, 20 mg, 30 mg, or 40 mg of the oligonucleotides of the complexes per kg of the subject. In some embodiments, the effective amount of each administration provides to the subject 5.03 mg, 10.06 mg, 20.13 mg, 30.2 mg, or 40.27 mg of the oligonucleotides of the complexes per kg of the subject. In some embodiments, formulation of a composition comprising complexes (e.g., in aqueous solutions) for administration to a subject in the methods described herein further comprise complexes that comprise a structure of formula (I): [R₁]ₙ₁—R², wherein n1 is 0 and wherein R¹ and R² are defined herein.

In some embodiments, in any one of the methods described herein, variations in the amount of oligonucleotides provided to the subject by administering the composition comprising an effective amount of the complexes described herein are contemplated. Accordingly, in some embodiments, the effective amount provides to the subject approximately 5 mg (e.g., ±up to 30%, ±up to 25%, ±up to 20%, ±up to 15%, ±up to 10%, ±up to 5%, ±up to 3%, or ±up to 1%) to approximately 40 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 5.03 mg (e.g., ±up to 30%, ±up to 25%, ±up to 20%, ±up to 15%, ±up to 10%, ±up to 5%, ±up to 3%, or ±up to 1%) to approximately 40.27 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, the effective amount provides to the subject approximately 5 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 5.03 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, the effective amount provides to the subject approximately 10 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 10.06 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, the effective amount provides to the subject approximately 20 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 20.13 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, the effective amount provides to the subject approximately 20 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 30.2 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, the effective amount provides to the subject approximately 40 mg (e.g., ±up to 30% up to 25%, ±up to 20%, ±up to 15% up to 10%, ±up to 5%, ±up to 3%, or ±up to 1%) of the oligonucleotides of the complexes per kg of the subject. In some embodiments, the effective amount provides to the subject approximately 40.27 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, a method described herein comprises administering via infusion an effective amount of a formulation of a composition comprising complexes (e.g., in an aqueous solution), wherein each complex comprises a structure of formula (I): [R¹]ₙ₁—R², wherein:
each R¹ comprises a group of the formula (Ib):

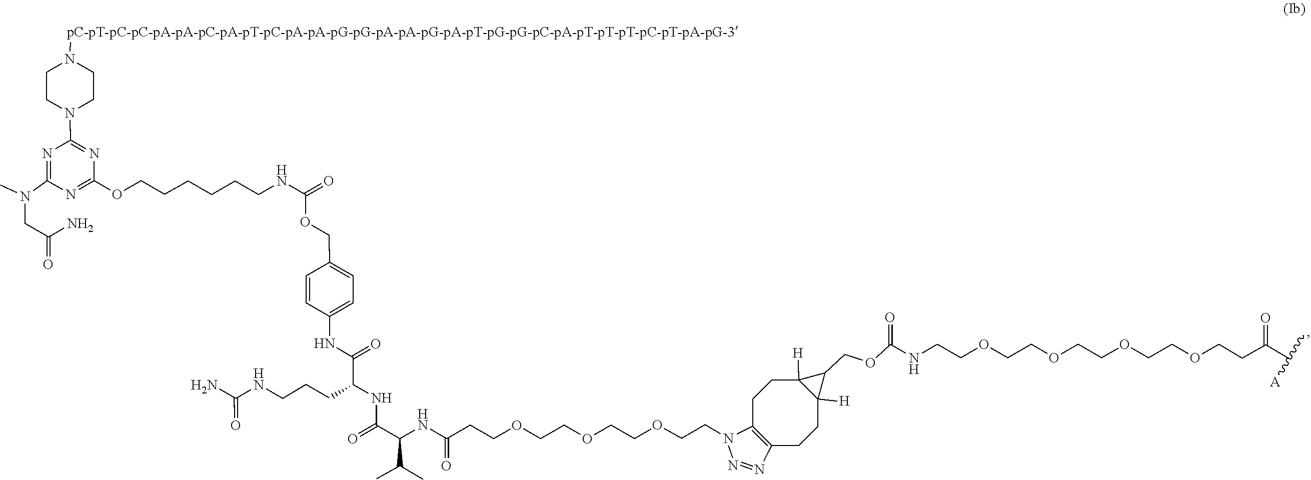

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject, optionally wherein the subject is administered the composition once every four weeks, eight weeks, or twelve weeks. In some embodiments, the effective amount of each administration provides to the subject 0.7 mg, 1.4 mg, 2.8 mg of the oligonucleotides of the complexes per kg of the subject. In some embodiments, formulation of a composition comprising complexes (e.g., in aqueous solutions) for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a formulation of a composition comprising complexes (e.g., in an aqueous solution), wherein each complex comprises a structure of formula (I): [R¹]ₙ₁—R², wherein:
each R¹ comprises a group of the formula (Ic):

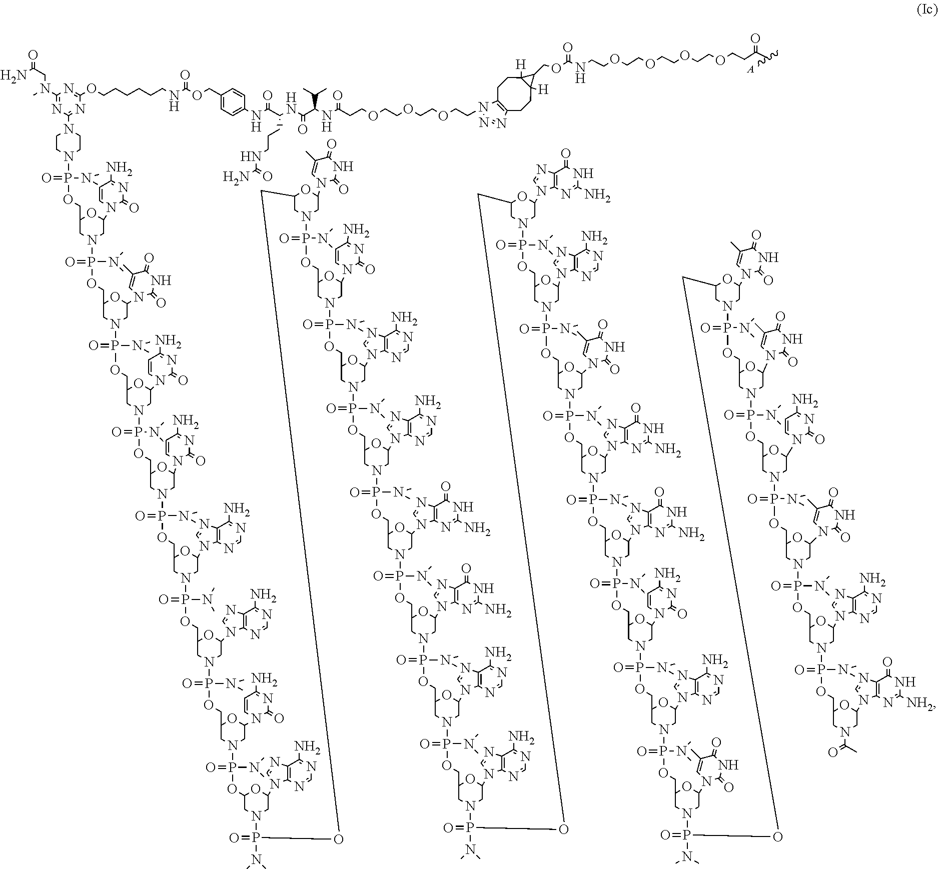

R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject, optionally wherein the subject is administered the composition once every four weeks, eight weeks, or twelve weeks. In some embodiments, the effective amount of each administration provides to the subject 0.7 mg, 1.4 mg, 2.8 mg of the oligonucleotides of the complexes per kg of the subject. In some embodiments, formulation of a composition comprising complexes (e.g., in aqueous solutions) for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a formulation of a composition comprising complexes (e.g., in an aqueous solution), wherein each complex comprises a structure of formula (Id):

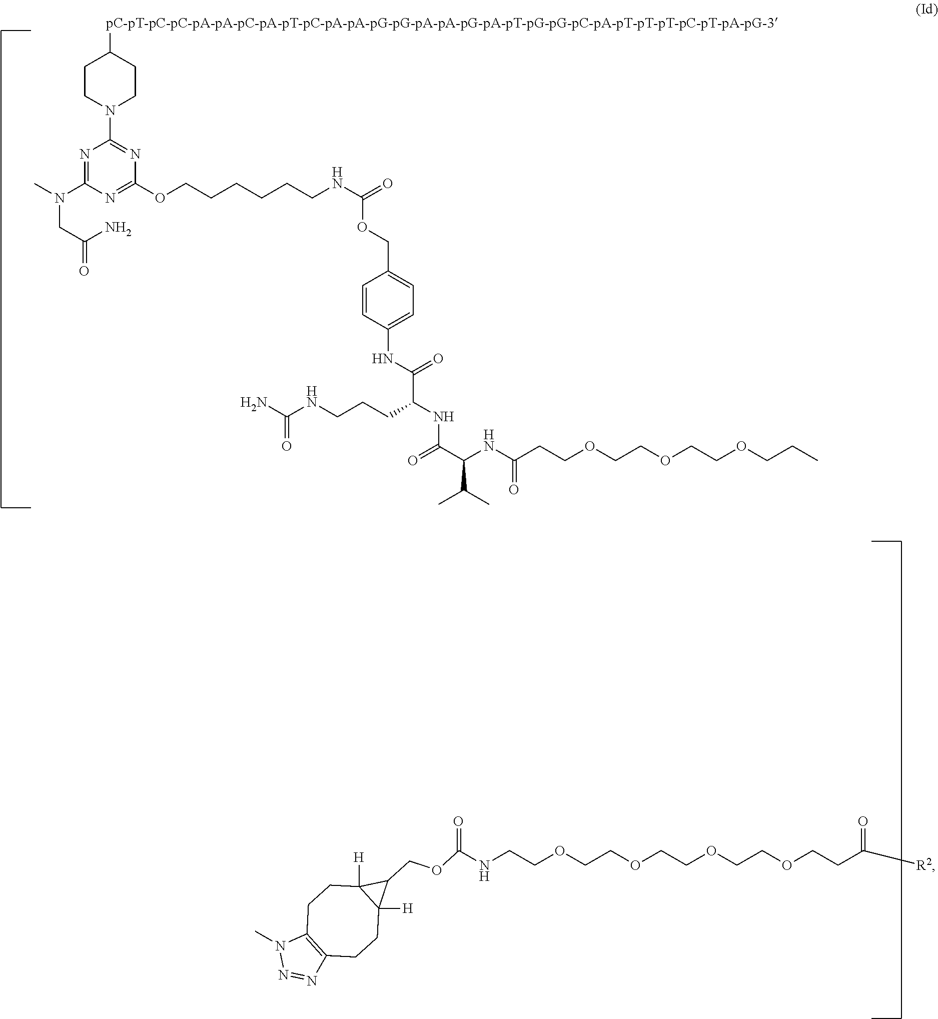

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; optionally wherein the anti-TfR1 antibody (e.g., Fab) is covalently linked (e.g., indirectly or directly linked, e.g., directly linked) via different amino acid residues of the antibody, optionally wherein each different amino acid residue is a lysine; and
wherein in each complex n1 is independently an integer of one or greater, optionally wherein the average value of n1 of the complexes of the composition is in the range of 1 to 5;
wherein the formulation is at a pH of 5.5-6.5 (e.g., 6.0) and comprises histidine (e.g., at a concentration of 25 mM), sucrose (e.g., at a concentration of 10 w/v %), and the complexes (e.g., at a concentration in the range of 10 mg/mL to 50 mg/mL);
wherein the effective amount of each administration provides to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject, optionally wherein the subject is administered the composition once every four weeks, eight weeks, or twelve weeks. In some embodiments, the effective amount of each administration provides to the subject 0.7 mg, 1.4 mg, 2.8 mg of the oligonucleotides of the complexes per kg of the subject. In some embodiments, formulation of a composition comprising complexes (e.g., in aqueous solutions) for administration to a subject in the methods described herein further comprise complexes that comprise a structure of formula (I): [R¹]ₙ₁—R², wherein n1 is 0 and wherein R¹ and R² are defined herein.

In some embodiments, in any one of the methods described herein, variations in the amount of oligonucleotides provided to the subject by administering the composition comprising an effective amount of the complexes described herein are contemplated. In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount provides to the subject approximately 0.5 mg (e.g., ±up to 30%, ±up to 25%, ±up to 20%, ±up to 15%, ±up to 10%, ±up to 5%, ±up to 3%, or ±up to 1%) to approximately 5 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount provides to the subject approximately 0.7 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 1.4 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject. In some embodiments, the effective amount provides to the subject approximately 2.8 mg (e.g., ±up to 30%, ±up to 25%, ±up to 20%, ±up to 15%, ±up to 10%, ±up to 5%, ±up to 3%, or ±up to 1%) of the oligonucleotides of the complexes per kg of the subject.

In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount for each administration provides to the subject 0.3 mg to 7 mg, (e.g., 0.35 mg, 0.49 mg, 0.5 mg, 0.65 mg, 0.7 mg, 0.91 mg, 0.98 mg, 1.4 mg, 1.82 mg, 1.96 mg, 2.10 mg, 2.8 mg, 3.0 mg, 3.50 mg, 3.64 mg, 3.90 mg, 5.0 mg, or 6.50 mg) of the oligonucleotides of the complexes per kg of the subject. In some embodiments, the effective amount for each administration provides to the subject 0.7 mg to 2.8 mg (e.g., 0.7 mg, 1.4 mg, or 2.8 mg) of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount for each administration provides to the subject 0.7 mg to 2.8 mg (e.g., 0.7 mg, 1.4 mg, or 2.8 mg) of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ta), (Tb), (Ic), or (Id)), wherein the effective amount for each administration provides to the subject 0.49 mg to 0.91 mg (e.g., 0.49 mg, 0.5 mg, 0.65 mg, 0.7 mg, 0.91 mg), of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount for each administration provides to the subject 0.7 mg of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ta), (Tb), (Ic), or (Id)), wherein the effective amount for each administration provides to the subject 0.98 mg to 1.82 mg (e.g., 0.98 mg, 1.4 mg, 1.82 mg), of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount for each administration provides to the subject 1.4 mg of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount for each administration provides to the subject 1.96 mg to 3.64 mg (e.g., 1.96 mg, 2.10 mg, 2.8 mg, 3.0 mg, 3.50 mg, 3.64 mg), of the oligonucleotides of the complexes per kg of the subject. In some embodiments, a method described herein comprises administering to the subject a composition comprising an effective amount of complexes described herein (e.g., complexes comprising a structure of formula (I): [R¹]ₙ₁—R², such as one comprising a group of the formula (Ia), (Ib), (Ic), or (Id)), wherein the effective amount for each administration provides to the subject 2.8 mg of the oligonucleotides of the complexes per kg of the subject.

In some embodiments, a method described herein comprises administering to a subject a composition comprising complexes comprising any one of the structures of formula (I): [R¹]ₙ₁—R² as described herein, wherein the composition comprises a sufficient amount of the anti-TfR1 antibody (e.g., Fab) of the complexes of the composition to deliver the oligonucleotides of the complexes to the subject (e.g., to deliver 0.5 mg to 100 mg, 0.5 mg to 150 mg, 0.5 mg to 10 mg, 0.5 mg to 3 mg, 0.5 mg to 5 mg, 5 mg to 100 mg, 5 mg to 50 mg, or 10 mg to 30 mg oligonucleotides of the complexes per kg of the subject). In some embodiments, in any one of the methods described herein, a composition for administering to a subject comprises complexes comprising any one of the structures of formula (I): [R¹]ₙ₁—R² as described herein, and the amount of anti-TfR1 antibody (e.g., Fab) of the complexes of the composition sufficient to deliver an amount of oligonucleotides of the complexes to the subject can be derived using the equation (Equation A) below:

$$anti TfR 1 antibody amount ( mg ) = ( \frac{( \frac{oligonucleotide amount ( mg )}{MW of oligonucleotide ( \frac{g}{mol} )} )}{average value of n 1} ) * MW of anti TfR 1 antibody ( \frac{g}{mol} ) ,$$

in which “MW” indicates molecular weight (g/mol), and “average value of n1” indicates the average value of n1 of the complexes of the composition.

In some embodiments, in any one of the methods described herein, a composition for administering to a subject comprises complexes comprising any one of the structures of formula (I): [R¹]ₙ₁—R² as described herein, and the amount of oligonucleotides of the complexes provided to the subject per kg of the subject can be derived using the equation (Equation B) below:

$$oligonucleotide amount ( mg ) = ( \frac{anti - TfR 1 antibody amount ( mg )}{MW of anti - TfR 1 antibody ( \frac{g}{mol} )} ) * average value of n 1 * MW of oligonucleotide ( \frac{g}{mol} ) ,$$

in which “MW” indicates molecular weight (g/mol), and “average value of n1” indicates the average value of n1 of the complexes of the composition.

In some embodiments, the present disclosure contemplates variation in the average value of n1 of up to 30% (e.g., ±up to 30%, ±up to 25%, ±up to 20%, ±up to 15%, ±up to 10%, ±up to 5%, ±up to 3%, or ±up to 1%). In some embodiments, the average value of n1 of the complexes of the composition is in the range of 1 to 5 (e.g., 1, 2, 3, 4, or 5). In some embodiments, a method provided herein comprises administering to the subject a composition comprising complexes comprising any one of the structures of formula (I): [R¹]ₙ₁—R² as described herein, wherein the average value of n1 of the complexes of the composition is in the range of 1 to 5 (e.g., 1, 2, 3, 4, or 5), with a variation of up to 30% (e.g., ±up to 30%, ±up to 25%, ±up to 20%, ±up to 15%, ±up to 10%, ±up to 5%, ±up to 3%, or ±up to 1%).

In some embodiments, the present disclosure contemplates variation in the average value of n1 of up to 20% (e.g., ±up to 20%, ±up to 15%, ±up to 10%, ±up to 5%, ±up to 3%, or ±up to 1%). In some embodiments, the average value of n1 of the complexes of the composition is in the range of 1 to 5 (e.g., 1, 2, 3, 4, or 5). In some embodiments, a method provided herein comprises administering to the subject a composition comprising complexes comprising any one of the structures of formula (I): [R¹]ₙ₁—R² as described herein, wherein the average value of n1 of the complexes of the composition is in the range of 1 to 5 (e.g., 1, 2, 3, 4, or 5), with a variation of up to 20% (e.g., ±up to 20%, ±up to 15%, ±up to 10%, ±up to 5%, ±up to 3%, or ±up to 1%).

It is to be understood that the average value of n1 needs not be an integer and can be a decimal. In some embodiments, the average value of n1 is about 1.6, 1.7, 1.8, 1.8, 2.0, 2.1, 2.2, 2.3, 2.4, 2.5, 2.6, or 2.7. In some embodiments, the average value of n1 is about 2. In some embodiments, the average value of n1 is about 2.1. In some embodiments, the average value of n1 is 2, 2.05, 2.1, 2.111, 2.112, 2.113, 2.114, 2.115, 2.12, 2.115, 2.13, 2.135, 2.14, 2.145, or 2.15.

It should be understood that any amount of oligonucleotide provided herein can be represented as an amount of anti-TfR1 antibody (e.g., Fab) according to Equation A. As such, it should be understood that description herein relating to providing an amount of any oligonucleotide alternatively can be understood as providing an amount of anti-TfR1 antibody (e.g., Fab) of a corresponding amount according to Equation A. Conversely, any amount of anti-TfR1 antibody (e.g., Fab) provided herein can be represented as an amount of oligonucleotide according to Equation B. As such, it should be understood that description herein relating to providing any amount of anti-TfR1 antibody (e.g., Fab) alternatively can be understood as providing oligonucleotide of a corresponding amount according to Equation B.

In some embodiments, for purposes of determining the molecular weight of an oligonucleotide, the oligonucleotide is represented by the group of formula (Ie):

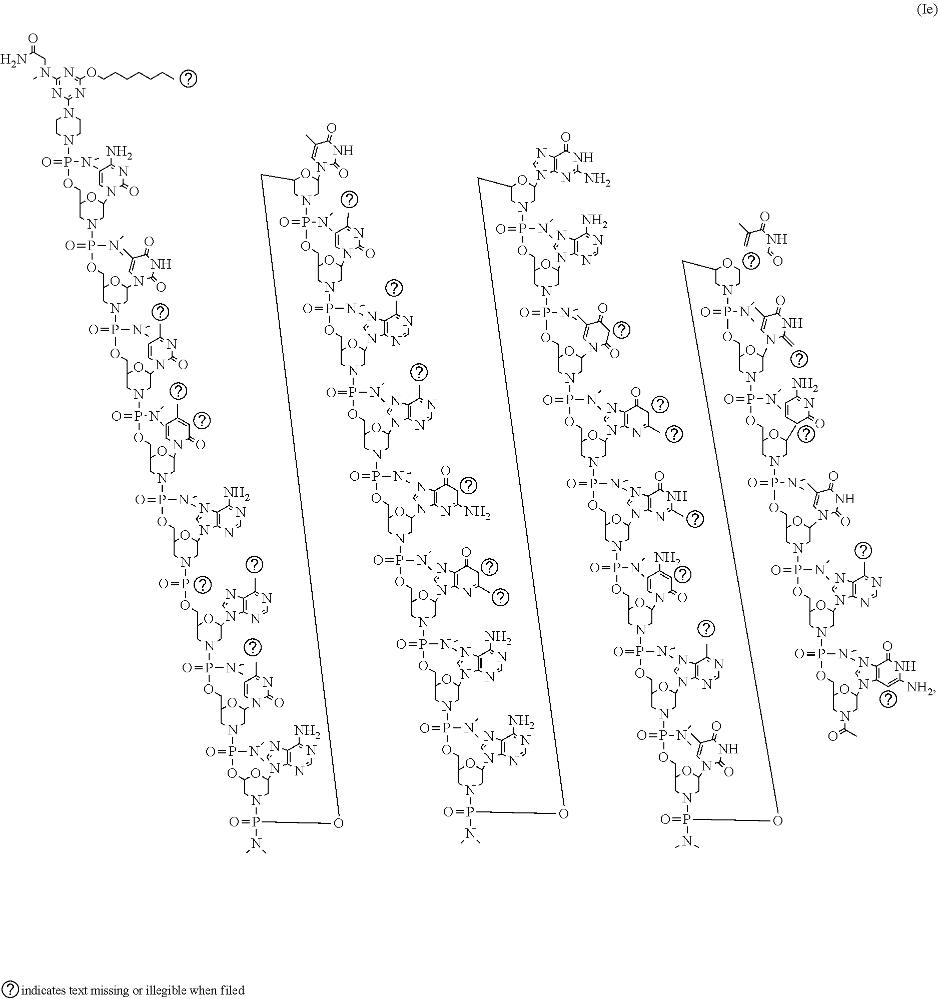

which corresponds to a molecular weight of 10452.09 g/mol.

In some embodiments, an anti-TfR1 Fab (e.g., an anti-TfR1 Fab comprising a heavy chain and a light chain sequences shown in Table 2) of a complex used in the methods described herein has a molecular weight of 47968.43 g/mol. Accordingly, in some embodiments, a method described herein comprises administering to a subject a composition comprising complexes comprising any one of the structures of formula (I): [R¹]ₙ₁—R² as described herein, and the amount of anti-TfR1 antibody (e.g., Fab) of the complexes of the composition sufficient to deliver an amount of oligonucleotides to the subject is, provided that the average value of n1 of the complexes of the composition is 2:

$$anti TfR 1 Fab amount ( mg ) = ( \frac{( \frac{oligonucleotide amount ( mg )}{10452.09 ( \frac{g}{mol} )} )}{2} ) * 47968.43 ( \frac{g}{mol} ) .$$

In some embodiments, a method described herein comprises administering to a subject a composition comprising complexes comprising any one of the structures of formula (I): [R¹]ₙ₁—R² as described herein, and the amount of anti-TfR1 antibody (e.g., Fab) of the complexes of the composition sufficient to deliver an amount of oligonucleotides to the subject is, provided that the average value of n1 of the complexes of the composition is 2.1:

$$anti TfR 1 Fab amount ( mg ) = ( \frac{( \frac{oligonucleotide amount ( mg )}{10452.09 ( \frac{g}{mol} )} )}{2.1} ) * 47968.43 ( \frac{g}{mol} ) .$$

In some embodiments, a method provided herein comprises administering to the subject a composition comprising complexes comprising any one of the structures of formula (I): [R¹]ₙ₁—R² as described herein, wherein the composition comprises a sufficient amount of the anti-TfR1 antibody (e.g., Fab) of the complexes of the composition to deliver to the subject the oligonucleotides of the complexes per kg of the subject, and wherein the sufficient amount of anti-TfR1 antibody (e.g., Fab) is derived using Equation A provided herein based on the average value of n1 of the complexes of the composition and the amount of oligonucleotides of the complexes to be delivered to the subject, optionally wherein the sufficient amount of anti-TfR1 antibody (e.g., Fab) varies up to 33% from the amount derived from Equation A (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to a subject a composition comprising an effective amount of complexes comprising any one of the structures of formula (I): [R¹]ₙ₁—R² as described herein, wherein the effective amount of complexes provides to the subject an amount of the anti-TfR1 Fab of the complexes per kg of the subject, and an amount of oligonucleotide per kg of the subject, wherein the amount of oligonucleotide is:

$$oligonucleotide amount ( mg ) = ( \frac{anti - TfR 1 antibody amount ( mg )}{47968.43 ( \frac{g}{mol} )} ) * 2 * 10452.09 ( \frac{g}{mol} ) ,$$

provided that the average value of n1 of the complexes of the composition is 2. In some embodiments, the amount of oligonucleotide varies up to 30% from the amount derived from Equation B (e.g., ±up to 30%, ±up to 25%, ±up to 20%, ±up to 15%, ±up to 10%, ±up to 5%, ±up to 3%, or ±up to 1%).

In some embodiments, a method provided herein comprises administering to a subject a composition comprising an effective amount of complexes comprising any one of the structures of formula (I): [R¹]ₙ₁—R² as described herein, wherein the effective amount of complexes provides to the subject an amount of the anti-TfR1 Fab of the complexes per kg of the subject, and an amount of oligonucleotide per kg of the subject, wherein the amount of oligonucleotide is:

$$oligonucleotide amount ( mg ) = ( \frac{anti - TfR 1 antibody amount ( mg )}{47968.43 ( \frac{g}{mol} )} ) * 2.1 * 10452.09 ( \frac{g}{mol} ) ,$$

provided that the average value of n1 of the complexes of the composition is 2.1. In some embodiments, the amount of oligonucleotide varies up to 30% from the amount derived from Equation B (e.g., ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the composition comprises a sufficient amount of the anti-TfR1 antibody (e.g., Fab) of the complexes of the composition to deliver to the subject the oligonucleotides of the complexes per kg of the subject, and wherein the sufficient amount of anti-TfR1 antibody (e.g., Fab) is derived using Equation A provided herein based on the average value of n1 of the complexes of the composition and the amount of oligonucleotides of the complexes to be delivered to the subject, optionally wherein the sufficient amount of anti-TfR1 antibody (e.g., Fab) varies up to 25% from the amount derived from Equation A (e.g., ±up to 25%, ±up to 20%, ±up to 15%, ±up to 10%, ±up to 5%, ±up to 3%, or ±up to 1%).

In some embodiments, a method provided herein comprises administering to the subject a composition comprising complexes described herein (e.g., complexes comprising a structure of formula (I): [R¹]ₙ₁—R², such as one comprising a group of the formula (Ia), (Ib), (Ic), or (Id)), wherein the composition comprises a sufficient amount of the anti-TfR1 antibody (e.g., Fab) of the complexes of the composition to deliver to the subject 0.5 mg to 5 mg of the oligonucleotides of the complexes per kg of the subject, and wherein the sufficient amount of anti-TfR1 antibody (e.g., Fab) is derived using Equation A provided herein based on the average value of n1 of the complexes of the composition, optionally wherein the sufficient amount of anti-TfR1 antibody (e.g., Fab) varies up to 33% from the amount derived from Equation A (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes described herein (e.g., complexes comprising a structure of formula (I): [R¹]ₙ₁—R², such as one comprising a group of the formula (Ia), (Ib), (Ic), or (Id)), wherein the average value of n1 of the complexes of the composition is 2.1, wherein the composition comprises a sufficient amount of the anti-TfR1 antibody (e.g., Fab) of the complexes of the composition to deliver to the subject 0.5 mg to 5 mg of the oligonucleotides of the complexes per kg of the subject, and wherein the sufficient amount of anti-TfR1 antibody (e.g., Fab) is approximately 1 mg (e.g., ±up to 33%, ±up to 30%, ±up to 25%, ±up to 20%, ±up to 15%, ±up to 10%, ±up to 5%, ±up to 3%, or ±up to 1%) to 11 mg (e.g., ±up to 25%, ±up to 20%, ±up to 15%, ±up to 10%, ±up to 5%, ±up to 3%, or ±up to 1%) per kg of the subject, respectively. In some embodiments, the sufficient amount of anti-TfR1 antibody (e.g., Fab) is approximately 1.09 mg (e.g., ±up to 33%, ±up to 30%, ±up to 25%, ±up to 20%, ±up to 15%, ±up to 10%, ±up to 5%, ±up to 3%, or ±up to 1%) to 10.93 mg (e.g., ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes described herein (e.g., complexes comprising a structure of formula (I): [R¹]ₙ₁—R², such as one comprising a group of the formula (Ia), (Ib), (Ic), or (Id)), wherein the average value of n1 of the complexes of the composition is 2.1, wherein the composition comprises a sufficient amount of the anti-TfR1 antibody (e.g., Fab) of the complexes of the composition to deliver to the subject 0.5 mg to 5 mg (e.g., 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 2 mg to 5 mg, 2 mg to 4.5 mg, 2 mg to 4 mg, 2 mg to 3.5 mg, 2 mg to 3 mg, 3 mg to 5 mg, 3 mg to 4.5 mg, 3 mg to 4 mg, 3.5 mg to 5 mg, 3.5 mg to 4.5 mg, or 4 mg to 5 mg) of the oligonucleotides of the complexes per kg of the subject, and wherein the sufficient amount of anti-TfR1 antibody (e.g., Fab) is approximately 1.09 mg to 10.93 mg (e.g., 1.09 mg to 10.93 mg, 1.09 mg to 9.83 mg, 1.09 mg to 9.18 mg, 1.09 mg to 7.65 mg, 1.09 mg to 6.56 mg, 1.09 mg to 5.46 mg, 2.19 mg to 10.93 mg, 2.19 mg to 9.83 mg, 2.19 mg to 9.18 mg, 2.19 mg to 7.65 mg, 2.19 mg to 6.56 mg, 2.19 mg to 5.46 mg, 4.37 mg to 10.93 mg, 4.37 mg to 9.83 mg, 4.37 mg to 9.18 mg, 4.37 mg to 7.65 mg, 4.37 mg to 6.56 mg, 66.56 mg to 10.93 mg, 6.56 mg to 9.83 mg, 6.56 mg to 9.18 mg, 7.65 mg to 10.93 mg, 7.65 mg to 9.83 mg, or 9.18 mg to 10.93 mg, respectively) per kg of the subject. In some embodiments, the sufficient amount of anti-TfR1 antibody (e.g., Fab) is approximately 1 mg to 8 mg (e.g., 1 mg, 1.5 mg, 2 mg, 3 mg, 4 mg, 5 mg, 6 mg, 7 mg, or 8 mg) of the anti-TfR1 antibody (e.g., Fab). In some embodiments, a method provided herein comprises administering to the subject a composition comprising complexes described herein (e.g., complexes comprising a structure of formula (I): [R¹]ₙ₁—R², such as one comprising a group of the formula (Ia), (Ib), (Ic), or (Id)), wherein the average value of n1 of the complexes of the composition is 2.1, wherein the composition comprises a sufficient amount of the anti-TfR1 antibody (e.g., Fab) of the complexes of the composition to deliver to the subject 0.7 mg to 2.8 mg (e.g., 0.7 mg, 1.4 mg, or 2.8 mg) of the oligonucleotides of the complexes per kg of the subject, and wherein the sufficient amount of anti-TfR1 antibody (e.g., Fab) is approximately 1.5 mg to 6 mg (e.g., 1.5 mg, 3 mg, or 6 mg) per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes described herein (e.g., complexes comprising a structure of formula (I): [R¹]ₙ₁—R², such as one comprising a group of the formula (Ia), (Ib), (Ic), or (Id)), wherein the average value of n1 of the complexes of the composition is 2.1, wherein the composition comprises a sufficient amount of the anti-TfR1 antibody (e.g., Fab) of the complexes of the composition to deliver to the subject 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, or 5 mg of the oligonucleotides of the complexes per kg of the subject, and wherein the sufficient amount of anti-TfR1 antibody (e.g., Fab) is approximately 1.09 mg, 1.31 mg, 1.53 mg, 1.75 mg, 1.97 mg, 2.19 mg, 2.40 mg, 2.62 mg, 2.84 mg, 3.06 mg, 3.28 mg, 3.5 mg, 3.72 mg, 3.93 mg, 4.15 mg, 4.37 mg, 4.59 mg, 5.03 mg, 5.25 mg, 5.46 mg, 5.68 mg, 5.90 mg, 6.12 mg, 6.34 mg, 6.56 mg, 6.77 mg, 6.99 mg, 7.21 mg, 7.43 mg, 7.65 mg, 7.87 mg, 8.09 mg, 8.30 mg, 8.52 mg, 8.74 mg, 8.96 mg, 9.18 mg, 9.40 mg, 9.62 mg, 9.83 mg, 10.05 mg, 10.27 mg, 10.49 mg, 10.71 mg, or 10.93 mg per kg of the subject, respectively. In some embodiments, the sufficient amount of anti-TfR1 antibody (e.g., Fab) is 1.5 mg, 3 mg, 6 mg, or 11 mg per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes described herein (e.g., complexes comprising a structure of formula (I): [R¹]ₙ₁—R², such as one comprising a group of the formula (Ta), (Ib), (Ic), or (Id)), wherein the average value of n1 of the complexes of the composition is 2.1, wherein the composition comprises a sufficient amount of the anti-TfR1 antibody (e.g., Fab) of the complexes to deliver to the subject 0.3 mg to 7 mg, (e.g., 0.35 mg, 0.49 mg, 0.5 mg, 0.65 mg, 0.7 mg, 0.91 mg, 0.98 mg, 1.4 mg, 1.82 mg, 1.96 mg, 2.10 mg, 2.8 mg, 3.0 mg, 3.50 mg, 3.64 mg, 3.90 mg, 5.0 mg, or 6.50 mg) of the oligonucleotides of the complexes per kg of the subject of the oligonucleotides of the complexes per kg of the subject, and wherein the sufficient amount of anti-TfR1 antibody (e.g., Fab) is approximately 0.66 mg (e.g., ±up to 33%, ±up to 30%, ±up to 25%, ±up to 20%, ±up to 15%, ±up to 10%, ±up to 5%, ±up to 3%, or ±up to 1%) to approximately 15.30 mg (e.g., ±up to 33%, ±up to 30%, ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes described herein (e.g., complexes comprising a structure of formula (I): [R¹]ₙ₁—R², such as one comprising a group of the formula (Ia), (Ib), (Ic), or (Id)), wherein the average value of n1 of the complexes of the composition is 2.1, wherein the composition comprises a sufficient amount of the anti-TfR1 antibody (e.g., Fab) of the complexes to deliver to the subject 0.7 mg to 2.8 mg, (e.g., 0.7 mg, 0.91 mg, 0.98 mg, 1.4 mg, 1.82 mg, 1.96 mg, 2.10 mg, or 2.8 mg) of the oligonucleotides of the complexes per kg of the subject of the oligonucleotides of the complexes per kg of the subject, and wherein the sufficient amount of anti-TfR1 antibody (e.g., Fab) is approximately 1.5 mg (e.g., ±up to 33%, ±up to 30%, ±up to 25%, ±up to 20%, ±up to 15%, ±up to 10%, ±up to 5%, ±up to 3%, or ±up to 1%) to approximately 6 mg (e.g., ±up to 33%, ±up to 30%, ±up to 25%, ±up to 20%, ±up to 15%, ±up to 10%, ±up to 5%, ±up to 3%, or ±up to 1%) per kg of the subject, respectively. In some embodiments, the sufficient amount of anti-TfR1 antibody (e.g., Fab) is approximately 1.53 mg (e.g., ±up to 33%, ±up to 30%, ±up to 25%, ±up to 20%, ±up to 15%, ±up to 10%, ±up to 5%, ±up to 3%, or ±up to 1%) to approximately 6.12 mg (e.g., ±up to 33%, ±up to 30%, ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes described herein (e.g., complexes comprising a structure of formula (I): [R¹]ₙ₁—R², such as one comprising a group of the formula (Ta), (Tb), (Ic), or (Id)), wherein the average value of n1 of the complexes of the composition is 2.1, wherein the composition comprises a sufficient amount of the anti-TfR1 antibody (e.g., Fab) of the complexes to deliver to the subject 0.7 mg of the oligonucleotides of the complexes per kg of the subject of the oligonucleotides of the complexes per kg of the subject, and wherein the sufficient amount of anti-TfR1 antibody (e.g., Fab) is approximately 1.07 mg to 2.03 mg (e.g., 1.07 mg, 1.09 mg, 1.45 mg, 1.53 mg, or 2.03 mg) per kg of the subject. In some embodiments, the sufficient amount of anti-TfR1 antibody (e.g., Fab) is approximately 1 mg to 2 mg (e.g., 1 mg, 1.5 mg, or 2 mg) per kg of the subject. In some embodiments, the sufficient amount of anti-TfR1 antibody (e.g., Fab) is approximately 1.5 mg per kg of the subject. In some embodiments, the sufficient amount of anti-TfR1 antibody (e.g., Fab) is approximately 1.53 mg per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject 1.4 mg of the oligonucleotides of the complexes per kg of the subject of the oligonucleotides of the complexes per kg of the subject, and wherein the sufficient amount of anti-TfR1 antibody (e.g., Fab) is approximately 2.14 mg to 4.07 mg (e.g., 2.14 mg, 3.06 mg, or 4.07 mg) per kg of the subject. In some embodiments, the sufficient amount of anti-TfR1 antibody (e.g., Fab) is approximately 2 mg to 4 mg (e.g., 2 mg, 3 mg, or 4 mg) per kg of the subject. In some embodiments, the sufficient amount of anti-TfR1 antibody (e.g., Fab) is approximately 3 mg per kg of the subject. In some embodiments, the sufficient amount of anti-TfR1 antibody (e.g., Fab) is approximately 3.06 mg per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%). In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes of the composition to deliver to the subject 2.8 mg of the oligonucleotides of the complexes per kg of the subject, and wherein the sufficient amount of anti-TfR1 antibody (e.g., Fab) is approximately 4.28 mg to 8.14 mg (e.g., 4.28 mg, 4.59 mg, 6.12 mg, 6.56 mg, 7.65 mg, or 8.14 mg) per kg of the subject. In some embodiments, the sufficient amount of anti-TfR1 antibody (e.g., Fab) is approximately 4 mg to 8 mg (e.g., 4 mg, 6 mg, or 8 mg) per kg of the subject. In some embodiments, the sufficient amount of anti-TfR1 antibody (e.g., Fab) is approximately 6 mg per kg of the subject. In some embodiments, the sufficient amount of anti-TfR1 antibody (e.g., Fab) is approximately 6.12 mg per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes of the composition to deliver to the subject 0.7 mg, 1.4 mg, or 2.8 mg of the oligonucleotides of the complexes per kg of the subject, and wherein the sufficient amount of anti-TfR1 antibody (e.g., Fab) is approximately 1.5 mg, 3 mg, or 6 mg per kg of the subject, respectively. In some embodiments, the sufficient amount of anti-TfR1 antibody (e.g., Fab) is approximately 1.53 mg, 3.06 mg, or 6.12 mg per kg of the subject, respectively. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the composition comprises a sufficient amount of the anti-TfR1 antibody (e.g., Fab) of the complexes of the composition to deliver to the subject 5 mg to 50 mg of the oligonucleotides of the complexes per kg of the subject, and wherein the sufficient amount of anti-TfR1 antibody (e.g., Fab) is derived using Equation A provided herein based on the average value of n1 of the complexes of the composition, optionally wherein the sufficient amount of anti-TfR1 antibody (e.g., Fab) varies up to 25% from the amount derived from Equation A (e.g., ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes of the composition to deliver to the subject 5 mg to 50 mg of the oligonucleotides of the complexes per kg of the subject, and wherein the sufficient amount of anti-TfR1 antibody (e.g., Fab) is approximately 11.47 mg (e.g., ±up to 25%, ±up to 20%, ±up to 15%, ±up to 10%, ±up to 5%, ±up to 3%, or ±up to 1%) to 114.73 mg (e.g., ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject 5 mg to 40 mg of the oligonucleotides of the complexes per kg of the subject, and wherein the sufficient amount of anti-TfR1 antibody (e.g., Fab) is approximately 11.47 mg (e.g., up to 25%, up to 20%, ±up to 15%, ±up to 10%, ±up to 5%, ±up to 3%, or ±up to 1%) to 91.79 mg (e.g., up to 25%, up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes of the composition to deliver to the subject 5 mg to 40 mg (e.g., 5 mg to 40 mg, 5 mg to 35 mg, 5 mg to 30 mg, 5 mg to 25 mg, 5 mg to 20 mg, 5 mg to 15 mg, 5 mg to 10 mg, 10 mg to 40 mg, 10 mg to 35 mg, 10 mg to 30 mg, 10 mg to 25 mg, 10 mg to 20 mg, 10 mg to 15 mg, 15 mg to 40 mg, 15 mg to 35 mg, 15 mg to 30 mg, 15 mg to 25 mg, 15 mg to 20 mg, 20 mg to 40 mg, 20 mg to 35 mg, 20 mg to 30 mg, 20 mg to 25 mg, 25 mg to 40 mg, 25 mg to 35 mg, 25 mg to 30 mg, 30 mg to 40 mg, 30 mg to 35 mg, or 35 mg to 40 mg) of the oligonucleotides of the complexes per kg of the subject, and wherein the sufficient amount of anti-TfR1 antibody (e.g., Fab) is approximately 11.47 mg to 91.79 mg (e.g., 11.47 mg to 91.79 mg, 11.47 mg to 80.31 mg, 11.47 mg to 68.84 mg, 11.47 mg to 57.38 mg, 11.47 mg to 45.89 mg, 11.47 mg to 34.42 mg, 11.47 mg to 22.95 mg, 22.95 mg to 91.79 mg, 22.95 mg to 80.31 mg, 22.95 mg to 68.84 mg, 22.95 mg to 57.37 mg, 22.95 mg to 45.89 mg, 22.95 mg to 34.42 mg, 34.42 mg to 91.79 mg, 34.42 mg to 80.31 mg, 34.42 mg to 68.84 mg, 34.42 mg to 57.37 mg, 34.42 mg to 45.89 mg, 45.89 mg to 91.79 mg, 45.89 mg to 80.31 mg, 45.89 mg to 68.84 mg, 45.89 mg to 57.37 mg, 57.37 mg to 91.79 mg, 57.37 mg to 80.31 mg, 57.37 mg to 68.84 mg, 68.84 mg to 91.79 mg, 68.84 mg to 80.31 mg, or 80.31 mg to 91.79 mg, respectively) per kg of the subject. In some embodiments, with respect to any of the preceding amounts of anti-TfR1 antibody (e.g., Fab), the values can vary by up to ±25% (e.g., ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes of the composition to deliver to the subject 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, or 40 mg of the oligonucleotides of the complexes per kg of the subject, and wherein the sufficient amount of anti-TfR1 antibody (e.g., Fab) is approximately 11.47 mg, 13.77 mg, 16.06 mg, 18.36 mg, 20.65 mg, 22.95 mg, 25.24 mg, 27.54 mg, 29.83 mg, 32.13 mg, 34.42 mg, 36.71 mg, 39.01 mg, 41.30 mg, 43.60 mg, 45.89 mg, 48.19 mg, 50.48 mg, 52.78 mg, 55.07 mg, 57.37 mg, 59.66 mg, 61.96 mg, 64.25 mg, 66.55 mg, 68.84 mg, 71.14 mg, 73.43 mg, 75.72 mg, 78.02 mg, 80.31 mg, 82.61 mg, 84.90 mg, 87.20 mg, 89.49 mg, or 91.79 mg per kg of the subject, respectively. In some embodiments, with respect to any of the preceding amounts of anti-TfR1 antibody (e.g., Fab), the values can vary by up to ±25% (e.g., ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject 8-12 mg of the oligonucleotides of the complexes per kg of the subject, and wherein the sufficient amount of anti-TfR1 antibody (e.g., Fab) is approximately 18.36 mg (e.g., ±up to 25%, ±up to 20%, ±up to 15%, ±up to 10%, ±up to 5%, ±up to 3%, or ±up to 1%) to approximately 27.54 mg (e.g., ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject 10 mg of the oligonucleotides of the complexes per kg of the subject, and wherein the sufficient amount of anti-TfR1 antibody (e.g., Fab) is approximately 22.95 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject 24 mg to 36 mg of the oligonucleotides of the complexes per kg of the subject, and wherein the sufficient amount of anti-TfR1 antibody (e.g., Fab) is approximately 55.07 mg (e.g., ±up to 25%, ±up to 20%, ±up to 15%, ±up to 10%, ±up to 5%, ±up to 3%, or ±up to 1%) to approximately 82.61 mg (e.g., ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject 30 mg of the oligonucleotides of the complexes per kg of the subject, and wherein the sufficient amount of anti-TfR1 antibody (e.g., Fab) is approximately 68.84 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes of the composition to deliver to the subject oligonucleotides of the complexes, and wherein the sufficient amount of anti-TfR1 antibody (e.g., Fab) is approximately 11.47 mg (e.g., ±up to 25%, ±up to 20%, ±up to 15%, ±up to 10%, ±up to 5%, ±up to 3%, or ±up to 1%) to 114.73 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 11.47 mg (e.g., ±up to 25%, ±up to 20%, ±up to 15%, ±up to 10%, ±up to 5%, ±up to 3%, or ±up to 1%) to 91.79 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the composition comprises a sufficient amount of the anti-TfR1 antibody (e.g., Fab) of the complexes of the composition to deliver to the subject 5 mg to 40 mg of the oligonucleotides of the complexes per kg of the subject, and wherein the sufficient amount of anti-TfR1 antibody (e.g., Fab) is derived using Equation A provided herein based on the average value of n1 of the complexes of the composition, optionally wherein the sufficient amount of anti-TfR1 antibody (e.g., Fab) varies up to 33% from the amount derived from Equation A (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes of the composition to deliver to the subject 5 mg to 40 mg of the oligonucleotides of the complexes per kg of the subject, and wherein the sufficient amount of anti-TfR1 antibody (e.g., Fab) is approximately 10 mg (e.g., ±up to 33%, ±up to 30%, ±up to 25%, ±up to 20%, ±up to 15%, ±up to 10%, ±up to 5%, ±up to 3%, or ±up to 1%) to 90 mg (e.g., ±up to 25%, ±up to 20%, ±up to 15%, ±up to 10%, ±up to 5%, ±up to 3%, or ±up to 1%) per kg of the subject, respectively.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes of the composition to deliver to the subject 5 mg, 10 mg, 20 mg, 30 mg, or 40 mg of the oligonucleotides of the complexes per kg of the subject, and wherein the sufficient amount of anti-TfR1 antibody (e.g., Fab) is approximately 11 mg, 22 mg, 44 mg, 66 mg, or 88 mg per kg of the subject, respectively. In some embodiments, the sufficient amount of anti-TfR1 antibody (e.g., Fab) is approximately 10.93 mg, 21.85 mg, 43.71 mg, 65.56 mg, or 87.42 mg per kg of the subject, respectively. In some embodiments, the sufficient amount of anti-TfR1 antibody (e.g., Fab) is approximately 10.93 mg, 21.86 mg, 43.72 mg, 65.56 mg, or 87.43 mg per kg of the subject, respectively. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject about 5 mg of the oligonucleotides of the complexes per kg of the subject, and wherein the sufficient amount of anti-TfR1 antibody (e.g., Fab) is approximately 11 mg (e.g., ±up to 33%, ±up to 30%, ±up to 25%, ±up to 20%, ±up to 15%, ±up to 10%, ±up to 5%, ±up to 3%, or ±up to 1%) per kg of the subject. In some embodiments, the sufficient amount of anti-TfR1 antibody (e.g., Fab) is approximately 10.93 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject about 10 mg of the oligonucleotides of the complexes per kg of the subject, and wherein the sufficient amount of anti-TfR1 antibody (e.g., Fab) is approximately 22 mg (e.g., ±up to 33%, ±up to 30%, ±up to 25%, ±up to 20%, ±up to 15%, ±up to 10%, ±up to 5%, ±up to 3%, or ±up to 1%) per kg of the subject. In some embodiments, the sufficient amount of anti-TfR1 antibody (e.g., Fab) is approximately 21.85 mg or 21.86 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject about 20 mg of the oligonucleotides of the complexes per kg of the subject, and wherein the sufficient amount of anti-TfR1 antibody (e.g., Fab) is approximately 44 mg (e.g., ±up to 33%, ±up to 30%, ±up to 25%, ±up to 20%, ±up to 15%, ±up to 10%, ±up to 5%, ±up to 3%, or ±up to 1%) per kg of the subject. In some embodiments, the sufficient amount of anti-TfR1 antibody (e.g., Fab) is approximately 43.71 mg or 43.72 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject about 30 mg of the oligonucleotides of the complexes per kg of the subject, and wherein the sufficient amount of anti-TfR1 antibody (e.g., Fab) is approximately 66 mg (e.g., ±up to 33%, ±up to 30%, ±up to 25%, ±up to 20%, ±up to 15%, ±up to 10%, ±up to 5%, ±up to 3%, or ±up to 1%) per kg of the subject. In some embodiments, the sufficient amount of anti-TfR1 antibody (e.g., Fab) is approximately 65.56 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject about 40 mg of the oligonucleotides of the complexes per kg of the subject, and wherein the sufficient amount of anti-TfR1 antibody (e.g., Fab) is approximately 88 mg (e.g., ±up to 33%, ±up to 30%, ±up to 25%, ±up to 20%, ±up to 15%, ±up to 10%, ±up to 5%, ±up to 3%, or ±up to 1%) per kg of the subject. In some embodiments, the sufficient amount of anti-TfR1 antibody (e.g., Fab) is approximately 87.42 mg or 87.43 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes of the composition to deliver to the subject oligonucleotides of the complexes, wherein the sufficient amount of anti-TfR1 antibody (e.g., Fab) is approximately 11.47 mg to 91.79 mg (e.g., 11.47 mg to 91.79 mg, 11.47 mg to 80.31 mg, 11.47 mg to 68.84 mg, 11.47 mg to 57.37 mg, 11.47 mg to 45.89 mg, 11.47 mg to 34.42 mg, 11.47 mg to 22.95 mg, 22.95 mg to 91.79 mg, 22.95 mg to 80.31 mg, 22.95 mg to 68.84 mg, 22.95 mg to 57.37 mg, 22.95 mg to 45.89 mg, 22.95 mg to 34.42 mg, 34.42 mg to 91.79 mg, 34.42 mg to 80.31 mg, 34.42 mg to 68.84 mg, 34.42 mg to 57.37 mg, 34.42 mg to 45.89 mg, 45.89 mg to 91.79 mg, 45.89 mg to 80.31 mg, 45.89 mg to 68.84 mg, 45.89 mg to 57.37 mg, 57.37 mg to 91.79 mg, 57.37 mg to 80.31 mg, 57.37 mg to 68.84 mg, 68.84 mg to 91.79 mg, 68.84 mg to 80.31 mg, or 80.31 mg to 91.79 mg) per kg of the subject. In some embodiments, with respect to any of the preceding amounts of anti-TfR1 antibody (e.g., Fab), the values can vary by up to ±25% (e.g., ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes of the composition to deliver to the subject oligonucleotides of the complexes, and wherein the sufficient amount of anti-TfR1 antibody (e.g., Fab) is approximately 11.47 mg, 13.77 mg, 16.06 mg, 18.36 mg, 20.65 mg, 22.95 mg, 25.24 mg, 27.54 mg, 29.83 mg, 32.13 mg, 34.42 mg, 36.71 mg, 39.01 mg, 41.30 mg, 43.60 mg, 45.89 mg, 48.19 mg, 50.48 mg, 52.78 mg, 55.07 mg, 57.37 mg, 59.66 mg, 61.96 mg, 64.25 mg, 66.55 mg, 68.84 mg, 71.14 mg, 73.43 mg, 75.72 mg, 78.02 mg, 80.31 mg, 82.61 mg, 84.90 mg, 87.20 mg, 89.49 mg, 91.79 mg per kg of the subject. In some embodiments, with respect to any of the preceding amounts of anti-TfR1 antibody (e.g., Fab), the values can vary by up to ±25% (e.g., ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 18.36 mg (e.g., ±up to 25%, ±up to 20%, ±up to 15%, ±up to 10%, ±up to 5%, ±up to 3%, or ±up to 1%) to approximately 27.54 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 22.95 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 55.07 mg (e.g., ±up to 25%, ±up to 20%, ±up to 15%, ±up to 10%, ±up to 5%, ±up to 3%, or ±up to 1%) to approximately 82.61 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 68.84 mg (e.g., ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes of the composition to deliver to the subject oligonucleotides of the complexes, wherein the sufficient amount of anti-TfR1 antibody (e.g., Fab) is approximately 10 mg to 90 mg (e.g., 11 mg, 22 mg, 44 mg, 66 mg, or 88 mg) per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes of the composition to deliver to the subject oligonucleotides of the complexes, and wherein the sufficient amount of anti-TfR1 antibody (e.g., Fab) is approximately 11 mg, 22 mg, 44 mg, 66 mg, or 88 mg per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 7 mg (e.g., ±up to 33%, ±up to 30%, ±up to 25%, ±up to 20%, ±up to 15%, ±up to 10%, ±up to 5%, ±up to 3%, or ±up to 1%) to approximately 15 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 11 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 10.93 mg (e.g., ±up to 33%, ±up to 30%, ±up to 25%, ±up to 20%, ±up to 15%, ±up to 10%, ±up to 5%, ±up to 3%, or ±up to 1%). In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 15 mg (e.g., ±up to 33%, ±up to 30%, ±up to 25%, ±up to 20%, ±up to 15%, ±up to 10%, ±up to 5%, ±up to 3%, or ±up to 1%) to approximately 30 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 22 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 21.85 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 21.86 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 30 mg (e.g., ±up to 33%, ±up to 30%, ±up to 25%, ±up to 20%, ±up to 15%, ±up to 10%, ±up to 5%, ±up to 3%, or ±up to 1%) to approximately 59 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 44 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 43.71 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 43.72 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 46 mg (e.g., ±up to 33%, ±up to 30%, ±up to 25%, ±up to 20%, ±up to 15%, ±up to 10%, ±up to 5%, ±up to 3%, or ±up to 1%) to approximately 88 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 65.56 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 61 mg (e.g., ±up to 33%, ±up to 30%, ±up to 25%, ±up to 20%, up to 15%, ±up to 10%, ±up to 5%, up to 3%, or ±up to 1%) to approximately 117 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 88 mg (e.g., ±up to 33%, ±up to 30%, ±up to 25%, ±up to 20%, ±up to 15%, ±up to 10%, ±up to 5%, ±up to 3%, or ±up to 1%) per kg of the subject.

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 87.42 mg (e.g., ±up to 33%, ±up to 30%, ±up to 25%, ±up to 20%, ±up to 15%, ±up to 10%, ±up to 5%, ±up to 3%, or ±up to 1%).

In some embodiments, a method provided herein comprises administering to the subject a composition comprising complexes comprising any one of the structures of formula (I): [R¹]ₙ₁—R² as described herein, wherein the average value of n1 of the complexes of the composition is 2.1, wherein the composition comprises a sufficient amount of the anti-TfR1 antibody (e.g., Fab) of the complexes to deliver to the subject oligonucleotides of the complexes, and wherein the sufficient amount of anti-TfR1 antibody (e.g., Fab) is approximately 87.43 mg (e.g., ±up to 33%, ±up to 30%, ±up to 25%, ±up to 20%, ±up to 15%, ±up to 10%, ±up to 5%, ±up to 3%, or ±up to 1%).

In some embodiments, in any one of the methods described herein, the effective amount provides to the subject about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54, mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg of the anti-TfR1 antibody (e.g., Fab) of the complexes per kg of the subject. In some embodiments, in any one of the methods described herein, the effective amount provides to the subject about 1.5 mg, 3 mg, 6 mg, 11 mg, 22 mg, 44 mg, or 88 mg of the anti-TfR1 antibody (e.g., Fab) of the complexes per kg of the subject. In some embodiments, in any one of the methods described herein, the effective amount provides to the subject about 114.7 mg, 108.0 mg, 102.0 mg, 96.62 mg, 91.79 mg, 87.42 mg, 86.05 mg, 83.44 mg, 80.99 mg, 79.82 mg, 76.49 mg, 76.44 mg, 73.43 mg, 72.46 mg, 70.61 mg, 68.84 mg, 67.99 mg, 65.56 mg, 62.58 mg, 59.86 mg, 57.37 mg, 55.07 mg, 53.99 mg, 52.95 mg, 50.99 mg, 48.31 mg, 45.89 mg, 43.71 mg, 41.72 mg, 39.91 mg, 38.24 mg, 36.71 mg, 35.30 mg, 34.00 mg, 28.68 mg, 27.0 mg, 25.50 mg, 24.15 mg, 22.95 mg, 21.85 mg, 20.86 mg, 19.95 mg, 19.12 mg, 18.36 mg, 17.65 mg, 17.00 mg, 14.34 mg, 13.50 mg, 12.75 mg, 12.08 mg, 11.47 mg, 10.93 mg, 10.43 mg, 9.98 mg, 9.56 mg, 9.18 mg, 8.83 mg, 8.50 mg, 8.03 mg, 7.56 mg, 7.14 mg, 6.76 mg, 6.43 mg, 6.12 mg, 5.84 mg, 5.59 mg, 5.35 mg, 5.14 mg, 4.94 mg, 4.76 mg, 4.02 mg, 3.78 mg, 3.57 mg, 3.38 mg, 3.21 mg, 3.06 mg, 2.92 mg, 2.79 mg, 2.68 mg, 2.57 mg, 2.47 mg, 2.38 mg, 2.01 mg, 1.89 mg, 1.78 mg, 1.69 mg, 1.61 mg, 1.53 mg, 1.46 mg, 1.40 mg, 1.34 mg, 1.29 mg, 1.24 mg, 1.19 mg of the anti-TfR1 antibody (e.g., Fab) of the complexes per kg of the subject. In some embodiments, in any one of the methods described herein, the effective amount provides to the subject about 1.53 mg, 3.06 mg, 6.12 mg, 10.93 mg, 21.85 mg, 43.71 mg, 65.56 mg, 87.42 mg of the anti-TfR1 antibody (e.g., Fab) of the complexes per kg of the subject.

In some embodiments, a method described herein comprises administering to the subject a composition comprising an effective amount of complexes, each complex comprising an anti-TfR1 antibody covalently linked to one or more oligonucleotides, wherein the effective amount provides to the subject 1.09 mg to 10.93 mg (e.g., 1.09 mg to 10.93 mg, 1.09 mg to 9.83 mg, 1.09 mg to 9.18 mg, 1.09 mg to 7.65 mg, 1.09 mg to 6.56 mg, 1.09 mg to 5.46 mg, 2.19 mg to 10.93 mg, 2.19 mg to 9.83 mg, 2.19 mg to 9.18 mg, 2.19 mg to 7.65 mg, 2.19 mg to 6.56 mg, 2.19 mg to 5.46 mg, 4.37 mg to 10.93 mg, 4.37 mg to 9.83 mg, 4.37 mg to 9.18 mg, 4.37 mg to 7.65 mg, 4.37 mg to 6.56 mg, 66.56 mg to 10.93 mg, 6.56 mg to 9.83 mg, 6.56 mg to 9.18 mg, 7.65 mg to 10.93 mg, 7.65 mg to 9.83 mg, or 9.18 mg to 10.93 mg) of the anti-TfR1 antibody (e.g., Fab) of the complexes per kg of the subject. In some embodiments, the effective amount provides to the subject 1.53 mg to 6.12 mg (e.g., 1.53 mg, 3.06 mg, or 6.12 mg) of the anti-TfR1 antibody (e.g., Fab) of the complexes per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%). In some embodiments, the subject is administered a single dose of a composition comprising the complexes described herein. In some embodiments, the subject is administered multiple doses of any one of the compositions comprising the complexes described herein.

In some embodiments, a method described herein comprises administering to the subject a composition comprising an effective amount (e.g., providing to the subject 1.09 mg to 10.93 mg (e.g., 1.09 mg to 10.93 mg (e.g., 1.09 mg to 10.93 mg, 1.09 mg to 9.83 mg, 1.09 mg to 9.18 mg, 1.09 mg to 7.65 mg, 1.09 mg to 6.56 mg, 1.09 mg to 5.46 mg, 2.19 mg to 10.93 mg, 2.19 mg to 9.83 mg, 2.19 mg to 9.18 mg, 2.19 mg to 7.65 mg, 2.19 mg to 6.56 mg, 2.19 mg to 5.46 mg, 4.37 mg to 10.93 mg, 4.37 mg to 9.83 mg, 4.37 mg to 9.18 mg, 4.37 mg to 7.65 mg, 4.37 mg to 6.56 mg, 66.56 mg to 10.93 mg, 6.56 mg to 9.83 mg, 6.56 mg to 9.18 mg, 7.65 mg to 10.93 mg, 7.65 mg to 9.83 mg, or 9.18 mg to 10.93) of the anti-TfR1 antibody (e.g., Fab) of the complexes per kg of the subject) of complexes described herein once a week to once every four months. For example, in some embodiments, a method described herein comprises administering to the subject a composition comprising an effective amount 1.09 mg to 10.93 mg (e.g., 1.09 mg to 10.93 mg, 1.09 mg to 9.83 mg, 1.09 mg to 9.18 mg, 1.09 mg to 7.65 mg, 1.09 mg to 6.56 mg, 1.09 mg to 5.46 mg, 2.19 mg to 10.93 mg, 2.19 mg to 9.83 mg, 2.19 mg to 9.18 mg, 2.19 mg to 7.65 mg, 2.19 mg to 6.56 mg, 2.19 mg to 5.46 mg, 4.37 mg to 10.93 mg, 4.37 mg to 9.83 mg, 4.37 mg to 9.18 mg, 4.37 mg to 7.65 mg, 4.37 mg to 6.56 mg, 66.56 mg to 10.93 mg, 6.56 mg to 9.83 mg, 6.56 mg to 9.18 mg, 7.65 mg to 10.93 mg, 7.65 mg to 9.83 mg, or 9.18 mg to 10.93 mg) of the anti-TfR1 antibody (e.g., Fab) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount of complexes (e.g., muscle targeting complexes) described herein, wherein the effective amount for each administration provides to the subject 1 mg to 8 mg (e.g., 1 mg, 1.5 mg, 2 mg, 3 mg, 4 mg, 5 mg, 6 mg, 7 mg, or 8 mg) of the anti-TfR1 antibody (e.g., Fab) of the complexes per kg of the subject) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime.

In some embodiments, a method described herein comprises administering to the subject a composition comprising an effective amount of complexes, each complex comprising an anti-TfR1 antibody covalently linked to one or more oligonucleotides, wherein the effective amount provides to the subject 1.5 mg to 6 mg (e.g., providing to the subject 1.5 mg to 6 mg (e.g., 1.5 mg to 6 mg, 1.5 mg to 5.5 mg, 1.5 mg to 5 mg, 1.5 mg to 4.5 mg, 1.5 mg to 4 mg, 15 mg to 3.5 mg, 1.5 mg to 3 mg, 1.5 mg to 2.5 mg, 1.5 mg to 2 mg) of the anti-TfR1 antibody (e.g., Fab) of the complexes per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount (e.g., providing to the subject about 1.5 mg, 3 mg, or 6 mg) of the anti-TfR1 antibody (e.g., Fab) of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount of the anti-TfR1 antibody (e.g., Fab) of the complexes per kg of the subject) of complexes (e.g., muscle targeting complexes) described herein once a week to once every four months, wherein the effective amount of the anti-TfR1 antibody (e.g., Fab) is 1.5 mg, 3 mg, 6 mg per kg of the subject. In some embodiments, a method described herein comprises administering to the subject a composition comprising an effective amount (e.g., providing to the subject about 1.53 mg, 3.06 mg, or 6.12 mg) of the anti-TfR1 antibody (e.g., Fab) of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, the effective amount of each administration provides to the subject 1.5 mg to 6 mg (e.g., 1.5 mg, 3 mg, or 6 mg) per kg of the subject. In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount of complexes (e.g., muscle targeting complexes) described herein, wherein the effective amount for each administration provides to the subject 1.07 mg to 2.03 mg (e.g., 1.07 mg, 1.09 mg, 1.45 mg, 1.5 mg, 1.53 mg, or 2.03 mg), of the anti-TfR1 antibody (e.g., Fab) of the complexes per kg of the subject. In some embodiments, the effective amount for each administration provides to the subject 1 mg to 2 mg (e.g., 1 mg, 1.5 mg, or 2 mg), of the anti-TfR1 antibody (e.g., Fab) of the complexes per kg of the subject. In some embodiments, a method described herein comprises administering to the subject a composition comprising an effective amount of complexes (e.g., muscle targeting complexes) described herein, wherein the effective amount for each administration provides to the subject 1.5 mg of the anti-TfR1 antibody (e.g., Fab) of the complexes per kg of the subject. In some embodiments, the effective amount for each administration provides to the subject 1.53 mg of the anti-TfR1 antibody (e.g., Fab) of the complexes per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes (e.g., muscle targeting complexes) described herein, wherein the effective amount for each administration provides to the subject approximately 2 mg to 4 mg (e.g., 2 mg, 3 mg, or 4 mg) of the anti-TfR1 antibody (e.g., Fab) of the complexes per kg of the subject. In some embodiments, the effective amount for each administration provides to the subject approximately 2.14 mg to 4.07 mg (e.g., 2.14 mg, 3.06 mg, or 4.07 mg) of the anti-TfR1 antibody (e.g., Fab) of the complexes per kg of the subject. In some embodiments, a method described herein comprises administering to the subject a composition comprising an effective amount of complexes (e.g., muscle targeting complexes) described herein, wherein the effective amount for each administration provides to the subject 3 mg of the anti-TfR1 antibody (e.g., Fab) of the complexes per kg of the subject. In some embodiments, the effective amount for each administration provides to the subject 3.06 mg of the anti-TfR1 antibody (e.g., Fab) of the complexes per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes (e.g., muscle targeting complexes) described herein, wherein the effective amount for each administration provides to the subject approximately 4 mg to 8 mg (e.g., 4 mg, 6 mg, or 8 mg), of the anti-TfR1 antibody (e.g., Fab) of the complexes per kg of the subject. In some embodiments, the effective amount for each administration provides to the subject approximately 4.28 mg to 8.14 mg (e.g., 4.28 mg, 4.59 mg, 6.12 mg, 6.56 mg, 7.65 mg, or 8.14 mg), of the anti-TfR1 antibody (e.g., Fab) of the complexes per kg of the subject. In some embodiments, a method described herein comprises administering to the subject a composition comprising an effective amount of complexes (e.g., muscle targeting complexes) described herein, wherein the effective amount for each administration provides to the subject 6 mg of the anti-TfR1 antibody (e.g., Fab) of the complexes per kg of the subject. In some embodiments, the effective amount for each administration provides to the subject 6.12 mg of the anti-TfR1 antibody (e.g., Fab) of the complexes per kg of the subject. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%). In some embodiments, a method described herein comprises administering to the subject a composition comprising an effective amount of complexes (e.g., muscle targeting complexes) described herein once every 4 weeks. In some embodiments, a method described herein comprises administering to the subject a composition comprising an effective amount of complexes (e.g., muscle targeting complexes) described herein once every 8 weeks.

In some embodiments, a method described herein comprises administering to the subject a composition comprising an effective amount of complexes, each complex comprising an anti-TfR1 antibody covalently linked to one or more oligonucleotides, wherein the effective amount provides to the subject 5 mg to 120 mg (e.g., 5 mg to 120 mg, 5 mg to 100 mg, 5 mg to 80 mg, 5 mg to 50 mg, 5 mg to 40 mg, 5 mg to 30 mg, 5 mg to 20 mg, 10 mg to 60 mg, 10 mg to 50 mg, 10 mg to 40 mg, 10 mg to 30 mg, 30 mg to 60 mg, 40 mg to 60 mg, 60 mg to 80 mg, 80 mg to 100 mg, or 100 mg to 120 mg) of the anti-TfR1 antibody (e.g., Fab) of the complexes per kg of the subject. In some embodiments, the effective amount provides 1.5 mg, 3 mg, 6 mg, 11 mg, 22 mg, 44 mg, or 88 mg of the anti-TfR1 antibody (e.g., Fab) of the complexes per kg of the subject. In some embodiments, the effective amount provides 1.53 mg, 3.06 mg, 6.12 mg, 10.93 mg, 21.85 mg, 43.71 mg, or 87.41 mg of the anti-TfR1 antibody (e.g., Fab) of the complexes per kg of the subject. In some embodiments, the subject is administered a single dose of a composition comprising the complexes described herein. In some embodiments, the subject is administered multiple doses of any one of the compositions comprising the complexes described herein.

In some embodiments, a method described herein comprises administering to the subject a composition comprising an effective amount (e.g., providing to the subject 1.5 mg to 6 mg (e.g., 1.5 mg to 6 mg, 1.5 mg to 5 mg, 1.5 mg to 4 mg, 1.5 mg to 3 mg, 1.5 mg to 2 mg) of the anti-TfR1 antibody (e.g., Fab) of the complexes per kg of the subject) of complexes described herein once a week to once every four months. For example, in some embodiments, a method described herein comprises administering to the subject a composition comprising an effective amount (e.g., providing to the subject 1.5 mg to 6 mg (e.g., 1.5 mg, 3 mg, 6 mg) of the anti-TfR1 antibody (e.g., Fab) of the complexes per kg of the subject) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount (e.g., providing to the subject 10 mg to 90 mg (e.g., 10 mg to 90, 10 mg to 10 mg, 20 mg to 80 mg, 20 mg to 40 mg, 30 mg to 70 mg, 40 mg to 60 mg, or 80 mg to 90 mg) of the anti-TfR1 antibody (e.g., Fab) of the complexes per kg of the subject) of complexes described herein once a week to once every four months. For example, in some embodiments, a method described herein comprises administering to the subject a composition comprising an effective amount (e.g., providing to the subject 10 mg to 90 mg (e.g., 11 mg, 22 mg, 44 mg, 66 mg, or 88 mg) of the anti-TfR1 antibody (e.g., Fab) of the complexes per kg of the subject) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks. In some embodiments, a method described herein comprises administering to the subject a composition comprising an effective amount of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, ±up to 5%, ±up to 3%, or ±up to 1%).

In some embodiments, a method described herein comprises administering to the subject a composition comprising an effective amount (e.g., providing to the subject about 1.5 mg, 3 mg, 6 mg) of the anti-TfR1 antibody (e.g., Fab) of the complexes per kg of the subject) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 1.53 mg, 3.06 mg, 6.12 mg) of the anti-TfR1 antibody (e.g., Fab) of the complexes per kg of the subject) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime.

In some embodiments, a method described herein comprises administering to the subject a composition comprising an effective amount (e.g., providing to the subject about 8.50 mg, 8.83 mg, 9.18 mg, 9.56 mg, 9.98 mg, 10.43 mg, 10.93 mg, 11.47 mg, 12.08 mg, 12.75 mg, 13.50 mg, 14.34 mg, 17.00 mg, 17.65 mg, 18.36 mg, 19.12 mg, 19.95 mg, 20.86 mg, 21.85 mg, 22.95 mg, 24.15 mg, 25.50 mg, 27.00 mg, 28.68 mg, 34.00 mg, 35.30 mg, 36.71 mg, 38.24 mg, 39.91 mg, 41.72 mg, 43.71 mg, 45.89 mg, 48.31 mg, 50.99 mg, 52.95 mg, 53.99 mg, 55.07 mg, 57.37 mg, 57.37 mg, 59.86 mg, 62.58 mg, 65.56 mg, 67.99 mg, 68.84 mg, 70.61 mg, 72.46 mg, 73.43 mg, 76.49 mg, 79.82 mg, 80.99 mg, 83.44 mg, 86.05 mg, 87.42 mg, 91.79 mg, 96.62 mg, 101.99 mg, 107.99 mg, 114.73 mg) of the anti-TfR1 antibody (e.g., Fab) of the complexes per kg of the subject) of complexes described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 10.93 mg, 21.85 mg, 21.86 mg, 43.71 mg, 43.72 mg, 65.56 mg, 87.42 mg, or 87.43 mg) of the anti-TfR1 antibody (e.g., Fab) of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, a method described herein comprises administering to the subject a composition comprising an effective amount (e.g., providing to the subject about 11 mg, 22 mg, 44 mg, or 88 mg) of the anti-TfR1 antibody (e.g., Fab) of the complexes per kg of the subject) of complexes (e.g., muscle targeting complexes) described herein once a week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks for the remainder of the subject's lifetime. In some embodiments, with respect to any of the preceding amounts of anti-TfR1 antibody (e.g., Fab), the values can vary by up to ±33% (e.g., ±up to 33%, ±up to 30%, ±up to 25%, ±up to 20%, ±up to 15%, ±up to 10%, up to 5%, ±up to 3%, or ±up to 1%).

In some embodiments, a method described herein comprises administering via infusion an effective amount of a composition comprising complexes, wherein each complex comprises a structure of formula (I): [R¹]ₙ₁—R², wherein:
each R¹ comprises a group of the formula (Ib):

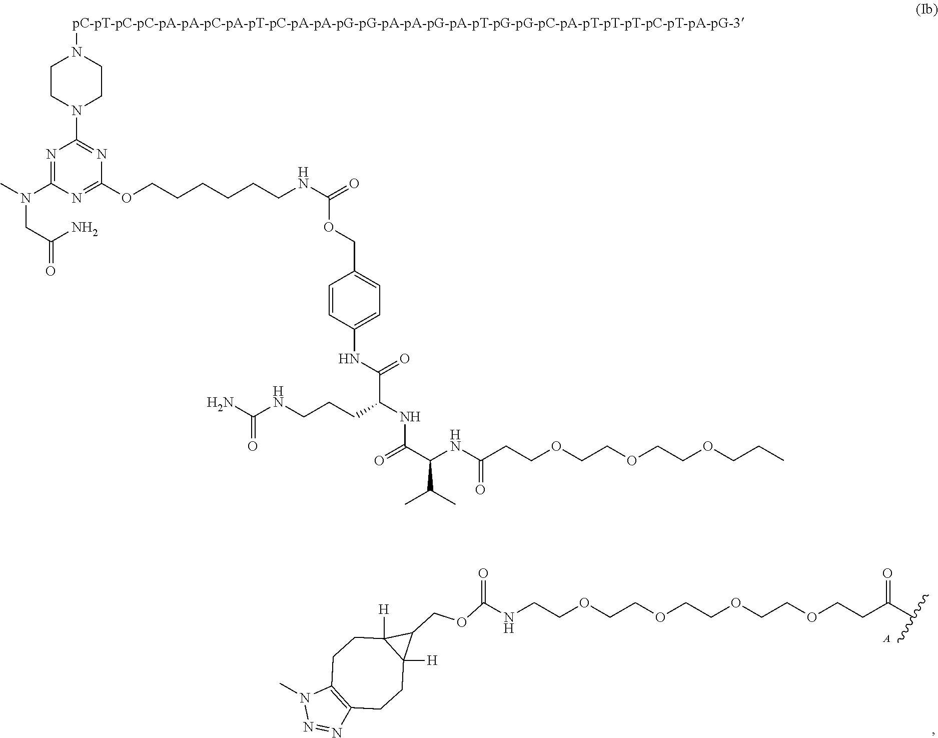

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the effective amount provides to the subject 1 mg to 8 mg (e.g., 1 mg, 1.5 mg, 2 mg, 3 mg, 4 mg, 5 mg, 6 mg, 7 mg, or 8 mg) of the anti-TfR1 antibody (e.g., Fab) of the complexes per kg of the subject. In some embodiments, the effective amount provides to the subject 1.5 mg to 6 mg (e.g., 1.5 mg, 3 mg, or 6 mg) per kg of the subject. In some embodiments, the subject is administered the composition once every four weeks, eight weeks, or twelve weeks. In some embodiments, the subject is administered the composition once every four weeks. In some embodiments, the subject is administered the composition once every eight weeks. In some embodiments, the effective amount provides to the subject 1 mg, 3 mg, or 6 mg of the anti-TfR1 antibody (e.g., Fab) of the complexes per kg of the subject. In some embodiments, the effective amount provides to the subject 1.5 mg, 3 mg, or 6 mg of the anti-TfR1 antibody (e.g., Fab) of the complexes per kg of the subject. In some embodiments, the effective amount provides to the subject 1.53 mg, 3.06 mg, or 6.12 mg of the anti-TfR1 antibody (e.g., Fab) of the complexes per kg of the subject. In some embodiments, a composition comprising complexes for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a composition comprising complexes (e.g., in an aqueous solution), wherein each complex comprises a structure of formula (I): [R¹]ₙ₁—R², wherein:
each R¹ comprises a group of the formula (Ic):

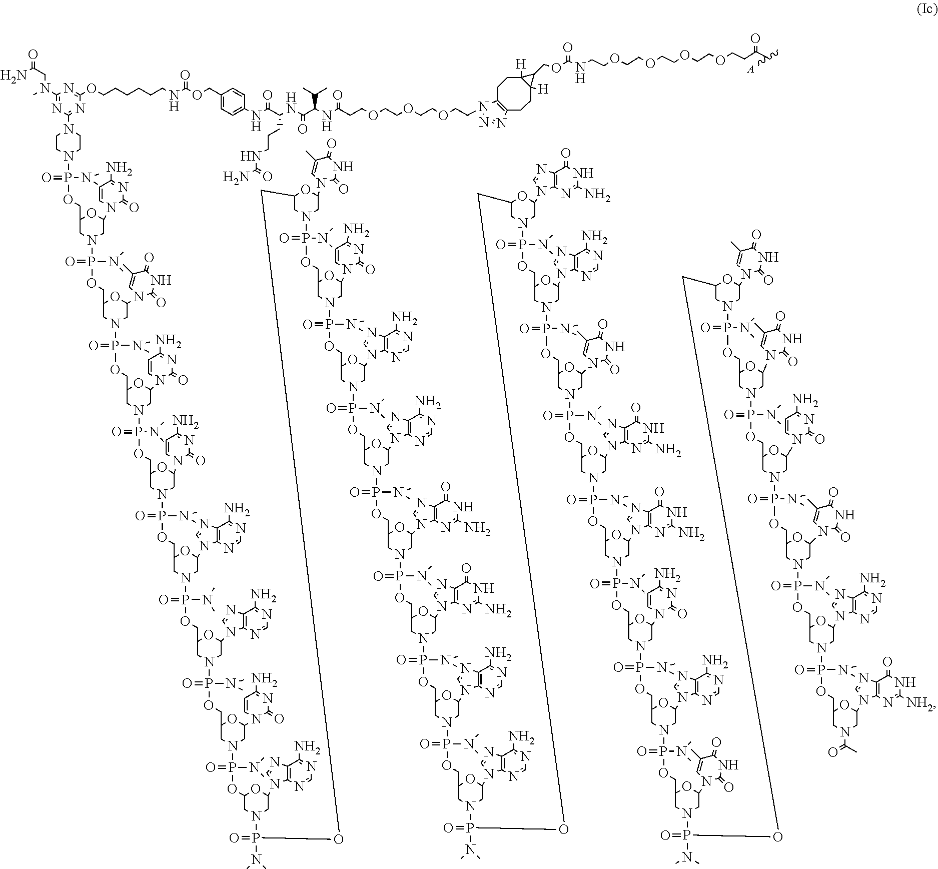

R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the effective amount provides to the subject 1 mg to 8 mg (e.g., 1 mg, 1.5 mg, 2 mg, 3 mg, 4 mg, 5 mg, 6 mg, 7 mg, or 8 mg) of the anti-TfR1 antibody (e.g., Fab) of the complexes per kg of the subject. In some embodiments, the effective amount provides to the subject 1.5 mg to 6 mg (e.g., 1.5 mg, 3 mg, or 6 mg) per kg of the subject. In some embodiments, the subject is administered the composition once every four weeks, eight weeks, or twelve weeks. In some embodiments, the subject is administered the composition once every four weeks. In some embodiments, the subject is administered the composition once every eight weeks. In some embodiments, the effective amount provides to the subject 1.5 mg, 3 mg, or 6 mg of the anti-TfR1 antibody (e.g., Fab) of the complexes per kg of the subject. In some embodiments, the effective amount provides to the subject 1.53 mg, 3.06 mg, or 6.12 mg of the anti-TfR1 antibody (e.g., Fab) of the complexes per kg of the subject. In some embodiments, a composition comprising complexes for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a composition comprising complexes (e.g., in an aqueous solution), wherein each complex comprises a structure of formula (Id):

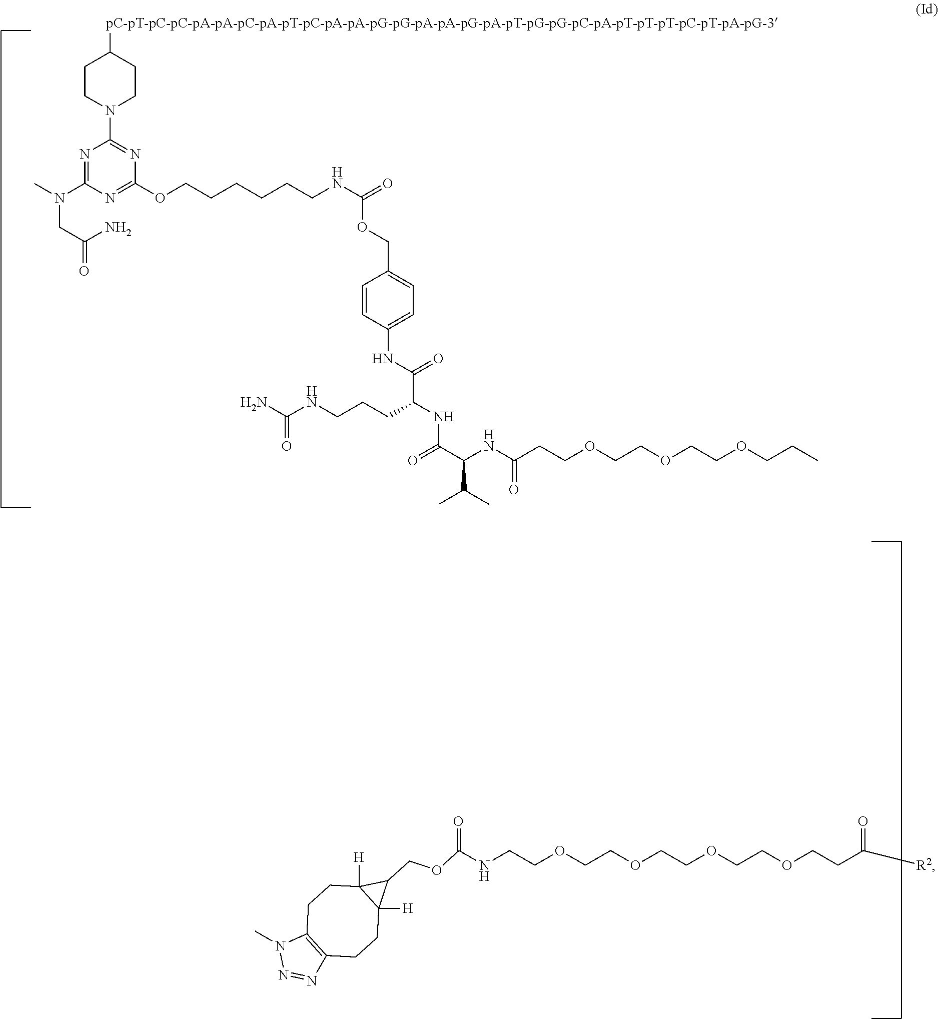

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; optionally wherein the anti-TfR1 antibody (e.g., Fab) is covalently linked (e.g., indirectly or directly linked, e.g., directly linked) via different amino acid residues of the antibody, optionally wherein each different amino acid residue is a lysine; and
wherein in each complex n1 is independently an integer of one or greater, optionally wherein the average value of n1 of the complexes of the composition is in the range of 1 to 5;
wherein the effective amount provides to the subject 1 mg to 8 mg (e.g., 1 mg, 1.5 mg, 2 mg, 3 mg, 4 mg, 5 mg, 6 mg, 7 mg, or 8 mg) of the anti-TfR1 antibody (e.g., Fab) of the complexes per kg of the subject. In some embodiments, the effective amount provides to the subject 1.5 mg to 6 mg (e.g., 1.5 mg, 3 mg, or 6 mg) per kg of the subject. In some embodiments, the subject is administered the composition once every four weeks, eight weeks, or twelve weeks. In some embodiments, the subject is administered the composition once every four weeks. In some embodiments, the subject is administered the composition once every eight weeks. In some embodiments, the effective amount provides to the subject 1.5 mg, 3 mg, or 6 mg of the anti-TfR1 antibody (e.g., Fab) of the complexes per kg of the subject. In some embodiments, the effective amount provides to the subject 1.53 mg, 3.06 mg, or 6.12 mg of the anti-TfR1 antibody (e.g., Fab) of the complexes per kg of the subject. In some embodiments, a composition comprising complexes for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a composition comprising complexes, wherein each complex comprises a structure of formula (I): [R¹]ₙ₁—R², wherein:
each R¹ comprises a group of the formula (Ib):

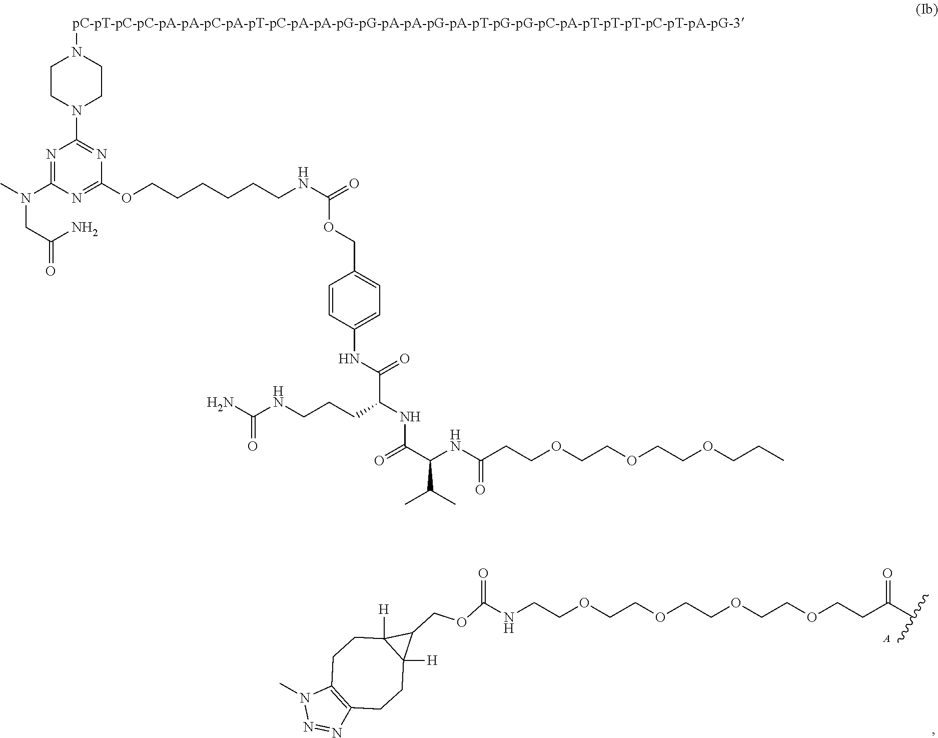

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the effective amount provides to the subject 5 mg to 120 mg of the anti-TfR1 antibody (e.g., Fab) of the complexes per kg of the subject. In some embodiments, the subject is administered the composition once every four weeks, eight weeks, or twelve weeks. In some embodiments, the effective amount provides to the subject 10 mg to 90 mg of the anti-TfR1 antibody (e.g., Fab) of the complexes per kg of the subject. In some embodiments, the effective amount provides to the subject 11 mg, 22 mg, 44 mg, or 88 mg of the anti-TfR1 antibody (e.g., Fab) of the complexes per kg of the subject. In some embodiments, the effective amount provides to the subject 10.93 mg, 21.85 mg, 43.71 mg, 65.56 mg, or 87.42 mg of the anti-TfR1 antibody (e.g., Fab) of the complexes per kg of the subject. In some embodiments, the effective amount provides to the subject 10.93 mg, 21.86 mg, 43.72 mg, 65.56 mg, or 87.43 mg of the anti-TfR1 antibody (e.g., Fab) of the complexes per kg of the subject. In some embodiments, a composition comprising complexes for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a composition comprising complexes (e.g., in an aqueous solution), wherein each complex comprises a structure of formula (I): [R¹]ₙ₁—R², wherein:
each R¹ comprises a group of the formula (Ic):

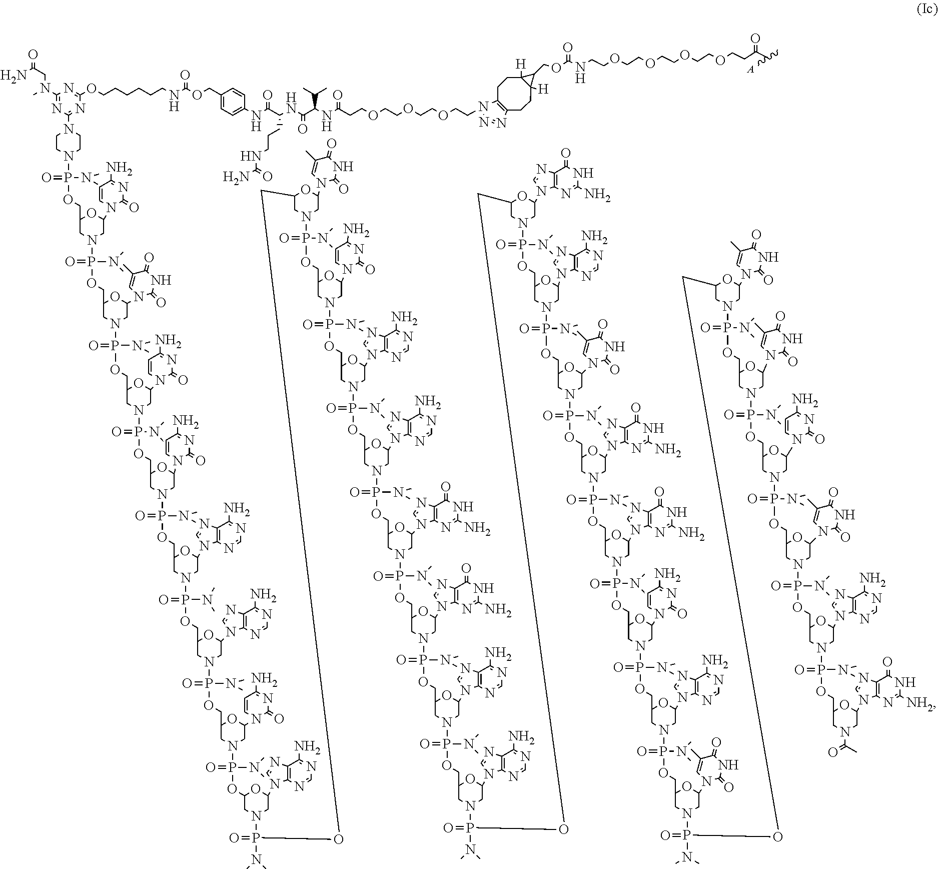

R² comprises an anti-TfR1 antibody (e.g., Fab) comprising a heavy chain comprising the amino acid sequence of SEQ ID NO: 19 and a light chain comprising the amino acid sequence of SEQ ID NO: 20; and
wherein in each complex n1 is independently an integer of one or greater representing the number of instances of R¹, wherein each instance of R¹ is covalently linked at attachment point A to a different lysine of the anti-TfR1 antibody (e.g., Fab), optionally wherein the average value of n1 of the complexes of the composition is in the range of 1 to 5;
wherein the effective amount provides to the subject 5 mg to 120 mg of the anti-TfR1 antibody (e.g., Fab) of the complexes per kg of the subject. In some embodiments, the subject is administered the composition once every four weeks, eight weeks, or twelve weeks. In some embodiments, the effective amount provides to the subject 10 mg to 90 mg of the anti-TfR1 antibody (e.g., Fab) of the complexes per kg of the subject. In some embodiments, the effective amount provides to the subject 11 mg, 22 mg, 44 mg, or 88 mg of the anti-TfR1 antibody (e.g., Fab) of the complexes per kg of the subject. In some embodiments, the effective amount provides to the subject 10.93 mg, 21.85 mg, 43.71 mg, 65.56 mg, or 87.42 mg of the anti-TfR1 antibody (e.g., Fab) of the complexes per kg of the subject. In some embodiments, the effective amount provides to the subject 10.93 mg, 21.86 mg, 43.72 mg, 65.56 mg, or 87.43 mg of the anti-TfR1 antibody (e.g., Fab) of the complexes per kg of the subject. In some embodiments, a composition comprising complexes for administration to a subject in the methods described herein further comprise complexes that comprise a structure of formula (I): [R¹]n1-R², wherein n1 is 0 and wherein R¹ and R² are defined herein.

In some embodiments, a method described herein comprises administering via infusion an effective amount of a composition comprising complexes (e.g., in an aqueous solution), wherein each complex comprises a structure of formula (Id:)

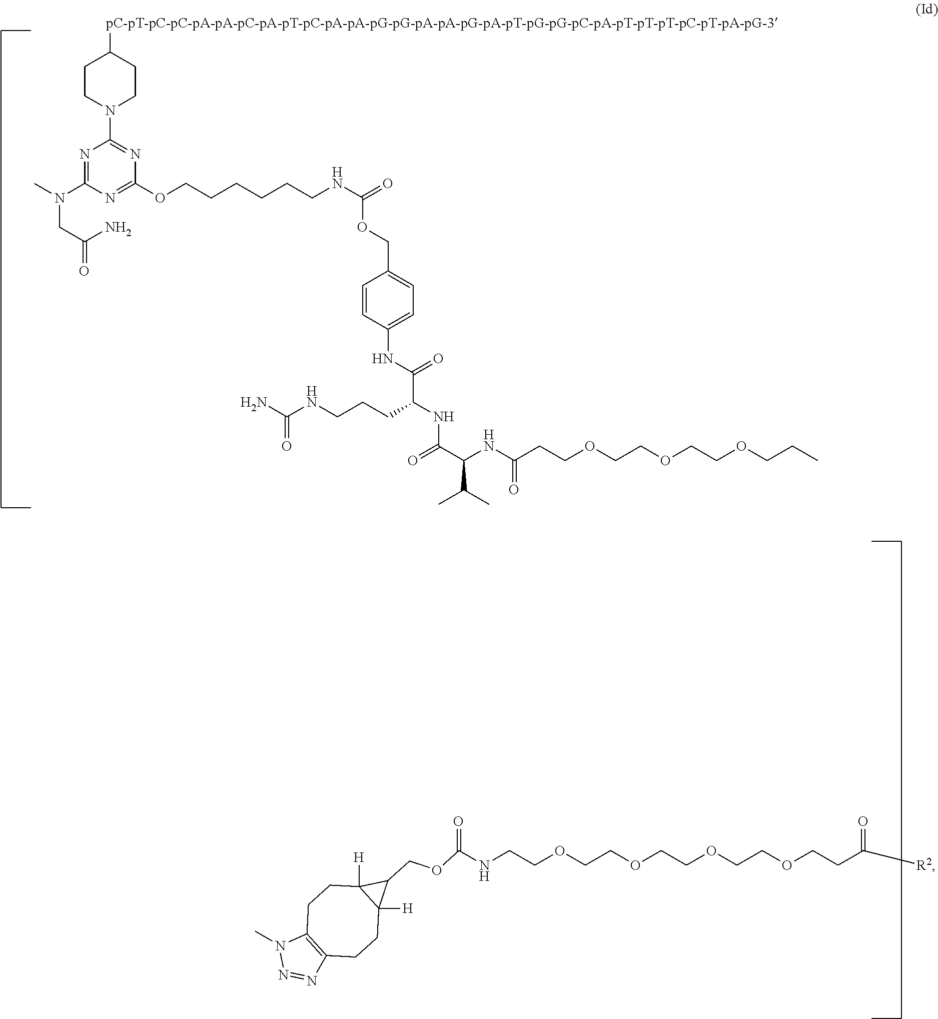

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 antibody (e.g., Fab) comprising a heavy chain comprising the amino acid sequence of SEQ ID NO: 19 and a light chain comprising the amino acid sequence of SEQ ID NO: 20; optionally wherein the anti-TfR1 antibody (e.g., Fab) is covalently linked (e.g., indirectly or directly linked, e.g., directly linked) via different amino acid residues of the antibody, optionally wherein each different amino acid residue is a lysine; and
wherein in each complex n1 is independently an integer of one or greater, optionally wherein the average value of n1 of the complexes of the composition is in the range of 1 to 5;
wherein the effective amount provides to the subject 5 mg to 120 mg of the anti-TfR1 antibody (e.g., Fab) of the complexes per kg of the subject. In some embodiments, the subject is administered the composition once every four weeks, eight weeks, or twelve weeks. In some embodiments, the effective amount provides to the subject 10 mg to 90 mg of the anti-TfR1 antibody (e.g., Fab) of the complexes per kg of the subject. In some embodiments, the effective amount provides to the subject 11 mg, 22 mg, 44 mg, or 88 mg of the anti-TfR1 antibody (e.g., Fab) of the complexes per kg of the subject. In some embodiments, the effective amount provides to the subject 10.93 mg, 21.85 mg, 43.71 mg, 65.56 mg, or 87.42 mg of the anti-TfR1 antibody (e.g., Fab) of the complexes per kg of the subject. In some embodiments, the effective amount provides to the subject 10.93 mg, 21.86 mg, 43.72 mg, 65.56 mg, or 87.43 mg of the anti-TfR1 antibody (e.g., Fab) of the complexes per kg of the subject. In some embodiments, a composition comprising complexes for administration to a subject in the methods described herein further comprise complexes that comprise a structure of formula (I): [R¹]n1-R², wherein n1 is 0 and wherein R¹ and R² are defined herein.

In some embodiments, in any one of the methods described herein, variations (e.g., ±up to 33%, ±up to 30%, ±up to 25%, ±up to 20%, ±up to 15%, ±up to 10%, ±up to 5%, ±up to 3%, or ±up to 1%) to approximately 50 mg (e.g., ±up to 20%, ±up to 15%, ±up to 10%, ±up to 5%, ±up to 3%, or ±up to 1%) in the amount of anti-TfR1 antibody (e.g., Fab) provided to the subject by administering the composition comprising an effective amount of the complexes described herein are contemplated.

In some embodiments, the composition is a formulation of an aqueous solution that is at a pH of 6.0 and comprises histidine at a concentration of 25 mM, sucrose at a concentration of 10 w/v %, and the complexes at a concentration in the range of 10 mg/mL to 50 mg/mL.

In some embodiments, the subject is administered the formulation once every 4 weeks, once every 8 weeks, or once every 12 weeks. In some embodiments, the subject is administered the formulation once every 4 weeks. In some embodiments, the subject is administered the formulation once every 8 weeks.

In some embodiments, administration of a composition comprising an effective amount of complexes (e.g., muscle targeting complexes) according to any one of the methods described herein promotes expression or activity of dystrophin protein (e.g., a truncated dystrophin protein) in the subject. The truncated dystrophin protein is functional (e.g., retains activities of a wild-type dystrophin protein). In some embodiments, the truncated dystrophin protein retains partial function of a wild-type dystrophin protein. In some embodiments, administration of a composition comprising an effective amount of complexes (e.g., muscle targeting complexes) according to any one of the methods described herein results in at least 1% (e.g., at least 1%, at least 2%, at least 3%, at least 4%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 30%, at least 40%, at least 50%, at least 60%, at least 70%, at least 80%, at least 90%) of dystrophin protein (e.g., a truncated dystrophin protein) in the subject relative to a control. In some embodiments, the control is dystrophin protein level in a healthy subject (e.g., a subject that does not have DMD). In some embodiments, the control is dystrophin protein level in a subject that does not have a mutated dystrophin allele, optionally wherein the subject that does not have a mutated dystrophin allele having a frameshift mutation.

In some embodiments, administration of a composition comprising an effective amount of complexes (e.g., muscle targeting complexes) according to any one of the methods described herein results in an increase (e.g., by at least 1%, at least 2%, at least 5%, at least 10%, at least 20%, at least 30%, at least 40%, at least 50%, at least 60%, at least 70%, at least 80%, at least 90%, 1-fold, 2-fold, 5-fold, 10-fold or more) dystrophin protein (e.g., a truncated dystrophin protein) in the subject. In some embodiments, the increase is observed before or at week 25 (e.g., week 10, week 15, week 20, or week 25) after initial administration of a composition described herein.

In some embodiments, administration of a composition comprising an effective amount of complexes (e.g., muscle targeting complexes) according to any one of the methods described herein results in an increase (e.g., by at least 1%, at least 2%, at least 5%, at least 10%, at least 20%, at least 30%, at least 40%, at least 50%, at least 60%, at least 70%, at least 80%, at least 90%, 1-fold, 2-fold, 5-fold, 10-fold or more) of the number of dystrophin positive fibers in the subject. In some embodiments, the increase is observed before or at week 25 (e.g., week 10, week 15, week 20, or week 25) after initial administration of a composition described herein.

In some embodiments, administration of a composition comprising an effective amount of complexes (e.g., muscle targeting complexes) according to any one of the methods described herein increases the number of dystrophin positive fibers to at least 10% (e.g., at least 10%, at least 20%, at least 30%, at least 40%, at least 50%, at least 60%, at least 70%, at least 80%, at least 90% or more) of total muscle fibers in an area of focus (e.g., an area of focus in the quadriceps, diaphragm, heart, gastrocnemius, or tibialis anterior) the subject. In some embodiments, the number of dystrophin positive fibers is quantified in an immunofluorescent image of immuno-stained (e.g., using anti-dystrophin antibody) muscle tissue of the subject. In some embodiments, administration of a composition comprising an effective amount of complexes (e.g., muscle targeting complexes) according to any one of the methods described herein alleviates one or more symptoms of DMD. In some embodiments, the increase is observed before or at week 25 (e.g., week 10, week 15, week 20, or week 25) after initial administration of a composition described herein.

In some embodiments, administration of a composition comprising an effective amount of complexes (e.g., muscle targeting complexes) according to any one of the methods described herein increases (e.g., by at least 0.5%, at least 1%, at least 5%, at least 10%, at least 20%, at least 30%, at least 40%, at least 50% or more) in exon (e.g., exon 51) skipping in a muscle tissue of the subject, relative to the level of exon (e.g., exon 51) skipping prior to the treatment using a method described herein. In some embodiments, the increase is observed before or at week 25 (e.g., week 10, week 15, week 20, or week 25) after initial administration of a composition described herein.

In some embodiments, administration of a composition comprising an effective amount of complexes (e.g., muscle targeting complexes) according to any one of the methods described herein reduces (e.g., by at least 0.5%, at least 1%, at least 5%, at least 10%, at least 20%, at least 30%, at least 40%, at least 50%, at least 60%, at least 70%, at least 80%, at least 90%, at least 95% or more) blood creatine kinase levels of the subject, relative to the blood creatine kinase levels prior to the treatment using a method described herein. In some embodiments, the reduction is observed over the course of up to 145 weeks (e.g., up to week 10, week 20, week 30, week 40, week 50, week 60, week 70, week 80, week 90, week 100, week 110, week 120, week 130, week 140, or week 145) after initial administration of a composition described herein. In some embodiments, the decrease is observed more than 145 weeks after initial administration of a composition described herein.

### EXAMPLES

### Example 1. Exon-Skipping Activity of Anti-TfR1 Conjugate in DMD Patient Myotubes

This study evaluated the exon-skipping activities of conjugates comprising the anti-TfR1 Fab having the heavy chain and light chain sequences shown in Table 2 covalently linked (through lysine conjugation) via a linker comprising a Valine-Citrulline sequence to a DMD exon 51-skipping oligonucleotide (oligo). The DMD exon 51-skipping oligonucleotide is a PMO and comprises the base sequence of SEQ ID NO: 21. The conjugates comprise a structure of formula (Id):

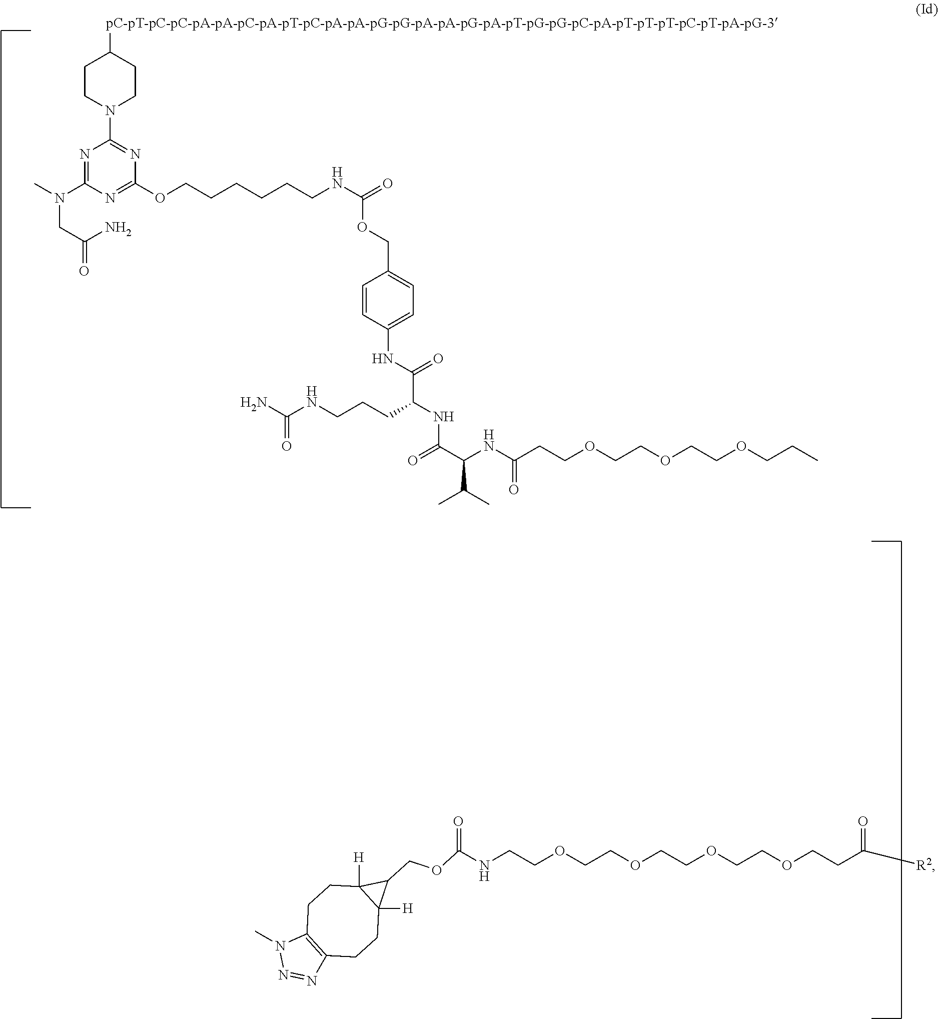

wherein R² is the anti-TfR1 Fab shown in Table 2, and wherein in each conjugate n1 is independently an integer of zero or greater.

Immortalized human myoblasts bearing an exon 52 deletion were thawed and seeded at a density of 1×10⁶ cell/flask in Promocell Skeletal Cell Growth Media (with 5% FBS and 1× Pen-Strep) and allowed to grow to confluency. Once confluent, cells were trypsinized and pelleted via centrifugation and resuspended in fresh Promocell Skeletal Cell Growth Media. The cell number was counted and cells were seeded into Matrigel-coated 96-well plates at a density of 50k cells/well. Cells were allowed to recover for 24 hours. Cells were induced to differentiate by aspirating the growth media and replacing with differentiation media with no serum. Cells were then treated with the DMD exon 51-skipping oligonucleotide (not covalently linked to an antibody —“naked”) at a concentration of 10 μM oligonucleotide or treated with the conjugate to a final concentration of 10 μM oligonucleotide equivalent. Cells were incubated with test articles for ten days then total RNA was harvested from the 96 well plates. cDNA synthesis was performed on 75 ng of total RNA, and mutation specific PCRs were performed to evaluate the degree of exon 51 skipping in each cell type. Mutation-specific PCR products were run on a 4% agarose gel and visualized using SYBR gold. Densitometry was used to calculate the relative amounts of the skipped and unskipped amplicon and exon skipping was determined as a ratio of the Exon 51 skipped amplicon divided by the total amount of amplicon present:

$$\% Exon Skipping = \frac{Skipped Amplicon}{( Skipped Amplicon + Unskipped Amplicon )} * 100$$

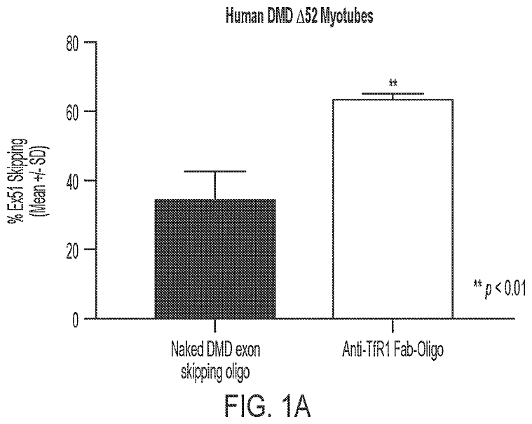

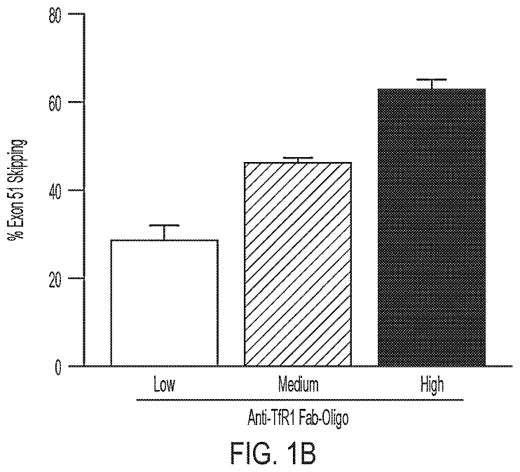

The results demonstrate that the conjugates resulted in enhanced exon skipping compared to the same DMD exon 51-skipping oligonucleotide that is not covalently linked to an antibody in patient myotubes (FIG. 1A). This indicates that an anti-TfR1 Fab (e.g., having a sequence set forth in Table 2) facilitated cellular internalization of the conjugate into muscle cells resulting in activity of the exon 51-skipping oligonucleotide in the muscle cells.

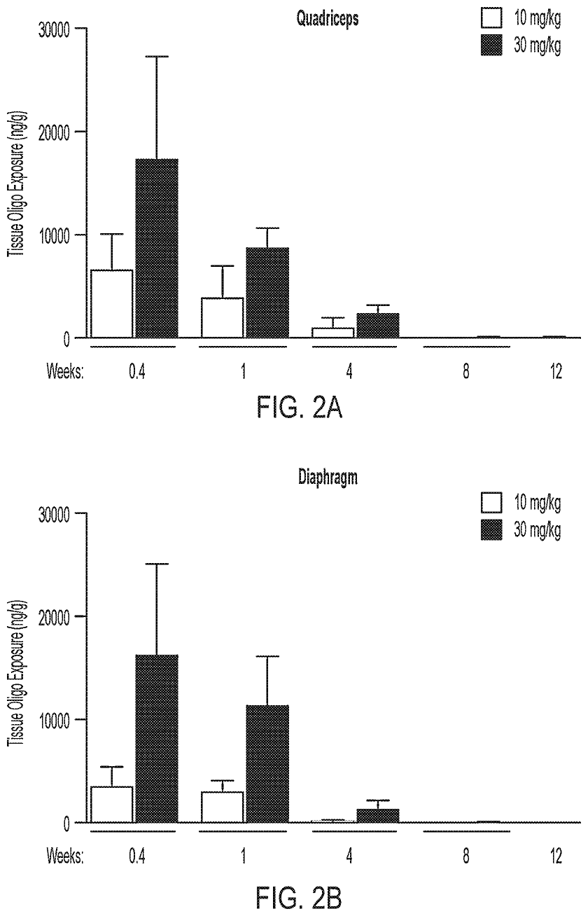

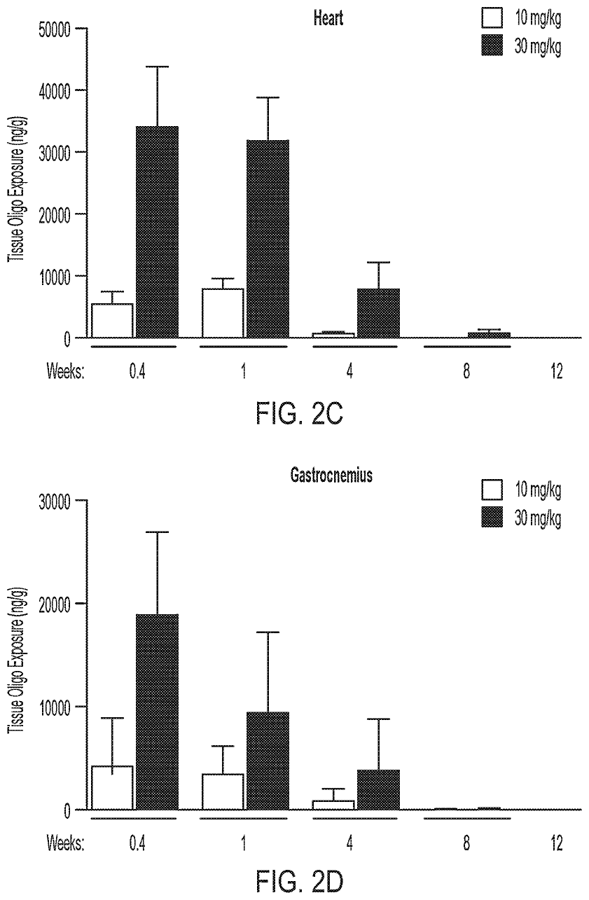

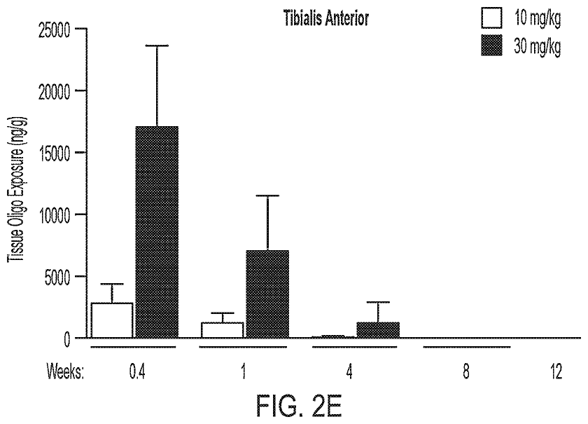

Additionally, cells were treated with anti-TfR1 Fab-oligo conjugates to a final concentration of 2.5 μM (low), 5 μM (medium), or 10 μM (high) oligonucleotide equivalent. Exon 51-skipping data is shown in FIG. 2B. The results demonstrate that the conjugates resulted in dose dependent exon 51 skipping in DMD patient myotubes.

### Example 2. Exon Skipping Activity of Anti-TfR1 Fab-Oligonucleotide Conjugate In Vivo in Cynomolgus Monkeys

The anti-TfR1-oligo conjugates described in Example 1 were tested for their exon skipping activity in vivo in healthy non-human primates. Naïve male cynomolgus monkeys (n=4-5 per group) were administered two doses of vehicle, 30 mg/kg naked oligo (i.e., not covalently linked to an antibody), or 122 mg/kg anti-TfR1 Fab covalently linked to the DMD exon 51-skipping oligonucleotide (30 mg/kg oligo equivalent) via intravenous infusion on days 1 and 8. Animals were sacrificed and tissues harvested either 2 weeks or 4 weeks after the first dose was administered. Total RNA was collected from tissue samples using a Promega Maxwell® RSC instrument and cDNA synthesis was performed using qScript cDNA SuperMix. Assessment of exon 51 skipping was performed using end-point PCR.

Capillary electrophoresis of the PCR products was used to assess exon skipping, and % exon 51 skipping was calculated using the following formula:

$$\% Exon Skipping = \frac{Molarity of Skipped Band}{Molarity of Skipped Band + Molarity of Unskipped Band} * 100.$$

Calculated exon 51 skipping results are shown in Table 4.

TABLE 4
Exon 51 skipping of dystrophin in cynomolgus monkey dystrophin

| | Time | | | | |
|---|---|---|---|---|---|
| | 2 weeks | | | 4 weeks | |
| | | Oligo | | Oligo | |
| Group | Vehicle | alone^{a} | Conjugate | alone^{a} | Conjugate |
| Conjugate dose^{b} | 0 | n/a | 122 | n/a | 122 |
| Oligo alone | 0 | 30 | 30 | 30 | 30 |
| Dose^{c} | | | | | |
| Quadriceps ^{d} | 0.00 | 1.216 | 4.906 | 0.840 | 1.708 |
| | (0.00) | (1.083) | (3.131) | (1.169) | (1.395) |
| Diaphragm ^{d} | 0.00 | 1.891 | 7.315 | 0.717 | 9.225 |
| | (0.00) | (2.911) | (1.532) | (1.315) | (4.696) |
| Heart ^{d} | 0.00 | 0.043 | 3.42 | 0.00 | 4.525 |
| | (0.00) | (0.096) | (1.192) | (0.00) | (1.400) |
| Biceps ^{d} | 0.00 | 0.607 | 3.129 | 1.214 | 4.863 |
| | (0.00) | (0.615) | (0.912) | (1.441) | (3.881) |
| Tibialis anterior ^{d} | 0.00 | 0.699 | 1.042 | 0.384 | 0.816 |
| | (0.00) | (0.997) | (0.685) | (0.615) | (0.915) |
| Gastrocnemius ^{d} | 0.00 | 0.388 | 2.424 | 0.00 | 5.393 |
| | (0.00) | (0.573) | (2.329) | (0.00) | (2.695) |

^{a}Oligo = Oligonucleotide.
^{b}Conjugate doses are listed as mg/kg of anti-TfR1 Fab-oligonucleotide conjugate.
^{c}Oligo doses are listed as mg/kg oligonucleotide equivalent of the anti-TfR1 Fab-oligonucleotide dose.
^{d} Exon skipping values are mean % exon 51 skipping with standard deviations (n = 5) in parentheses.

Tissue oligonucleotide (oligo) accumulation was quantified using a hybridization ELISA with a probe complementary to the oligonucleotide sequence. A standard curve was generated and oligonucleotide levels (in ng/g) were derived from a linear regression of the standard curve. The oligonucleotide was distributed to all tissues evaluated at a higher level following the administration of the anti-TfR1 Fab-oligonucleotide conjugate as compared to the administration of unconjugated oligonucleotide (not covalently linked to antibody). Intravenous administration of unconjugated oligonucleotide resulted in levels of oligonucleotide that were close to background levels in all tissues evaluated at 2 and 4 weeks after the first dose was administered. Administration of the conjugate resulted in distribution of oligonucleotide through the tissues evaluated with a rank order of heart>diaphragm>bicep>quadriceps>gastrocnemious>tibialis anterior 2 weeks after first dosing.

The duration of tissue concentration was also assessed. Oligonucleotide levels were detectable at 4 weeks post dose in all tissues (Table 5). This indicates that the anti-TfR1 Fab shown in Table 2 enabled cellular internalization of the conjugate into muscle cells in vivo, resulting in activity of the exon skipping oligonucleotide in the muscle cells.

TABLE 5
Tissue distribution of DMD exon 51 skipping
oligonucleotide in cynomolgus monkeys

| | Time | | | | |
|---|---|---|---|---|---|
| | 2 weeks | | | 4 weeks | |
| | | Oligo | | Oligo | |
| Group | Vehicle | alone^{a} | Conjugate | alone^{a} | Conjugate |
| Conjugate Dose^{b} | 0 | n/a | 122 | n/a | 122 |
| Oligo alone Dose^{c} | 0 | 30 | 30 | 30 | 30 |
| Quadriceps ^{d} | 0 | 696.8 | 2436 | 197 | 682 |
| | (59.05) | (868.15) | (954.0) | (134) | (281) |
| Diaphragm ^{d} | 0± | 580.02 | 6750 | 60 | 3131 |
| | (144.3) | (360.11) | (2256) | (120) | (1618) |
| Heart ^{d} | 0 | 1449 | 27138 | 943 | 30410 |
| | (396.03) | (1337) | (6315) | (1803) | (9247) |
| Biceps ^{d} | 0 | 615.63 | 2840 | 130 | 1326 |
| | (69.58) | (335.17) | (980.31) | (80) | (623) |
| Tibialis anterior ^{d} | 0 | 564.71 | 1591 | 169 | 1087 |
| | (76.31) | (327.88) | (253.50) | (110) | (514) |
| Gastrocnemius ^{d} | 0 | 705.47 | 2096 | 170 | 1265 |
| | (41.15) | (863.75) | (474.04) | (69) | (272) |

^{a}Oligo = Oligonucleotide.
^{b}Conjugate doses are listed as mg/kg of anti-TfR1 Fab-oligonucleotide conjugate.
^{c}Oligo doses are listed as mg/kg oligonucleotide or oligonucleotide equivalent of the anti-TfR1 Fab-oligonucleotide conjugate dose.
^{d} Oligo values are mean concentrations of oligonucleotide in tissue as ng/g with standard deviations (n = 5) in parentheses.

### Example 3: Exon Skipping Activity of Anti-TfR1 Fab-Oligonucleotide Conjugate In Vivo in Mdx Mice

Anti-TfR1 Fab-oligonucleotide conjugates were generated an anti-transferrin receptor (anti-TfR1) antibody (RI7 217 (Fab)) covalently linked via a linker comprising a Valine-Citrulline sequence to a dystrophin (DMD) exon 23-skipping oligonucleotide (oligo). The exon 23 skipping oligonucleotide is a phosphorodiamidate morpholino oligomer (PMO) of 25 nucleotides in length and comprises a base sequence of GGCCAAACCTCGGCTTACCTGAAAT (SEQ ID NO: 36).

The mdx mouse model was used as a surrogate model because no practical animal model currently exists for the disease state of subjects that are amenable for DMD exon 51 skipping. The mdx mouse model is illustrative of how the anti-TfR1-oligo conjugates behave in vivo. In this study, 5 week old C57BL/10ScSn-Dmd^{mdx}/J (mdx) mice were intravenously injected with a single bolus dose of the conjugate at either 67.7 mg/kg (equivalent to 30 mg/kg oligonucleotide) or 22.6 mg/kg (equivalent to 10 mg/kg oligonucleotide). Five mice were sacrificed and targeted muscle tissues were collected at each of 3 days, 1 week, 4 weeks, and 8 weeks, in the 22.6 mg/kg (equivalent to 10 mg/kg oligonucleotide) group. Five mice were sacrificed and targeted muscle tissues were collected at each of 3 days, 1 week, 4 weeks, 8 weeks, and 12 weeks, in the 67.7 mg/kg (equivalent to 30 mg/kg oligonucleotide) group.

Individual muscle tissue samples were subsequently assayed using hybridization ELISA (hELISA) for tissue oligonucleotide exposure. Exon 23 skipping of the dystrophin gene was assessed via end-point capillary electrophoresis, and percent dystrophin protein restoration via quantitative Western blot was performed at all time points. Additionally, dystrophin localization was determined by assessing the percent dystrophin positive fibers (PDPF) at selected time points and quantified.

### Measurement of Tissue Exposure

Tissue oligonucleotide exposure was also quantified using a hybridization ELISA (hELISA). Tissue oligonucleotide levels were measured in tissue samples collected 3 days, 1 week, 4 weeks, 8 weeks, and 12 weeks following administration. The administration of anti-TfR1 Fab-oligo conjugate resulted in a dose dependent, substantial accumulation of oligonucleotide (oligo) within quadriceps (FIG. 2A), diaphragm (FIG. 2B), heart (FIG. 2C), gastrocnemius (FIG. 2D), and tibialis anterior (FIG. 2E). These results demonstrate that administration of anti-TfR1 Fab-oligo conjugates at both doses (equivalent to 10 mg/kg oligonucleotide and 30 mg/kg oligonucleotide) resulted in measurable quantities of oligonucleotides in each of the tissues tested up to 4 weeks following administration. At the 30 mg/kg oligo dose, oligonucleotides in muscles were detectable even at 12 weeks post dose. The absolute levels are low, making it difficult to view in the bar graphs; however, oligonucleotides were detectable.

### Measurement of Exon 23 Skipping

Tissue was homogenized and RNA isolation was performed on the lysate using the Promega Maxwell RSC instrument and the Maxwell RSC simplyRNA Tissue kit per manufacturer's protocol. cDNA was generated from 75 ng of total RNA using the Quantabio qScript cDNA Supermix using manufacturer's protocol. End-point PCR was performed using primers to amplify the region of interest. Capillary electrophoresis of the PCR products was run on the LabChip HT Touch II instrument and percent exon skipping was quantified per the following equation:

$$\% Exon 23 Skipping = \frac{molarity of skipped transcript}{molarity of skipped + unskipped transcripts} \times 100$$

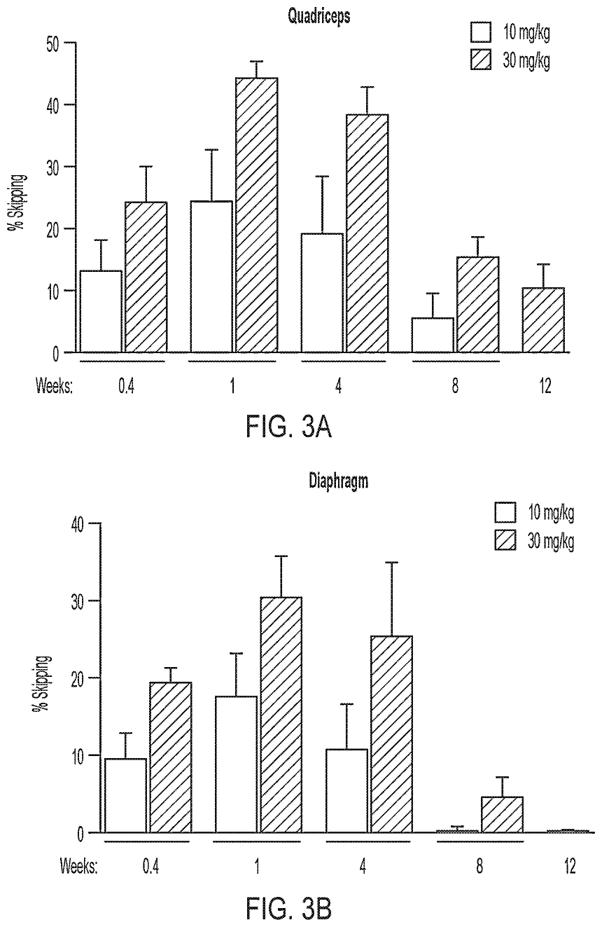

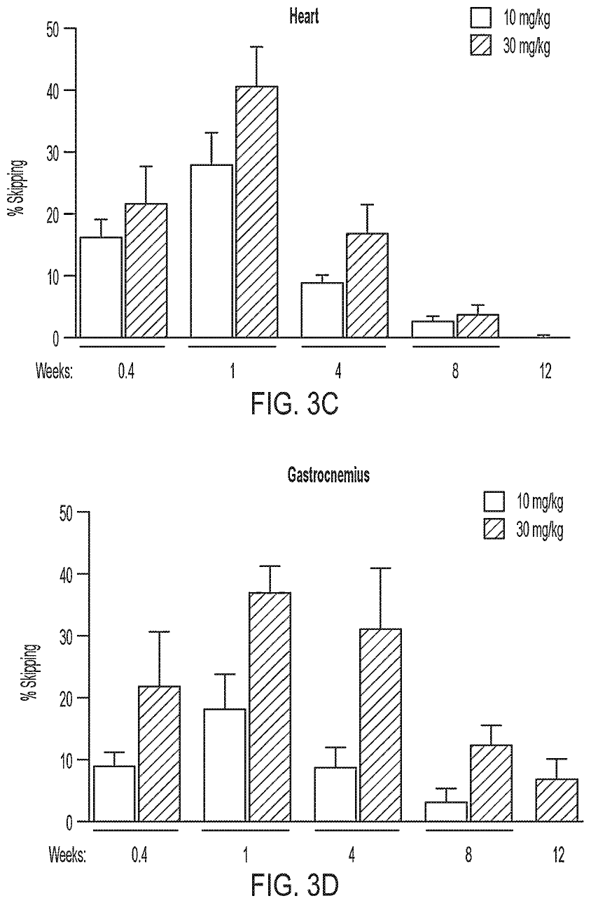

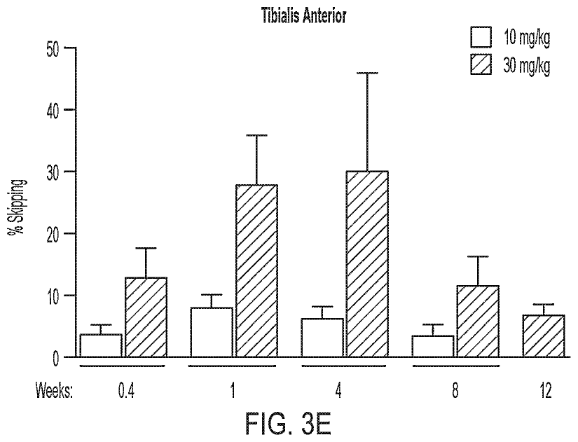

Mice treated with anti-TfR1 Fab-oligo conjugate demonstrated robust and dose-dependent exon skipping of exon 23 quadriceps (FIG. 3A), diaphragm (FIG. 3B), heart (FIG. 3C), gastrocnemius (FIG. 3D), and tibialis anterior (FIG. 3E).

### Measurement of Dystrophin Protein Expression

Tissue was homogenized and total protein content was evaluated using the Pierce Rapid Gold BCA Protein Assay Kit per manufacturer's protocol. Western blots were run with 25 μg of protein per well. After transfer, blots were imaged using anti-dystrophin primary antibody (Abcam, Ab15277; 1:500) and anti-alpha-actinin (Ab9465; 1:10,000). Secondary antibodies were IRDye 800CW Goat anti-Rabbit IgG and IRDye 680RD Goat anti-Mouse IgG, respectively. Blots were imaged on Licor Odyssey CLX instrument. Quantification was performed based on a linear standard curve of wild-type protein admixed with dystrophin-lacking mdx protein tissue at known percentages.

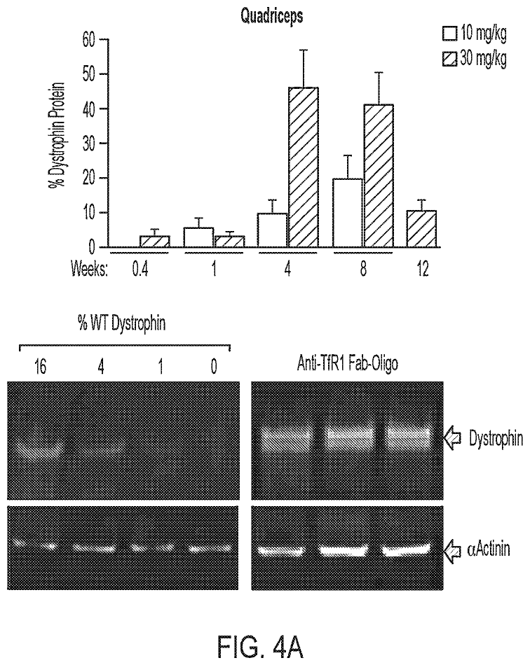

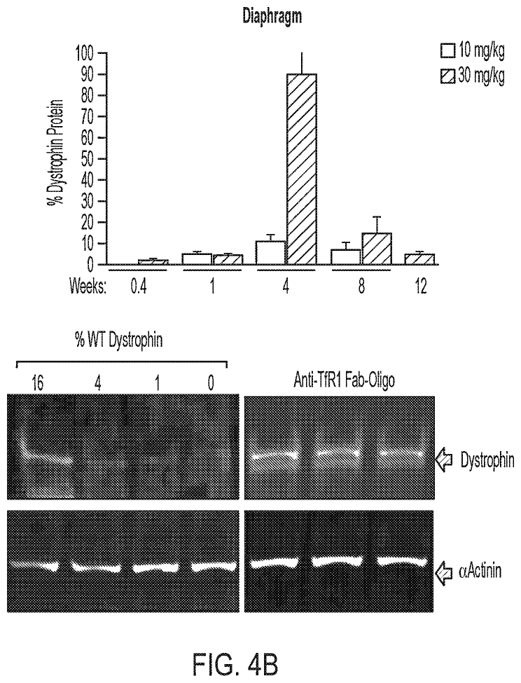

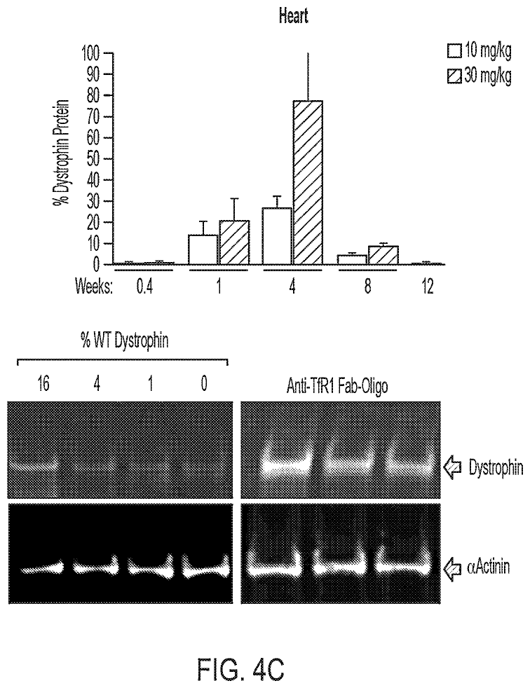

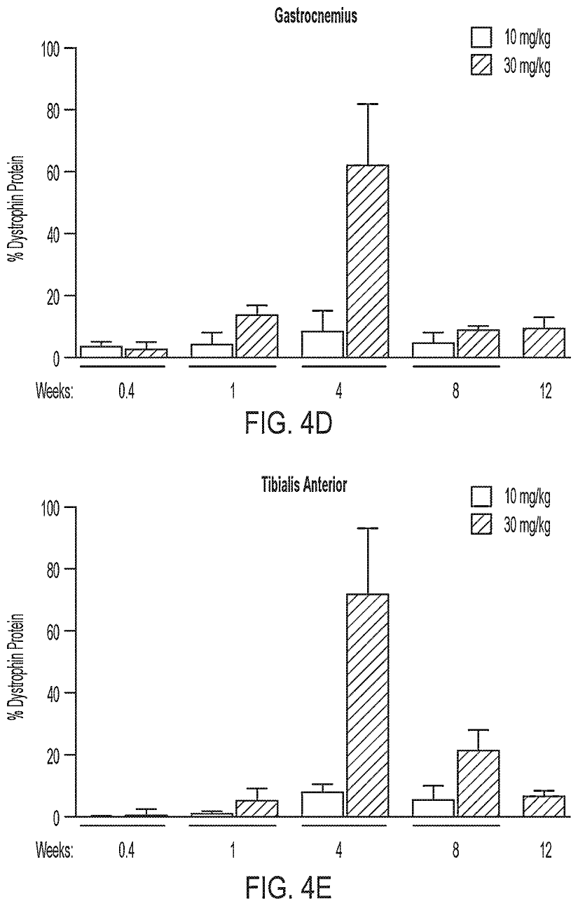

Images of Western blots are shown for quadriceps (FIG. 4A), diaphragm (FIG. 4B), heart (FIG. 4C) muscle tissues collected 3 days, 1 week, 4 weeks, 8 weeks, and 12 weeks following administration of anti-TfR1 Fab-oligo conjugate at the 67.7 mg/kg (equivalent to 30 mg/kg oligonucleotide). Quantification of the Western blot results following administration of anti-TfR1 Fab-oligo conjugate at the dose equivalent to 10 mg/kg oligonucleotide or equivalent to 30 mg/kg oligonucleotide are shown for all muscle tissues tested in FIGS. 4A-4E. Mice treated with anti-TfR1 Fab-oligo conjugate demonstrated durable and dose-dependent expression of dystrophin protein in all muscle tissues tested. Results in FIG. 4A (quadriceps), FIG. 4B (diaphragm), FIG. 4C (heart), FIG. 4D (gastrocnemius), and FIG. 4E (tibialis anterior) show that anti-TfR1 Fab-oligo conjugate facilitated robust dystrophin restoration in all muscle tissues post administration of either dose of anti-TfR1 Fab-oligo conjugate. For example, at 8 weeks post administration of the dose equivalent to 30 mg/kg oligonucleotide, dystrophin level in the mice reach 41% of wild-type dystrophin in quadriceps (see FIG. 4A), at 4 weeks post administration of the dose equivalent to 30 mg/kg oligonucleotide, dystrophin level in the mice reach 90% of wild-type dystrophin in diaphragm (see FIG. 4B), at 4 weeks post administration of the dose equivalent to 30 mg/kg oligonucleotide, dystrophin level in the mice reach 78% of wild-type dystrophin in heart (see FIG. 4C), at 4 weeks post administration of the dose equivalent to 30 mg/kg oligonucleotide, dystrophin level in the mice reach 60% of wild-type dystrophin in gastrocnemius (see FIG. 4D), and at 4 weeks post administration of the dose equivalent to 30 mg/kg oligonucleotide, dystrophin level in the mice reach 70% of wild-type dystrophin in tibialis anterior (see FIG. 4E). The results show that anti-TfR1 Fab-oligo conjugate achieved high level dystrophin expression across cardiac and skeletal muscles.

### Dystrophin Localization by Immunofluorescence Imaging

Dystrophin localization to the sarcolemma (muscle cell membrane) was assessed in quadriceps, diaphragm, and heart tissues of the mdx mice at 3 days, 1 week, 4 weeks, 8 weeks, and 12 weeks following administration of anti-TfR1 Fab-oligo conjugate. The indicated muscle tissues (quadriceps, diaphragm, heart) were flash-frozen in OCT mounting medium, sectioned into 10 μm serial sections, and were mounted on coverslips. Sections were then fixed in 4% PFA and stained with rabbit pAb anti-dystrophin primary antibody (abcam15277; 1:500) and counter-stained with rat mAb anti-Laminin (abcam11576; 1:1000). Micrographs were acquired on a Zeiss LSM 800 Confocal Microscope at 20× magnification. Post-processing of micrographs completed using ImageJ.

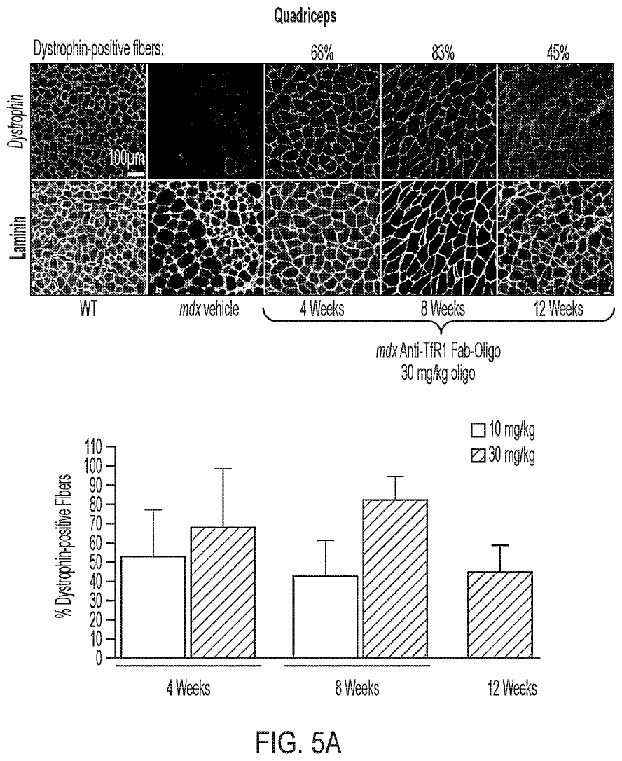

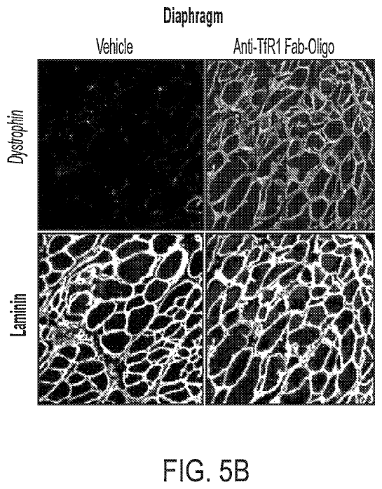

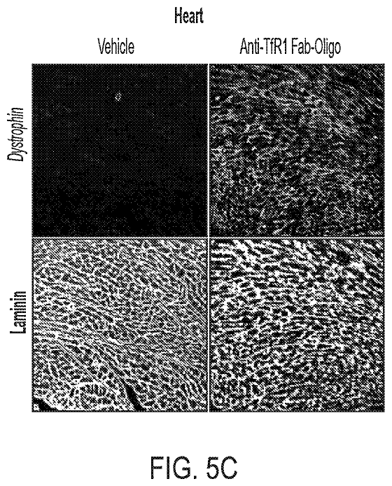

Immunofluorescence (IF) images in FIG. 5A (quadriceps), FIG. 5B (heart), and FIG. 5C (diaphragm) show percent dystrophin positive fibers (PDPF). Quantification of the quadriceps IF data is shown in FIG. 5A and demonstrates that there are 83% dystrophin-positive fibers in the sarcolemma of the quadriceps at 8 weeks post treatment. The IF images in FIG. 5B and FIG. 5C show that there is approximately 80% dystrophin-positive fibers in the sarcolemma of both the diaphragm and heart at 4 weeks post treatment. The results show that anti-TfR1 Fab-oligo conjugate achieved long lasting dystrophin localization to the sarcolemma in the quadriceps, diaphragm, and heart of the mdx mice.

### Example 4. Exon Skipping Activity of Anti-TfR1 Fab-Oligonucleotide Conjugate In Vivo in Cynomolgus Monkeys

This study evaluated the exon-skipping activities of anti-TfR1 conjugates comprising the anti-TfR1 Fab having the heavy chain and light chain sequences shown in Table 2 covalently linked (through lysine conjugation) via a linker comprising a Valine-Citrulline sequence to a dystrophin (DMD) exon 51-skipping oligonucleotide (oligo). The exon 51-skipping oligonucleotide is a phosphorodiamidate morpholino oligomer (PMO) and comprises the base sequence of SEQ ID NO: 21. The conjugates comprise a structure of formula (Id):

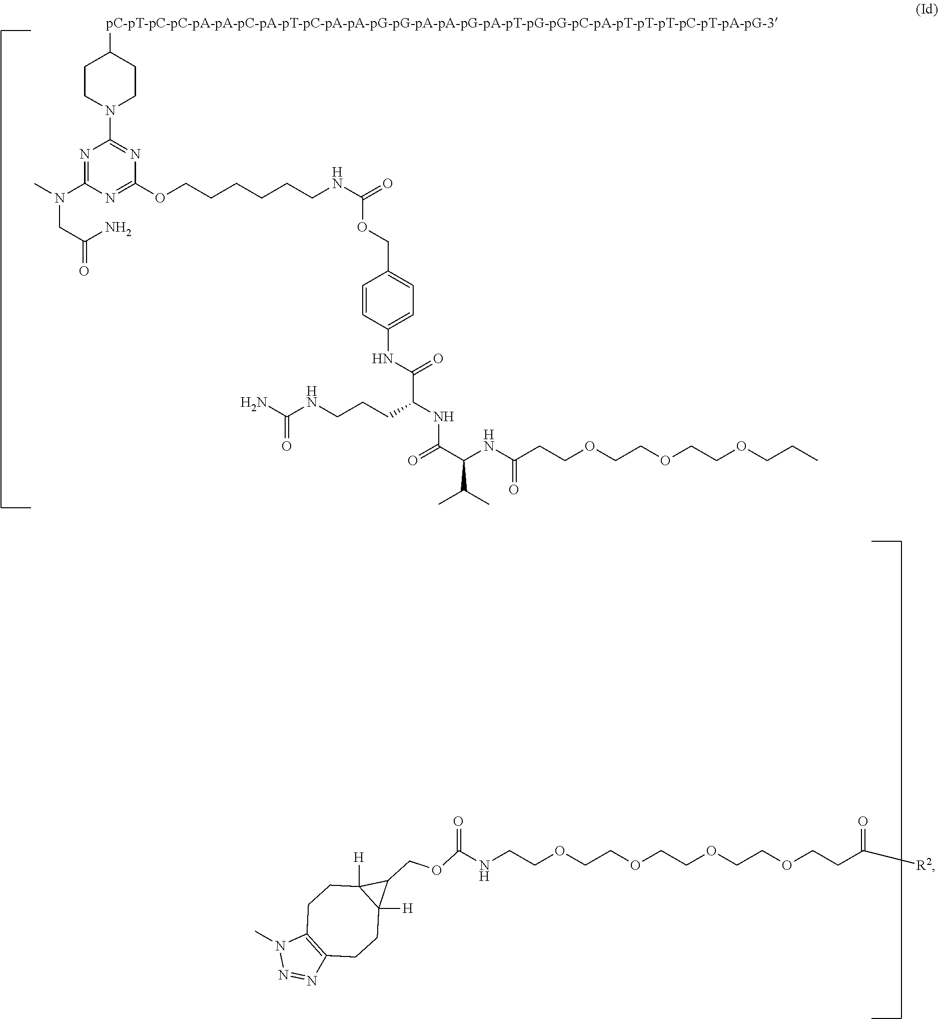

wherein R² is the anti-TfR1 Fab shown in Table 2, and wherein in each conjugate n1 is independently an integer of zero or greater.

The exon skipping activity of conjugate was tested in vivo in healthy cynomolgus monkeys. Naïve, adolescent male cynomolgus monkeys were treated with a single intravenous infusion of anti-TfR1 Fab-oligonucleotide conjugate at a dose equivalent to 60 mg/kg oligonucleotide) and sacrificed after two weeks or were treated with two intravenous infusions of anti-TfR1 Fab-oligonucleotide conjugate at a dose equivalent to 30 mg/kg oligonucleotide on days 1 and 15, and sacrificed two weeks after the last dose.

Tissue was homogenized and RNA isolation was performed on the lysate using the Promega Maxwell RSC instrument and the Maxwell RSC simplyRNA Tissue kit per manufacturer's protocol. cDNA was generated from 75 ng of total RNA using the Quantabio qScript cDNA Supermix using manufacturer's protocol. End-point PCR was performed using primers to amplify the region of interest. PCR products were run on the LabChip HT Touch II capillary electrophoresis instrument and percent exon skipping was quantified per the following equation:

$$\% Exon 51 Skipping = \frac{molarity of skipped transcript}{molarity of skipped + unskipped transcripts} \times 100$$

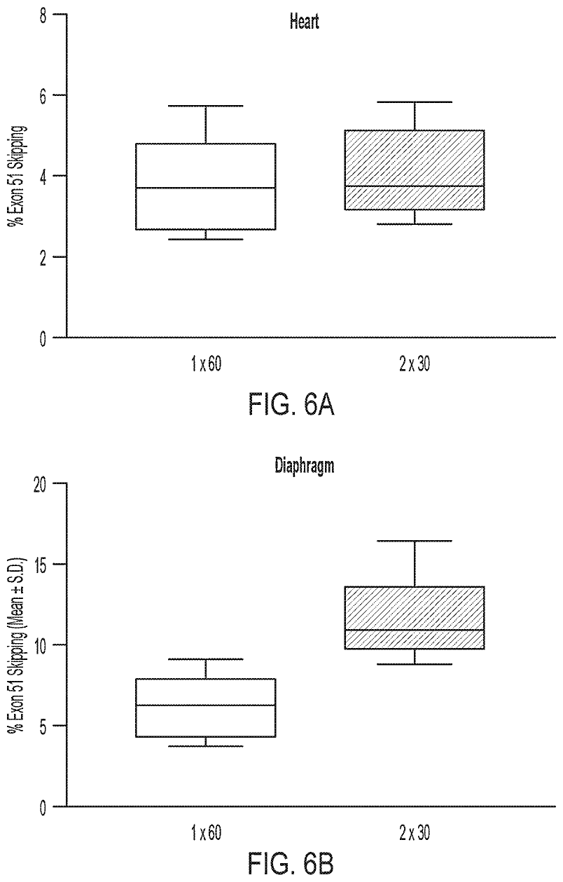

Results show that two intravenous infusions on days 1 and 15 at a dose of 65.7 mg/kg of anti-TfR1 Fab-oligonucleotide conjugate (equivalent to 30 mg/kg oligonucleotide) achieved comparable efficacy in heart and better efficacy in diaphragm in in cynomolgus monkey, compared with a single dose of 131.4 mg/kg anti-TfR1 Fab-oligo conjugate, equivalent to 60 mg/kg oligonucleotide (see FIGS. 6A and 6B).

### Example 5. Exon Skipping Activity of Anti-TfR1 Fab-Oligonucleotide Conjugate In Vivo in Cynomolgus Monkeys

This study evaluated the exon-skipping activities of anti-TfR1 conjugates comprising the anti-TfR1 Fab having the heavy chain and light chain sequences shown in Table 2 covalently linked (through lysine conjugation) via a linker comprising a Valine-Citrulline sequence to a dystrophin (DMD) exon 51-skipping oligonucleotide (oligo). The exon 51-skipping oligonucleotide is a phosphorodiamidate morpholino oligomer (PMO) and comprises the base sequence of SEQ ID NO: 21. The conjugates comprise a structure of formula (Id):

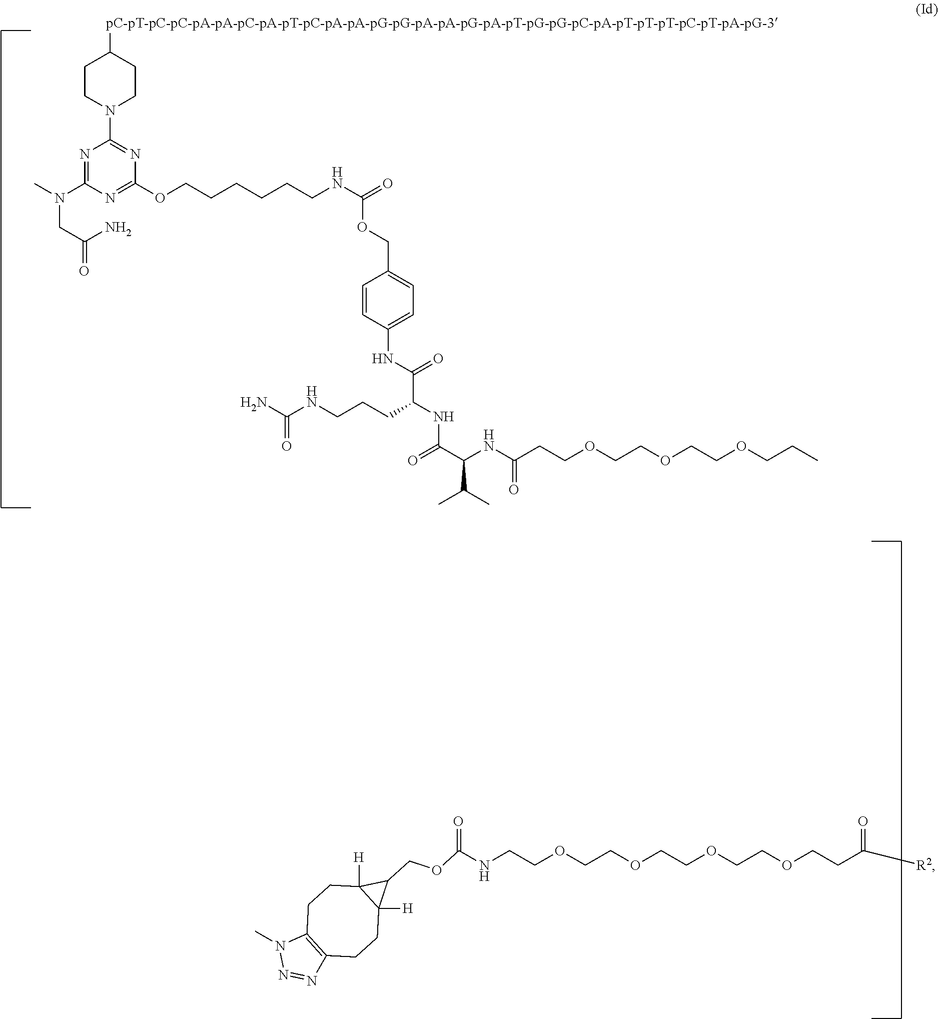

wherein R² is the anti-TfR1 Fab shown in Table 2, and wherein in each conjugate n1 is independently an integer of zero or greater.

The exon skipping activity of conjugate was tested in vivo in healthy cynomolgus monkeys. Naïve, adolescent male cynomolgus monkeys were (i) treated with two intravenous infusions of anti-TfR1 Fab-oligonucleotide conjugate at a dose equivalent to 30 mg/kg oligonucleotide on days 1 and 15, and sacrificed two weeks after the last dose, (ii) treated with intravenous infusions of anti-TfR1 Fab-oligonucleotide conjugate at a dose equivalent to 30 mg/kg oligonucleotide once a week for four weeks and sacrificed four weeks after the first dose, or (iii) treated with intravenous infusions of anti-TfR1 Fab-oligonucleotide conjugate at a dose equivalent to 30 mg/kg oligonucleotide once a week for five weeks and sacrificed eight weeks after the first dose.

Tissue was homogenized and RNA isolation was performed on the lysate using the Promega Maxwell RSC instrument and the Maxwell RSC simplyRNA Tissue kit per manufacturer's protocol. cDNA was generated from 75 ng of total RNA using the Quantabio qScript cDNA Supermix using manufacturer's protocol. End-point PCR was performed using primers to amplify the region of interest. PCR products were run on the LabChip HT Touch II capillary electrophoresis instrument and percent exon skipping was quantified per the following equation:

$$\% Exon 51 Skipping = \frac{molarity of skipped transcript}{molarity of skipped + unskipped transcripts} \times 100$$

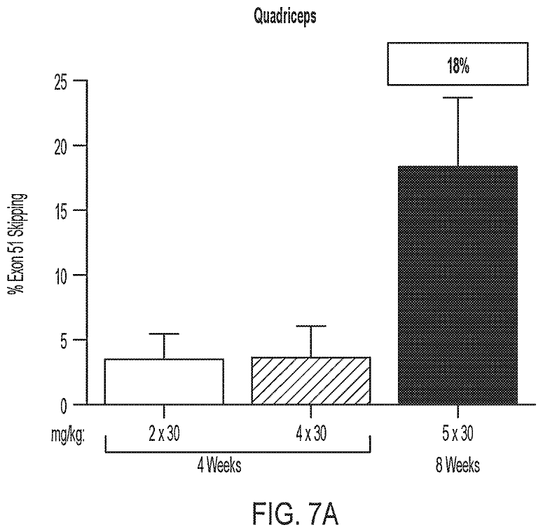

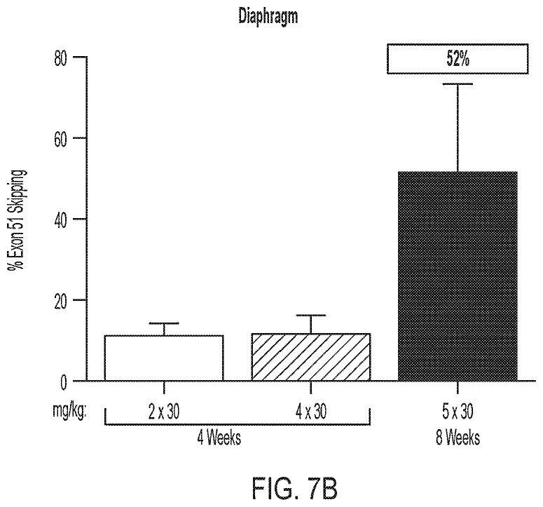

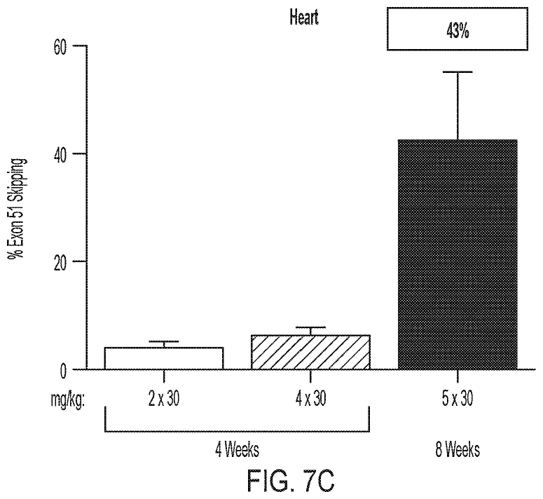

Results show that anti-TfR1 Fab-oligonucleotide conjugate achieved significant exon 51 skipping in cynomolgus monkey cardiac and skeletal muscles (FIGS. 7A-7C). Weekly intravenous infusions of anti-TfR1 Fab-oligonucleotide conjugate for five weeks at a dose equivalent to 30 mg/kg oligonucleotide achieved 18% exon 51 skipping in quadriceps (FIG. 7A), 52% exon 51 skipping in diaphragm (FIG. 7B), and 43% exon 51 skipping in heart (FIG. 7C).

### Example 6. Enhanced Exon Skipping and Prolonged Dystrophin Restoration Achieved by Targeted Delivery of Antisense Oligonucleotide Using Anti-TfR1 Fab-Oligonucleotide Conjugation in Mdx Mice

Current therapies for Duchenne muscular dystrophy (DMD) use oligonucleotides such as phosphorodiamidate morpholino oligomers (PMO) to induce exon skipping in the dystrophin pre-mRNA, enabling the translation of a shortened but functional dystrophin protein. This strategy has been hampered by insufficient delivery of PMO to cardiac and skeletal muscle. To overcome these limitations, a conjugate comprising an antigen-binding fragment, which binds the transferrin receptor 1 (TfR1), conjugated to an oligonucleotide was developed. It was demonstrated that a single dose of the anti-TfR1 Fab-oligonucleotide conjugate comprising an antibody that binds to a mouse TfR1 Fab conjugated to an oligonucleotide, enhances muscle delivery of an exon 23 skipping oligonucleotide (a PMO comprising the nucleobase sequence of SEQ ID NO: 36) in mdx mice, achieving dose-dependent and robust exon skipping and durable dystrophin restoration. Anti-TfR1 Fab-oligonucleotide conjugate-induced dystrophin expression reached peaks of 51%, 72%, 62%, 90%, and 77%, of wild-type levels in quadriceps, tibialis anterior, gastrocnemius, diaphragm, and heart, respectively, with a single dose of anti-TfR1 Fab-oligonucleotide conjugate at a dose equivalent to 30 mg/kg oligonucleotide. The shortened dystrophin localized to the sarcolemma, indicating expression of a functional protein. Conversely, a single 30 mg/kg dose of unconjugated exon 23-skipping oligonucleotide displayed poor muscle delivery resulting in marginal levels of exon skipping and dystrophin expression. Importantly, anti-TfR1 Fab-oligonucleotide conjugate treatment resulted in improved functional outcomes compared with administration of unconjugated exon 23-skipping oligonucleotide. The results suggest that the anti-TfR1 Fab-oligonucleotide conjugates are an effective approach for the treatment of DMD.

### Materials And Methods

### Reagents

RNAlater (Cat No. AM7021), iBright FL1000 Imaging System (Cat No. A32752), Rapid Gold BCA bicinchoninic acid assay (Cat No. A53225), Superscript III Platinum Taq polymerase (Cat No. 12574018), anti-rabbit Alexa647 (Cat No. A-21245) and anti-rat Alexa488 (Cat No. A-21208) antibodies were purchased from ThermoFisher Scientific/Invitrogen, Carlsbad, CA. The Maxwell RSC instrument (Cat No. AS8500) and Maxwell RSC simplyTissue extraction kit (Cat No. AS1340) were obtained from Promega, Madison, WI, USA. LabChip GXII Touch HT (CLS138160) and the DNA 5K/RNA/Charge Variant LabChip (Cat No. 760435) were purchased from Perkin Elmer, Santa Clara, CA, USA. Creatinine kinase (CK) activity assay kit (Colorimetric; Cat No. ab155901) and the following antibodies were purchased from Abcam (Cambridge, UK): anti-dystrophin (Cat No. Ab15277), anti-alpha-actinin (Cat No. Ab9465), anti-Laminin (Cat No. Ab11576). IRDye 800CW Goat anti-Rabbit IgG (Cat No. 926-32211) and IRDye 680RD Goat anti-Mouse IgG (Cat No. 926-68070) antibodies and Odyssey CLX instrument (Cat No. 9140-09) were purchased from Licor, Lincoln, NE, USA. Sepharose 4 Fast Flow Protein G resin (Cat No. 17061802) and Butyl HP resin (Cat No. 28411005) were acquired from Cytiva, Marlborough, MA, USA. Bio-Gel P-4 (Cat No. 1504124) and ceramic hydroxyapatite (CHT) (Cat No. 1582000) were purchased from Bio-Rad Laboratories, Hercules, CA, USA. The remaining reagents/instruments were purchased as follows: Vivaflow 50 cassette (Sartorius, Göttingen, Germany; Cat No. VF05P2); PES Rapidflow (Thermo Scientific Nalgene, Rochester, NY, USA; Cat No. 09-741-03); Endosafe limulus amebocyte lysate kinetic chromogenic assay (Charles River Laboratories International, Inc., Wilmington, MA, USA; Cat No. PTS20F); phosphorothioate/DNA capture probe (Integrated DNA Technologies Inc., Coralville, IA, USA); Synergy Neo2 Multi-Mode Microplate Reader (BioTek, Winooski, VT, USA, Cat No. NEO2); Precellys Evolution homogenizer (Bertin, Montigny-le-bretonneux, France, Cat No. P000062-PEVO0-A); hard-tissue homogenizer (Omni International, Inc., Kennesaw, GA, USA, Cat No. TH115); optimal cutting temperature (OCT) mounting medium (Tissue-tek, Torrance, CA, USA, Cat No. 4583); ZEISS laser scanning 800 confocal microscope (Carl Zeiss Meditec AG, Jena, Germany); Image J software (US National Institutes of Health, Bethesda, MD, USA); M3 multimode plate reader (Molecular Devices, San Jose, CA, USA, Cat No. M3); Mouse Activity Wheels with dual lickometers (Lafayette Instrument Co., Lafayette, IN, USA, Model 80822); plexiglass square chambers (Med Associates Inc., St Albans, VT, USA). A monoclonal rat anti-mouse IgG2a antibody targeting mouse TfR1 Anti-mTfR1 Fab (RI7217-rlgG2a) was purchased from Genescript Biotech Corporation, Piscataway, NJ, USA. The PMO antisense oligonucleotide for mouse dystrophin exon 23 (5′-NH2-GGCCAAACCTCGGCTTACCTGAAAT-3′) (SEQ ID NO: 36) (26,27) was purchased from Gene Tools, LLC (Philomath, OR, USA). Nucleic acid-hybridization enzyme-linked immunosorbent assay (hELISA) capture probe, dual labeled with digoxigenin at the 5′ end and biotin at the 3′ end (5′/DigN/ATTTCAGGTAAG CCGAGGTTTGGCC/3Bio/3′ (SEQ ID NO: 38); the phosphorothioate ends are highlighted in bold), and PCR primers (forward primer: 5′-CACATCTTTGATGGTGTGAGG-3′ (SEQ ID NO: 39); reverse primer: 5′-CAACTTCAGCCATCCATTTCTG-3′) (SEQ ID NO: 40) were acquired from Integrated DNA Technologies (Coralville, IA, USA).

### Biological Resources

Mdx (C57Bl/10ScSn-Dmd<mdx>/J, Jackson Laboratory Stock #1801) and C57Bl/10ScSn wild-type mice were obtained from The Jackson Laboratory, Bar Harbor, ME, USA. CHO-K1SP cells were purchased from Genescript Biotech Corporation, Piscataway, NJ, USA.

### Anti-TfR1 Fab-Oligonucleotide Conjugate

### Anti-TfR1 Fab

A monoclonal rat anti-mouse IgG2a antibody Fab targeting mouse TfR1 (RI7217-rIgG2a) was stably expressed in CHO-K1SP cells. Supernatant was harvested and purified on Protein G resin, eluted, and buffer-exchanged into Dulbecco's phosphate-buffered saline (pH 7.4).

### Linker-Oligonucleotide Intermediate

To pre-form the linker-oligonucleotide intermediate, the exon 23 skipping oligonucleotide (SEQ ID NO: 36) was dissolved at 50 mg/mL in dimethyl sulfoxide. The bifunctional valine-citrulline linker generated in house was dissolved in dimethyl sulfoxide at 30 mg/mL and mixed at a 4-molar excess with the exon 23 skipping oligonucleotide for 120 minutes at room temperature. Reaction completion was measured using the ninhydrin test. Excess reagents were removed using P-4 gel permeation chromatography. The concentration of linker-oligonucleotide was measured by ultraviolet-visible spectrophotometry (265 nm. ¿=259210 mM-1 cm-1) at 0.1N hydrochloric acid; purity was evaluated by reverse-phase, high-performance liquid chromatography (HPLC).

### Anti-TfR1 Fab-Linker-Oligonucleotide Conjugate

Before conjugation with linker-oligonucleotide, the anti-TfR1 Fab was diluted with 1/10 v/v acetonitrile (HPLC grade). Anti-TfR1 Fab was mixed with a 3-molar excess of linker-oligonucleotide for 18 hours at room temperature. Reaction was confirmed with sodium dodecyl sulphate-polyacrylamide gel electrophoresis (SDS-PAGE) and analytical size-exclusion chromatography. Unreacted Fab was removed using hydrophilic interaction chromatography (Butyl HP) with an ammonium sulfate gradient. Pooled factions were diluted with nuclease-free water to reduce conductivity to <8 mS and purified using hydroxyapatite chromatography (CHT) to remove unconjugated linker-oligonucleotide. The final anti-TfR1 Fab-linker-oligonucleotide conjugate was eluted directly into formulation buffer (100 mM disodium phosphate, 100 mM sodium chloride, pH 7.2) and concentrated using 10 kDa tangential flow filtration (Vivaflow 50), followed by sterile filtration (0.22 μm, PES Rapidflow). The concentration of the conjugate was measured by bicinchoninic acid assay (Rapid Gold BCA) and the drug antibody ratio was determined by densitometry of an SDS-PAGE gel (iBright FL1000). Endotoxin levels were measured using the Endosafe limulus amebocyte lysate kinetic chromogenic assay.

### Study Designs

### Kinetics Study

To analyze exon skipping and dystrophin restoration, 5-week-old male mdx mice (C57B1/10ScSn-Dmd<mdx>/J) were injected via tail vein with a single intravenous (IV) dose of vehicle, 30 mg/kg unconjugated exon 23-skipping oligonucleotide, or anti-TfR1 Fab-oligonucleotide conjugate containing the equivalent of 10 or 30 mg/kg oligonucleotide (n=5 animals per treatment). Vehicle-treated mice were necropsied 4-, 8-, or 12-weeks post-dose. Mice treated with 30 mg/kg unconjugated exon 23-skipping oligonucleotide were necropsied 1-, 4-, or 8-weeks post-dose. Mice treated with anti-TfR1 Fab-oligonucleotide conjugate were necropsied at 3-days, 1-, 4-, or 8-weeks post-dose for the 10 mg/kg oligonucleotide-equivalent cohort and at 3-days, 1-, 4-. 8-, 12-, or 24-weeks post-dose for the 30 mg/kg oligonucleotide-equivalent cohort. All mice were anesthetized with a sublethal dose of pentobarbital prior to necropsy. Whole blood was collected using cardiac puncture and processed to produce serum for creatine kinase (CK) analysis. Mice were then perfused with saline, and muscle collected; the left side of each muscle was processed with RNAlater and the right side was snap frozen in liquid nitrogen for subsequent molecular analysis. Samples were removed from RNAlater after approximately 48 hours, frozen using dry ice, and stored at −80° C.

### Functional Study

To assess functional performance, 7-8-week-old male mdx mice and C57B1/10ScSn wild-type controls were injected via tail vein with a single intravenous dose of vehicle (n=10 each mdx and wild-type mice), 30 mg/kg exon 23-skipping oligonucleotide (n=10 mdx mice), or anti-TfR1 Fab-oligonucleotide conjugate at a dose equivalent to 30 mg/kg oligonucleotide (n=10 mdx mice). Voluntary running wheel and open-field activity were assessed 2 weeks post-dose (n=10) and 4 weeks post-dose (n=5) for each treatment group. At the desired time points, mice were anesthetized with a sublethal dose of pentobarbital. Whole blood was collected using cardiac puncture and processed to produce serum for CK analysis.

### Animal Husbandry

The environmental temperature was maintained between 20° C. and 23° C. and the relative humidity at approximately 50%. A light cycle of 12 hours light: 12 hours dark was maintained. Food and water were provided ad libitum. Animals were acclimated to handling and testing procedures prior to functional testing.

### Enzyme-Linked Immunosorbent Assay for Oligonucleotide Detection

An hELISA method was adapted from the method of Burki et al. (29) A phosphorothioate/DNA reverse complement capture probe for exon 23-skipping oligonucleotide was dual labeled with digoxigenin at the 5′ end and biotin at the 3′ end. The signal was read on a Synergy Neo2 Multi-Mode Microplate Reader at A450. Concentrations of exon 23-skipping oligonucleotide were calculated using a standard curve of known concentrations ranging from 0-500 pM.

Exon skipping analysis

Total RNA was isolated from tissue samples using cryo-pulverization and subsequent RNA extraction using a Maxwell RSC instrument per manufacturer's protocol. Briefly, tissue homogenization was performed on tissue fragments from muscle prepared with RNALater. Tissue fragments were weighed and an appropriate amount of 1-Thioglycerol/Maxwell homogenization buffer was added to achieve a final tissue concentration of 200 mg/mL buffer. Using the manufacturer's pre-specified “hard tissue” program on a Precellys Evolution, the samples were homogenized in grinding tubes. Tubes were then centrifuged for 1 minute at 5,000 rpm to reduce foaming; homogenization and centrifuging were repeated twice. Total RNA was extracted using the Maxwell RSC simply Tissue extraction kit per the manufacturer's protocol. Total RNA isolated from tissue samples was used as a template to assay exon 23 skipping. Total RNA (15-75 ng) was used for a 25 μL RT-PCR with Superscript III Platinum Taq. After 35 cycles were completed, the reaction was diluted 1:10 in PCR buffer and was analyzed on a LabChip® GXII Touch HT using a DNA 5K/RNA/Charge Variant LabChip® according to the manufacturer's protocol. The full-length dystrophin transcript yields a PCR product of 445 base pairs; the exon 23-skipped RNA produces a PCR product of 232 base pairs. Percent exon skipping was quantified using the equation:

$$\% Exon 23 Skipping = \frac{Molarity of Skipped Band}{Molarity of Skipped Band + Molarity of Unskipped band} * 100$$

### Protein Extraction and Western Blot

Muscles were homogenized in a buffer containing 8M urea, 0.5M Tris (pH 7.5), 10% sodium dodecyl sulfate, and protease inhibitor in deionized water using a hard-tissue homogenizer at a medium speed for 30 seconds, four times per sample. Protein concentrations were measured by bicinchoninic acid assay. Total protein (25 μg) was loaded onto a 3%-8% Tris-acetate protein gel and run at 60 V for 30 minutes and 100 V for 1 hour. The gel was then transferred to a polyvinylidene difluoride membrane for 120 minutes at 4° C. at 200 mAmps. The membrane was incubated overnight with anti-dystrophin antibody at a 1:500 dilution and anti-alpha-actinin at a dilution of 1:10,000 at 4° C. The next day, the membrane was washed and incubated with IRDye 800CW Goat anti-Rabbit IgG and IRDye 680RD Goat anti-Mouse IgG secondary antibodies at 1:10,000 dilution to visualize dystrophin and alpha-actinin, respectively. Blots were visualized on an Odyssey® CLX with detection of dystrophin at 800 nm and alpha-actinin at 680 nm.

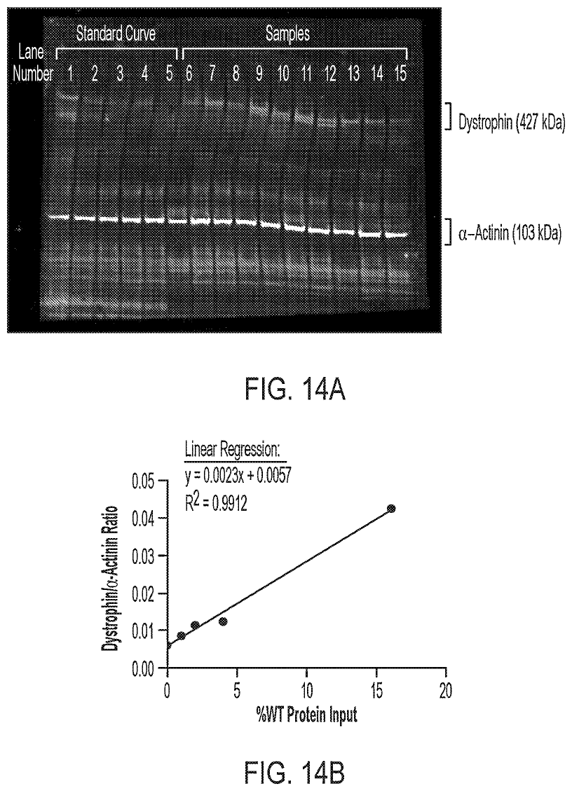

To quantify the amount of dystrophin protein present in each sample, each gel was run with a standard curve of matched muscle matrix from wild-type mice diluted into muscle matrix from mdx mice. Raw fluorescence was measured in the dystrophin and alpha-actinin channels for each standard and unknown sample. The dystrophin/alpha-actinin ratio of the standards was then generated and plotted against the known percent wild-type protein in each standard to generate a linear equation. Unknown samples were then interpolated from their dystrophin/alpha-actinin ratio based on the equation of the standard curve. If the raw dystrophin signal of the unknown sample was above the range of the standard curve, the sample was diluted until it fell within the standard curve. A representative western blot image is provided in FIG. 14A and a representative standard curve calculation is provided in FIG. 14B.

### Immunofluorescent Microscopy

Quadriceps flash-frozen in OCT mounting medium were sectioned into 10-μm serial sections and mounted on coverslips. Slides were allowed to equilibrate at room temperature before being placed in 1′ PBS for 5 minutes at room temperature. Slides were then fixed in 4% paraformaldehyde and stained with rabbit polyclonal antibody anti-dystrophin primary antibody (1:500) and counter-stained with rat monoclonal antibody anti-laminin (1:1,000). Laminin was used as the counterstain because it stains a large glycoprotein just outside of the sarcolemma; this method showed the outline of the muscle fiber without overlapping the membrane, which is relevant when targeting the myofiber outline while avoiding complex image deconvolution of overlapping staining on the membrane. To visualize dystrophin, slides were stained with anti-rabbit Alexa647 (1:500); to visualize laminin, slides were stained with anti-rat Alexa488 (1:500) secondary antibodies prior to imaging. Micrographs were acquired on a ZEISS laser scanning 800 confocal microscope at 20′ magnification. Post-processing of micrographs was completed using Image J software. (30)

### Serum CK Assay

Analysis of CK activity was performed using a CK activity assay kit (Colorimetric) according to the manufacturer's protocol. Serum samples were thawed on ice and diluted 1:100 in CK assay buffer (4° C.). In a 96-well microplate, diluted nicotinamide adenine dinucleotide standards, positive control, and diluted serum samples were incubated with reaction master mix (CK assay buffer, enzyme mix, developer, ATP, and substrate) at room temperature. Background control reactions using diluted serum samples without the enzyme mix were incubated at room temperature. Readings were obtained at OD₄₅₀ using an M3 multimode plate reader in kinetic mode every 5 minutes for a total of 30 minutes at 37° C. OD₄₅₀ readings were linear between T₁ (time 0) and T₂ (10 minutes). A standard curve was constructed by plotting the OD₄₅₀ of the standard against the concentration of nicotinamide adenine dinucleotide. Optical density values were used to calculate the CK activity of the serum samples.

### Muscle Function Tests

### Running Wheel

Voluntary running wheel assessments were conducted on Mouse Activity Wheels with dual lickometers. The polycarbonate chamber measured 35.3×23.5×20 cm. The aluminum running wheel had an inside diameter of 12.7 cm and an inside width of 5.72 cm for a run distance of 0.40 meters/revolution. The run surface consisted of 38 rods each approximately 4.8 mm in diameter on 10.9-mm centers with a 6.14-mm gap. The running wheel was connected to an interface that electronically recorded the animal's activity, including total distance travelled.

### Hindlimb Fatigue Challenge

Locomotor activity, including distance traveled (horizontal activity) was evaluated using an automated open-field test. Activity was measured in a 10-minute test session both before and after either a postural hind limb fatigue challenge (HLFC) session of 10 minutes or placement in the home cage for 10 minutes. Open field activity was performed in plexiglass square chambers measuring 27.3×27.3×20.3 cm surrounded by infrared photobeam sources used as open-field chambers. Horizontal activity was measured from consecutive beam breaks. The postural HLFC is a pre-test exercise performed to exacerbate the muscle phenotype of the mdx mouse line. Briefly, the mice were restrained such that they must stand bipedal on their hindlimbs with the majority of their body weight for 10 minutes. The mice were then subjected to testing as described.

### Statistical Analysis

A significance level of 0.05 was selected for all inferential statistics. For all molecular analyses, a two-way ANOVA was performed with a Dunnett's Multiple Comparison post-hoc test to evaluate the difference between unconjugated exon 23-skipping oligonucleotide and anti-TfR1 Fab-oligonucleotide conjugate data at the 1-, 4- and 8-weeks post-dose timepoints. For all behavioral assessments, statistical significance was assessed by one-way or two-way analysis of variance with appropriate post hoc comparisons. Outliers were excluded from each of the behavioral assessments. For the running wheel analysis, the total distance traveled during the dark cycle of the first 24 hours was assessed. An animal was excluded from the running wheel analysis if its total distance traveled was greater or less than 2 standard deviations (SDs) from the mean compared with the mean performance of all animals in the same treatment group.

For the HLFC, the percent change in total distance traveled in the open field was calculated before and after home cage rest or postural HLFC. For each treatment group, home cage data and HLFC data were analyzed separately. An animal was excluded from either home cage or HLFC analysis if the percent change in its total distance traveled was greater or less than 2 SDs from the mean compared with the mean performance of all animals in the same treatment group.

### Results

Anti-TfR1 Fab-Oligonucleotide Conjugate Delivers Significantly More Exon Skipping Oligonucleotide to Skeletal and Cardiac Muscle of Mdx Mice than Administration of Unconjugated Exon 23-Skipping Oligonucleotide

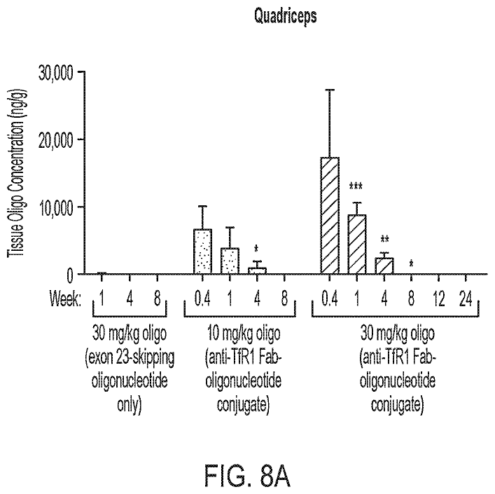

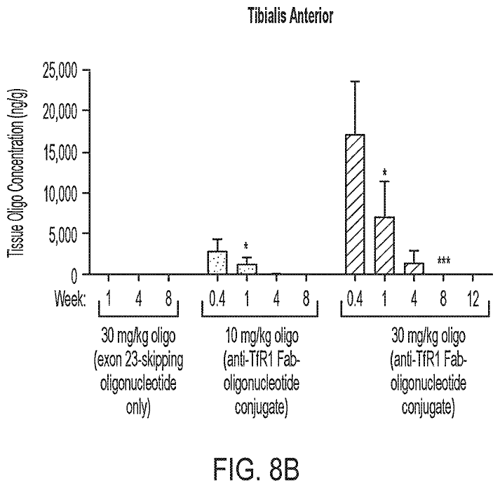

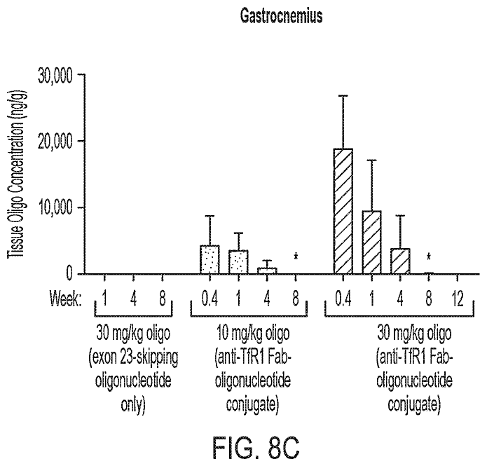

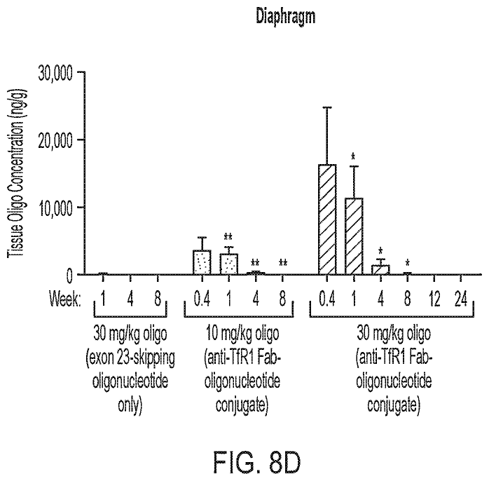

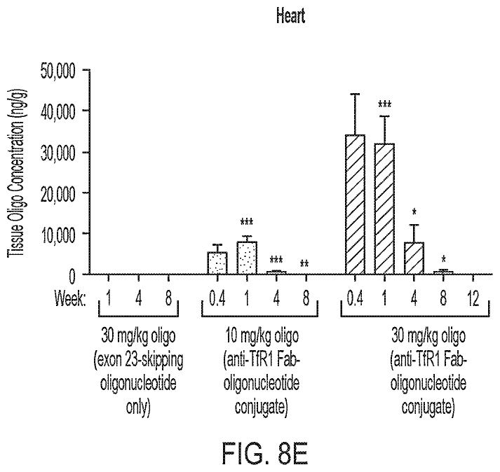

To examine the ability of the anti-TfR1 Fab-oligonucleotide conjugate to restore dystrophin protein expression in the mdx mouse model of DMD, the exon 23 skipping oligonucleotide (SEQ ID NO: 36) was conjugated to the anti-TfR1 Fab. To determine whether the anti-TfR 1 Fab-oligonucleotide conjugate delivers oligonucleotide to muscle, the kinetics of total muscle oligonucleotide levels in mdx mice were measured by hELISA after a single intravenous administration of anti-TfR1 Fab-oligonucleotide conjugate at doses delivering either 10 mg/kg or 30 mg/kg oligonucleotide. A parallel cohort of mdx mice was injected intravenously with unconjugated exon 23-skipping oligonucleotide at a dose of 30 mg/kg, anti-TfR1 Fab-oligonucleotide conjugate led to a dose-dependent distribution of oligonucleotide to cardiac and skeletal muscle, and significantly higher levels of exposure than unconjugated exon 23-skipping oligonucleotide alone. Peak oligonucleotide levels were detected in quadriceps (FIG. 8A) 3 days post-dose and declined steadily thereafter at a similar rate for both dose levels. Oligonucleotide exposure kinetics in other skeletal muscles, including tibialis anterior (TA; FIG. 8B), gastrocnemius (FIG. 8C), and diaphragm (FIG. 8D) were similar to the kinetics in quadriceps. In the heart, peak oligonucleotide levels were achieved between 3 days and 1-week post-dose (FIG. 8E). Minimal oligonucleotide exposure was detectable in the skeletal and cardiac muscles of mdx mice treated with unconjugated exon 23-skipping oligonucleotide at 1- and 4-weeks post-dose and no payload was detectable at 8 weeks post-dose (FIGS. 8A-8E). These data indicate that the anti-TfR1 Fab-oligonucleotide conjugate enhances delivery of an oligonucleotide payload to muscle.

### A Single Dose of Anti-TfR1 Fab-Oligonucleotide Conjugate, but not of Unconjugated Exon 23-Skipping Oligonucleotide, Leads to Robust and Durable Exon 23 Skipping in Muscles of Mdx Mice

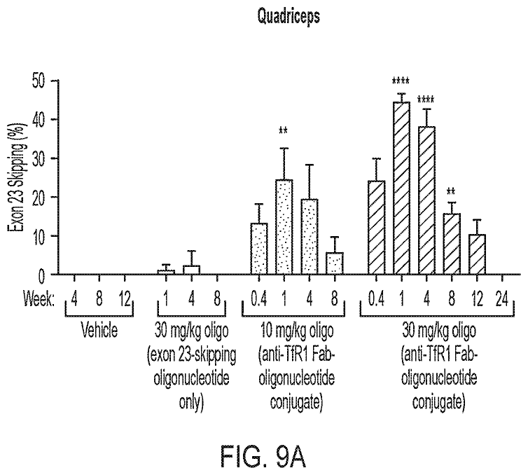

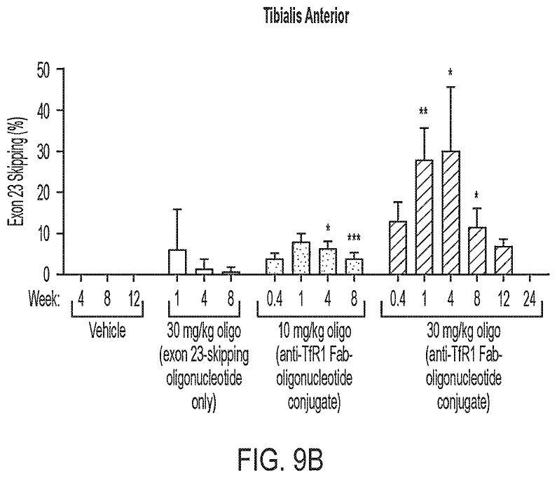

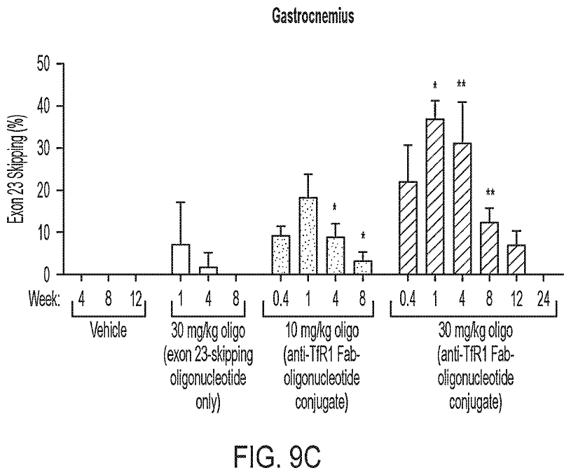

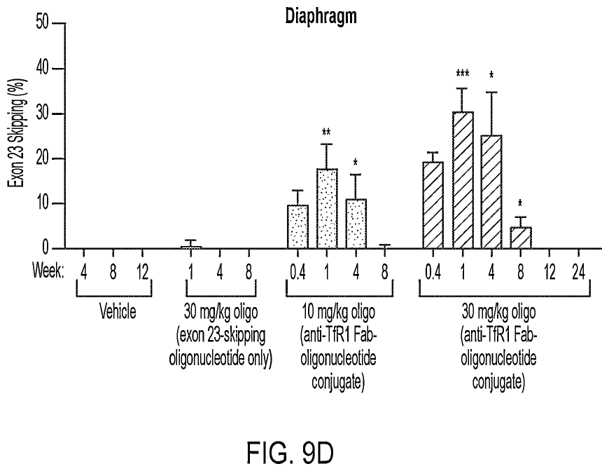

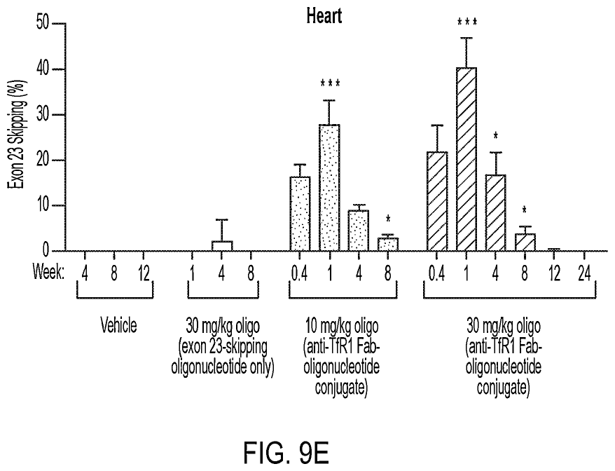

To investigate whether the anti-TfR1 Fab-oligonucleotide conjugate delivered an active oligonucleotide payload to muscle. Dmd exon 23 skipping was assessed using endpoint-RT-PCR and capillary electrophoresis. Dose-dependent exon 23 skipping was detectable 3 days after administration of anti-TfR1 Fab-oligonucleotide conjugate and peaked at 1-week post-dose in all muscles evaluated. Mean maximum levels of exon 23 skipping in quadriceps (FIG. 9A), tibialis anterior (FIG. 9B), gastrocnemius (FIG. 9C), diaphragm (FIG. 9D), and heart (FIG. 9E) were 24%, 8%, 18%, 18%, and 28%, respectively, after a single dose of anti-TfR1 Fab-oligonucleotide conjugate at a dose equivalent to 10 mg/kg oligonucleotide. Mean maximum levels in the same muscles following treatment with a single dose of anti-TfR1 Fab-oligonucleotide conjugate at a dose equivalent to 30 mg/kg oligonucleotide were 44%, 30%, 30%, 37%, and 40% (FIGS. 9A-9E). Administration of a matched 30 mg/kg dose of exon 23-skipping oligonucleotide alone resulted in significantly lower exon skipping. Exon 23 skipping was only detectable in a single mouse in the 30 mg/kg unconjugated exon 23-skipping oligonucleotide cohort at 1- and 4-weeks post-dose with no detectable skipping evident by 8 weeks post-dose (FIGS. 9A-9E). No exon 23 skipping was detectable in any muscle evaluated in the vehicle-treated control mice at 4-, 8-, and 12-weeks post-dose (FIGS. 9A-9E). Importantly, anti-TfR1 Fab-oligonucleotide conjugate-induced skipping was durable and detectable out to 8 weeks in cardiac muscle and diaphragm and up to 12 weeks in quadriceps, tibialis anterior, and gastrocnemius of mice administered a single dose of anti-TfR1 Fab-oligonucleotide conjugate at a dose equivalent to 30 mg/kg oligonucleotide. These data demonstrate that enhanced oligonucleotide delivery by the anti-TfR1 Fab-oligonucleotide conjugate translates into increased levels of exon skipping in cardiac and skeletal muscle.

### A Single Dose of Anti-TfR1 Fab-Oligonucleotide Conjugate, but not of Unconjugated Exon 23-Skipping Oligonucleotide, Induces Robust and Durable Dystrophin Protein Expression in Muscles of Mdx Mice

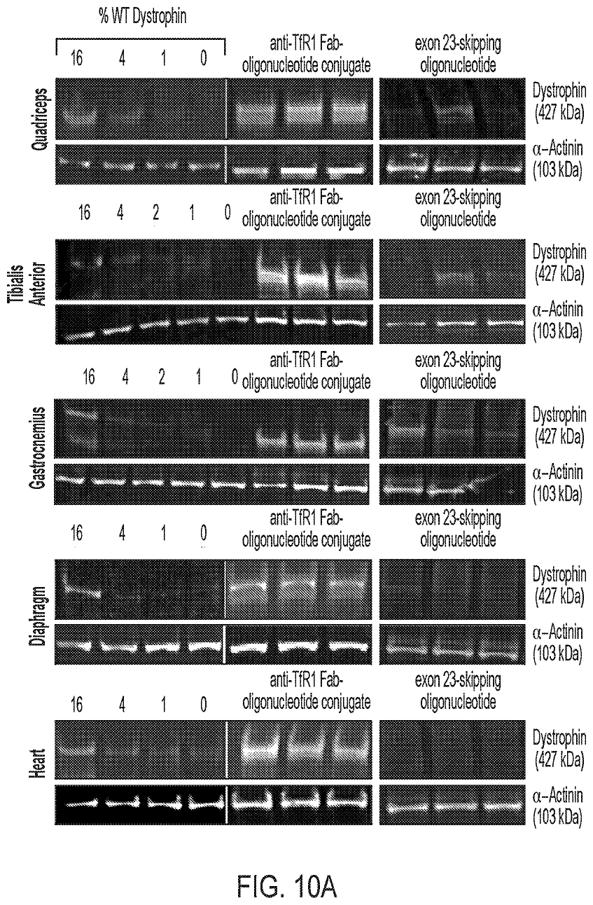

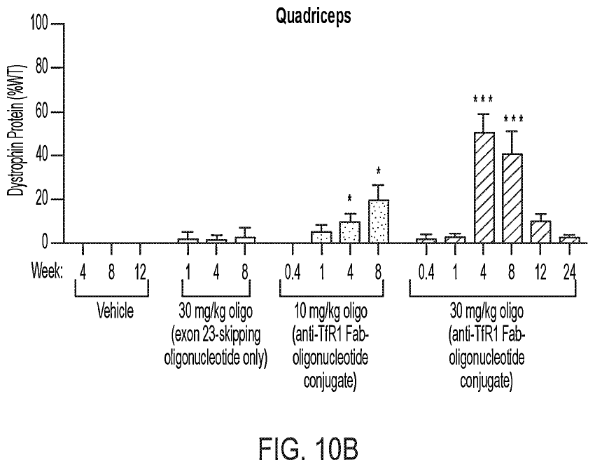

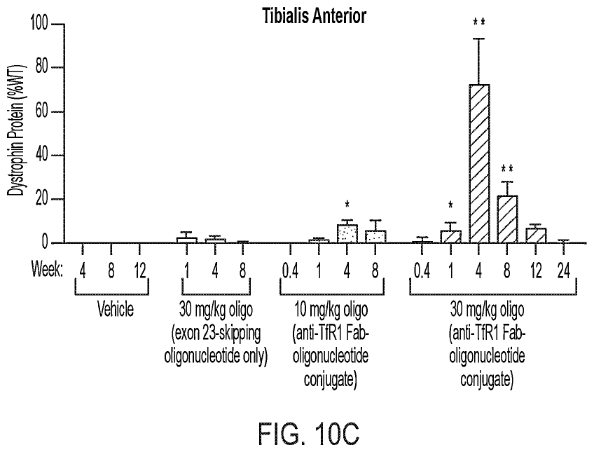

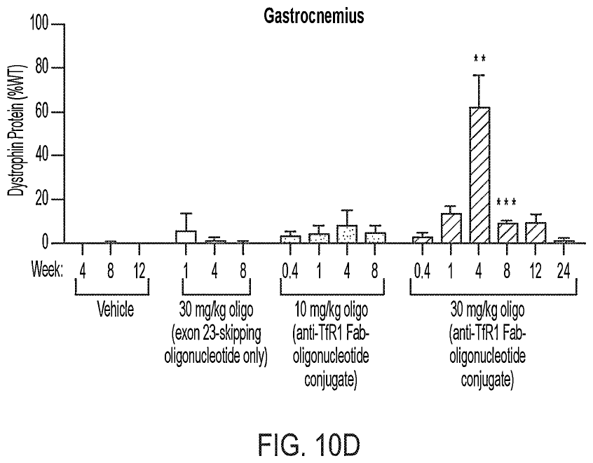

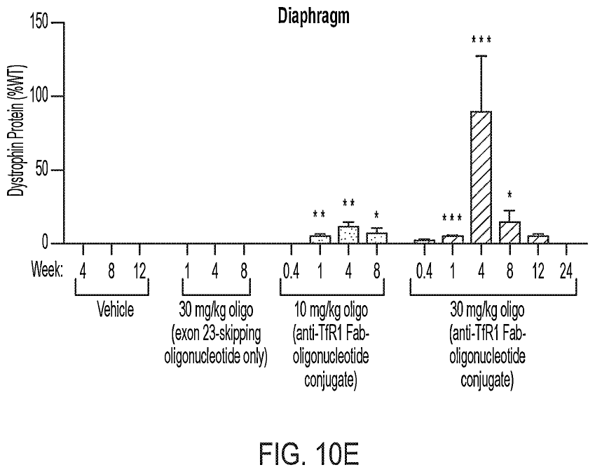

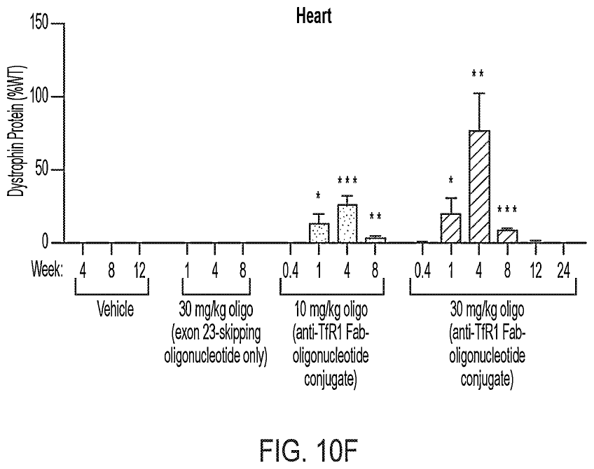

Consistent with the induction of exon 23 skipping, treatment with a single dose of anti-TfR1 Fab-oligonucleotide conjugate resulted in a durable and dose-dependent restoration of dystrophin protein expression. Representative western blots using undiluted muscle samples of quadriceps, TA, gastrocnemius, diaphragm, and heart of animals treated with anti-TfR1 Fab-oligonucleotide conjugate at a dose equivalent to 30 mg/kg oligonucleotide showed dystrophin restoration exceeded the upper limit of the wild-type dystrophin standard curve (FIG. 10A; samples were subsequently diluted into the range of the standard curve to generate quantification). Conversely, most vehicle-treated mice had minimal to no detectable dystrophin (FIGS. 10B-10F) consistent with the presence of a low percentage of dystrophin-positive revertant fibers in mdx mice. (31)

In the quadriceps (FIG. 10B), tibialis anterior (FIG. 10C), gastrocnemius (FIG. 10D) diaphragm (FIG. 10E), and heart (FIG. 10F), anti-TfR1 Fab-oligonucleotide conjugate-induced dystrophin expression reached peaks of 20%, 8%, 8%, 11%, and 27%, respectively, of wild-type levels after administration of anti-TfR1 Fab-oligonucleotide conjugate at a dose equivalent to 10 mg/kg oligonucleotide. Peak dystrophin expression after administration of anti-TfR1 Fab-oligonucleotide conjugate at a dose equivalent to 30 mg/kg oligonucleotide was 51%, 72%, 62%, 90%, and 77% of wild-type levels in the same muscles (FIGS. 10B-10F). Durable dystrophin expression was noted up to 8 weeks in the heart and diaphragm and up to 12 weeks in skeletal muscles of the hindlimb after a single dose of anti-TfR1 Fab-oligonucleotide conjugate at a dose equivalent to 30 mg/kg oligonucleotide. In contrast, treatment with 30 mg/kg unconjugated exon 23-skipping oligonucleotide led to lower levels of dystrophin production in limb muscles of a minority of mdx mice 1-, 4-, and 8-weeks post-dose (FIGS. 10A-10B, and 10D). Dystrophin protein was detected only sporadically in diaphragm and heart of mdx mice 1- and 4-weeks after administration of unconjugated exon 23-skipping oligonucleotide but was no longer measurable after 8 weeks (FIGS. 10E-10F). These data corroborate the observations on Dmd exon skipping and support the superiority of the anti-TfR1 Fab-oligonucleotide conjugate as a delivery platform for oligonucleotides.

Treatment with Anti-TfR1 Fab-Oligonucleotide Conjugate Increases Dystrophin-Positive Myofibers in Muscle

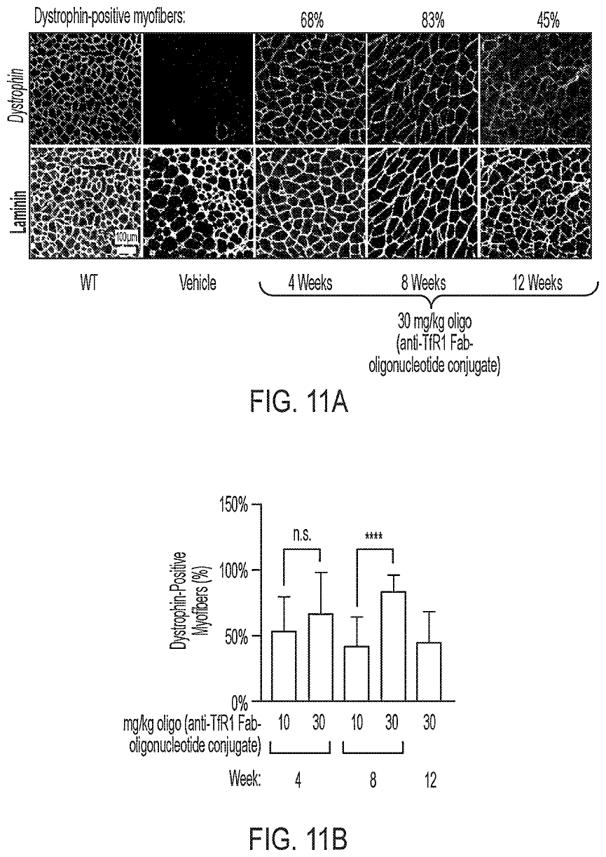

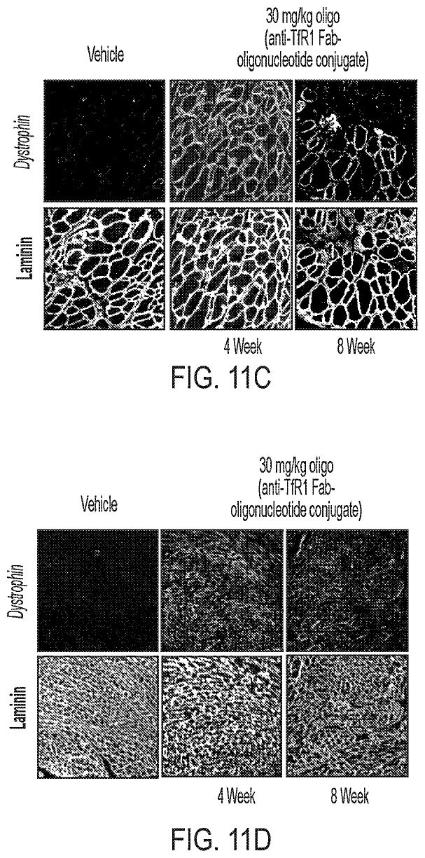

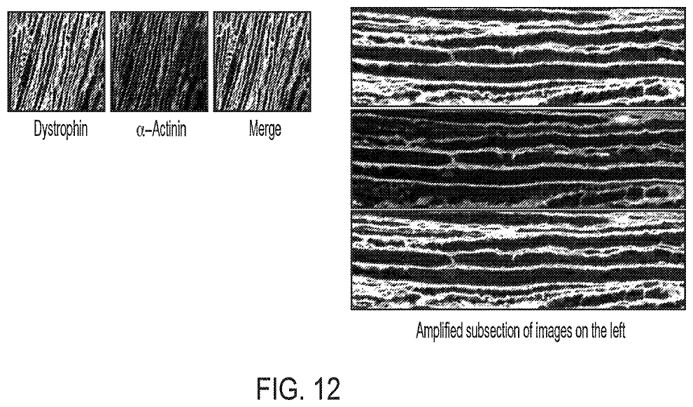

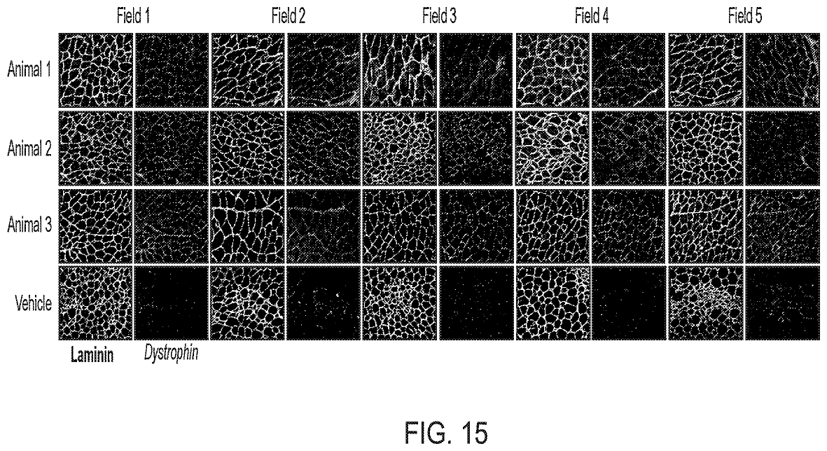

To evaluate dystrophin localization to the sarcolemma, its functional niche in skeletal and cardiac muscle, muscle cross-sections were visualized by immunofluorescent micrographs using anti-dystrophin and anti-laminin antibodies. Laminin is a large glycoprotein localized immediately outside the sarcolemma, (32) and laminin staining is commonly used to delineate the muscle myofibers and enable calculation of percent dystrophin-positive myofibers. (33) In the same anti-TfR1 Fab-oligonucleotide conjugate-treated mice in which exon skipping and dystrophin restoration were induced, dystrophin was localized to the sarcolemma in quadriceps, with a dose-dependent increase of dystrophin-positive fibers that was durable up to 12 weeks (FIG. 11A; FIG. 15 show individual fields quantified for 30 mg/kg oligonucleotide group at 8 weeks). A single dose of anti-TfR1 Fab-oligonucleotide conjugate at a dose equivalent to 30 mg/kg oligonucleotide resulted in 68%, 83%, and 45% dystrophin-positive fibers in quadriceps at 4, 8, and 12 weeks, respectively (FIG. 11B). Similarly, dystrophin was highly localized to the sarcolemma of diaphragm (FIG. 11C) and heart (FIG. 11D) 4 and 8 weeks after administration of a single dose of anti-TfR1 Fab-oligonucleotide conjugate at a dose equivalent to 30 mg/kg oligonucleotide. Of note, dystrophin localized to the sarcolemma along the length of the myofibers of the diaphragm indicated that dystrophin restoration was not a local event but extended to the entirety of the myofiber (FIG. 12).

### A Single Dose of Anti-TfR1 Fab-Oligonucleotide Conjugate, but not of Unconjugated Exon 23-Skipping Oligonucleotide, Improves Selected Functional Outcomes in Mdx Mice

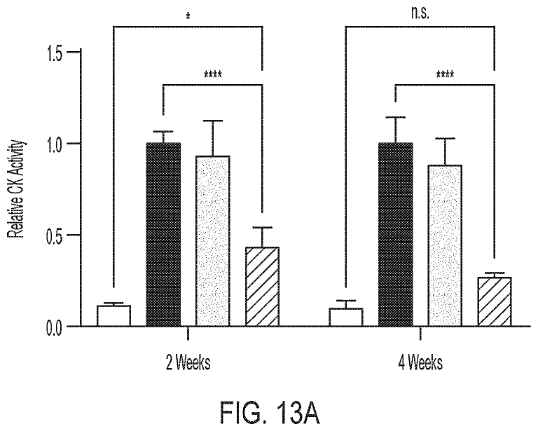

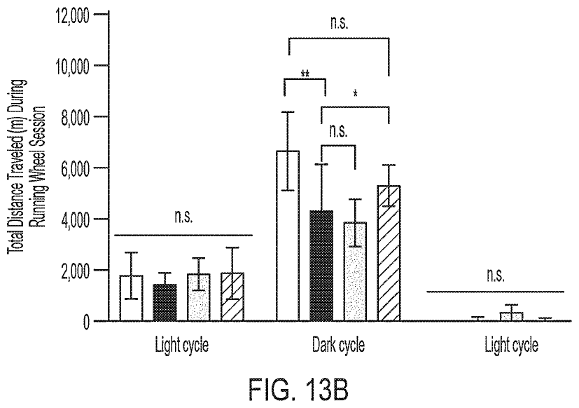

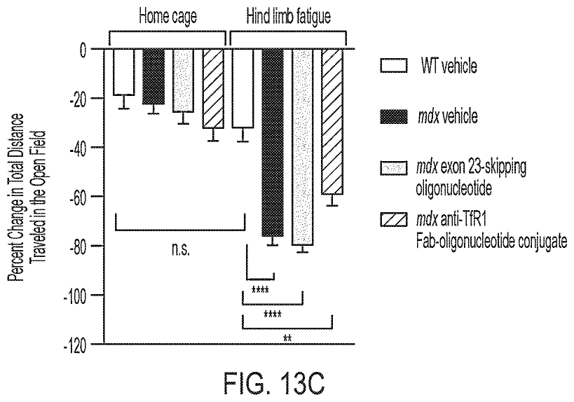

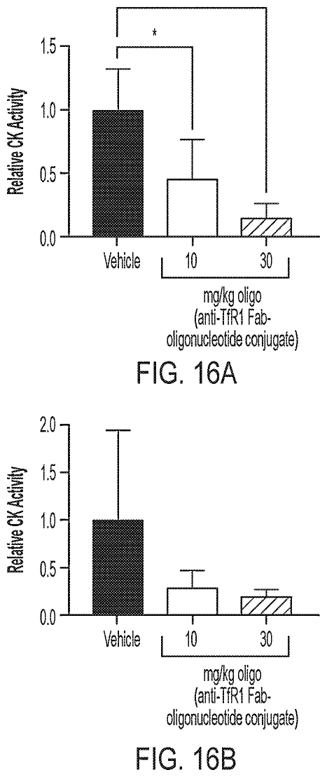

To determine whether anti-TfR1 Fab-oligonucleotide conjugate is superior to its unconjugated exon-skipping oligonucleotide in its ability to reduce muscle damage, mdx mice were injected with a single dose of anti-TfR1 Fab-oligonucleotide conjugate at a dose equivalent to 30 mg/kg oligonucleotide or a 30 mg/kg dose of unconjugated exon 23-skipping oligonucleotide. CK levels were measured 2- or 4-weeks after injection and compared with CK levels in wild-type or mdx mice treated with vehicle. Consistent with the compromised sarcolemma of mdx mice, serum CK levels were higher in these mice compared with their wild-type counterparts (FIG. 13A). A single dose of anti-TfR1 Fab-oligonucleotide conjugate, but not of unconjugated exon 23-skipping oligonucleotide, lowered serum CK in mdx mice at both 2- and 4-weeks post-dose compared with vehicle-treated mdx mice (p<0.0001). At 4-weeks post-dose, serum CK levels in mdx mice treated with anti-TfR1 Fab-oligonucleotide conjugate were similar to levels in vehicle-treated wild-type mice, indicating a correction of the mdx phenotype after a single dose of anti-TfR1 Fab-oligonucleotide conjugate. Similar observations at 4- and 8-weeks post-dose confirmed the ability of anti-TfR1 Fab-oligonucleotide conjugate to reduce CK serum levels and indicated that the effect is dose-dependent (FIGS. 16A-16B).

To assess whether the ability of anti-TfR1 Fab-oligonucleotide conjugate to ameliorate dystrophy-associated increases in serum CK translates into improved functional outcomes compared with unconjugated exon 23-skipping oligonucleotide, mdx mice (n=10 per group) received either 30 mg/kg unconjugated exon 23-skipping oligonucleotide or anti-TfR1 Fab-oligonucleotide conjugate at a dose equivalent to 30 mg/kg oligonucleotide; age-matched control mdx and wild-type mice (n=10 per group) were injected with vehicle. Voluntary running wheel and open-field activity after hindlimb fatigue challenge were evaluated 2-(n=10 mice per treatment group) and 4-weeks (n=5 mice per treatment group) post-dose.

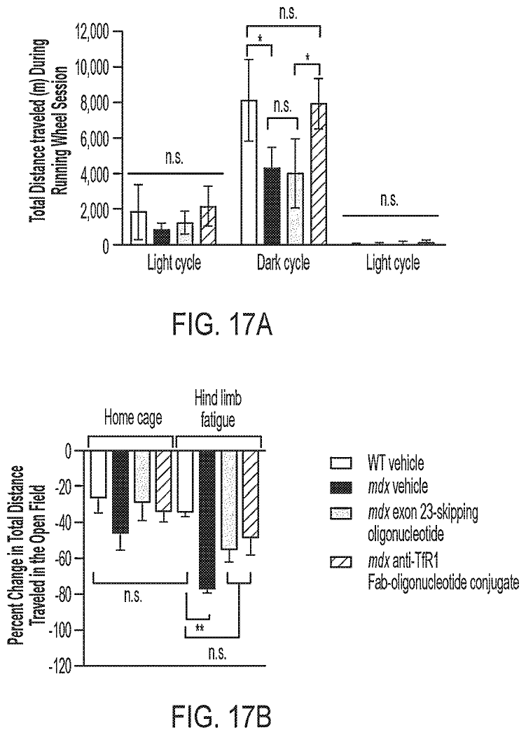

In a spontaneous running-wheel activity test 2-weeks post-dose, during the dark cycle (active time for mice), mdx mice treated with vehicle traveled a significantly shorter distance than wild-type mice receiving the same treatment (p=0.0022) (FIG. 13B). Conversely, mdx mice treated with a single dose of anti-TfR1 Fab-oligonucleotide conjugate traveled significantly more than did mdx mice treated with vehicle alone (p=0.0135). The distance traveled by mdx mice treated with anti-TfR1 Fab-oligonucleotide conjugate was similar to that traveled by wild-type mice treated with vehicle, indicating a rescue of the mdx phenotype by a single administration of anti-TfR1 Fab-oligonucleotide conjugate at a dose equivalent to 30 mg/kg oligonucleotide. Furthermore, compared with unconjugated exon 23-skipping oligonucleotide, anti-TfR1 Fab-oligonucleotide conjugate led to increased voluntary running wheel at 4 weeks (FIG. 17A).

Open-field activity was used to complement the running wheel functional measurements. Without hind-limb fatigue challenge, there was no significant difference in the total distance traveled between wild-type and mdx mice (FIG. 13C). Following hindlimb fatigue challenge which was performed to exacerbate the functional phenotype of the mdx mouse, there was a significant decrease (p<0.0001) in open-field activity in mdx compared with wild-type mice analyzed as a percent change in total distance traveled (FIG. 13C). A nonsignificant but positive trend toward increased open-field activity after hindlimb challenge was observed in mdx mice treated with a single dose of anti-TfR1 Fab-oligonucleotide conjugate, but not with unconjugated exon 23-skipping oligonucleotide, suggesting that anti-TfR1 Fab-oligonucleotide conjugate increases the muscle delivery and activity of an oligonucleotide (FIG. 13C, FIG. 17B).

### DISCUSSION

Therapies based on oligonucleotide-induced exon skipping have been approved in the United States for the treatment of patients with DMD who have mutations amenable to skipping of exons 51, 53, and 45. (12-15) While exon skipping approaches hold great promise in the treatment of DMD, approved therapies have shown very limited increases in dystrophin protein restoration and marginal, if any, functional benefit. Achieving efficient delivery to muscle has remained an insurmountable obstacle to the development of clinically effective therapies. (17)

Antisense oligonucleotide delivery has been improved by chemical modifications, use of alternative chemical backbones, and conjugation to delivery moieties such as peptides. (34) PMOs, in which a morpholine ring is substituted for the deoxyribose moiety and a phosphorodiamidate linkage replaces the phosphodiester intersubunit linkage, are more stable against nuclease and protease activity, compared with native DNA or RNA. (35,36) Nonetheless, a large body of published work revealed drawbacks of PMOs as therapies for DMD due to the poor uptake into cardiac and skeletal muscle. Consequently, duration of action of a single PMO dose is short, and treatment necessitates high drug dosages and/or repeated administration to achieve a therapeutic effect that is still hampered by inconsistent dystrophin protein restoration. (36)

The present study demonstrates that a Fab designed to bind to the murine TfR1 delivers an oligonucleotide (a PMO comprising a nucleobase sequence of SEQ ID NO: 36) capable of inducing Dmd exon 23 skipping (anti-TfR1 Fab-oligonucleotide conjugate) to skeletal and cardiac muscle on systemic administration to mdx mice. PMO tissue concentrations achieved a peak at the earliest timepoints measured after a single dose, followed closely by peak levels of exon 23 skipping. While both tissue exposure and exon 23 skipping levels declined shortly after achieving maximum levels, high levels of dystrophin protein restoration were observed at 4- and 8-weeks post-dose in cardiac and skeletal muscle, including the diaphragm. This is consistent with the relatively long half-life of dystrophin in skeletal muscle. Administration of anti-TfR1 Fab-oligonucleotide conjugate led to significantly more exposure and a greater pharmacological effect than an equimolar dose of unconjugated exon 23-skipping oligonucleotide. Although not every comparison between the two treatments achieved statistical significance, this is likely due to individual variability in mdx mice. Moreover, in agreement with the known limitations of unconjugated PMO delivery, unconjugated exon 23-skipping oligonucleotide led to an inconsistent, marginal exposure in cardiac and skeletal muscle and molecular as well as functional pharmacologic response.

Immunofluorescence analysis of quadriceps sections complemented the observations on protein expression and revealed that dystrophin localized at the sarcolemma, its physiological site of action, indicating that the shortened dystrophin is functionally sound. Importantly, abundant levels of dystrophin localized to the cell membrane in diaphragm and heart sections, although the observations were qualitative in nature. Interestingly, unlike quadriceps, total dystrophin restoration in the diaphragm and heart appears to decrease rapidly by 8 weeks post-dose. This observation is likely due to the sustained mechanical stress of constant contraction in both the diaphragm and heart. Collectively, these findings indicate that anti-TfR1 Fab-oligonucleotide conjugate delivers substantial amounts of PMO to cardiac and skeletal muscle, and that the dystrophin protein translated from the skipped transcript is likely functionally competent as it localizes to its physiological niche.

The duration of the pharmacodynamic response and the extent of dystrophin localization at the sarcolemma observed after administration of a single dose of anti-TfR1 Fab-oligonucleotide conjugate correlated with improvements in membrane integrity. Additionally, the reduction in serum CK levels combined with the correction of the functional mdx phenotype suggests that treatment with anti-TfR1 Fab-oligonucleotide conjugate restored a meaningful amount of functional dystrophin protein. anti-TfR1 Fab-oligonucleotide conjugate led to a superior molecular and phenotypic correction compared with the modest effect achieved by treatment with unconjugated exon 23-skipping oligonucleotide, confirms the notion that the anti-TfR1 Fab-oligonucleotide conjugate enhances delivery of an oligonucleotide therapeutic to skeletal and cardiac muscle. Based on these observations, treating DMD patients with a therapy based on the anti-TfR1 Fab-oligonucleotide conjugate could lead to superior benefit compared with the clinical improvement achieved with infusion of a non-muscle targeting PMO.

To address the limitations of current exon skipping PMO molecules and improve their potential as therapeutics for DMD, conjugation strategies to cell-penetrating peptides (PPMOs) have been implemented. Findings from multiple in vitro and in vivo reports have demonstrated that PPMOs display improved internalization into cells, efficiency in systemic delivery to target tissues, sustained dystrophin production, and enhanced potency at lower doses compared with unconjugated PMOs. (36) Gao et al. (39) reported that mdx mice administered with an exon skipping PPMO at 25 mg/kg once per week for 3 weeks or a single dose at 75 mg/kg showed effective exon skipping and dystrophin expression in skeletal muscles, but not in the heart, at 2 weeks after the last dose. In a different report, using a PMO-internalizing peptide (Pip6-PMO), Betts et al. (40) reported exon skipping and dystrophin expression in the hearts of mdx mice. Because modest dystrophin expression was seen after a single dose of 10 mg/kg, a longer treatment regimen totaling nine doses of Pip-PMO was instituted resulting in high levels of exon 23 skipping in the diaphragm (77%) and TA (86%). However, effects on the heart were modest, reaching only 32% skipping and 28% restoration of dystrophin expression. Thus, although systemic delivery of PMOs has been greatly improved by peptide conjugation, efficacy in cardiac tissues remains a challenge. In contrast, a single dose of anti-TfR1 Fab-oligonucleotide conjugate at a dose equivalent to 30 mg/kg oligonucleotide in mdx mice produced 40% exon skipping and a 77% dystrophin protein expression peak in the hearts at 4 weeks after administration. These observations suggest that the anti-TfR1 Fab-oligonucleotide conjugate has a high potential to deliver therapeutic levels of PMO payload to cardiac tissue.

While PPMO conjugation can increase delivery to muscle, the peptides moieties have known safety liabilities in organs such as the liver and kidneys, potentially leading to toxic side effects that are likely driven by the cationic nature of the peptide. (36) A multitude of additional factors such as the duration of treatment, dosage, frequency of systemic administration, and the animal model used to test the PPMOs could also contribute to the observed side effects. Therefore, using lower doses or allowing for longer intervals between dosing could help mitigate some of these risks. (36) It is important to note that a single dose of anti-TfR1 Fab-oligonucleotide conjugate achieved similar levels of skipping to those observed in studies with PPMOs in mdx mice, with pharmacological effects in skeletal and cardiac muscles noted up to 12 weeks. The prolonged duration of action combined with lower active dose levels supports the notion that the anti-TfR1 Fab-oligonucleotide conjugate may have a broader therapeutic index than a delivery mechanism that relies on cell-penetrating peptides.

Nevertheless, these findings demonstrate that a single dose of an anti-TfR1 Fab-oligonucleotide conjugate provides superior exon skipping and dystrophin expression, as well as greater improvement in muscle function, compared with approaches that leverage unconjugated oligonucleotides. In conclusion, the anti-TfR1 Fab-oligonucleotide conjugate combines the pharmacological properties of a biologic drug with the features of an oligonucleotide therapy and has the potential to provide an effective, re-dosable, and titratable treatment for patients with DMD.

### REFERENCES

1. Emery. A. E. (1991) Population frequencies of inherited neuromuscular diseases—a world survey. Neuromuscul Disord, 1, 19-29.
2. Gatheridge, M. A., Kwon, J. M., Mendell, J. M., Scheuerbrandt, G., Moat, S. J., Eyskens, F., Rockman-Greenberg, C., Drousiotou, A. and Griggs, R. C. (2016) Identifying Non-Duchenne Muscular Dystrophy-Positive and False Negative Results in Prior Duchenne Muscular Dystrophy Newborn Screening Programs: A Review. JAMA Neurol, 73, 111-116.
3. Bladen, C. L., Salgado, D., Monges, S., Foncuberta, M. E., Kekou, K., Kosma, K., Dawkins, H., Lamont, L., Roy, A. J., Chamova, T. et al. (2015) The TREAT-NMD DMD Global Database: analysis of more than 7,000 Duchenne muscular dystrophy mutations. Hum Mutat, 36, 395-402.
4. Flanigan, K. M., Dunn, D. M., von Niederhausern, A., Soltanzadch, P., Gappmaier, E., Howard, M. T., Sampson, J. B., Mendell, J. R., Wall, C., King, W. M. et al. (2009) Mutational spectrum of DMD mutations in dystrophinopathy patients: application of modern diagnostic techniques to a large cohort. Hum Mutat, 30, 1657-1666.
5. Koenig, M., Beggs, A. H., Moyer, M., Scherpf, S., Heindrich, K., Bettecken, T., Meng, G., Muller, C. R., Lindlof, M., Kaariainen, H. et al. (1989) The molecular basis for Duchenne versus Becker muscular dystrophy: correlation of severity with type of deletion. Am J Hum Genet, 45, 498-506.
6. Garcia-Rodriguez, R., Hiller, M., Jimenez-Gracia, L., van der Pal, Z., Balog, J., Adamzek, K., Aartsma-Rus, A. and Spitali, P. (2020) Premature termination codons in the DMD gene cause reduced local mRNA synthesis. Proc Natl Acad Sci USA, 117, 16456-16464.
7. Muntoni, F., Torelli, S. and Ferlini, A. (2003) Dystrophin and mutations: one gene, several proteins, multiple phenotypes. Lancet Neurol, 2, 731-740.
8. Straub, V. and Campbell, K. P. (1997) Muscular dystrophies and the dystrophin-glycoprotein complex. Curr Opin Neurol, 10, 168-175.
9. Duncan, C. J. (1989) Dystrophin and the integrity of the sarcolemma in Duchenne muscular dystrophy. Experientia, 45, 175-177.
10. Brogna, C., Lucibello, S., Coratti, G., Vita, G., Sansone, V. A., Messina, S., Albamonte, E., Salmin, F., Ferrantini, G., Pede, E. et al. (2020) Respiratory function and therapeutic expectations in DMD: families experience and perspective. Acta Myol, 39, 121-129.
11. Kole, R. and Krieg, A. M. (2015) Exon skipping therapy for Duchenne muscular dystrophy. Adv Drug Deliv Rev, 87, 104-107.
12. Lim, K. R., Maruyama, R. and Yokota, T. (2017) Eteplirsen in the treatment of Duchenne muscular dystrophy. Drug Des Devel Ther, 11, 533-545.
13. Heo, Y. A. (2020) Golodirsen: First Approval. Drugs, 80, 329-333.
14. Dhillon, S. (2020) Viltolarsen: First Approval. Drugs, 80, 1027-1031.
15. Shirley, M. (2021) Casimersen: First Approval. Drugs, 81, 875-879.
16. Dowdy, S. F. (2017) Overcoming cellular barriers for RNA therapeutics. Nat Biotechnol, 35, 222-229.
17. Godfrey, C., Desviat, L. R., Smedsrod, B., Pietri-Rouxel, F., Denti, M. A., Disterer, P., Lorain, S., Nogales-Gadea, G., Sardone, V., Anwar, R. et al. (2017) Delivery is key: lessons learnt from developing splice-switching antisense therapies. EMBO Mol Med, 9, 545-557.
18. Arechavala-Gomeza, V., Anthony, K., Morgan, J. and Muntoni, F. (2012) Antisense oligonucleotide-mediated exon skipping for Duchenne muscular dystrophy: progress and challenges. Curr Gene Ther, 12, 152-160.
19. Daniels, T. R., Delgado, T., Helguera, G. and Penichet, M. L. (2006) The transferrin receptor part II: targeted delivery of therapeutic agents into cancer cells. Clin Immunol, 121, 159-176.
20. Barrientos, T., Laothamatas, I., Koves, T. R., Soderblom, E. J., Bryan, M., Moseley, M. A., Muoio, D. M. and Andrews, N. C. (2015) Metabolic Catastrophe in Mice Lacking Transferrin Receptor in Muscle. EBioMedicine, 2, 1705-1717.
21. Li, Y., Cheng, J. X., Yang, H. H., Chen, L. P., Liu, F. J., Wu, Y., Fan, M. and Wu, H. T. (2021) Transferrin receptor 1 plays an important role in muscle development and denervation-induced muscular atrophy. Neural Regen Res, 16, 1308-1316.
22. Ciechanover, A., Schwartz, A. L., Dautry-Varsat, A. and Lodish, H. F. (1983) Kinetics of internalization and recycling of transferrin and the transferrin receptor in a human hepatoma cell line. Effect of lysosomotropic agents. J Biol Chem, 258, 9681-9689.
23. Lesley, J., Schulte, R. and Woods, J. (1989) Modulation of transferrin receptor expression and function by anti-transferrin receptor antibodies and antibody fragments. Exp Cell Res, 182, 215-233.
24 Tsui, C. K., Barfield, R. M., Fischer, C. R., Morgens, D. W., Li, A., Smith, B. A. H., Gray, M. A., Bertozzi, C. R., Rabuka, D. and Bassik, M. C. (2019) CRISPR-Cas9 screens identify regulators of antibody-drug conjugate toxicity. Nat Chem Biol, 15, 949-958.
25. McCombs. J. R. and Owen, S. C. (2015) Antibody drug conjugates: design and selection of linker, payload and conjugation chemistry. AAPS J, 17, 339-351.
26. Jearawiriyapaisarn, N., Moulton, H. M., Buckley, B., Roberts, J., Sazani, P., Fucharoen, S., Iversen, P. L. and Kole, R. (2008) Sustained dystrophin expression induced by peptide-conjugated morpholino oligomers in the muscles of mdx mice. Mol Ther, 16, 1624-1629.
27. Yin, H., Moulton, H. M., Scow. Y., Boyd. C., Boutilier, J., Iverson, P. and Wood, M. J. (2008) Cell-penetrating peptide-conjugated antisense oligonucleotides restore systemic muscle and cardiac dystrophin expression and function. Hum Mol Genet, 17, 3909-3918.
28. Bulfield, G., Siller, W. G., Wight, P. A. and Moore, K. J. (1984) X chromosome-linked muscular dystrophy (mdx) in the mouse. Proc Natl Acad Sci USA, 81, 1189-1192.
29. Burki, U., Keane, J., Blain, A., O'Donovan, L., Gait, M. J., Laval, S. H. and Straub, V. (2015) Development and Application of an Ultrasensitive Hybridization-Based ELISA Method for the Determination of Peptide-Conjugated Phosphorodiamidate Morpholino Oligonucleotides. Nucleic Acid Ther, 25, 275-284.
30. Schneider, C. A., Rasband, W. S. and Eliceiri, K. W. (2012) NIH Image to ImageJ: 25 years of image analysis. Nat Methods, 9, 671-675.
31. Echigoya, Y., Lec, J., Rodrigues, M., Nagata, T., Tanihata, J., Nozohourmehrabad, A., Panesar, D., Miskew, B., Aoki, Y. and Yokota, T. (2013) Mutation types and aging differently affect revertant fiber expansion in dystrophic mdx and mdx52 mice. PLOS One, 8, c69194.
32. Durbecj. M. (2010) Laminins. Cell Tissue Res, 339, 259-268.
33 Tiger, C. F., Champliaud, M. F., Pedrosa-Domellof, F., Thornell, L. E., Ekblom, P. and Gullberg, D. (1997) Presence of laminin alpha5 chain and lack of laminin alphal chain during human muscle development and in muscular dystrophies. J Biol Chem, 272, 28590-28595.
34. Godfrey, C., Desviat, L. R., Smedsrød, B., Piétri-Rouxel, F., Denti, M. A., Disterer, P., Lorain, S., Nogales-Gadea, G., Sardone, V., Anwar, R. et al. (2017) Delivery is key: lessons learnt from developing splice-switching antisense therapies. EMBO Mol. Med., 9, 545-557.
35. Sazani, P., Ness, K. P., Weller, D. L., Poage, D., Nelson, K. and Shrewsbury, A. S. (2011) Chemical and mechanistic toxicology evaluation of exon skipping phosphorodiamidate morpholino oligomers in mdx mice. Int J Toxicol, 30, 322-333.
36. Tsoumpra, M. K., Fukumoto, S., Matsumoto, T., Takeda, S., Wood, M. J. A. and Aoki, Y. (2019) Peptide-conjugate antisense based splice-correction for Duchenne muscular dystrophy and other neuromuscular diseases. EBioMedicine, 45, 630-645.
37. McNally, E. M. (2007) New approaches in the therapy of cardiomyopathy in muscular dystrophy. Annu Rev Med, 58, 75-88.
38. E. Ballard, N. G., H. Jungbluth, E. Wraige, S. Kapetanakis, C. Davidson, N. Hart (2012) Observation cohort study of cause of death in patients with Duchenne muscular dystrophy (DMD). Eur Respiratory J, 40, 1720.
39 Gao, X., Zhao, J., Han, G., Zhang, Y., Dong, X., Cao, L., Wang, Q., Moulton, H. M. and Yin, H. (2014) Effective dystrophin restoration by a novel muscle-homing peptide-morpholino conjugate in dystrophin-deficient mdx mice. Mol Ther, 22, 1333-1341.
40. Betts, C. A., Salch, A. F., Carr, C. A., Hammond, S. M., Coenen-Stass, A. M., Godfrey, C., McClorey, G., Varela, M. A., Roberts, T. C., Clarke, K. et al. (2015) Prevention of exercised induced cardiomyopathy following Pip-PMO treatment in dystrophic mdx mice. Sci Rep, 5, 8986.

### Additional Embodiments

1. A method of promoting expression or activity of a dystrophin protein in a subject, comprising administering to the subject a composition comprising an effective amount of complexes, each complex comprising an anti-transferrin receptor 1 (TfR1) antibody covalently linked to one or more oligonucleotides, wherein the effective amount provides to the subject 3 mg to 80 mg of the oligonucleotides of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the nucleotide sequence of CTCCAACATCAAGGAAGATGGCATTTCTAG (SEQ ID NO: 21) and is a phosphorodiamidate morpholino oligomer (PMO).

2. A method of treating Duchenne Muscular Dystrophy (DMD) in a subject, comprising administering to the subject a composition comprising an effective amount of complexes, each complex comprising an anti-transferrin receptor 1 (TfR1) antibody covalently linked to one or more oligonucleotides, wherein the effective amount provides to the subject 3 mg to 80 mg of the oligonucleotides of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the nucleotide sequence of CTCCAACATCAAGGAAGATGGCATTTCTAG (SEQ ID NO: 21) and is a phosphorodiamidate morpholino oligomer (PMO).

3. The method of embodiment 1 or embodiment 2, wherein each complex comprises a structure of formula (I): [R¹]ₙ₁—R², wherein:
each R′ comprises a group of the formula (Ib):

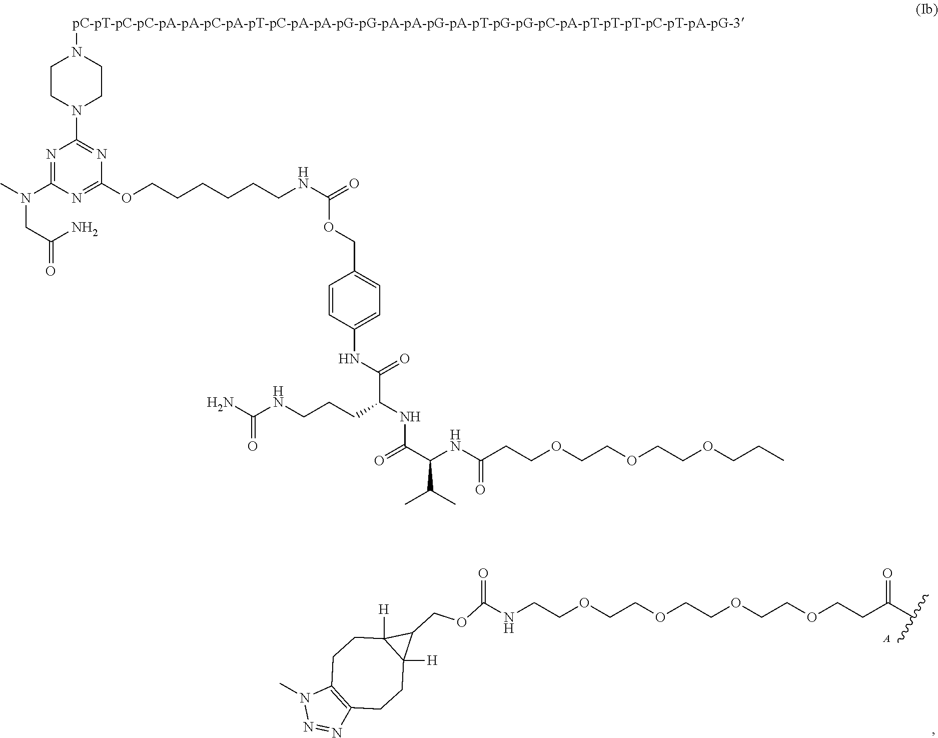

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises the anti-TfR1 antibody; and in each complex, n1 is independently an integer of one or greater representing the number of instances of R¹, wherein each instance of R′ is covalently linked via attachment point A to a different lysine of the anti-TfR1 antibody, optionally wherein the average value of n1 of the complexes of the composition is in the range of 1 to 5.

4. The method of embodiment 1 or embodiment 2, wherein each complex comprises a structure of formula (I): [R¹]ₙ₁—R², wherein:
each R¹ comprises a group of the formula (Ic):

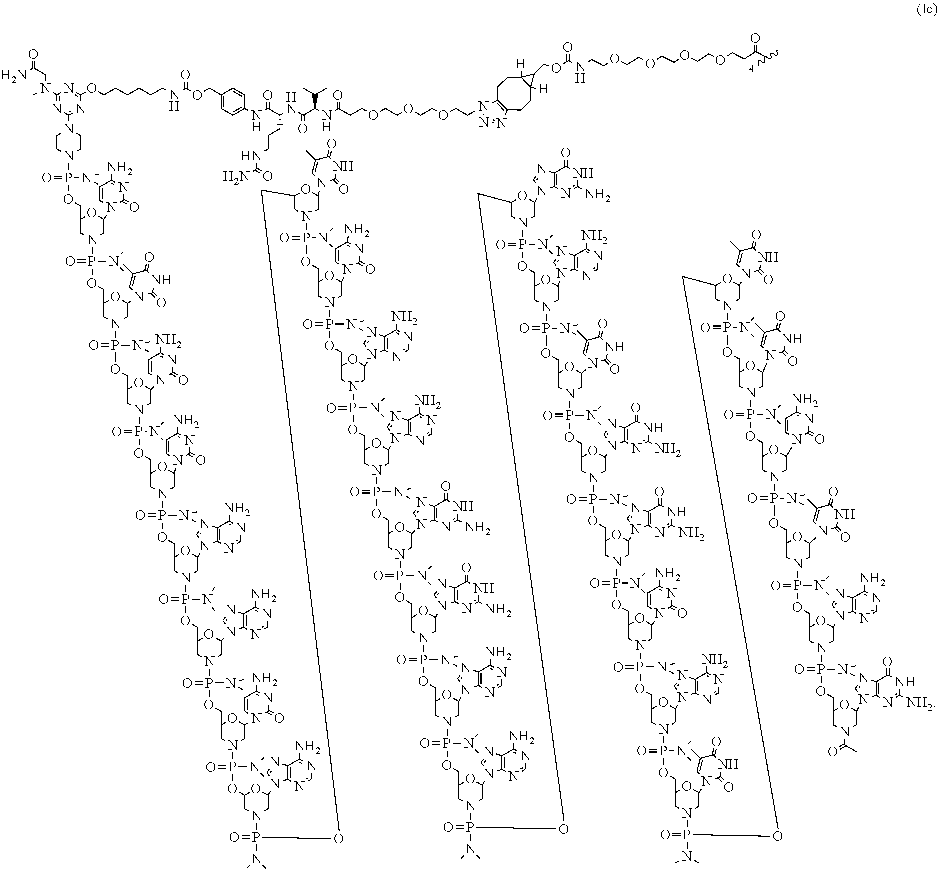

R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 1 to 5.

5. The method of any one of embodiments 1-4, wherein the anti-TfR1 antibody is a Fab fragment.

6. The method of any one of embodiments 1-5, wherein the anti-TfR1 antibody comprises a heavy chain variable region (VH) comprising the amino acid sequence of SEQ ID NO: 17 and a light chain variable region (VL) comprising the amino acid sequence of SEQ ID NO: 18.

7. The method of any one of embodiments 1-6, wherein the anti-TfR1 antibody comprises a heavy chain comprising the amino acid sequence of SEQ ID NO: 19 and a light chain comprising the amino acid sequence of SEQ ID NO: 20.

8. The method of any one of embodiments 1-7, wherein the effective amount of each administration provides to the subject 10 mg to 50 mg of the oligonucleotides of the complexes per kg of the subject.

9. The method of any one of embodiments 1-7, wherein the effective amount of each administration provides to the subject 8-36 mg of the oligonucleotides of the complexes per kg of the subject, optionally wherein the effective amount of each administration provides to the subject 10-30 mg of the oligonucleotides of the complexes per kg of the subject.

10. The method of any one of embodiments 1-7, wherein the effective amount of each administration provides to the subject 3-52 mg of the oligonucleotides of the complexes per kg of the subject, optionally wherein the effective amount of each administration provides to the subject 5-40 mg of the oligonucleotides of the complexes per kg of the subject.

11. The method of any one of embodiments 1-10, wherein the composition is administered once every 4 weeks.

12. The method of any one of embodiments 1-10, wherein the composition is administered once every 8 weeks.

13. The method of any one of embodiments 1-10, wherein the composition is administered once every 12 weeks.

14. The method of any one of embodiments 1-11, wherein the administration is once every four weeks and wherein the effective amount of each administration provides to the subject 8-12 mg of the oligonucleotides of the complexes per kg of the subject, optionally wherein the effective amount of each administration provides to the subject 10 mg of the oligonucleotides of the complexes per kg of the subject.

15. The method of any one of embodiments 1-11, wherein the administration is once every four weeks and wherein the effective amount of each administration provides to the subject 24-36 mg of the oligonucleotides of the complexes per kg of the subject, optionally wherein the effective amount of each administration provides to the subject 30 mg of the oligonucleotides of the complexes per kg of the subject.

16. The method of any one of embodiments 1-11, wherein the administration is once every four weeks and wherein the effective amount of each administration provides to the subject 3-7 mg of the oligonucleotides of the complexes per kg of the subject, optionally wherein the effective amount of each administration provides to the subject 5.03 mg of the oligonucleotides of the complexes per kg of the subject.

17. The method of any one of embodiments 1-11, wherein the administration is once every four weeks and wherein the effective amount of each administration provides to the subject 7-13 mg of the oligonucleotides of the complexes per kg of the subject, optionally wherein the effective amount of each administration provides to the subject 10.06 mg of the oligonucleotides of the complexes per kg of the subject.

18. The method of any one of embodiments 1-11, wherein the administration is once every four weeks and wherein the effective amount of each administration provides to the subject 16-26 mg of the oligonucleotides of the complexes per kg of the subject, optionally wherein the effective amount of each administration provides to the subject 20.13 mg of the oligonucleotides of the complexes per kg of the subject.

19. The method of any one of embodiments 1-11, wherein the administration is once every four weeks and wherein the effective amount of each administration provides to the subject 31-52 mg of the oligonucleotides of the complexes per kg of the subject, optionally wherein the effective amount of each administration provides to the subject 40.27 mg of the oligonucleotides of the complexes per kg of the subject.

20. The method of any one of embodiments 1-7, wherein the composition is administered to the subject once every two weeks in the first four weeks and subsequently administered once every four weeks.

21. The method of embodiment 20, wherein the effective amount of each administration in the first four weeks provides to the subject 24-36 mg of the oligonucleotides of the complexes per kg of the subject, optionally wherein the effective amount of each administration in the first four weeks provides to the subject 30 mg of the oligonucleotides of the complexes per kg of the subject.

22. The method of embodiment 20, wherein the effective amount of the first two administrations in the first four weeks provides to the subject a total of 24-36 mg of the oligonucleotides of the complexes per kg of the subject, optionally wherein the effective amount of the first two administrations in the first four weeks provides to the subject a total of 30 mg of the oligonucleotides of the complexes per kg of the subject.

23. The method of any one of embodiments 20-22, wherein the effective amount of each subsequent administration provides to the subject 24-36 mg of the oligonucleotides of the complexes per kg of the subject, optionally wherein the effective amount of each subsequent administration provides to the subject 30 mg of the oligonucleotides of the complexes per kg of the subject.

24. The method of embodiment 20, wherein the effective amount of each administration in the first four weeks provides to the subject 3-52 mg of the oligonucleotides of the complexes per kg of the subject, optionally wherein the effective amount of each administration in the first four weeks provides to the subject 5-40 mg of the oligonucleotides of the complexes per kg of the subject.

25. The method of embodiment 20, wherein the effective amount of the first two administrations in the first four weeks provides to the subject a total of 3-52 mg of the oligonucleotides of the complexes per kg of the subject, optionally wherein the effective amount of the first two administrations in the first four weeks provides to the subject a total of 5-40 mg of the oligonucleotides of the complexes per kg of the subject.

26. The method of any one of embodiments 20, 24, and 25, wherein the effective amount of each subsequent administration provides to the subject 3-52 mg of the oligonucleotides of the complexes per kg of the subject, optionally wherein the effective amount of each subsequent administration provides to the subject 5-40 mg of the oligonucleotides of the complexes per kg of the subject.

27. The method of any one of embodiments 1-26, wherein the composition is in an aqueous solution and further comprises histidine and sucrose.

28. The method of embodiment 27, wherein the histidine is present in the aqueous solution at a concentration of 25 mM, the sucrose is present in the aqueous solution at a concentration of 10 w/v %, and the aqueous solution is at a pH of 6.0.

29. The method of embodiment 27 or embodiment 28, wherein the complexes are present in the composition at a concentration in the range of 10 mg/mL to 50 mg/mL.

30. The method of any one of embodiments 1-29, wherein the subject has a mutated dystrophin allele comprising a mutation amenable to exon 51 skipping.

31. The method of embodiment 30, wherein the mutated dystrophin allele comprises a frameshift mutation in exon 51.

32. The method of any one of embodiments 1-31, wherein the complex promotes expression or activity of dystrophin protein in the subject.

33. The method of embodiment 32, wherein the dystrophin protein is a truncated dystrophin protein.

34. The method of any one of embodiments 1-33, wherein administration of the composition results in greater than 1% of dystrophin protein in the subject relative to a control.

35. The method of embodiment 34, wherein the control is dystrophin protein level in a healthy subject.

36 The method of embodiment 34, wherein the control is dystrophin protein level in a subject that does not have a mutated dystrophin allele, optionally wherein the subject that does not have a mutated dystrophin allele having a frameshift mutation.

37. The method of any one of embodiments 1-36, wherein administration of the composition increases the number of dystrophin positive fibers to about 50% of total muscle fibers in a muscle tissue sample of the subject.

38. The method of any one of embodiments 1-37, wherein the subject is a human subject, optionally wherein the human subject is between 2-60 years of age.

39. The method of any one of embodiments 1-38, wherein the subject is a human subject that is between 4-16 years of age.

40. The method of any one of embodiments 1-37, wherein the subject is a rodent.

41. The method of any one of embodiments 1-37, wherein the subject is a non-human primate.

42. The method of any one of embodiments 1-41, wherein the complex is administered systemically, optionally wherein the complex is administered by infusion.

43. A method of promoting expression or activity of a dystrophin protein in a subject, comprising administering via infusion an effective amount of a formulation of a composition comprising complexes in an aqueous solution, wherein each complex comprises a structure of formula (I): [R¹]ₙ₁—R², wherein:
each R¹ comprises a group of the formula (Ib):

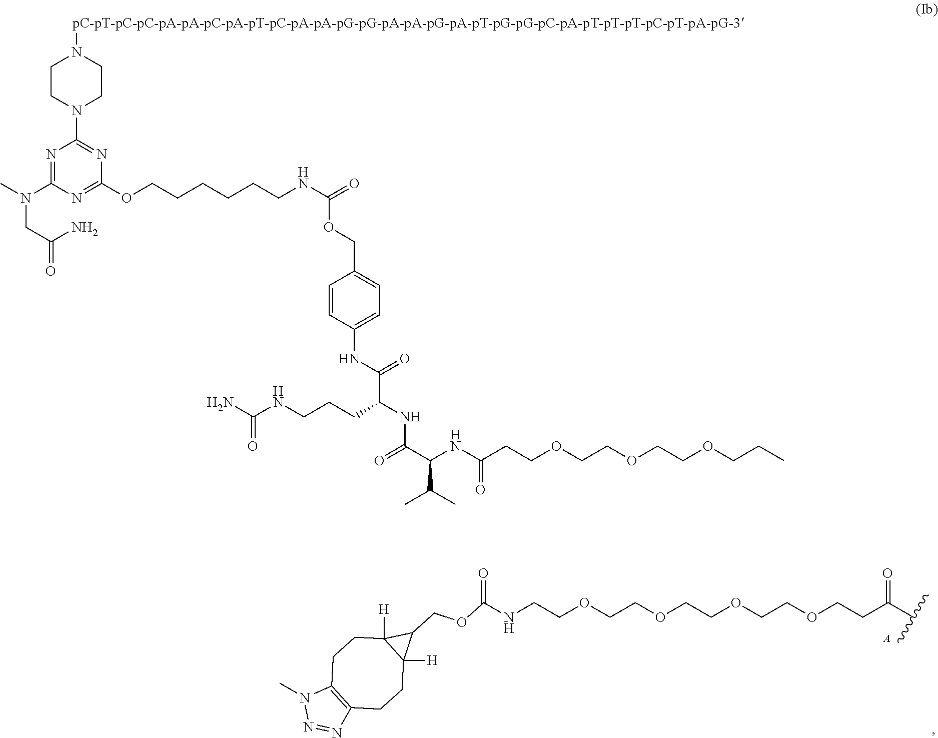

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex n1 is independently an integer of one or greater representing the number of instances of R¹, wherein each instance of R¹ is covalently linked via attachment point A to a different lysine of the anti-TfR1 Fab, optionally wherein the average value of n1 of the complexes of the composition is in the range of 1 to 5;
wherein the aqueous solution is at a pH of 6.0 and comprises histidine at a concentration of 25 mM, sucrose at a concentration of 10 w/v %, and the complexes at a concentration in the range of 10 mg/mL to 50 mg/mL;
wherein the effective amount provides to the subject 8 mg to 36 mg of the oligonucleotides of the complexes per kg of the subject, optionally wherein the effective amount provides to the subject 10 mg to 30 mg of the oligonucleotides of the complexes per kg of the subject.

44. A method of promoting expression or activity of a dystrophin protein in a subject, comprising administering via infusion an effective amount of a formulation of a composition comprising complexes in an aqueous solution, wherein each complex comprises a structure of formula (I): [R¹]ₙ₁—R², wherein:
each R¹ comprises a group of the formula (Ic):

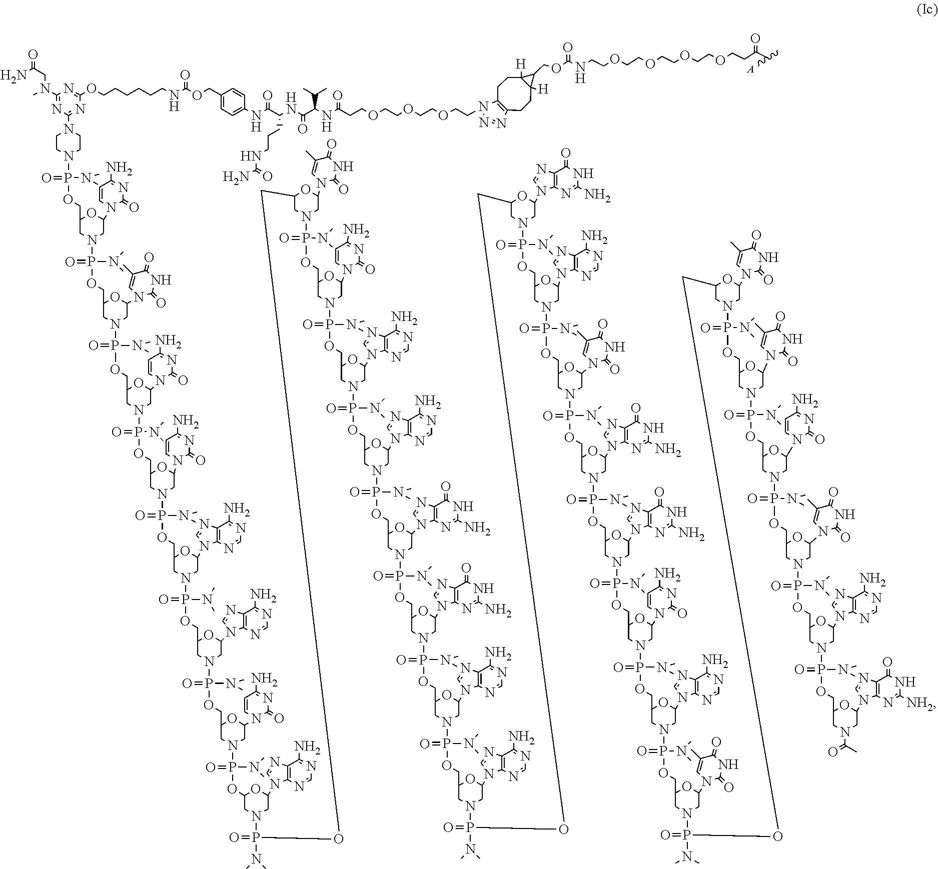

wherein R² comprises an anti-TfR1 Fab comprising a heavy chain comprising the amino acid sequence of SEQ ID NO: 19 and a light chain comprising the amino acid sequence of SEQ ID NO: 20; and
in each complex n1 is independently an integer of one or greater representing the number of instances of R¹, wherein each instance of R¹ is covalently linked via attachment point A to a different lysine of the anti-TfR1 Fab, optionally wherein the average value of n1 of the complexes of the composition is in the range of 1 to 5;
wherein the aqueous solution is at a pH of 6.0 and comprises histidine at a concentration of 25 mM, sucrose at a concentration of 10 w/v %, and the complexes at a concentration in the range of 10 mg/mL to 50 mg/mL;
wherein the effective amount provides to the subject 8 mg to 36 mg of the oligonucleotides of the complexes per kg of the subject, optionally wherein the effective amount provides to the subject 10 mg to 30 mg of the oligonucleotides of the complexes per kg of the subject.

45. A method of promoting expression or activity of a dystrophin protein in a subject, comprising administering via infusion an effective amount of a formulation of a composition comprising complexes in an aqueous solution, wherein the complex comprises a structure of formula (Id):

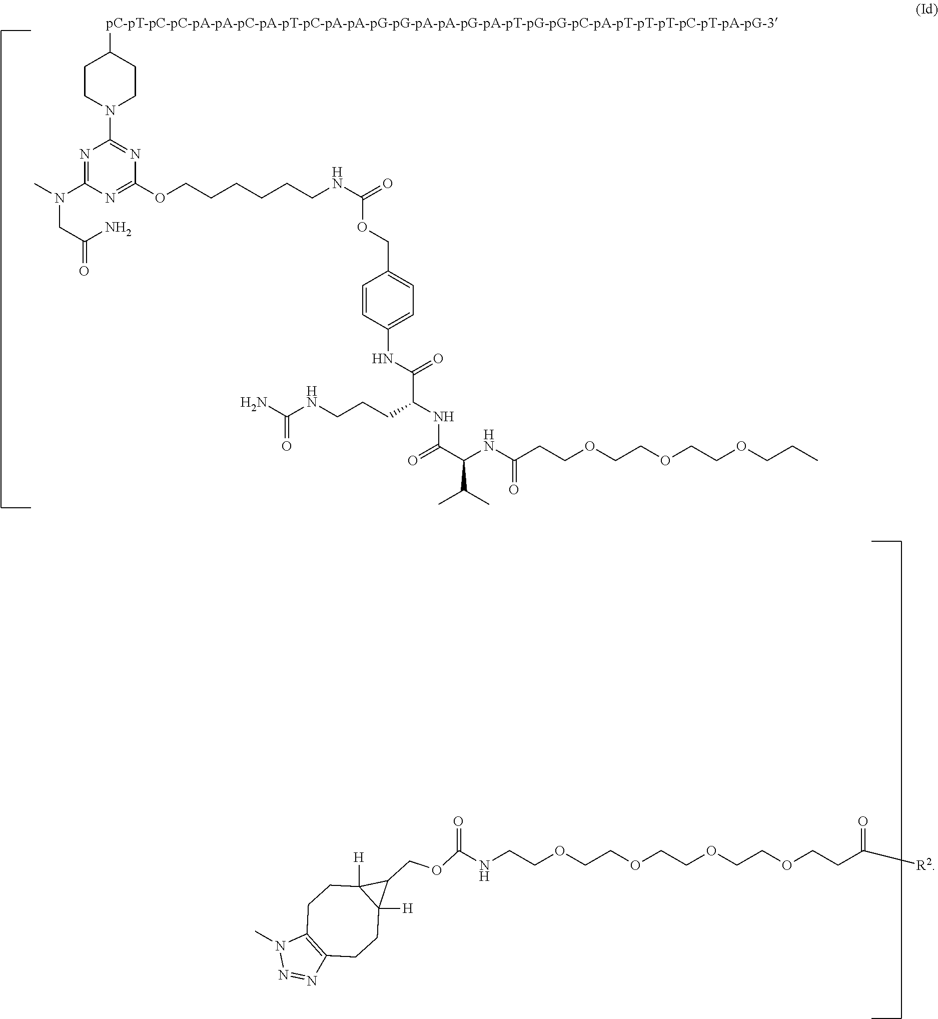

wherein R² comprises an anti-TfR1 Fab comprising a heavy chain comprising the amino acid sequence of SEQ ID NO: 19 and a light chain comprising the amino acid sequence of SEQ ID NO: 20; and
wherein the aqueous solution is at a pH of 6.0 and comprises histidine at a concentration of 25 mM, sucrose at a concentration of 10 w/v %, and the complexes at a concentration in the range of 10 mg/mL to 50 mg/mL;
wherein the effective amount provides to the subject 8 mg to 36 mg of the oligonucleotides of the complexes per kg of the subject, optionally wherein the effective amount provides to the subject 10 mg to 30 mg of the oligonucleotides of the complexes per kg of the subject.

46. A method of promoting expression or activity of a dystrophin protein in a subject, comprising administering via infusion an effective amount of a formulation of a composition comprising complexes in an aqueous solution, wherein each complex comprises a structure of formula (I): [R¹]ₙ₁—R², wherein:
each R¹ comprises a group of the formula (Ib):

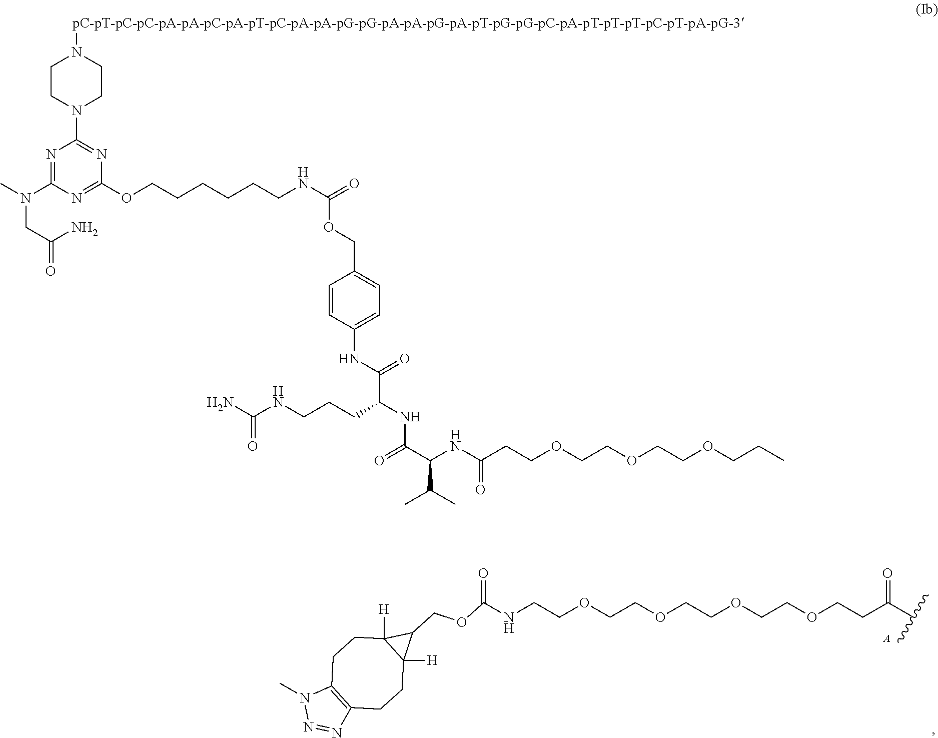

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex n1 is independently an integer of one or greater representing the number of instances of R¹, wherein each instance of R¹ is covalently linked via attachment point A to a different lysine of the anti-TfR1 Fab, optionally wherein the average value of n1 of the complexes of the composition is in the range of 1 to 5;
wherein the aqueous solution is at a pH of 6.0 and comprises histidine at a concentration of 25 mM, sucrose at a concentration of 10 w/v %, and the complexes at a concentration in the range of 10 mg/mL to 50 mg/mL;
wherein the effective amount provides to the subject 3 mg to 52 mg of the oligonucleotides of the complexes per kg of the subject, optionally wherein the effective amount provides to the subject 5 mg to 40 mg of the oligonucleotides of the complexes per kg of the subject.

47. A method of promoting expression or activity of a dystrophin protein in a subject, comprising administering via infusion an effective amount of a formulation of a composition comprising complexes in an aqueous solution, wherein each complex comprises a structure of formula (I): [R¹]ₙ₁—R², wherein:
each R¹ comprises a group of the formula (Ic):

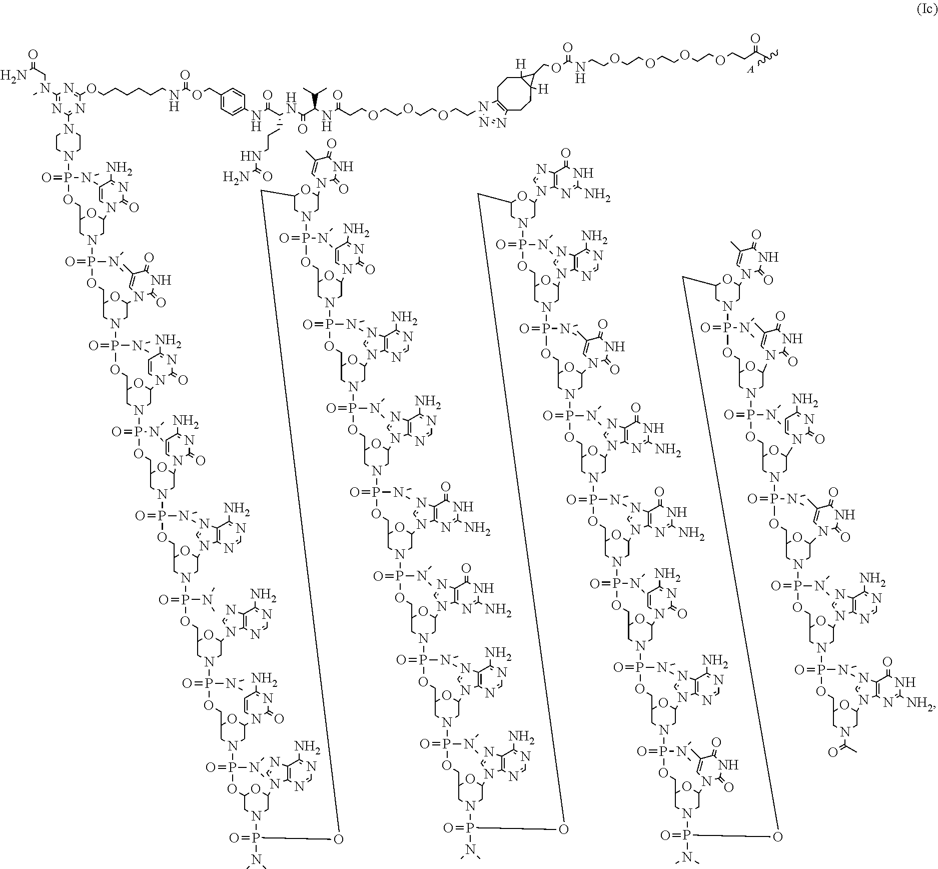

wherein R² comprises an anti-TfR1 Fab comprising a heavy chain comprising the amino acid sequence of SEQ ID NO: 19 and a light chain comprising the amino acid sequence of SEQ ID NO: 20; and
in each complex n1 is independently an integer of one or greater representing the number of instances of R¹, wherein each instance of R¹ is covalently linked via attachment point A to a different lysine of the anti-TfR1 Fab, optionally wherein the average value of n1 of the complexes of the composition is in the range of 1 to 5;
wherein the aqueous solution is at a pH of 6.0 and comprises histidine at a concentration of 25 mM, sucrose at a concentration of 10 w/v %, and the complexes at a concentration in the range of 10 mg/mL to 50 mg/mL;
wherein the effective amount provides to the subject 3 mg to 52 mg of the oligonucleotides of the complexes per kg of the subject, optionally wherein the effective amount provides to the subject 5 mg to 40 mg of the oligonucleotides of the complexes per kg of the subject.

48. A method of promoting expression or activity of a dystrophin protein in a subject, comprising administering via infusion an effective amount of a formulation of a composition comprising complexes in an aqueous solution, wherein the complex comprises a structure of formula (Id):

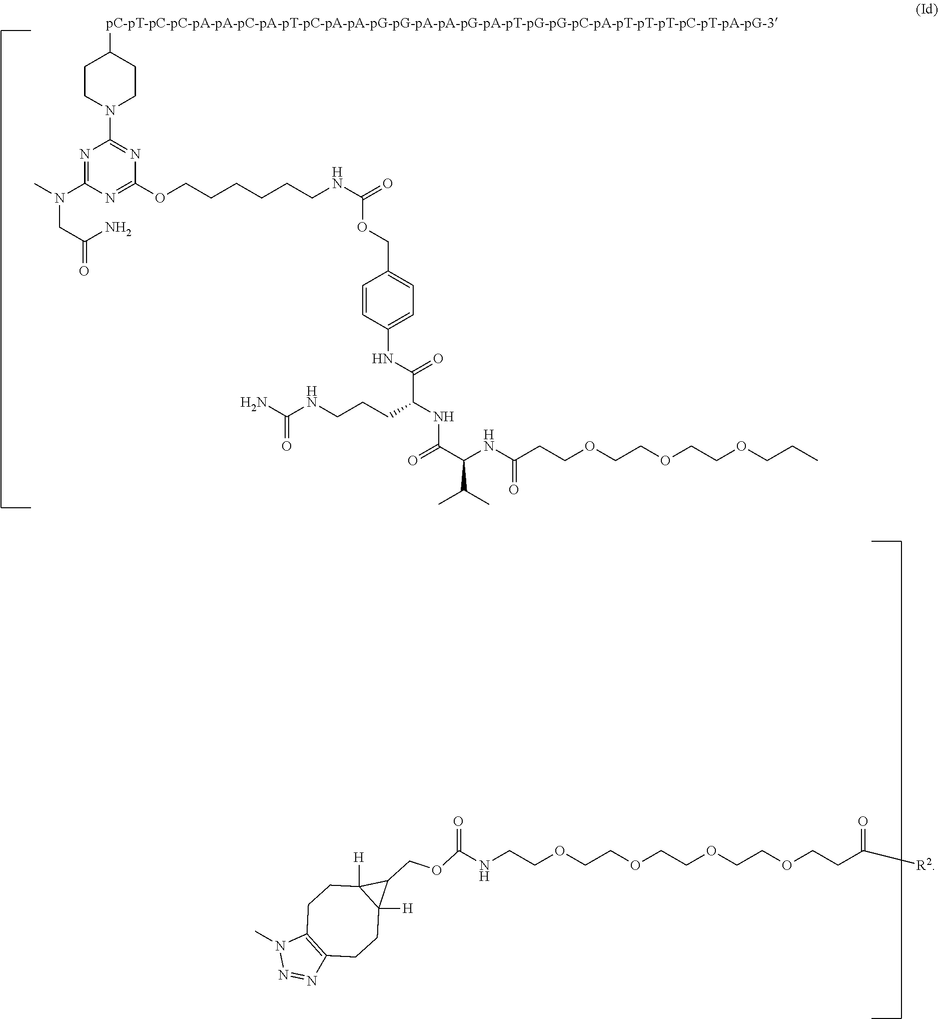

wherein R² comprises an anti-TfR1 Fab comprising a heavy chain comprising the amino acid sequence of SEQ ID NO: 19 and a light chain comprising the amino acid sequence of SEQ ID NO: 20; and
wherein the aqueous solution is at a pH of 6.0 and comprises histidine at a concentration of 25 mM, sucrose at a concentration of 10 w/v %, and the complexes at a concentration in the range of 10 mg/mL to 50 mg/mL;
wherein the effective amount provides to the subject 3 mg to 52 mg of the oligonucleotides of the complexes per kg of the subject, optionally wherein the effective amount provides to the subject 5 mg to 40 mg of the oligonucleotides of the complexes per kg of the subject.

49. The method of any one of embodiments 43-48, wherein the administration is once every four weeks, once every eight weeks, or once every twelve weeks.

50. The method of any one of embodiments 43-49, wherein the subject has Duchenne Muscular Dystrophy (DMD).

51. A method of promoting expression or activity of a dystrophin protein or treating Duchenne Muscular Dystrophy (DMD) in a subject, comprising administering to the subject a composition comprising an effective amount of complexes, each complex comprising an anti-transferrin receptor 1 (TfR1) antibody covalently linked to one or more oligonucleotides, wherein the effective amount provides to the subject 5 mg to 120 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the nucleotide sequence of CTCCAACATCAAGGAAGATGGCATTTCTAG (SEQ ID NO: 21) and is a phosphorodiamidate morpholino oligomer (PMO).

52. A method of promoting expression or activity of a dystrophin protein in a subject, comprising administering via infusion an effective amount of a composition comprising complexes, wherein each complex comprises a structure of formula (I): [R¹]ₙ₁—R², wherein:
each R¹ comprises a group of the formula (Ib):

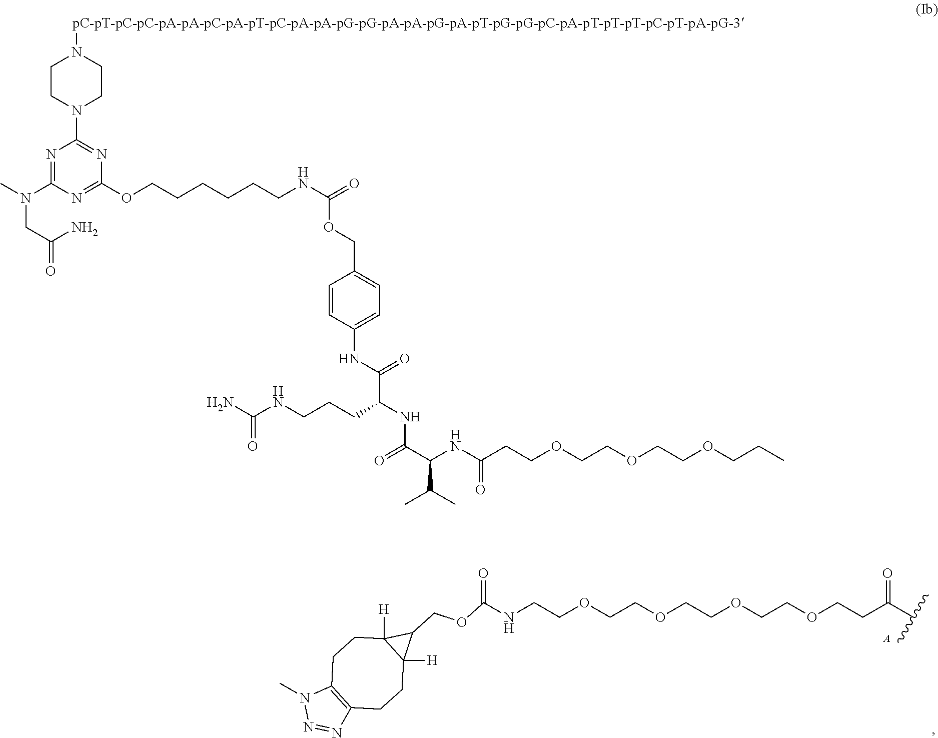

in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises an anti-TfR1 Fab comprising a heavy chain comprising the amino acid sequence of SEQ ID NO: 19 and a light chain comprising the amino acid sequence of SEQ ID NO: 20; and
in each complex n1 is independently an integer of one or greater representing the number of instances of R¹, wherein each instance of R¹ is covalently linked via attachment point A to a different lysine of the anti-TfR1 Fab, optionally wherein the average value of n1 of the complexes of the composition is in the range of 1 to 5;
wherein the effective amount provides to the subject 5 mg to 120 mg of the anti-TfR1 Fab of the complexes per kg of the subject.

53. A method of promoting expression or activity of a dystrophin protein in a subject, comprising administering via infusion an effective amount of a composition comprising complexes, wherein each complex comprises a structure of formula (I): [R¹]ₙ₁—R², wherein:
each R¹ comprises a group of the formula (Ic):

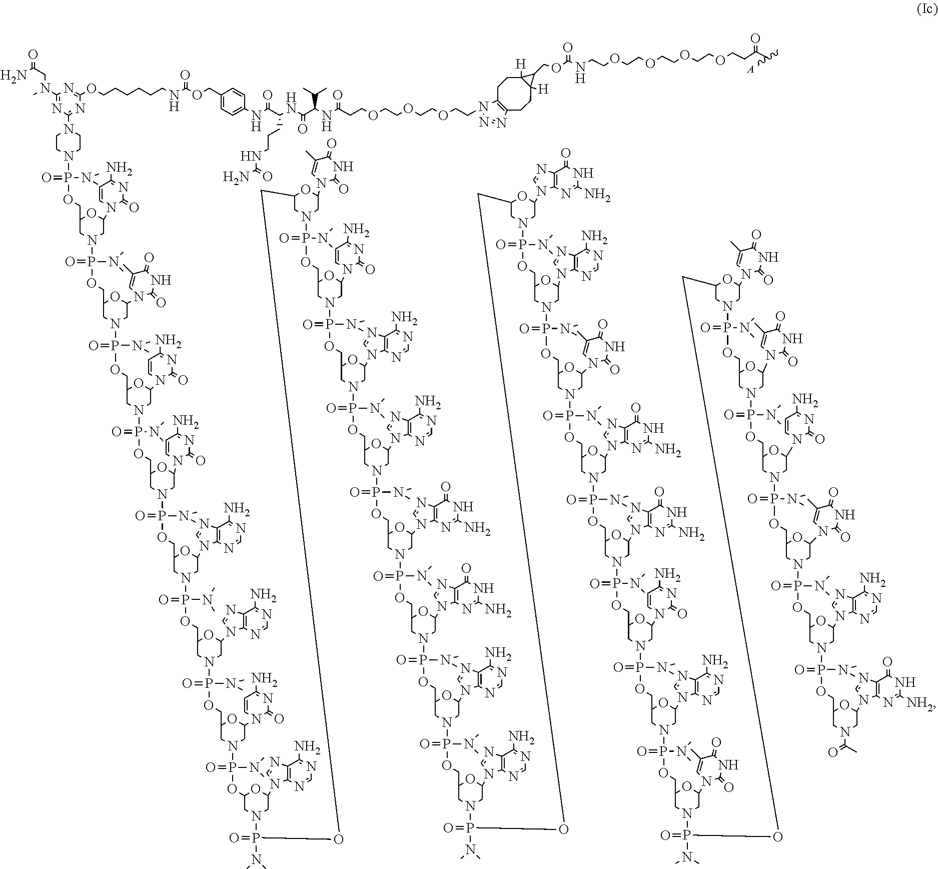

wherein R² comprises an anti-TfR1 Fab comprising a heavy chain comprising the amino acid sequence of SEQ ID NO: 19 and a light chain comprising the amino acid sequence of SEQ ID NO: 20; and
in each complex n1 is independently an integer of one or greater representing the number of instances of R¹, wherein each instance of R¹ is covalently linked via attachment point A to a different lysine of the anti-TfR1 Fab, optionally wherein the average value of n1 of the complexes of the composition is in the range of 1 to 5;
wherein the effective amount provides to the subject 5 mg to 120 mg of the anti-TfR1 Fab of the complexes per kg of the subject.

54. A method of promoting expression or activity of a dystrophin protein in a subject, comprising administering via infusion an effective amount of a composition comprising complexes, wherein the complex comprises a group of formula (Id):

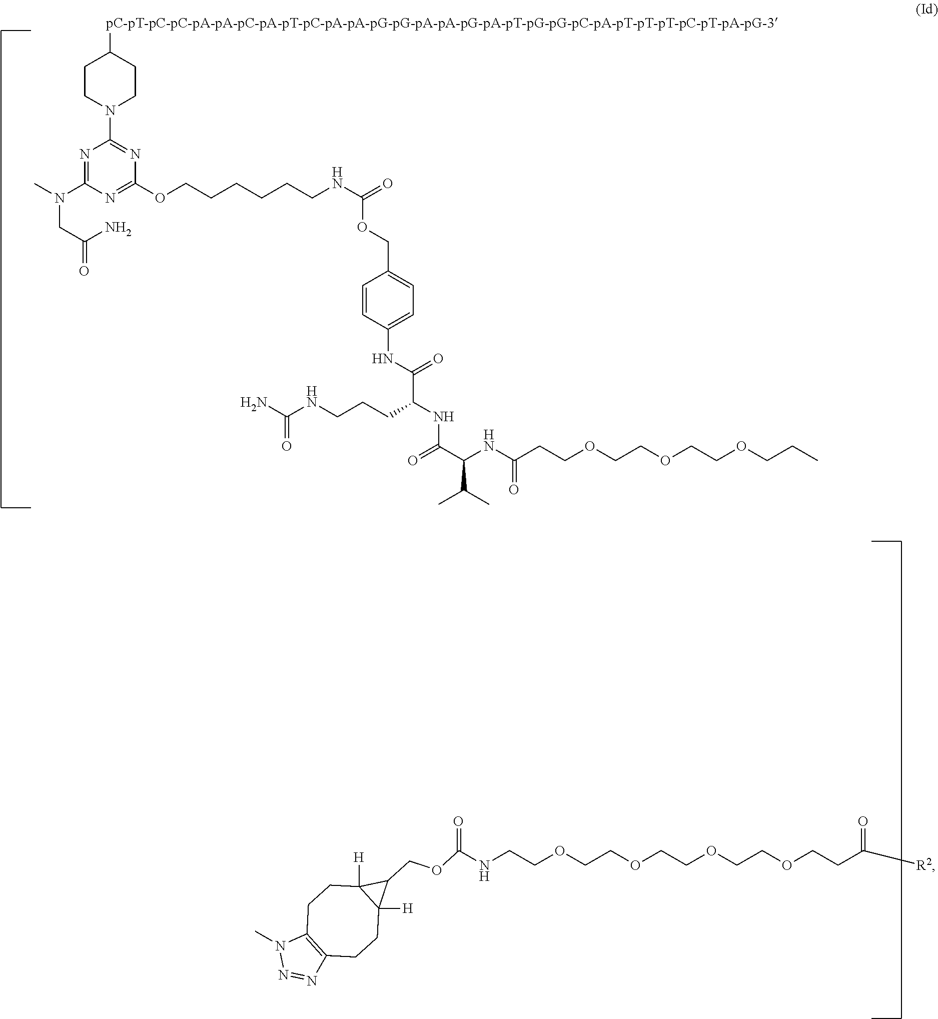

wherein R² comprises an anti-TfR1 Fab comprising a heavy chain comprising the amino acid sequence of SEQ ID NO: 19 and a light chain comprising the amino acid sequence of SEQ ID NO: 20; and
wherein the effective amount provides to the subject 5 mg to 120 mg of the anti-TfR1 Fab of the complexes per kg of the subject.

55. The method of any one of embodiments 51-54, wherein the effective amount provides to the subject 10 mg to 90 mg of the anti-TfR1 Fab of the complexes per kg of the subject, optionally wherein the effective amount provides to the subject 11 mg, 22 mg, 44 mg, or 88 mg of the anti-TfR1 Fab of the complexes per kg of the subject.

56. The method of any one of embodiments 51-54, wherein the effective amount provides to the subject 10.93 mg, 21.85 mg, 43.71 mg, 65.56 mg, or 87.42 mg of the anti-TfR1 Fab of the complexes per kg of the subject.

57. The method of any one of embodiments 51-54, wherein the effective amount provides to the subject 10.93 mg, 21.86 mg, 43.72 mg, 65.56 mg, or 87.43 mg of the anti-TfR1 Fab of the complexes per kg of the subject.

58. The method of any one of embodiments 51-57, wherein the composition is a formulation of an aqueous solution that is at a pH of 6.0 and comprises histidine at a concentration of 25 mM, sucrose at a concentration of 10 w/v %, and the complexes at a concentration in the range of 10 mg/mL to 50 mg/mL.

59. The method of any one of embodiments 51-58, wherein the subject is administered the formulation once every 4 weeks, once every 8 weeks, or once every 12 weeks, optionally wherein the subject is administered the formulation once every 4 weeks.

60. The method of any one of embodiments 51-59, wherein the subject has Duchenne Muscular Dystrophy (DMD).

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleotide linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising.” “having.” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

What is claimed is:

1. A method of promoting expression or activity of a dystrophin protein or treating Duchenne Muscular Dystrophy (DMD) in a subject, comprising administering to the subject a composition comprising an effective amount of complexes, each complex comprising an anti-transferrin receptor 1 (TfR1) antibody covalently linked to one or more oligonucleotides, wherein the effective amount provides to the subject 1 mg to 90 mg of the anti-TfR1 antibody of the complexes per kg of the subject, wherein the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide comprises the nucleotide sequence of CTCCAACATCAAGGAAGATGGCATTTCTAG (SEQ ID NO: 21) and is a phosphorodiamidate morpholino oligomer (PMO).

2. The method of claim 1, wherein each complex comprises a structure of formula (I): [R¹] n1-R², wherein:
each R¹ comprises a group of the formula (Ib): 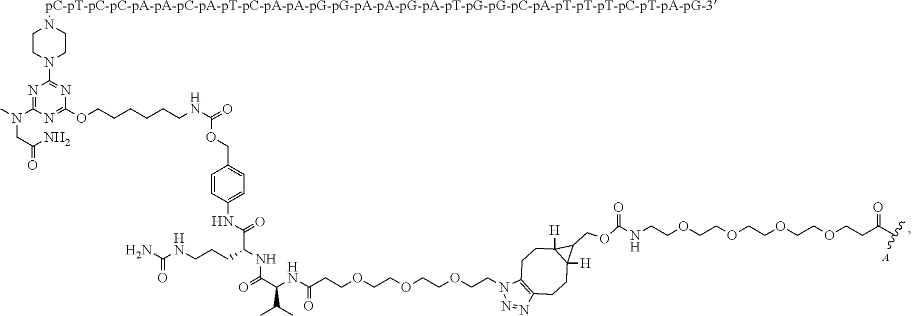
in which -pN indicates a base position of a phosphorodiamidate morpholino oligomer (PMO), wherein -p reflects a phosphorodiamidate linkage, and wherein N corresponds to a nucleobase of adenine (A), cytosine (C), guanine (G), or thymine (T), such that the PMO has a base sequence of CTCCAACATCAAGGAAGATGGCATTTCTAG (SEQ ID NO: 21);
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 1 to 5.

3. The method of claim 1 or claim 2, wherein each complex comprises a structure of formula (I): [R¹]ₙ₁—R², wherein:
each R¹ comprises a group of the formula (Ic): 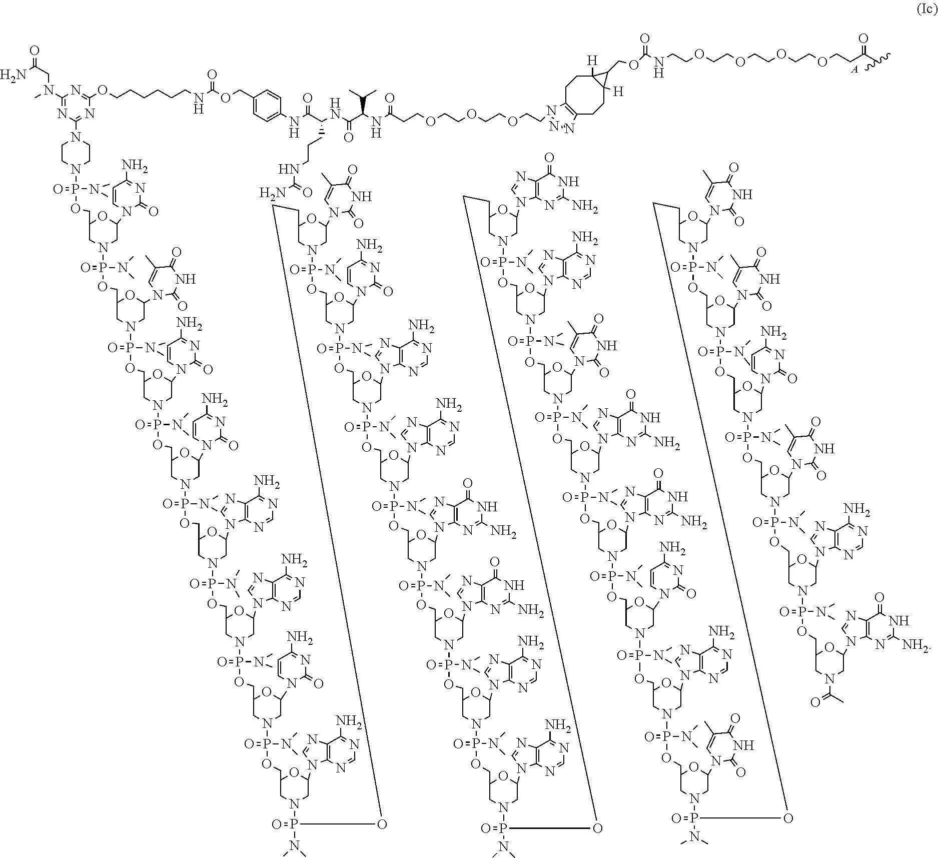
R² comprises the anti-TfR1 antibody; and
in each complex, n1 is independently an integer of one or greater representing the number of instances of R¹, wherein each instance of R¹ is covalently linked via attachment point A to a different lysine of the anti-TfR1 antibody, optionally wherein the average value of n1 of the complexes of the composition is in the range of 1 to 5.

4. The method of any one of claims 1-3, wherein the anti-TfR1 antibody is a Fab fragment.

5. The method of any one of claims 1-4, wherein the anti-TfR1 antibody comprises a heavy chain variable region (VH) comprising the amino acid sequence of SEQ ID NO: 17 and a light chain variable region (VL) comprising the amino acid sequence of SEQ ID NO: 18.

6. The method of any one of claims 1-5, wherein the anti-TfR1 antibody comprises a heavy chain comprising the amino acid sequence of SEQ ID NO: 19 and a light chain comprising the amino acid sequence of SEQ ID NO: 20.

7. The method of any one of claims 1-6, wherein the effective amount of each administration provides to the subject 1-8 mg of the anti-TfR1 antibody of the complexes per kg of the subject.

8. The method of any one of claims 1-7, wherein the effective amount of each administration provides to the subject 1-2 mg of the anti-TfR1 antibody of the complexes per kg of the subject, optionally wherein the effective amount of each administration provides to the subject 1.5 mg of the anti-TfR1 antibody of the complexes per kg of the subject.

9. The method of any one of claims 1-7, wherein the effective amount of each administration provides to the subject 2-4 mg of the anti-TfR1 antibody of the complexes per kg of the subject, optionally wherein the effective amount of each administration provides to the subject 3 mg of the anti-TfR1 antibody of the complexes per kg of the subject.

10. The method of any one of claims 1-7, wherein the effective amount of each administration provides to the subject 4-8 mg of the anti-TfR1 antibody of the complexes per kg of the subject, optionally wherein the effective amount of each administration provides to the subject 6 mg of the anti-TfR1 antibody of the complexes per kg of the subject.

11. The method of any one of claims 1-6, wherein the effective amount of each administration provides to the subject 3-52 mg of the anti-TfR1 antibody of the complexes per kg of the subject, optionally wherein the effective amount of each administration provides to the subject 5-40 mg of the anti-TfR1 antibody of the complexes per kg of the subject.

12. The method of any one of claims 1-6, wherein the effective amount of each administration provides to the subject 7-15 mg of the anti-TfR1 antibody of the complexes per kg of the subject, optionally wherein the effective amount of each administration provides to the subject 11 mg of the anti-TfR1 antibody of the complexes per kg of the subject.

13. The method of any one of claims 1-6, wherein the effective amount of each administration provides to the subject 15-30 mg of the anti-TfR1 antibody of the complexes per kg of the subject, optionally wherein the effective amount of each administration provides to the subject 22 mg of the anti-TfR1 antibody of the complexes per kg of the subject.

14. The method of any one of claims 1-6, wherein the effective amount of each administration provides to the subject 30-59 mg of the anti-TfR1 antibody of the complexes per kg of the subject, optionally wherein the effective amount of each administration provides to the subject 44 mg of the anti-TfR1 antibody of the complexes per kg of the subject.

15. The method of any one of claims 1-6, wherein the effective amount of each administration provides to the subject 61-117 mg of the anti-TfR1 antibody of the complexes per kg of the subject, optionally wherein the effective amount of each administration provides to the subject 88 mg of the anti-TfR1 antibody of the complexes per kg of the subject

16. The method of any one of claims 1-15, wherein the composition is administered once every 2 weeks, once every 4 weeks, once every 8 weeks or once every 12 weeks.

17. The method of any one of claims 1-16, wherein the composition is administered once every 4 weeks.

18. The method of any one of claims 1-16, wherein the composition is administered once every 8 weeks.

19. The method of any one of claims 1-18, wherein the composition is in an aqueous solution and further comprises histidine and sucrose, optionally wherein the histidine is present in the aqueous solution at a concentration of 25 mM, optionally wherein the sucrose is present in the aqueous solution at a concentration of 10 w/v %, and optionally wherein the aqueous solution is at a pH of 6.0.

20. The method of any one of claims 1-19, wherein the subject has a mutated dystrophin allele comprising a mutation amenable to exon 51 skipping or the mutated dystrophin allele comprises a frameshift mutation in exon 51.

21. The method of any one of claims 1-20, wherein the complex promotes expression or activity of a truncated dystrophin protein in the subject.

22. The method of any one of claims 1-21, wherein the subject is a human subject, optionally wherein the human subject is between 2-60 years of age.
